(19) United States
(12) Patent Application Publication
(10) Pub. No.: US 2022/0040207 A1
(43) Pub. Date: Feb. 10, 2022

(54) REMODILINS TO PREVENT OR TREAT CANCER METASTASIS, GLAUCOMA, AND HYPOXIA

(71) Applicants: THE UNIVERSITY OF CHICAGO, Chicago, IL (US); THE UNITED STATES OF AMERICA as represented by THE SECRETARY DEPARTMENT OF HEALTH AND HUMAN SERVICES, Bethesda, MD (US); PRESIDENT AND FELLOWS OF HARVARD COLLEGE, Cambridge, MA (US); IIT RESEARCH INSTITUTE, Chicago, IL (US); BETH ISRAEL DEACONESS MEDICAL CENTER, INC., Boston, MA (US); REGENTS OF THE UNIVESITY OF MINNESOTA, Minneapolis, MA (US); THE TRUSTEES OF PURDUE UNIVERSITY, West Lafayette,, IN (US)

(72) Inventors: Julian SOLWAY, (US); Nickolai DULIN, (US); Marsha ROSNER, (US); Gokhan MUTLU, (US); Diane LUCI, (US); David MALONEY, (US); Chan Young PARK, (US); Jeffrey FREDBERG, (US); David MCCORMICK, (US); Ramaswamy KRISHNAN, (US)

(73) Assignees: The University of Chicago, Chicago, IL (US); The United States of America as Represented by the Secretary Department of Health and Human Services, Bethesda, MD (US); President and Fellows of Harvard College, Cambridge, MA (US); IIT Research Institute, Chicago, IL (US); Beth Israel Deaconess Medical Center, Inc., Boston, MA (US)

(21) Appl. No.: 17/594,090

(22) PCT Filed: Apr. 2, 2020
(86) PCT No.: PCT/US2020/026383

§ 371 Date: Oct. 1, 2021

Related U.S. Application Data
(60) Provisional application No. 62/828,122, filed on Apr. 2, 2019.

(51) Int. Cl. A61K 31/635; A61K 31/63; C07C 311/16; C07C 311/21; A61K 31/18; C07D 277/52; C07D 211/34; C07D 211/96; C07D 295/26; C07D 279/12; C07D 309/14; C07D 487/08; C07D 491/113; C07D 295/13; C07D 239/42; C07D 471/04; A61P 35/04; C07D 401/12; C07D 207/48; C07D 241/04; C07D 265/30

(52) U.S. Cl. CPC A61K 31/635; C07C 2601/02; C07C 311/16; C07C 311/21; A61K 31/18; C07D 277/52; C07D 211/34; C07D 211/96; C07D 295/26; C07D 279/12; C07D 309/14; C07D 487/08; C07D 491/113; C07D 295/13; C07D 239/42; C07D 471/04; A61P 35/04; C07D 401/12; C07D 207/48; C07D 241/04; C07D 265/30; C07C 2601/16; C07C 2601/14; C07C 2601/04; C07C 2601/08; A61K 31/63

(57) ABSTRACT

Disclosed herein is a class of molecules termed remodilins that inhibit serum response factor (SRF). By inhibiting SRF, a number of downstream pathways can be targeted. The remodilins can be used to treat glaucoma, inhibit tumor cell growth, inhibit tumor metastasis, inhibit hypoxia-induced response, and/or reduce cellular metabolism.

### CROSS-REFERENCE TO RELATED APPLICATIONS

This application claims the benefit of priority of U.S. Provisional Patent Application No. 62/828,122 filed Apr. 2, 2019, which is hereby incorporated by reference in its entirety.

This application is related by subject matter to U.S. Provisional Patent Application No. 62/872,980 filed Apr. 2, 2019, entitled Remodilinsfor Airway Remodeling and Organ Fibrosis by Julian Solway et al., which is incorporated by reference.

### STATEMENT OF GOVERNMENT SUPPORT

This invention was made with government support under grant numbers P50 HL107171, HL123816, P01 HL120839, and R01 EY019696 awarded by the National Institutes of Health. The government has certain rights in the invention.

### FIELD OF THE INVENTION

The present invention relates generally to the fields of medicine, medicinal chemistry, organic chemistry, and pharmacology.

### BACKGROUND

Human serum response factor (SRF) is a transcription factor which binds to a serum response element (SRE) associated with a variety of genes including proto-oncogenes such as c-fos, fosB, and, junB, and muscle genes such as smooth muscle α- and γ-actins, myosin light chain, and α- and β-myosin heavy chains.

SRF-binding sites were initially identified in growth-related genes. Gene inactivation or knockdown studies in species ranging from unicellular eukaryotes to mice have consistently shown that SRF plays a crucial role in cellular migration and normal actin cytoskeleton and contractile biology.

Cell migration is central to myriad developmental (e.g., gastrulation) and pathological (e.g., metastasis) processes. The actin cytoskeleton, long thought to be a static scaffold for the maintenance of cell shape, polarity, and mechanical support, undergoes dynamic remodeling involving scores of proteins that regulate the cytoskeleton. The driving force for membrane protrusion, one of the first steps in metastasis-associated cellular migration, is localized polymerization of submembrane actin filaments.

Glaucoma is the second leading cause of irreversible blindness and it affects over 70 million people worldwide. In glaucoma, progressive fibrosis and malfunctioning of the trabecular meshwork, in particular the aberrant production of extracellular matrix, leads to increased resistance to aqueous outflow and glaucomatous damage. SRF is involved in the regulation of genes that are involved in a variety of fibrosis phenotypes, including vascular, lung, and ocular fibrosis.

It has been estimated that the transcription of as many as 300 genes is under the control of SRF signaling, and of these, more than 200 are directly targeted by the protein. SRF is therefore integral to a vast number of cellular processes, from metastasis-associated cellular migration, to maintenance of ocular cell matrix dynamics. Given the role of SRF in cell migration and ocular matrix maintenance, inhibition of SRF is a new therapeutic approach for treating aberrant cell growth, metastasis, and glaucoma.

### SUMMARY OF THE INVENTION

The present disclosure provides compositions and methods for addressing the SRF-mediated disorders discussed above. The inventors have identified a series of novel small organic compounds, referred to herein as remodilins, that are useful for inhibiting SRF activity and affecting the downstream pathways discussed above. The inventors have discovered that these novel remodilins inhibit activation of SRF. By inhibiting a transcription factor that is upstream of these pathologies, the remodilins provide a novel route for inhibiting tumor cell growth, inhibiting migration of cancer cells (metastasis), and treating glaucoma by softening eye cells and inhibiting smooth muscle alpha actin and fibronectin expression.

Certain aspects of the disclosure are directed to a method for inhibiting serum response factor activity in a cell, a method for inhibiting smooth muscle contractile protein accumulation in a cell, and/or a method for inhibiting smooth muscle contractile protein expression in a cell comprising administering to the cell a composition comprising an effective amount of a compound of Formula I as described herein. Some aspects of the disclosure are directed to a method for reducing cellular contractile force, a method for inhibiting tumor cell growth, a method for inhibiting spreading, migration, and metastasis in a subject having a tumor, a method for reducing cellular metabolism, a method for attenuating hypoxia-induced response, a method for inhibiting HIF1α accumulation, a method for treating sleep apnea, and/or a method for treating glaucoma comprising administering to a subject a composition comprising an effective amount of a compound of Formula I as described herein. In aspects, a compound of Formula I inhibits expression of smooth muscle myosin heavy chains. In some embodiments, a compound of Formula I inhibits expression of smooth muscle alpha actin. In some embodiments, a compound of Formula I inhibits localized accumulation of smooth muscle myosin heavy chains. In some aspects, a compound of Formula I inhibits localized accumulation of smooth muscle alpha actin. In some embodiments, a compound of Formula I stimulates generation of trabecular meshwork cells. In some aspects, a compound of Formula I stimulates generation of Schlemm's Canal cells. In some aspects, a compound of Formula I inhibits contractile force generation of trabecular meshwork cells. In some embodiments, a compound of Formula I inhibits contractile force generation of Schlemm's canal endothelial cells. In some aspects, a compound of Formula I inhibits fibronectin expression.

In embodiments where a compound of Formula I is used for inhibiting tumor cell growth, administration of the compound may be done prior to, concurrently with, or subsequent to chemotherapy, surgical treatment, immunotherapy, or radiation treatment. In some aspects, a compound of Formula I inhibits human serum response factor activity. In some embodiments, inhibition of serum response factor activity affects at least one of cell cycle regulation, apoptosis, cell growth, and differentiation.

Certain aspects of the disclosure are directed towards compositions comprising a compound of Formula I:

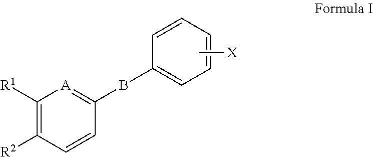

where A is —CH— or —N—, B is —C(O)—NH—, —NH—C(O)—, —CH₂—NH—, or —C(NH)—NH—, X is —(Y)—NR³R⁴ or NHSO₂Me, Y is —SO₂—, —C(O)—, or —(CH₂)—, R1 and R2 are each independently hydrogen, hydroxyl, substituted or unsubstituted alkyl, substituted or unsubstituted alkynyl, alkoxy, halide, nitrile, amine, acylamine, substituted or unsubstituted aryl, 4-6 member carbocycle, substituted or unsubstituted heterocycle, and R3 and R4 are each independently hydrogen, substituted or unsubstituted alkyl, substituted or unsubstituted aromatic, substituted or unsubstituted carbocycle, substituted or unsubstituted heterocycle, substituted or unsubstituted bicyclic, or may join to form a carbocycle or heterocycle. In some embodiments, the compound is further defined as:

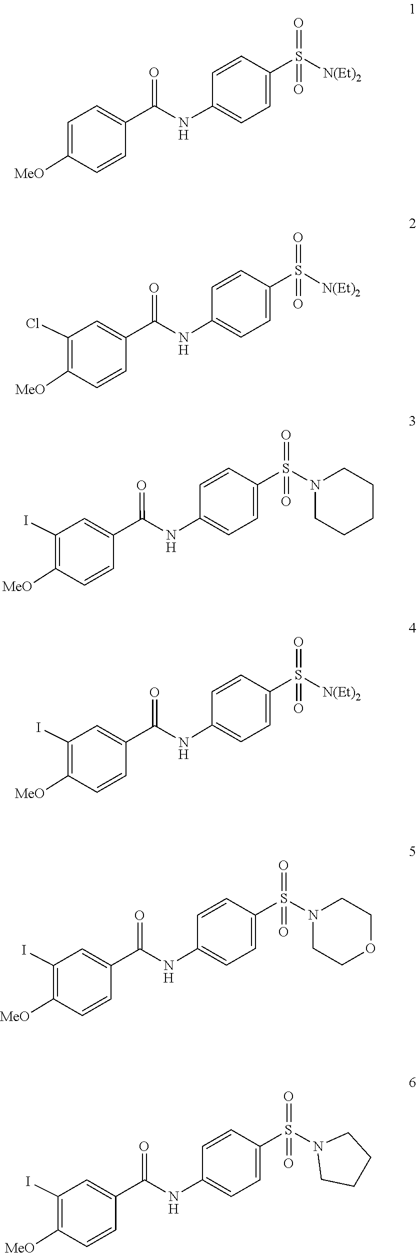

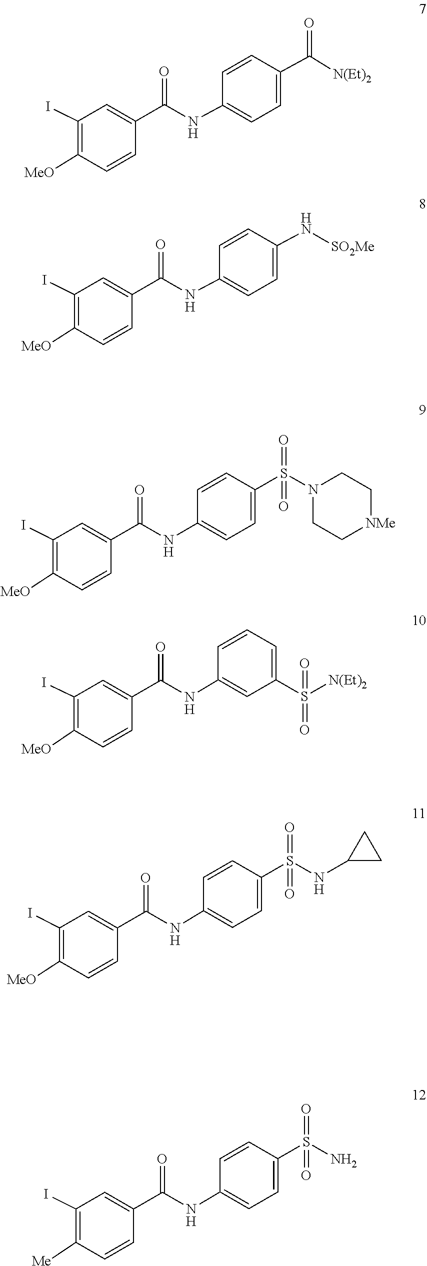

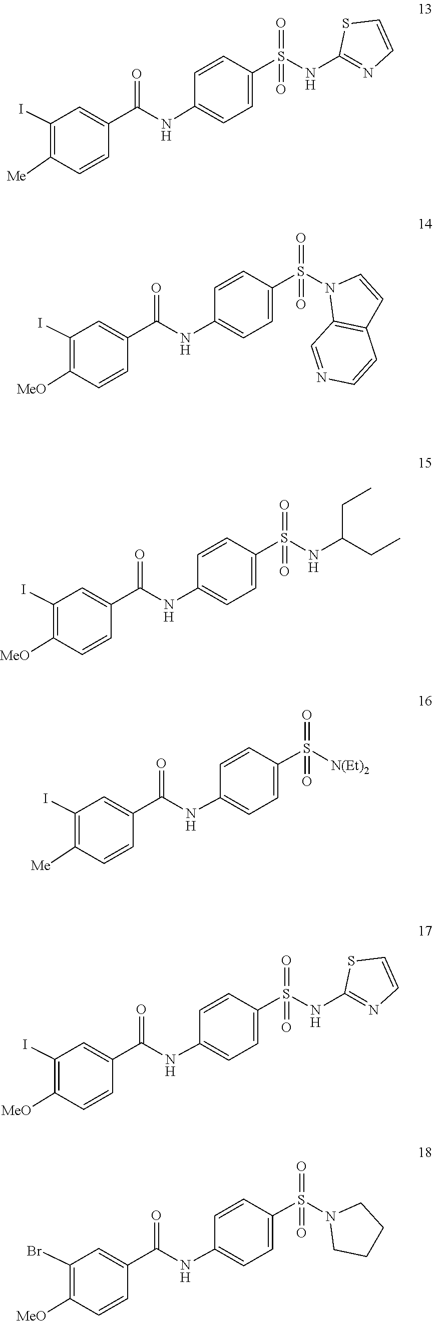

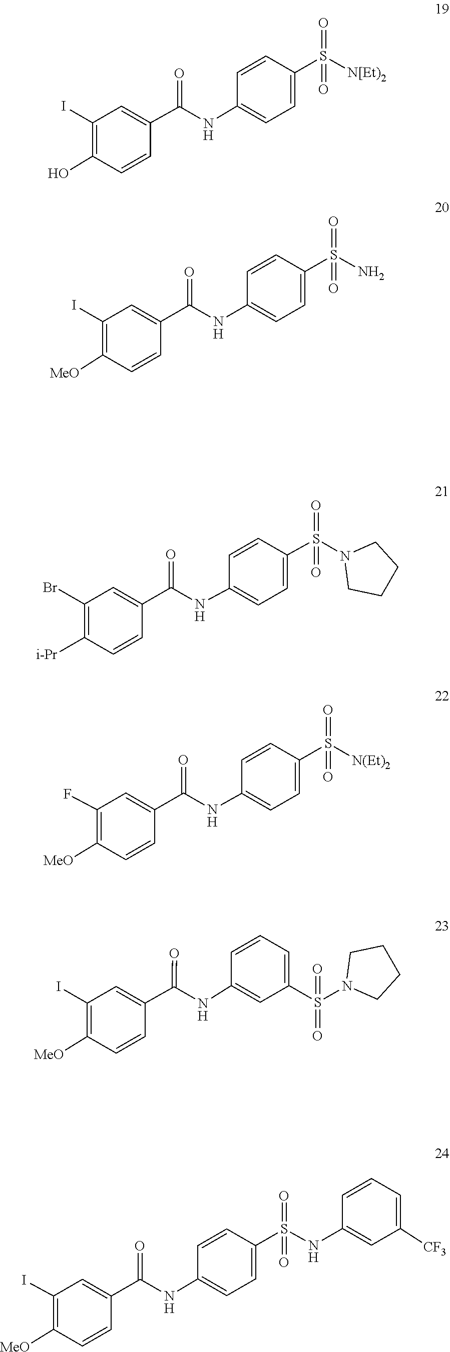

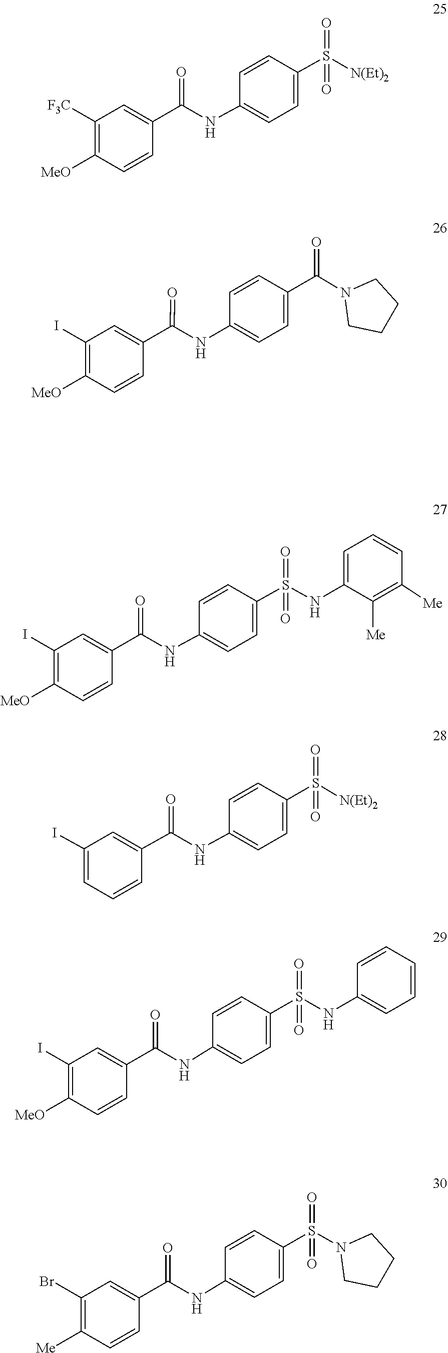

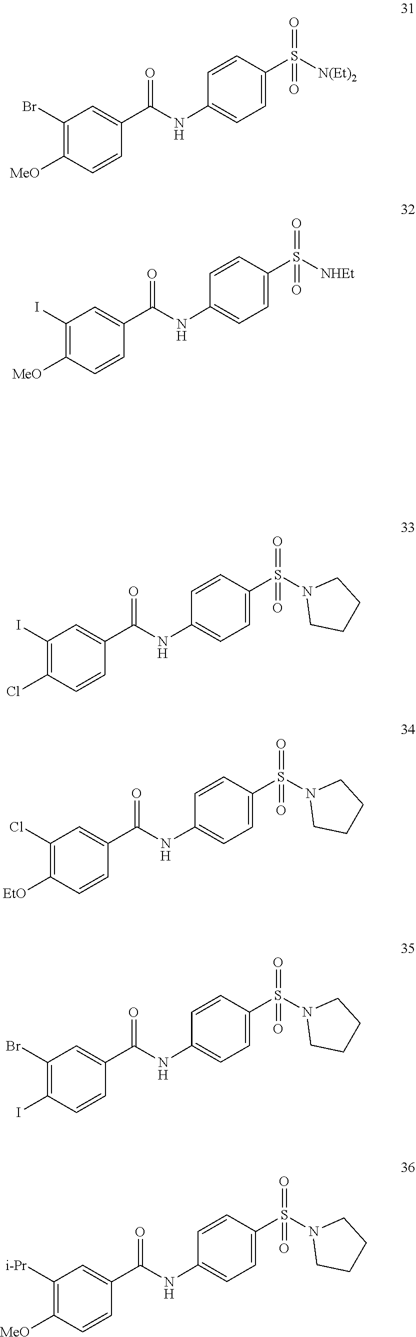

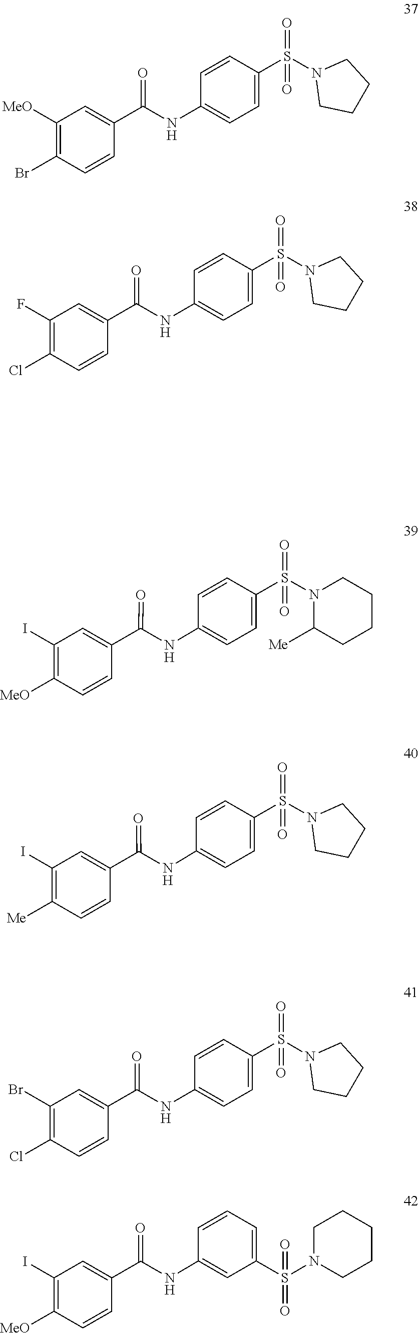 [SMILES: COc1cc(C(=O)Nc2ccc(S(=O)(=O)N3CCCC3)cc2)ccc1Br ; O=C(Nc1ccc(S(=O)(=O)N2CCCC2)cc1)c1ccc(Cl)c(F)c1 ; COc1ccc(C(=O)Nc2ccc(S(=O)(=O)N3CCCCC3C)cc2)cc1I ; Cc1ccc(C(=O)Nc2ccc(S(=O)(=O)N3CCCC3)cc2)cc1I ; O=C(Nc1ccc(S(=O)(=O)N2CCCC2)cc1)c1ccc(Cl)c(Br)c1 ; COc1ccc(C(=O)Nc2cccc(S(=O)(=O)N3CCCCC3)c2)cc1I]

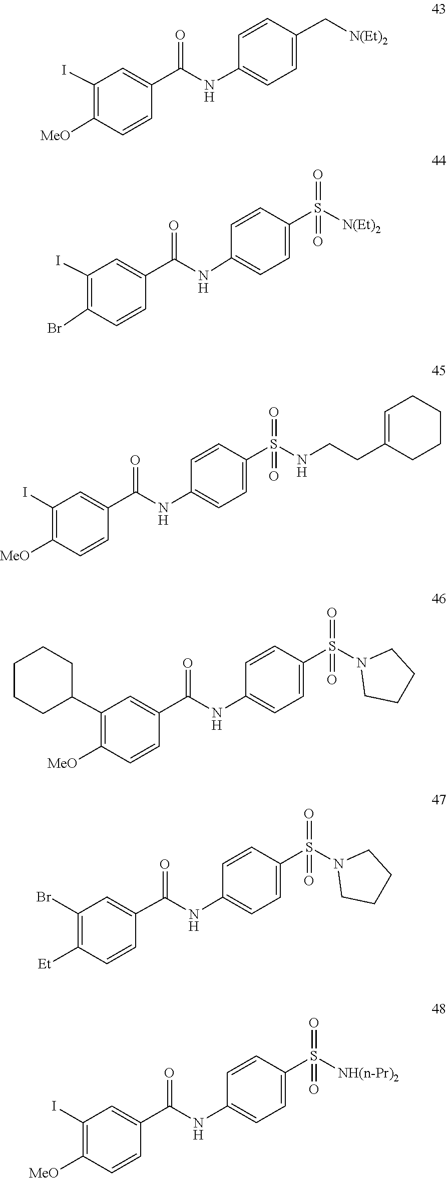

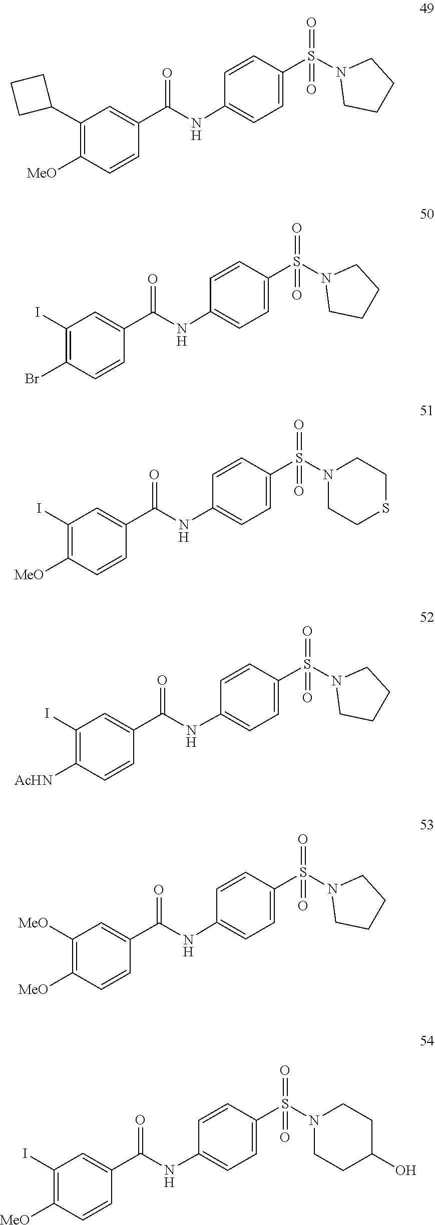 [SMILES: COc1ccc(C(=O)Nc2ccc(S(=O)(=O)N3CCCC3)cc2)cc1C1CCC1 ; O=C(Nc1ccc(S(=O)(=O)N2CCCC2)cc1)c1ccc(Br)c(I)c1 ; COc1ccc(C(=O)Nc2ccc(S(=O)(=O)N3CCSCC3)cc2)cc1I ; CC(=O)Nc1ccc(C(=O)Nc2ccc(S(=O)(=O)N3CCCC3)cc2)cc1I ; COc1ccc(C(=O)Nc2ccc(S(=O)(=O)N3CCCC3)cc2)cc1OC ; COc1ccc(C(=O)Nc2ccc(S(=O)(=O)N3CCC(O)CC3)cc2)cc1I]

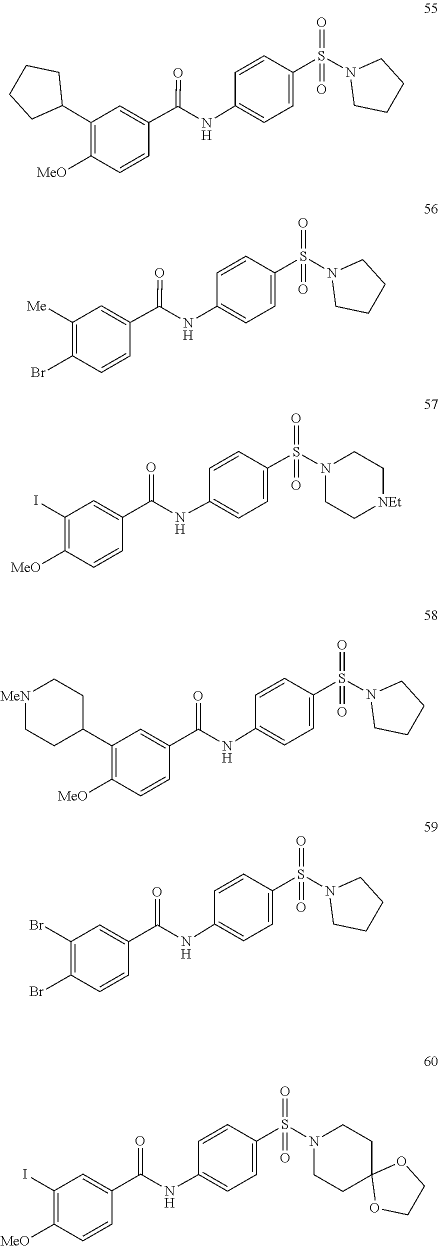

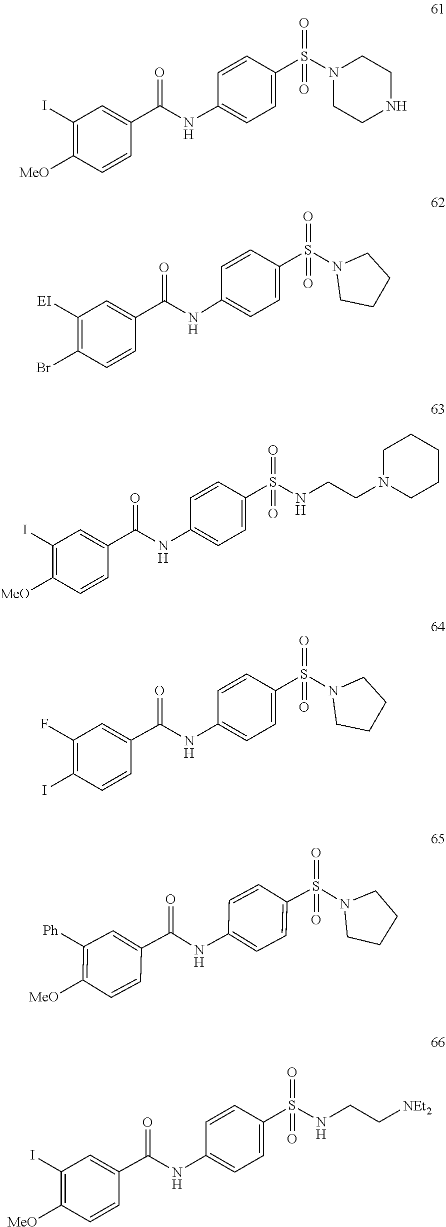

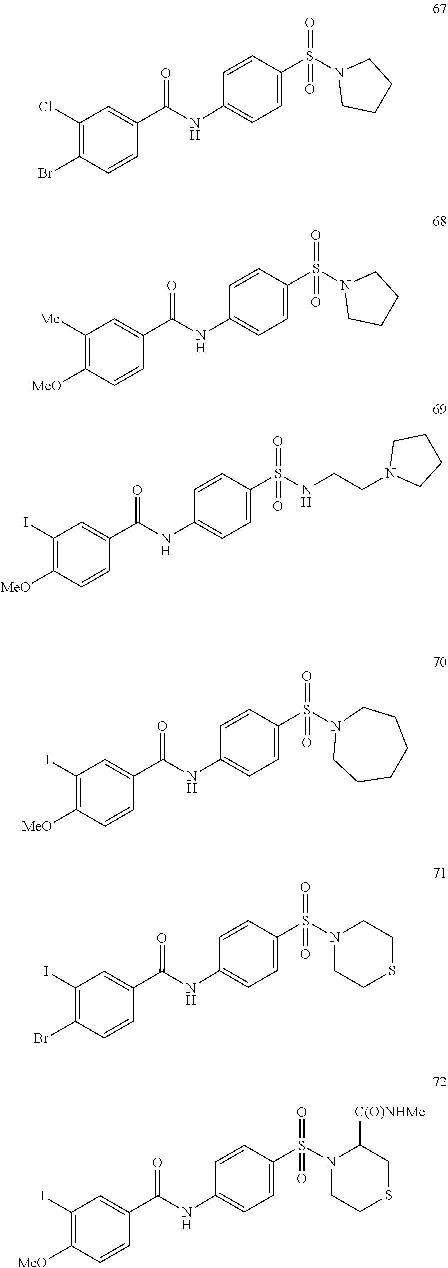

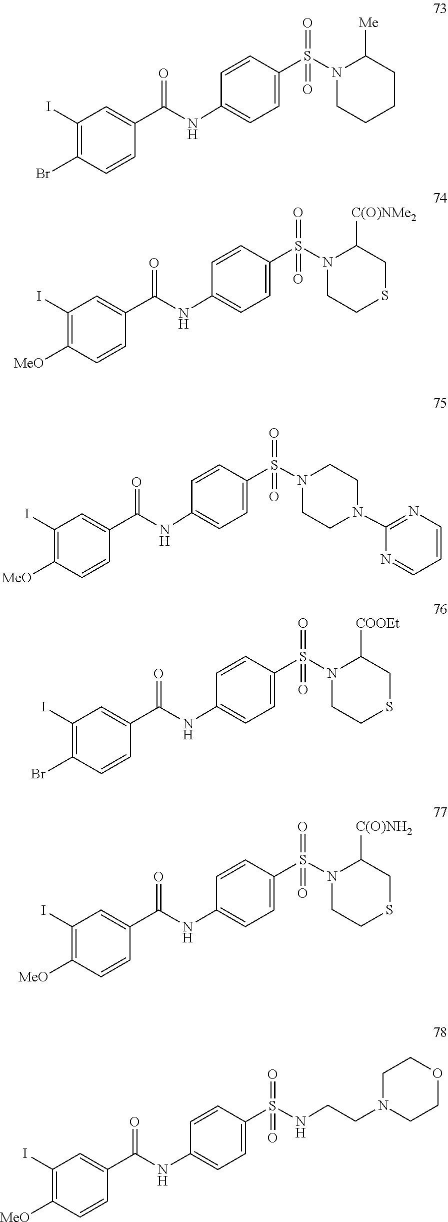

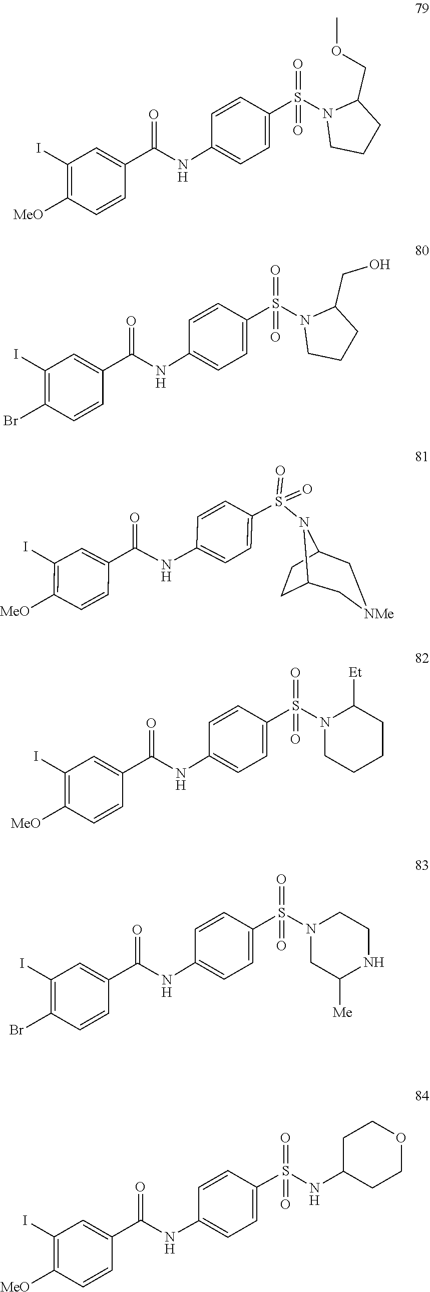 [SMILES: COCC1CCCN1S(=O)(=O)c1ccc(NC(=O)c2ccc(OC)c(I)c2)cc1 ; O=C(Nc1ccc(S(=O)(=O)N2CCCC2CO)cc1)c1ccc(Br)c(I)c1 ; COc1ccc(C(=O)Nc2ccc(S(=O)(=O)N3C4CCC3CN(C)C4)cc2)cc1I ; CCC1CCCCN1S(=O)(=O)c1ccc(NC(=O)c2ccc(OC)c(I)c2)cc1 ; CC1CN(S(=O)(=O)c2ccc(NC(=O)c3ccc(Br)c(I)c3)cc2)CCN1 ; COc1ccc(C(=O)Nc2ccc(S(=O)(=O)NC3CCOCC3)cc2)cc1I]

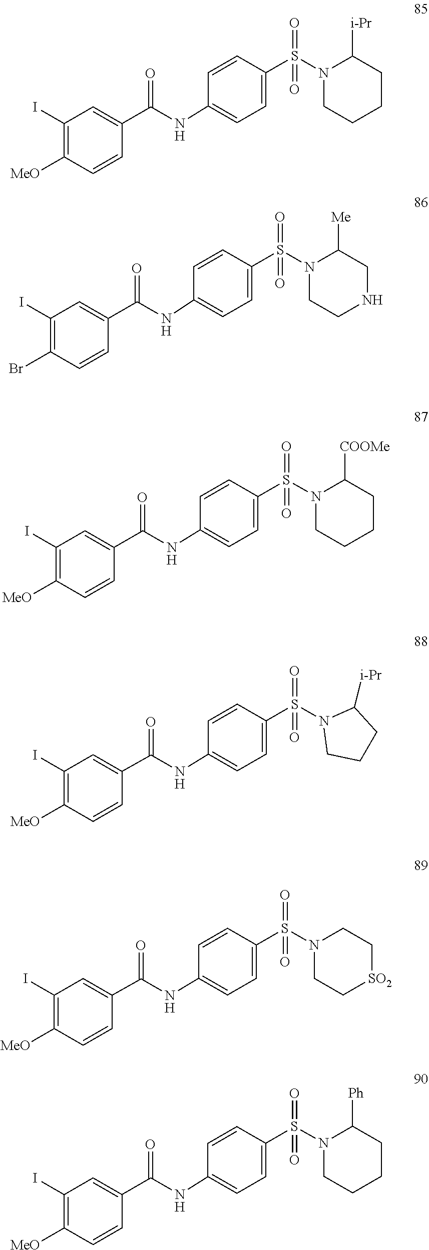 [SMILES: COc1ccc(C(=O)Nc2ccc(S(=O)(=O)N3CCCCC3C(C)C)cc2)cc1I ; CC1CNCCN1S(=O)(=O)c1ccc(NC(=O)c2ccc(Br)c(I)c2)cc1 ; COC(=O)C1CCCCN1S(=O)(=O)c1ccc(NC(=O)c2ccc(OC)c(I)c2)cc1 ; COc1ccc(C(=O)Nc2ccc(S(=O)(=O)N3CCCC3C(C)C)cc2)cc1I ; COc1ccc(C(=O)Nc2ccc(S(=O)(=O)N3CCS(=O)(=O)CC3)cc2)cc1I ; COc1ccc(C(=O)Nc2ccc(S(=O)(=O)N3CCCCC3c3ccccc3)cc2)cc1I]

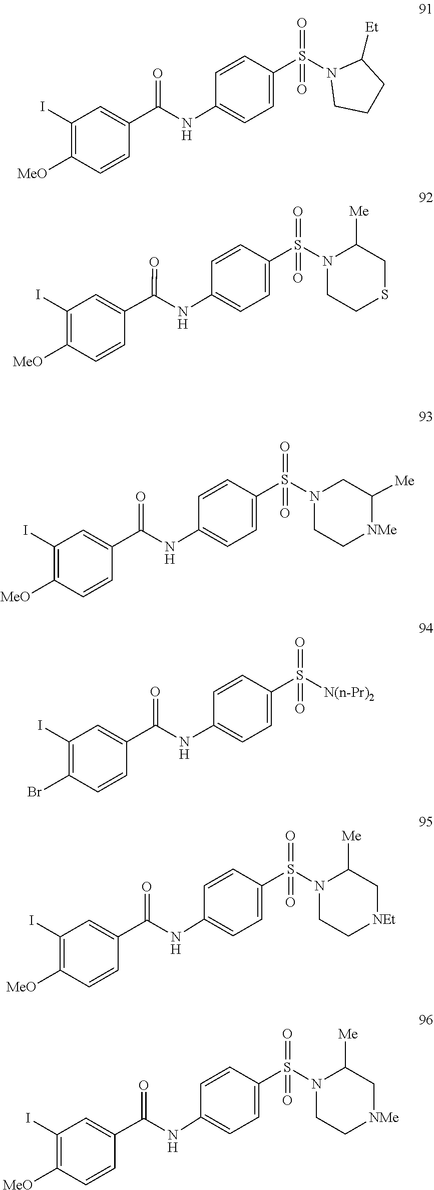

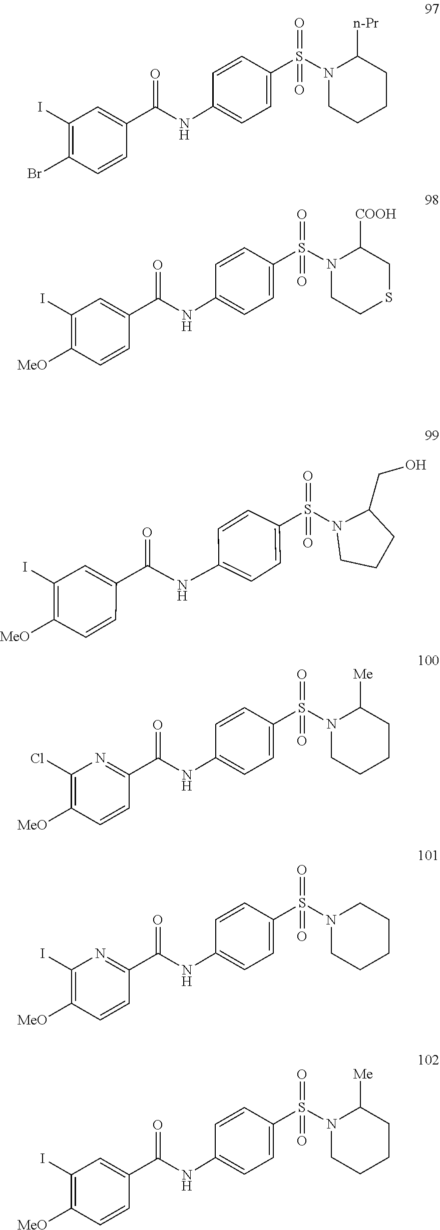

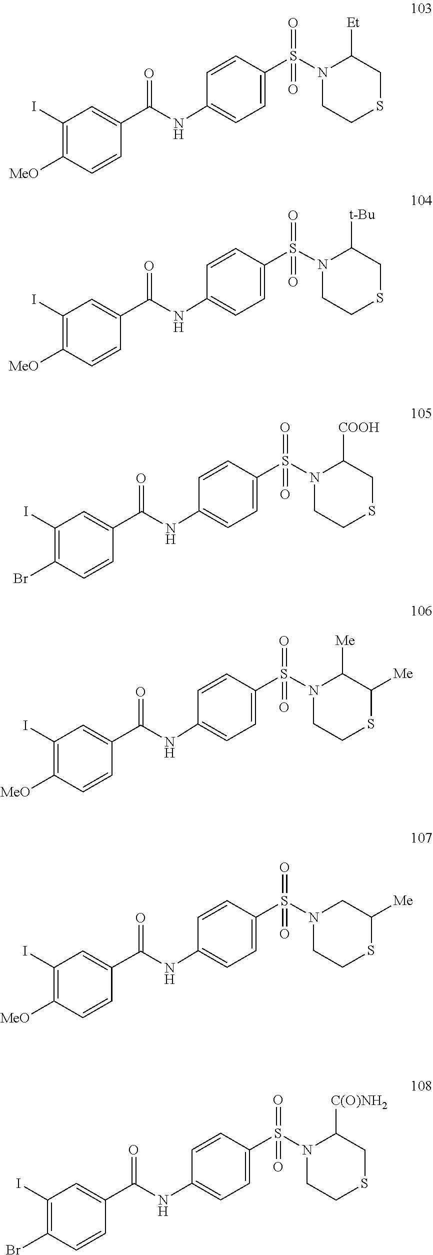

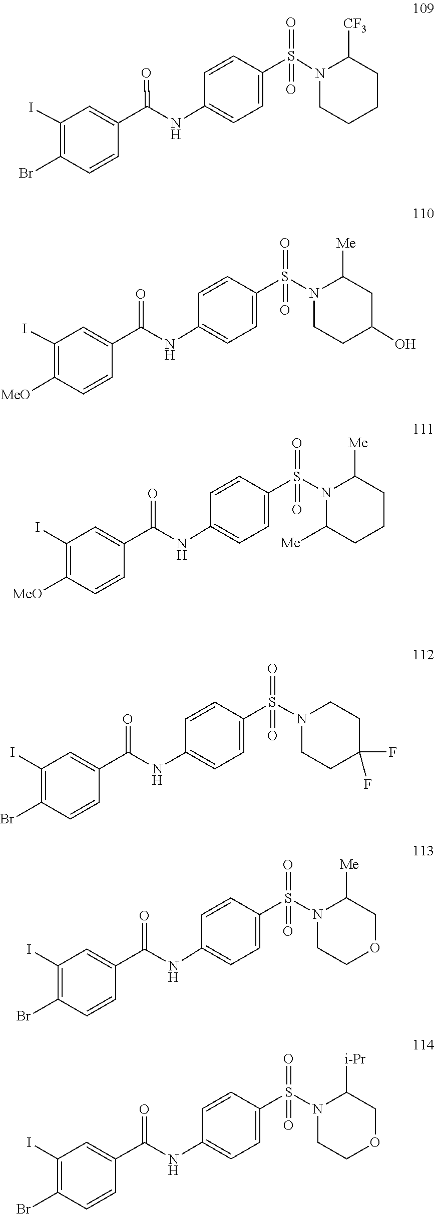 [SMILES: O=C(Nc1ccc(S(=O)(=O)N2CCCCC2C(F)(F)F)cc1)c1ccc(Br)c(I)c1 ; COc1ccc(C(=O)Nc2ccc(S(=O)(=O)N3CCC(O)CC3C)cc2)cc1I ; COc1ccc(C(=O)Nc2ccc(S(=O)(=O)N3C(C)CCCC3C)cc2)cc1I ; O=C(Nc1ccc(S(=O)(=O)N2CCC(F)(F)CC2)cc1)c1ccc(Br)c(I)c1 ; CC1COCCN1S(=O)(=O)c1ccc(NC(=O)c2ccc(Br)c(I)c2)cc1 ; CC(C)C1COCCN1S(=O)(=O)c1ccc(NC(=O)c2ccc(Br)c(I)c2)cc1]

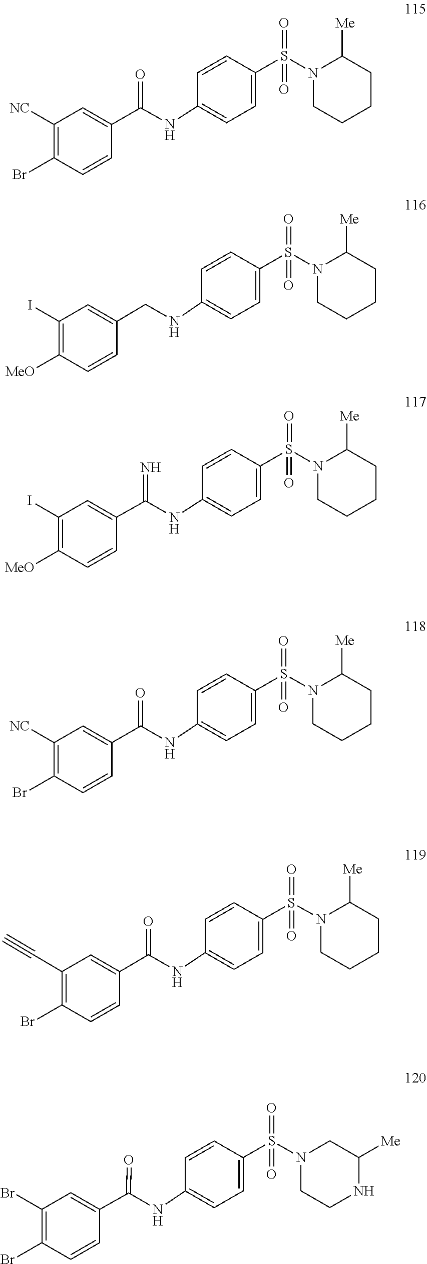 [SMILES: CC1CCCCN1S(=O)(=O)c1ccc(NC(=O)c2ccc(Br)c(C#N)c2)cc1 ; COc1ccc(CNc2ccc(S(=O)(=O)N3CCCCC3C)cc2)cc1I ; COc1ccc(C(=N)Nc2ccc(S(=O)(=O)N3CCCCC3C)cc2)cc1I ; CC1CCCCN1S(=O)(=O)c1ccc(NC(=O)c2ccc(Br)c(C#N)c2)cc1 ; C#Cc1cc(C(=O)Nc2ccc(S(=O)(=O)N3CCCCC3C)cc2)ccc1Br ; CC1CN(S(=O)(=O)c2ccc(NC(=O)c3ccc(Br)c(Br)c3)cc2)CCN1]

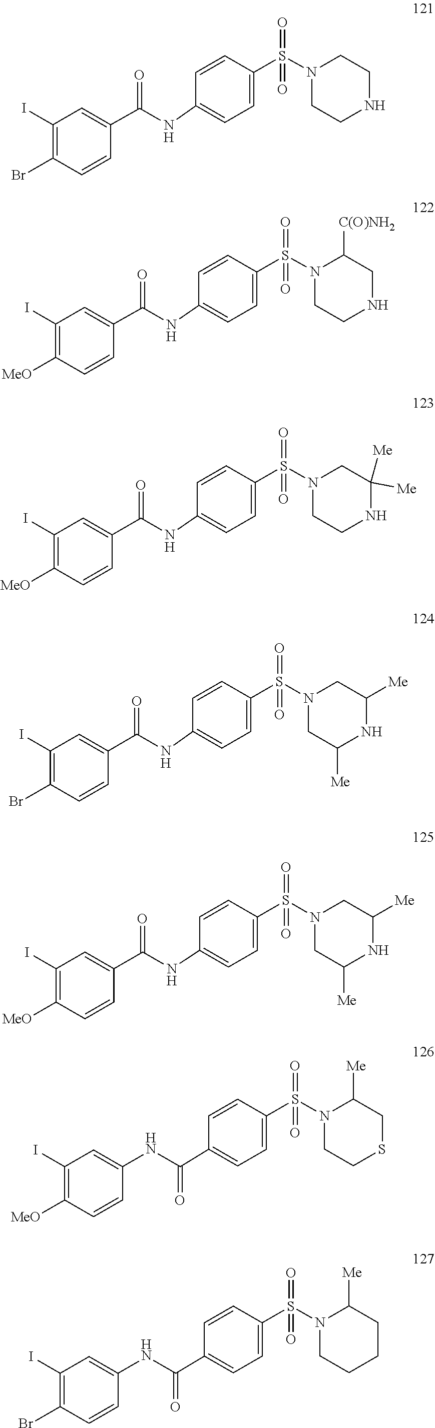

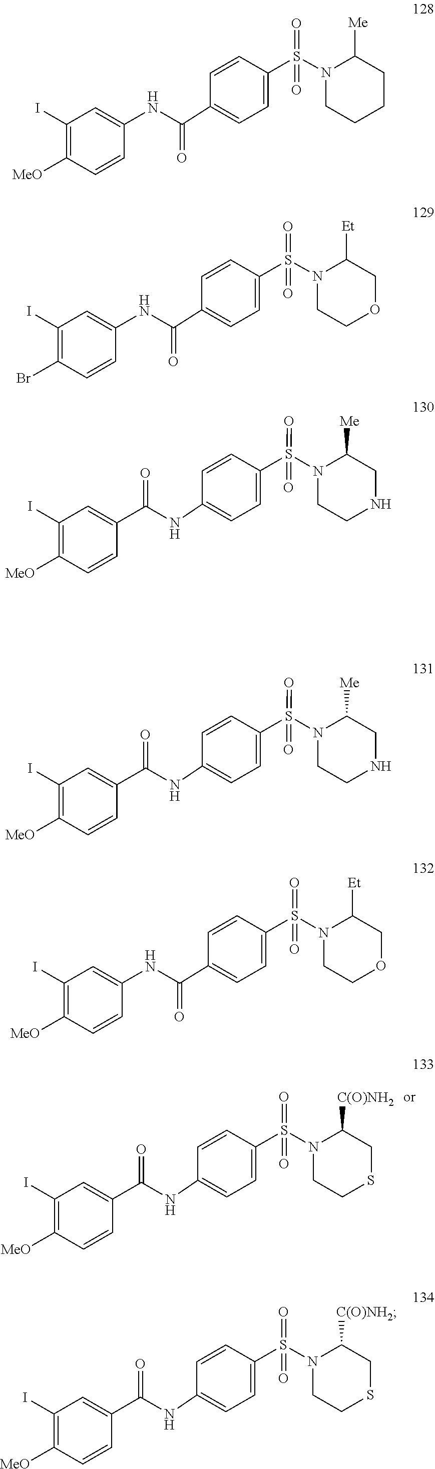

or a salt, enantiomer, diastereomer, or prodrug thereof. It is specifically contemplated that any one or more of these compounds may be excluded in an embodiment described herein.

Certain aspects of the disclosure are directed to a method for inhibiting serum response factor activity in a cell, a method for inhibiting smooth muscle contractile protein accumulation in a cell, and/or a method for inhibiting smooth muscle contractile protein expression in a cell comprising administering to the cell a composition comprising an effective amount of a compound of Formula II as described herein. Some aspects of the disclosure are directed to a method for reducing cellular contractile force, a method for inhibiting tumor cell growth, a method for inhibiting spreading, migration, and metastasis in a subject having a tumor, a method for reducing cellular metabolism, a method for attenuating hypoxia-induced response, a method for inhibiting HIF1α accumulation, a method for treating sleep apnea, and/or a method for treating glaucoma comprising administering to a subject a composition comprising an effective amount of a compound of Formula II as described herein. In aspects, a compound of Formula II inhibits expression of smooth muscle myosin heavy chains. In some embodiments, a compound of Formula II inhibits expression of smooth muscle alpha actin. In some embodiments, a compound of Formula II inhibits localized accumulation of smooth muscle myosin heavy chains. In some aspects, a compound of Formula II inhibits localized accumulation of smooth muscle alpha actin. In some embodiments, a compound of Formula II stimulates generation of trabecular meshwork cells. In some aspects, a compound of Formula II stimulates generation of Schlemm's Canal cells. In some aspects, a compound of Formula II inhibits contractile force generation of trabecular meshwork cells. In some embodiments, a compound of Formula II inhibits contractile force generation of Schlemm's canal endothelial cells. In some aspects, a compound of Formula II inhibits fibronectin expression.

In embodiments where a compound of Formula II is used for inhibiting tumor cell growth, administration of the compound may be done prior to, concurrently with, or subsequent to chemotherapy, surgical treatment, immunotherapy, or radiation treatment. In some aspects, a compound of Formula II inhibits human serum response factor activity. In some embodiments, inhibition of serum response factor activity affects at least one of cell cycle regulation, apoptosis, cell growth, and differentiation.

Certain aspects of the disclosure are directed towards compositions comprising a compound of Formula II:

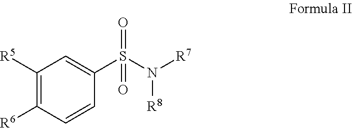

where R⁵ and R⁶ are each independently hydrogen, halide, substituted or unsubstituted alkyl, alkoxy, amine, alkylamine, sulfonamide, or join together to form a 5 or 6 member carbocycle or heterocycle; R⁷, and R⁸ are each independently hydrogen alkyl, substituted or unsubstituted aryl, wherein the substituted aryl may be substituted with amide, sulfonamide, substituted or unsubstituted alkyl, or two adjacent carbon atoms on the substituted aryl ring form a carbocycle or heterocycle ring. In some aspects, a compound of Formula II is further defined as:

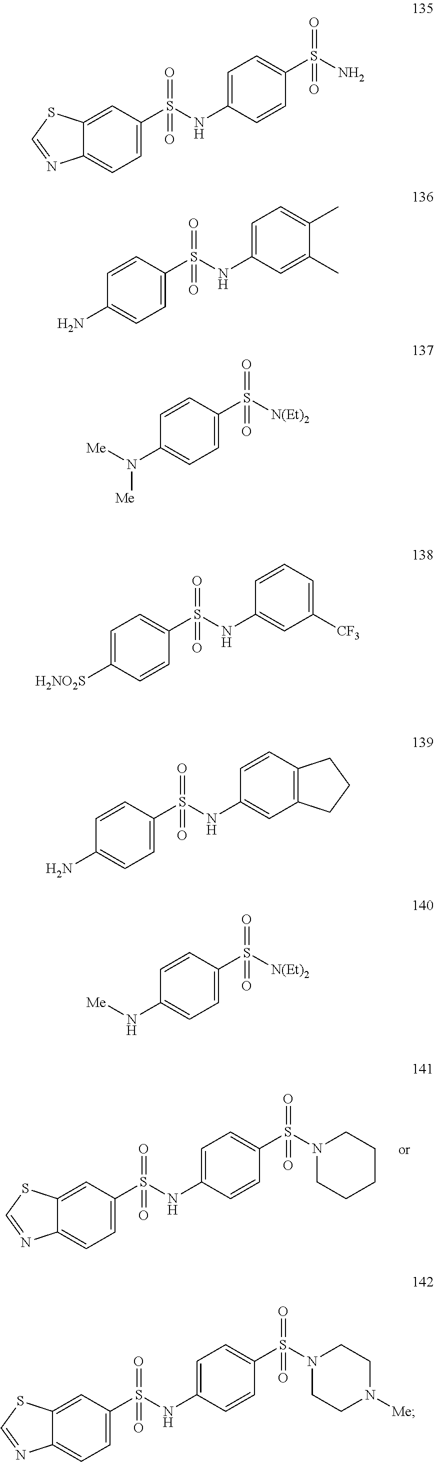

or a salt, enantiomer, diastereomer, or prodrug thereof. It is specifically contemplated that one or more of these compounds may be excluded in an embodiment disclosed herein.

Other embodiments of the invention are discussed throughout this application. Any embodiment discussed with respect to one aspect applies to other aspects as well and vice versa. Each embodiment described herein is understood to be embodiments that are applicable to all aspects. It is contemplated that any embodiment discussed herein can be implemented with respect to any method or composition, and vice versa. Furthermore, compositions and kits can be used to achieve methods disclosed herein.

The terms “effective amount” or “therapeutically effective amount” refer to that amount of a composition of the disclosure that is sufficient to effect treatment, as defined herein, when administered to a mammal in need of such treatment. This amount will vary depending upon the subject and disease condition being treated, the weight and age of the subject, the severity of the disease condition, the particular composition of the disclosure chosen, the dosing regimen to be followed, timing of administration, manner of administration and the like, all of which can readily be determined by one of ordinary skill in the art.

The term “remodilin” refers to any compound represented by Formula I or Formula II. The term “glaucoma” refers to glaucoma caused by high intraocular pressure that damages the eye's optic nerve and can result in vision loss and blindness. The term “metastasis” refers to the spread of cancer cells from the place where they first formed to another part of the body. In metastasis, cancer cells break away from the original (primary) tumor and invade adjacent tissues directly, or cancer cells travel through the blood or lymph system, and form a new tumor in other organs or tissues of the body.

Smooth muscle contractile proteins include actin and myosin. “Smooth muscle contractile protein accumulation” refers to localized aggregation of actin and myosin that enables localized contractile events in the cytoplasm, including but not limited to motile activity. “Trabecular meshwork cells” are those cells located near the base of the cornea. Trabecular meshwork cells make layers of beams, part of a fibrous basement membrane containing extracellular matrix and cells. In this area of high outflow resistance, trabecular meshwork cells regulate eye pressure by controlling drainage of fluid into Schlemm's canals that flow into the bloodstream.

The “numerical values” and “ranges” provided for the various substituents are intended to encompass all integers within the recited range. For example, when defining n as an integer representing a value including from about 1 to 100, where the value typically encompasses the integer specified as n+10% (or for smaller integers from 1 to about 25, ±3), it should be understood that n can be an integer from 1 to 100 (e.g., 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, 105 or 110, or any between those listed). The combined terms “about” and “±10%” or “±3” should be understood to disclose and provide specific support for equivalent ranges wherever used.

The term “optional” or “optionally” means that the subsequently described event or circumstance may or may not occur, and that the description includes instances where said event or circumstance occurs and instances in which it does not.

As used herein, “pharmaceutically acceptable carrier” or “pharmaceutically acceptable excipient” includes any and all solvents, dispersion media, coatings, antibacterial and antifungal agents, isotonic and absorption delaying agents and the like. In several embodiments, these media and agents can be used in combination with pharmaceutically active substances. Except insofar as any conventional media or agent is incompatible with the active ingredient, its use in the therapeutic compositions is contemplated. Supplementary active ingredients can also be incorporated into the compositions.

The term “treatment” or “treating” means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device and/or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

A “disease” is defined as a pathological condition of a body part, an organ, or a system resulting from any cause, such as infection, genetic defect, or environmental stress. In particular embodiments, the disease or condition is related to glaucoma, cancer, or hypoxia.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset.

The terms “inhibit,” “inhibiting,” and “inhibition,” (and grammatical equivalents) are used according to their plain and ordinary meaning in the area of medicine and biology. In the context of a physiological phenomena, e.g., a symptom, in an untreated subject relative to a treated subject, these terms mean to limit, prevent, or block a biological/chemical reaction to achieve a reduction in the quantity and/or magnitude of the physiological phenomena in the treated subject as compared to a differentially treated subject (such as an untreated subject or a subject treated with a different dosage or mode of administration) by any amount that is detectable and/or recognized as clinically relevant by any medically trained personnel. In some embodiments, the quantity and/or magnitude of the physiological phenomena in the treated subject is about, at least about, or at most about 5, 10, 15, 20, 25, 30, 35, 40, 45, 50, 55, 60, 65, 70, 75, 80, 85, 90, 95, or 100% (or any range derivable therein) lower than the quantity and/or magnitude of the physiological phenomena in the differentially treated subject. Alternatively, in other embodiments, the quantity and/or magnitude of the physiological phenomena in the treated subject is about, at least about, or at most about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0 times (or any range derivable therein) lower than the quantity and/or magnitude of the physiological phenomena in the differentially treated subject.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Some aspects of the disclosure are directed towards the use of a composition as disclosed herein in any method disclosed herein. Some embodiments provide for the use of any composition disclosed herein for treating glaucoma, inhibiting tumor cell growth, inhibiting metastasis, reducing cellular metabolism, attenuating hypoxia-induced response, inhibiting HIF1α accumulation, or any method disclosed herein. It is specifically contemplated that any step or element of an embodiment may be implemented in the context of any other step(s) or element(s) of a different embodiment disclosed herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 Remodilins inhibit myofibroblast transformation (MFT). Serum deprived human lung-derived fibroblasts were treated with 1 ng/mL TGFβ1 (or not, left lane) and 0, 1, 3, or 10 μM remodilin for 2d. Four remodilins each inhibited smooth muscle α-actin (ACTA2) or fibronectin-1 (FN1) protein expression (markers of MFT).

FIG. 2 Signaling pathway targeted by remodilins. Schematic diagram (center) of signaling from TGFβ to (SRF); red proteins are some, but not all, potential targets. TGFβ stimulates Smad-dependent transcription in human lung fibroblasts that is unchanged by 10 μM remodilin 4 (left), but TGFβ-stimulated SRF-dependent transcription is inhibited by the remodilin (right). SBE—Smad binding element; Luc—luciferase; TK-RL—constitutively active thymidine kinase promoter-driven renilla luciferase (used to control for transfection efficiency).

FIG. 3 MDA-MB-231 cells were grown into spheroids, then allowed to migrate into collagen gels in DMEM containing 0.5% FBS and remodilin (10 μM remodilin 39 [top row] or remodilin 83 [bottom row]) or diluent (0.1% DMSO) for 48 hrs (representative 48 hr images shown). Remodilins 39 and 83 inhibited invasion of MB-231 cells into collagen (2 mg/mL) gels.

FIG. 4 Remodilins 39 and 83 inhibited 10% FBS-directed invasion migration of MDA-MB-231-derived BM1 cells through a Matrigel-coated transwell mem-brane, in a dose-dependent fashion. Mean+SEM shown; n=3/condition. **P<0.01; ***P<0.001; ****P<0.0001.

FIG. 5 Remodilins 39 and 83 inhibited migration of confluent MDA-MB-231 cells into a circular scratch “wound” in a dose-dependent fashion. Mean+SD shown; n=8 wells/condition. P<0.0001 for 1, 3, & 10 uM remodilin 39 vs DMSO (control) by 2-way ANOVA/Dunnett's.

FIG. 6 Tissue and plasma remodilin concentrations after single oral doses (filled circles—50 mg/kg; open circles—10 mg/kg). Dotted line shows concentrations corresponding to 10 uM. The greater oral bioavailability, longer half-life, and inter-tissue variation of remodilin 83 concentration are readily evident. Mean+SEM shown; n=3/condition.

FIG. 7 After 14d BID dosing, concentrations of remodilin 83 vary among tissues, but are relatively steady within each tissue throughout the 12-hour dosing period when the remodilin is given IP. There is greater variation over time when remodilin 83 is given PO. Mean+SEM shown; n=3/condition. Dotted lines show concentration corresponding to 10 μM. There was no mortality or gross clinical evidence of toxicity observed during this study.

FIG. 8 In vivo pharmacokinetics of remodilin 39 after single 10 mg/kg IP dose (blue symbols; n=3 mice at each time point; mean+SEM shown) or after 15 daily IP doses (red symbol; n=1).

FIG. 9 Anti-fibrogenic effects of remodilin 50 in human primary trabecular meshwork cells. Remodilin inhibited alpha smooth muscle actin expression in TM cells treated with TGFβ2 [5 ng/mL] for 48 hr compared to DMSO.

FIG. 10 Treatment with either remodilin 50 or remodilin 82 alone induced relaxation in TM cells after 1 hr treatment in a dose dependent manner.

FIGS. 11A-11C Anti-fibrogenic effects of remodilins 50, 73, and 82 in human primary Schlemm's canal endothelial cells. FIG. 11A Remodilin inhibited fibronectin expression in SC cells treated with TGFβ2 [2.5 ng/mL] for 48 hr compared to DMSO. FIG. 11B Remodilin also inhibited the elevation of cellular contractile force in SC cells treated with TGFβ2 [2.5 ng/mL] for 48 hr compared to DMSO in a dose-dependent manner. FIG. 11C Remodilin treatment alone induced relaxation in SC cells after 1 hr treatment in a dose dependent manner.

FIG. 12A-12B Remodilins inhibit accumulation of hypoxia-inducible factor-1 alpha (HIF1α) protein. FIG. 12A Effect of remodilin 83 on TGFβ-induced phosphorylation of AKT and ERK1/2 and TGFβ-induced HIF1α accumulation in human fibroblasts. FIG. 12B Both remodilins 39 and 83 inhibited HIF1α accumulation in HEK293 cells exposed to 6 hrs of hypoxia (1% 02).

FIG. 13A-13B Effect of remodilin 83 on oxygen consumption rate (OCR) and extracellular acidification rate (ECAR). FIG. 13A A549 cells were treated with remodilin 83 (open circles) or its vehicle (filled circles) at time=30 min, and oxygen consumption rate (OCR) was continuously measured over time. FIG. 13B A549 cells were treated with remodilin 83 (open circles) or its vehicle (filled circles) at time=30 min, and extracellular acidification rate (ECAR) was measured continuously over time. Cells were sequentially treated with oligomycin (ATP synthase inhibitor), FCCP (uncoupler) and antimycin A/rotenone (A+R) (complex III and I inhibitor). Results indicate that remodilins decrease both mitochondrial respiration and glycolysis.

FIG. 14A-14B Remodilins' effects on hypoxia-induced accumulation of HIF1α. FIG. 14A Western blot demonstrating that two remodilins (39 and 83 at 3 or 10 μM as indicated) each inhibit the accumulation of HIF1α in cultured HEK293T cells exposed to 6 hrs steady hypoxia (H). FIG. 14B HIF1α was absent in cells ex-posed to 6 hrs normoxia (N).

FIG. 15A-15B Remodilin's effect on hypertension and weight. FIG. 15A Remodilin 83 (20 mg/kg BID IP, filled circles) blocks the systemic hypertension otherwise induced by 10 days of IH (8 hrs/day) in Sprague Dawley rats in vehicle-treated rats (open circles). FIG. 15B R187 had little effect on blood pressure in rats unexposed to IH (filled squares) and had no obvious effect on health as judged by clinical observation or weight gain. N=2/group; D0-Day 0 (prior to IH), D11-Day 11.

### DETAILED DESCRIPTION

The present invention overcomes the deficiencies of the prior art by providing remodilin compositions that inhibit serum response factor activity. Because serum response factor activity regulates expression of oncogenes, smooth muscle proteins, and cell matrix maintenance proteins, the remodilins disclosed herein provide novel small molecules for treating cancer, metastasis, and glaucoma.

Hypoxia-inducible factor 1-alpha (HIF1a) plays an important role in cellular responses to systemic oxygen levels. The remodilins disclosed herein inhibit TGFb-induced HIF1α accumulation in fibroblasts and inhibit hypoxia-induced accumulation of HIF1a. In clinical settings, remodilins may be used to inhibit hypoxia-induced responses, and may be useful for treating ischemia and hypoxia-related diseases, including sleep apnea. Remodilins also inhibit glycolysis and cellular metabolism.

### A. CHEMICAL DEFINITIONS

As used herein, a “small molecule” refers to an organic compound that is frequently synthesized via conventional organic chemistry methods (e.g., in a laboratory). Typically, a small molecule is characterized in that it contains several carbon-carbon bonds, and has a molecular weight of less than 1500 grams/mole. In certain embodiments, small molecules are less than 1000 grams/mole. In certain embodiments, small molecules are less than 550 grams/mole. In certain embodiments, small molecules are between 200 and 550 grams/mole. In certain embodiments, small molecules exclude peptides (e.g., compounds comprising 2 or more amino acids joined by a peptidyl bond). In certain embodiments, small molecules exclude nucleic acids.

As used herein, the term “amino” means —NH2; the term “nitro” means —NO2; the term “halo” or “halogen” designates —F, —Cl, —Br or —I; the term “mercapto” means —SH; the term “cyano” means —CN; the term “azido” means —N3; the term “silyl” means —SiH3, and the term “hydroxy” means —OH. In certain embodiments, a halogen may be —Br or —I.

As used herein, a “monovalent anion” refers to anions of a −1 charge. Such anions are well-known to those of skill in the art. Non-limiting examples of monovalent anions include halides (e.g., F—, Cl—, Br— and I—), NO2-, NO3-, hydroxide (OH—) and azide (N3-).

As used herein, the structure [symbol] indicates that the bond may be a single bond or a double bond. Those of skill in the chemical arts understand that in certain circumstances, a double bond between two particular atoms is chemically feasible and in certain circumstances, a double bond is not. The present invention therefore contemplates that a double bond may be formed only when chemically feasible.

The term “alkyl” includes straight-chain alkyl, branched-chain alkyl, cycloalkyl (alicyclic), cyclic alkyl, heteroatom-unsubstituted alkyl, heteroatom-substituted alkyl, heteroatom-unsubstituted Cn-alkyl, and heteroatom-substituted Cn-alkyl. In certain embodiments, lower alkyls are contemplated. The term “lower alkyl” refers to alkyls of 1-6 carbon atoms (that is, 1, 2, 3, 4, 5 or 6 carbon atoms). The term “heteroatom-unsubstituted Cn-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C1-C10-alkyl has 1 to 10 carbon atoms. The groups, —CH3 (Me), —CH2CH3 (Et), —CH2CH2CH3 (n-Pr), —CH(CH3)2 (iso-Pr), —CH(CH2)2 (cyclopropyl), —CH2CH2CH2CH3 (n-Bu), —CH(CH3)CH2CH3 (sec-butyl), —CH2CH(CH3)2 (iso-butyl), —C(CH3)3 (tert-butyl), —CH2C(CH3)3 (neo-pentyl), cyclobutyl, cyclopentyl, and cyclohexyl, are all non-limiting examples of heteroatom-unsubstituted alkyl groups. The term “heteroatom-substituted Cn-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C1-C10-alkyl has 1 to 10 carbon atoms. The following groups are all non-limiting examples of heteroatom-substituted alkyl groups: trifluoromethyl, —CH2F, —CH2Cl, —CH2Br, —CH2OH, —CH2OCH3, —CH2OCH2CF3, —CH2OC(O)CH3, —CH2NH2, —CH2NHCH3, —CH2N(CH3)2, —CH2CH2Cl, —CH2CH2OH, CH2CH2OC(O)CH3, —CH2CH2NHCO2C(CH3)3, and —CH2Si(CH3)3.

The term “alkenyl” includes straight-chain alkenyl, branched-chain alkenyl, cycloalkenyl, cyclic alkenyl, heteroatom-unsubstituted alkenyl, heteroatom-substituted alkenyl, heteroatom-unsubstituted Cn-alkenyl, and heteroatom-substituted Cn-alkenyl. In certain embodiments, lower alkenyls are contemplated. The term “lower alkenyl” refers to alkenyls of 1-6 carbon atoms (that is, 1, 2, 3, 4, 5 or 6 carbon atoms). The term “heteroatom-unsubstituted Cn-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C2-C10-alkenyl has 2 to 10 carbon atoms. Heteroatom-unsubstituted alkenyl groups include: —CH═CH2 (vinyl), —CH═CHCH3, —CH═CHCH2CH3, —CH2CH═CH2 (allyl), —CH2CH═CHCH3, and —CH═CH—C6H5. The term “heteroatom-substituted Cn-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C2-C10-alkenyl has 2 to 10 carbon atoms. The groups, —CH═CHF, —CH═CHCl and —CH═CHBr, are non-limiting examples of heteroatom-substituted alkenyl groups.

The term “aryl” includes heteroatom-unsubstituted aryl, heteroatom-substituted aryl, heteroatom-unsubstituted Cn-aryl, heteroatom-substituted Cn-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cn-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C6-C10-aryl has 6 to 10 carbon atoms. Non-limiting examples of heteroatom-unsubstituted aryl groups include phenyl (Ph), methylphenyl, (dimethyl)phenyl, —C6H4CH2CH3, —C6H4CH2CH2CH3, —C6H4CH(CH3)2, —C6H4CH(CH2)2, —C6H3(CH3)CH2CH3, —C6H4CH═CH2, —C6H4CH≡CHCH3, —C6H4C≡CH, —C6H4C≡CCH3, naphthyl, and the radical derived from biphenyl. The term “heteroatom-substituted Cn-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C1-C10-heteroaryl has 1 to 10 carbon atoms. Non-limiting examples of heteroatom-substituted aryl groups include the groups: —C6H4F, —C6H4C1, —C6H4Br, —C6H4I, —C6H40H, —C6H4OCH3, —C6H4OCH2CH3, —C6H4OC(O)CH3, —C6H4NH2, —C6H4NHCH3, —C6H4N(CH3)2, —C6H4CH2OH, —C6H4CH2OC(O)CH3, —C6H4CH2NH2, —C6H4CF3, —C6H4CN, —C6H4CHO, —C6H4CHO, —C6H4C(O)CH3, —C6H4C(O)C6H5, —C6H4CO2H, —C6H4CO2CH3, —C6H4CONH2, —C6H4CONHCH3, —C6H4CON(CH3)2, furanyl, thienyl, pyridyl, pyrrolyl, pyrimidyl, pyrazinyl, quinolyl, indolyl, and imidazoyl. In certain embodiments, heteroatom-substituted aryl groups are contemplated. In certain embodiments, heteroatom-unsubstituted aryl groups are contemplated. In certain embodiments, an aryl group may be mono-, di-, tri-, tetra- or penta-substituted with one or more heteroatom-containing substituents.

The term “aralkyl” includes heteroatom-unsubstituted aralkyl, heteroatom-substituted aralkyl, heteroatom-unsubstituted Cn-aralkyl, heteroatom-substituted Cn-aralkyl, heteroaralkyl, and heterocyclic aralkyl groups. In certain embodiments, lower aralkyls are contemplated. The term “lower aralkyl” refers to aralkyls of 7-12 carbon atoms (that is, 7, 8, 9, 10, 11 or 12 carbon atoms). The term “heteroatom-unsubstituted Cn-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C7-C10-aralkyl has 7 to 10 carbon atoms. Non-limiting examples of heteroatom-unsubstituted aralkyls are: phenylmethyl (benzyl, Bn) and phenylethyl. The term “heteroatom-substituted Cn-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structures, further wherein each heteroatom is independently selected from the group consisting of N, O, F, Cl, Br, I, Si, P, and S. For example, a heteroatom-substituted C2-C10-heteroaralkyl has 2 to 10 carbon atoms.

The term “acyl” includes straight-chain acyl, branched-chain acyl, cycloacyl, cyclic acyl, heteroatom-unsubstituted acyl, heteroatom-substituted acyl, heteroatom-unsubstituted Cn-acyl, heteroatom-substituted Cn-acyl, alkylcarbonyl, alkoxycarbonyl and aminocarbonyl groups. In certain embodiments, lower acyls are contemplated. The term “lower acyl” refers to acyls of 1-6 carbon atoms (that is, 1, 2, 3, 4, 5 or 6 carbon atoms). The term “heteroatom-unsubstituted Cn-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C1-C10-acyl has 1 to 10 carbon atoms. The groups, —CHO, —C(O)CH3, —C(O)CH2CH3, —C(O)CH2CH2CH3, —C(O)CH(CH3)2, —C(O)CH(CH2)2, —C(O)C6H5, —C(O)C6H4CH3, —C(O)C6H4CH2CH3, and —COC6H3(CH3)2, are non-limiting examples of heteroatom-unsubstituted acyl groups. The term “heteroatom-substituted Cn-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom, in addition to the oxygen of the carbonyl group, wherein each additional heteroatom is independently selected from the group consisting of N, O, F, Cl, Br, I, Si, P, and S. For example, a heteroatom-substituted C1-C10-acyl has 1 to 10 carbon atoms. The groups, —C(O)CH2CF3, —CO2H, —CO2-, —CO2CH3, —CO2CH2CH3, —CO2CH2CH2CH3, —CO2CH(CH3)2, —CO2CH(CH2)2, —C(O)NH2 (carbamoyl), —C(O)NHCH3, —C(O)NHCH2CH3, —CONHCH(CH3)2, —CONHCH(CH2)2, —CON(CH3)2, and —CONHCH2CF3, are non-limiting examples of heteroatom-substituted acyl groups.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cn-alkoxy, and heteroatom-substituted Cn-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cn-alkoxy” refers to a group, having the structure —OR, in which R is a heteroatom-unsubstituted Cn-alkyl, as that term is defined above. Heteroatom-unsubstituted alkoxy groups include: —OCH3, —OCH2CH3, —OCH2CH2CH3, —OCH(CH3)2, and —OCH(CH2)2. The term “heteroatom-substituted Cn-alkoxy” refers to a group, having the structure —OR, in which R is a heteroatom-substituted Cn-alkyl, as that term is defined above. For example, —OCH2CF3 is a heteroatom-substituted alkoxy group.

The term “alkenyloxy” includes straight-chain alkenyloxy, branched-chain alkenyloxy, cycloalkenyloxy, cyclic alkenyloxy, heteroatom-unsubstituted alkenyloxy, heteroatom-substituted alkenyloxy, heteroatom-unsubstituted Cn-alkenyloxy, and heteroatom-substituted Cn-alkenyloxy. The term “heteroatom-unsubstituted Cn-alkenyloxy” refers to a group, having the structure —OR, in which R is a heteroatom-unsubstituted Cn-alkenyl, as that term is defined above. The term “heteroatom-substituted Cn-alkenyloxy” refers to a group, having the structure —OR, in which R is a heteroatom-substituted Cn-alkenyl, as that term is defined above.

The term “alkynyloxy” includes straight-chain alkynyloxy, branched-chain alkynyloxy, cycloalkynyloxy, cyclic alkynyloxy, heteroatom-unsubstituted alkynyloxy, heteroatom-substituted alkynyloxy, heteroatom-unsubstituted Cn-alkynyloxy, and heteroatom-substituted Cn-alkynyloxy. The term “heteroatom-unsubstituted Cn-alkynyloxy” refers to a group, having the structure —OR, in which R is a heteroatom-unsubstituted Cn-alkynyl, as that term is defined above. The term “heteroatom-substituted Cn-alkynyloxy” refers to a group, having the structure —OR, in which R is a heteroatom-substituted Cn-alkynyl, as that term is defined above.

The term “aryloxy” includes heteroatom-unsubstituted aryloxy, heteroatom-substituted aryloxy, heteroatom-unsubstituted Cn-aryloxy, heteroatom-substituted Cn-aryloxy, heteroaryloxy, and heterocyclic aryloxy groups. The term “heteroatom-unsubstituted Cn-aryloxy” refers to a group, having the structure —OAr, in which Ar is a heteroatom-unsubstituted Cn-aryl, as that term is defined above. A non-limiting example of a heteroatom-unsubstituted aryloxy group is —OC6H5. The term “heteroatom-substituted Cn-aryloxy” refers to a group, having the structure —OAr, in which Ar is a heteroatom-substituted Cn-aryl, as that term is defined above.

The term “aralkyloxy” includes heteroatom-unsubstituted aralkyloxy, heteroatom-substituted aralkyloxy, heteroatom-unsubstituted Cn-aralkyloxy, heteroatom-substituted Cn-aralkyloxy, heteroaralkyloxy, and heterocyclic aralkyloxy groups. The term “heteroatom-unsubstituted Cn-aralkyloxy” refers to a group, having the structure —OAr, in which Ar is a heteroatom-unsubstituted Cn-aralkyl, as that term is defined above. The term “heteroatom-substituted Cn-aralkyloxy” refers to a group, having the structure —OAr, in which Ar is a heteroatom-substituted Cn-aralkyl, as that term is defined above.

The term “acyloxy” includes straight-chain acyloxy, branched-chain acyloxy, cycloacyloxy, cyclic acyloxy, heteroatom-unsubstituted acyloxy, heteroatom-substituted acyloxy, heteroatom-unsubstituted Cn-acyloxy, heteroatom-substituted Cn-acyloxy, alkylcarbonyloxy, arylcarbonyloxy, alkoxycarbonyloxy, aryloxycarbonyloxy, and carboxylate groups. The term “heteroatom-unsubstituted Cn-acyloxy” refers to a group, having the structure —OAc, in which Ac is a heteroatom-unsubstituted Cn-acyl, as that term is defined above. For example, —OC(O)CH3 is a non-limiting example of a heteroatom-unsubstituted acyloxy group. The term “heteroatom-substituted Cn-acyloxy” refers to a group, having the structure —OAc, in which Ac is a heteroatom-substituted Cn-acyl, as that term is defined above. For example, —OC(O)OCH3 and —OC(O)NHCH3 are non-limiting examples of heteroatom-unsubstituted acyloxy groups.

The term “alkylamino” includes straight-chain alkylamino, branched-chain alkylamino, cycloalkylamino, cyclic alkylamino, heteroatom-unsubstituted alkylamino, heteroatom-substituted alkylamino, heteroatom-unsubstituted Cn-alkylamino, and heteroatom-substituted Cn-alkylamino. The term “heteroatom-unsubstituted Cn-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C1-C10-alkylamino has 1 to 10 carbon atoms. The term “heteroatom-unsubstituted Cn-alkylamino” includes groups, having the structure —NHR, in which R is a heteroatom-unsubstituted Cn-alkyl, as that term is defined above. A heteroatom-unsubstituted alkylamino group would include —NHCH3, —NHCH2CH3, —NHCH2CH2CH3, —NHCH(CH3)2, —NHCH(CH2)2, —NHCH2CH2CH2CH3, —NHCH(CH3)CH2CH3, —NHCH2CH(CH3)2, —NHC(CH3)3, —N(CH3)2, —N(CH3)CH2CH3, —N(CH2CH3)2, N-pyrrolidinyl, and N-piperidinyl. The term “heteroatom-substituted Cn-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C1-C10-alkylamino has 1 to 10 carbon atoms. The term “heteroatom-substituted Cn-alkylamino” includes groups, having the structure —NHR, in which R is a heteroatom-substituted Cn-alkyl, as that term is defined above.

The term “alkenylamino” includes straight-chain alkenylamino, branched-chain alkenylamino, cycloalkenylamino, cyclic alkenylamino, heteroatom-unsubstituted alkenylamino, heteroatom-substituted alkenylamino, heteroatom-unsubstituted Cn-alkenylamino, heteroatom-substituted Cn-alkenylamino, dialkenylamino, and alkyl(alkenyl)amino groups. The term “heteroatom-unsubstituted Cn-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C2-C10-alkenylamino has 2 to 10 carbon atoms. The term “heteroatom-unsubstituted Cn-alkenylamino” includes groups, having the structure —NHR, in which R is a heteroatom-unsubstituted Cn-alkenyl, as that term is defined above. The term “heteroatom-substituted Cn-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C2-C10-alkenylamino has 2 to 10 carbon atoms. The term “heteroatom-substituted Cn-alkenylamino” includes groups, having the structure —NHR, in which R is a heteroatom-substituted Cn-alkenyl, as that term is defined above.

The term “alkynylamino” includes straight-chain alkynylamino, branched-chain alkynylamino, cycloalkynylamino, cyclic alkynylamino, heteroatom-unsubstituted alkynylamino, heteroatom-substituted alkynylamino, heteroatom-unsubstituted Cn-alkynylamino, heteroatom-substituted Cn-alkynylamino, dialkynylamino, alkyl(alkynyl)amino, and alkenyl(alkynyl)amino groups. The term “heteroatom-unsubstituted Cn-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C2-C10-alkynylamino has 2 to 10 carbon atoms. The term “heteroatom-unsubstituted Cn-alkynylamino” includes groups, having the structure —NHR, in which R is a heteroatom-unsubstituted Cn-alkynyl, as that term is defined above. The term “heteroatom-substituted Cn-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C2-C10-alkynylamino has 2 to 10 carbon atoms. The term “heteroatom-substituted Cn-alkynylamino” includes groups, having the structure —NHR, in which R is a heteroatom-substituted Cn-alkynyl, as that term is defined above.

The term “arylamino” includes heteroatom-unsubstituted arylamino, heteroatom-substituted arylamino, heteroatom-unsubstituted Cn-arylamino, heteroatom-substituted Cn-arylamino, heteroarylamino, heterocyclic arylamino, and alkyl(aryl)amino groups. The term “heteroatom-unsubstituted Cn-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C6-C10-arylamino has 6 to 10 carbon atoms. The term “heteroatom-unsubstituted Cn-arylamino” includes groups, having the structure —NHR, in which R is a heteroatom-unsubstituted Cn-aryl, as that term is defined above. The term “heteroatom-substituted Cn-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom-substituted C6-C10-arylamino has 6 to 10 carbon atoms. The term “heteroatom-substituted Cn-arylamino” includes groups, having the structure —NHR, in which R is a heteroatom-substituted Cn-aryl, as that term is defined above.

The term “aralkylamino” includes heteroatom-unsubstituted aralkylamino, heteroatom-substituted aralkylamino, heteroatom-unsubstituted Cn-aralkylamino, heteroatom-substituted Cn-aralkylamino, heteroaralkylamino, heterocyclic aralkylamino groups, and diaralkylamino groups. The term “heteroatom-unsubstituted Cn-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C7-C10-aralkylamino has 7 to 10 carbon atoms. The term “heteroatom-unsubstituted Cn-aralkylamino” includes groups, having the structure —NHR, in which R is a heteroatom-unsubstituted Cn-aralkyl, as that term is defined above. The term “heteroatom-substituted Cn-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C7-C10-aralkylamino has 7 to 10 carbon atoms. The term “heteroatom-substituted Cn-aralkylamino” includes groups, having the structure —NHR, in which R is a heteroatom-substituted Cn-aralkyl, as that term is defined above.

The term “amido” includes straight-chain amido, branched-chain amido, cycloamido, cyclic amido, heteroatom-unsubstituted amido, heteroatom-substituted amido, heteroatom-unsubstituted Cn-amido, heteroatom-substituted Cn-amido, alkylcarbonylamino, arylcarbonylamino, alkoxycarbonylamino, aryloxycarbonylamino, acylamino, alkylaminocarbonylamino, arylaminocarbonylamino, and ureido groups. The term “heteroatom-unsubstituted Cn-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C1-C10-amido has 1 to 10 carbon atoms. The term “heteroatom-unsubstituted Cn-amido” includes groups, having the structure —NHR, in which R is a heteroatom-unsubstituted Cn-acyl, as that term is defined above. The group, —NHC(O)CH3, is a non-limiting example of a heteroatom-unsubstituted amido group. The term “heteroatom-substituted Cn-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C1-C10-amido has 1 to 10 carbon atoms. The term “heteroatom-substituted Cn-amido” includes groups, having the structure —NHR, in which R is a heteroatom-unsubstituted Cn-acyl, as that term is defined above. The group, —NHCO2CH3, is a non-limiting example of a heteroatom-substituted amido group.

The term “alkylthio” includes straight-chain alkylthio, branched-chain alkylthio, cycloalkylthio, cyclic alkylthio, heteroatom-unsubstituted alkylthio, heteroatom-substituted alkylthio, heteroatom-unsubstituted Cn-alkylthio, and heteroatom-substituted Cn-alkylthio. The term “heteroatom-unsubstituted Cn-alkylthio” refers to a group, having the structure —SR, in which R is a heteroatom-unsubstituted Cn-alkyl, as that term is defined above. The group, —SCH3, is an example of a heteroatom-unsubstituted alkylthio group. The term “heteroatom-substituted Cn-alkylthio” refers to a group, having the structure —SR, in which R is a heteroatom-substituted Cn-alkyl, as that term is defined above.

The term “alkenylthio” includes straight-chain alkenylthio, branched-chain alkenylthio, cycloalkenylthio, cyclic alkenylthio, heteroatom-unsubstituted alkenylthio, heteroatom-substituted alkenylthio, heteroatom-unsubstituted Cn-alkenylthio, and heteroatom-substituted Cn-alkenylthio. The term “heteroatom-unsubstituted Cn-alkenylthio” refers to a group, having the structure —SR, in which R is a heteroatom-unsubstituted Cn-alkenyl, as that term is defined above. The term “heteroatom-substituted Cn-alkenylthio” refers to a group, having the structure —SR, in which R is a heteroatom-substituted Cn-alkenyl, as that term is defined above.

The term “alkynylthio” includes straight-chain alkynylthio, branched-chain alkynylthio, cycloalkynylthio, cyclic alkynylthio, heteroatom-unsubstituted alkynylthio, heteroatom-substituted alkynylthio, heteroatom-unsubstituted Cn-alkynylthio, and heteroatom-substituted Cn-alkynylthio. The term “heteroatom-unsubstituted Cn-alkynylthio” refers to a group, having the structure —SR, in which R is a heteroatom-unsubstituted Cn-alkynyl, as that term is defined above. The term “heteroatom-substituted Cn-alkynylthio” refers to a group, having the structure —SR, in which R is a heteroatom-substituted Cn-alkynyl, as that term is defined above.

The term “arylthio” includes heteroatom-unsubstituted arylthio, heteroatom-substituted arylthio, heteroatom-unsubstituted Cn-arylthio, heteroatom-substituted Cn-arylthio, heteroarylthio, and heterocyclic arylthio groups. The term “heteroatom-unsubstituted Cn-arylthio” refers to a group, having the structure —SAr, in which Ar is a heteroatom-unsubstituted Cn-aryl, as that term is defined above. The group, —SC6H5, is an example of a heteroatom-unsubstituted arylthio group. The term “heteroatom-substituted Cn-arylthio” refers to a group, having the structure —SAr, in which Ar is a heteroatom-substituted Cn-aryl, as that term is defined above.

The term “aralkylthio” includes heteroatom-unsubstituted aralkylthio, heteroatom-substituted aralkylthio, heteroatom-unsubstituted Cn-aralkylthio, heteroatom-substituted Cn-aralkylthio, heteroaralkylthio, and heterocyclic aralkylthio groups. The term “heteroatom-unsubstituted Cn-aralkylthio” refers to a group, having the structure —SAr, in which Ar is a heteroatom-unsubstituted Cn-aralkyl, as that term is defined above. The group, —SCH2C6H5, is an example of a heteroatom-unsubstituted aralkyl group. The term “heteroatom-substituted Cn-aralkylthio” refers to a group, having the structure —SAr, in which Ar is a heteroatom-substituted Cn-aralkyl, as that term is defined above.

The term “acylthio” includes straight-chain acylthio, branched-chain acylthio, cycloacylthio, cyclic acylthio, heteroatom-unsubstituted acylthio, heteroatom-substituted acylthio, heteroatom-unsubstituted Cn-acylthio, heteroatom-substituted Cn-acylthio, alkylcarbonyloxy, arylcarbonyloxy, alkoxycarbonyloxy, aryloxycarbonyloxy, and carboxylate groups. The term “heteroatom-unsubstituted Cn-acylthio” refers to a group, having the structure —SAc, in which Ac is a heteroatom-unsubstituted Cn-acyl, as that term is defined above. The group, —SCOCH3, is an example of a heteroatom-unsubstituted acylthio group. The term “heteroatom-substituted Cn-acylthio” refers to a group, having the structure —SAc, in which Ac is a heteroatom-substituted Cn-acyl, as that term is defined above.

The term “alkylsilyl” includes straight-chain alkylsilyl, branched-chain alkylsilyl, cycloalkylsilyl, cyclic alkylsilyl, heteroatom-unsubstituted alkylsilyl, heteroatom-substituted alkylsilyl, heteroatom-unsubstituted Cn-alkylsilyl, and heteroatom-substituted Cn-alkylsilyl. The term “heteroatom-unsubstituted Cn-alkylsilyl” refers to a radical, having a single silicon atom as the point of attachment, further having one, two, or three saturated carbon atoms attached to the silicon atom, further having a linear or branched, cyclic or acyclic structure, containing a total of n carbon atoms, all of which are nonaromatic, 5 or more hydrogen atoms, a total of 1 silicon atom, and no additional heteroatoms. For example, a heteroatom-unsubstituted C1-C10-alkylsilyl has 1 to 10 carbon atoms. An alkylsilyl group includes dialkylamino groups. The groups, —Si(CH3)3 and —Si(CH3)2C(CH3)3, are non-limiting examples of heteroatom-unsubstituted alkylsilyl groups. The term “heteroatom-substituted Cn-alkylsilyl” refers to a radical, having a single silicon atom as the point of attachment, further having at least one, two, or three saturated carbon atoms attached to the silicon atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the silicon atom at the point of attachment, wherein each additional heteroatom is independently selected from the group consisting of N, O, F, Cl, Br, I, Si, P, and S. For example, a heteroatom-substituted C1-C10-alkylsilyl has 1 to 10 carbon atoms.

The term “phosphonate” includes straight-chain phosphonate, branched-chain phosphonate, cyclophosphonate, cyclic phosphonate, heteroatom-unsubstituted phosphonate, heteroatom-substituted phosphonate, heteroatom-unsubstituted Cn-phosphonate, and heteroatom-substituted Cn-phosphonate. The term “heteroatom-unsubstituted Cn-phosphonate” refers to a radical, having a single phosphorous atom as the point of attachment, further having a linear or branched, cyclic or acyclic structure, further having a total of n carbon atoms, 2 or more hydrogen atoms, a total of three oxygen atom, and no additional heteroatoms. The three oxygen atoms are directly attached to the phosphorous atom, with one of these oxygen atoms doubly bonded to the phosphorous atom. For example, a heteroatom-unsubstituted C0-C10-phosphonate has 0 to 10 carbon atoms. The groups, —P(O)(OH)2, —P(O)(OH)OCH3, —P(O)(OH)OCH2CH3, —P(O)(OCH3)2, and —P(O)(OH)(OC6H5) are non-limiting examples of heteroatom-unsubstituted phosphonate groups. The term “heteroatom-substituted Cn-phosphonate” refers to a radical, having a single phosphorous atom as the point of attachment, further having a linear or branched, cyclic or acyclic structure, further having a total of n carbon atoms, 2 or more hydrogen atoms, three or more oxygen atoms, three of which are directly attached to the phosphorous atom, with one of these three oxygen atoms doubly bonded to the phosphorous atom, and further having at least one additional heteroatom in addition to the three oxygen atoms, wherein each additional heteroatom is independently selected from the group consisting of N, O, F, Cl, Br, I, Si, P, and S. For example, a heteroatom-unsubstituted C0-C10-phosphonate has 0 to 10 carbon atoms.

The term “phosphinate” includes straight-chain phosphinate, branched-chain phosphinate, cyclophosphinate, cyclic phosphinate, heteroatom-unsubstituted phosphinate, heteroatom-substituted phosphinate, heteroatom-unsubstituted Cn-phosphinate, and heteroatom-substituted Cn-phosphinate. The term “heteroatom-unsubstituted Cn-phosphinate” refers to a radical, having a single phosphorous atom as the point of attachment, further having a linear or branched, cyclic or acyclic structure, further having a total of n carbon atoms, 2 or more hydrogen atoms, a total of two oxygen atom, and no additional heteroatoms. The two oxygen atoms are directly attached to the phosphorous atom, with one of these oxygen atoms doubly bonded to the phosphorous atom. For example, a heteroatom-unsubstituted C0-C10-phosphinate has 0 to 10 carbon atoms. The groups, —P(O)(OH)H, —P(O)(OH)CH3, —P(O)(OH)CH2CH3, —P(O)(OCH3)CH3, and —P(O)(OC6H5)H are non-limiting examples of heteroatom-unsubstituted phosphinate groups. The term “heteroatom-substituted Cn-phosphinate” refers to a radical, having a single phosphorous atom as the point of attachment, further having a linear or branched, cyclic or acyclic structure, further having a total of n carbon atoms, 2 or more hydrogen atoms, two or more oxygen atoms, two of which are directly attached to the phosphorous atom, with one of these two oxygen atoms doubly bonded to the phosphorous atom, and further having at least one additional heteroatom in addition to the two oxygen atoms, wherein each additional heteroatom is independently selected from the group consisting of N, O, F, Cl, Br, I, Si, P, and S. For example, a heteroatom-unsubstituted C0-C10-phosphinate has 0 to 10 carbon atoms.

Any apparently unfulfilled valency is to be understood to be properly filled by hydrogen atom(s). For example, a compound with a substituent of —O or —N is to be understood to be —OH or —NH2, respectively.

Any genus, subgenus, or specific compound discussed herein is specifically contemplated as being excluded from any embodiment described herein.

Compounds described herein may be prepared synthetically using conventional organic chemistry methods known to those of skill in the art and/or are commercially available (e.g., ChemBridge Co., San Diego, Calif.).

Embodiments are also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present invention are contemplated.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

Derivatives of compounds of the present invention are also contemplated. In certain aspects, “derivative” refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds and structures disclosed herein include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, heteroatoms such as N, S, or O can be substituted into the structure instead of a carbon atom.

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include 13C and 14C.

As noted above, compounds of the present invention may exist in prodrug form. As used herein, “prodrug” is intended to include any covalently bonded carriers which release the active parent drug or compounds that are metabolized in vivo to an active drug or other compounds employed in the methods of the invention in vivo when such prodrug is administered to a subject. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a subject, cleaves to form a free hydroxyl, free amino, or carboxylic acid, respectively. Other examples include, but are not limited to, acetate, formate, and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (2002), which is incorporated herein by reference.

### B. PHARMACEUTICAL FORMULATIONS AND ADMINISTRATION THEREOF

1. Pharmaceutical Formulations and Routes of Administration

Pharmaceutical compositions are provided herein that comprise an effective amount of one or more substances and/or additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of the invention may comprise different types of carriers depending on whether it is to be administered in solid, liquid or aerosol form, and whether it need to be sterile for such routes of administration as injection. The present invention can be administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or by other method or any combination of the foregoing as would be known to one of ordinary skill in the art (see, for example, Remington's Pharmaceutical Sciences, 1990). Administration of the compositions disclosed herein may be done prior to, concurrently with, or subsequent to chemotherapy, surgical treatment, immunotherapy, or radiation treatment.

When administered for the treatment of glaucoma, the compounds disclosed herein may be administered ocularly, parenterally, transmucosally, transdermally, intramuscularly, intravenously, intradermally, intravascularly, or subcutaneously, intraperitoneally, by topical drops or ointment, periocular injection, systemically by intravenous injection or orally, intracamerally into the anterior chamber or vitreous, via a depot attached to the intraocular lens implant inserted during surgery, or via a depot placed in the eye sutured in the anterior chamber or vitreous. In some embodiments, a polymeric composition, e.g., a contact lens, contains one or more compounds disclosed herein and releases the one or more compounds over a pre-determined period of time. In some aspects, a microneedle array may be used to deliver one or more compounds disclosed herein to a desired location. In some embodiments, implantable extended-release microparticles or nanoparticles may be used to deliver one or more compounds disclosed herein. In some aspects, one or more compounds disclosed herein may be incorporated in and released from a tear duct plug. In some embodiments, one or more compounds disclosed herein may be incorporated into a biodegradable polymer that can degrade and release the one or more compounds over time.

The actual dosage amount of a composition administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0% of an active ingredient (or any range derivable therein). In other embodiments, the active ingredient may comprise between about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% of the weight of the unit, or between about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60%, for example, and any range derivable therein.

In certain embodiments, pharmaceutical compositions may comprise, for example, at least about 0.1% of a compound described herein. In other embodiments, the compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Methods may involve administering to the patient or subject at least or at most 1, 2, 3, 4, 5, 6, 7, 8, 9, 10 or more doses of a therapeutic composition. A dose may be a composition comprising about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or g/ml or micrograms/ml or mM or μM (or any range derivable therein) of each remodilin or the total amount of a combination of remodelins.

The composition may be administered in a dose of 1-100 (this such range includes intervening doses) or more μg or any number in between the foregoing amount per dose. Each dose may be in a volume of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more μl or ml or any number in between the foregoing.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of an infection are exhibited or felt by a patient or after a clinician evaluates the patient for an infection. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of an infection (or any range derivable therein). The patient may be treated for 1, 2, 3, 4, 5, 6, 7, 8, 9, 10 or more days (or any range derivable therein) or until symptoms of an infection have disappeared or been reduced or after 6, 12, 18, or 24 hours or 1, 2, 3, 4, or 5 days after symptoms of an infection have disappeared or been reduced.

Compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months. Compositions may also be administered 30 seconds, 1, 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months or more.

It is specifically contemplated that the composition may be administered once daily, twice daily, three times daily, four times daily, five times daily, or six times daily (or any range derivable therein) and/or as needed to the patient. Alternatively, the composition may be administered every 2, 4, 6, 8, 12 or 24 hours (or any range derivable therein) to or by the patient. In some embodiments, the patient is administered the composition for a certain period of time or with a certain number of doses after experiencing symptoms of a disease or disorder.

In additional embodiments, the composition may be administered to (or taken by) the patient about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 l/min, l/hour, l/day, l/week, l/month, ml/min, ml/hour, ml/day, ml/week, ml/month, g/min, g/hour, g/day, g/week, g/month, mg/min, mg/hour, mg/day, mg/week, mg/month or any range derivable therein.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride or combinations thereof.

In other embodiments, one may use eye drops, nasal solutions or sprays, aerosols or inhalants. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Additional formulations which are suitable for other modes of administration include suppositories. Suppositories are solid dosage forms of various weights and shapes, usually medicated, for insertion into the rectum, vagina, or urethra. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin, or combinations thereof.

2. Combination Therapy

The compositions and methods disclosed herein may be used in combination, i.e., a composition comprising a compound of Formula I may include at least one compound of Formula II and/or at least one additional compound of Formula I. A composition comprising a compound of Formula II may include at least one compound of Formula I and/or at least one additional compound of Formula II.

The compositions and related methods of the present invention, particularly administration of a remodilin of Formula I or Formula II, may also be used in combination with the administration of other glaucoma or cancer therapies. In glaucoma, exemplary treatments include prostaglandins (latanoprost (Xalatan)), travoprost (Travatan Z), tafluprost (Zioptan), bimatoprost (Lumigan) and latanoprostene bunod (Vyzulta); beta blockers timolol (Betimol, Istalol, Timoptic) and betaxolol (Betoptic); alpha-adrenergic agonists apraclonidine (Iopidine) and brimonidine (Alphagan P, Qoliana); carbonic anhydrase inhibitors dorzolamide (Trusopt) and brinzolamide (Azopt); Rho kinase inhibitors netarsudil (rhopressa); and miotic or cholinergic agents pilocarpine (Isopto Carpine).

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

Additional cancer therapies include factors that cause DNA damage, such asas γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Additional cancer therapies that may be used in combination with remodelins include immunotherapeutics that rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Inducers of cellular proliferation may be used in combination with remodelins. The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

Inhibitors of cellular proliferation may be used in combination with remodelins. The tumor suppressor oncogenes function to inhibit excessive cellular proliferation. The inactivation of these genes destroys their inhibitory activity, resulting in unregulated proliferation. Exemplary tumor suppressors include p53, p16 and C-CAM.

The compositions and related methods of the present invention may be used in combination with therapies that regulate cell death (apoptosis), by inducing apoptosis. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists. Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BclXL, BlcW, BlcS, Mcl-1, A1, Bfl-1) or counteract Bcl-2 function and promote cell death (e.g., Bax, Bak, Bik, Bim, Bid, Bad, Harakiri).

Compounds discussed herein may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the candidate substance. In other aspects, one or more remodilins may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to administering a different glaucoma, anti-proliferative, or anti-metastatic therapeutic. In some embodiments, more than one course of therapy may be employed. It is contemplated that multiple courses may be implemented.

### C. ORGANISMS AND CELL SOURCE

Methods can involve cells, tissues, or organs involving the heart, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord.

Moreover, methods can be employed in cells of the following type: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell (totipotent, pluripotent or multipotent), unfertilized or fertilized oocyte, or sperm.

### D. CANCER

TGFβ plays a key role in promoting breast cancer metastasis, and both anti-TGFβ antibodies and pharmacological inactivation of the TGFβ receptor inhibit experimental breast cancer metastasis in mice. Antibodies and small-molecule TGFβ receptor antagonists inhibit TGFβ function globally, thereby preventing TGFβ from exerting its beneficial physiological activities at sites unrelated to the cancer or its metastases.

A new class of small molecules termed remodilins affect some downstream targets of TGFβ cell stimulation (e.g., activation of serum response factor SRF) without affecting proximal TGFβ cell signaling. Importantly, remodilins blunt the invasive and migratory capabilities of human triple negative breast cancer cells in vitro, and they blunt TGFβ-stimulated myofibroblast transformation, a process associated with fibroblast metastasis-promoting capability. Given the role of SRF in cell migration, inhibition of SRF is a new therapeutic approach for treating aberrant cell growth and metastasis.

In certain aspects, a composition comprising at least one remodelin as disclosed herein may be administered to treat a cancer. The cancer may be a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### E. GLAUCOMA

The aqueous humor of primary open angle glaucoma (POAG) patients contains an elevated level of transforming growth factor β2 (TGF-β2) as compared with controls. In perfused human anterior segments, TGF-β2 causes a decrease in outflow facility and an increase in intraocular pressure (IOP). The effects of TGF-β2 were further confirmed in murine models where outflow facility is decreased and IOP increased after adenoviral gene transfer of active human TGF-β2. TGF-β2 is known to activate pro-fibrogenic activities in many parts of the body including trabecular meshwork (TM) cells. In TM cells, TGF-β2 induces elevated expression of extracellular proteins (collagen, fibronectin and laminin) and contractile proteins like alpha-smooth muscle actin (α-SMA). TGF-β2 also induces pro-fibrogenic activities in SC cells and these profibrogenic activities likely make SC cells stiffer. In POAG, the increased stiffness of SC cells has been shown to be correlated with reduced pore formation in SC inner walls and concomitantly increased outflow resistance. Taken together, elevation of TGF-β2 has detrimental effects on aqueous humor outflow likely through pro-fibrogenic activations of TM cells and SC cells. By inhibiting pro-fibrogenic activation by TGF-β2, TGF-β2-induced decreases in outflow facility can be prevented and those structural changes made by TGF-β2 potentially reversed.

A novel class of small molecules (remodilins) inhibit TGF-β1 induced myofibroblast differentiation in vitro in human lung fibroblasts and human airway smooth muscle cells. In murine models in vivo, these remodilins mitigate airways hyperresponsiveness and inhibit aberrant airway remodeling.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### General Chemistry Methods

All air or moisture sensitive reactions were performed under positive pressure of nitrogen with oven-dried glassware. Chemical reagents and anhydrous solvents were obtained from commercial sources and used as-is. Preparative purification was performed on a Waters semi-preparative HPLC. The column used was a Phenomenex Luna C18 (5 micron, 30×75 mm) at a flow rate of 45 mL/min. The mobile phase consisted of acetonitrile and water (each containing 0.1% trifluoroacetic acid). A gradient of 10% to 50% acetonitrile over 8 minutes was used during the purification. Fraction collection was triggered by UV detection (220 nm). Analytical analysis for purity was determined by a Final QC Method:

Final QC Method analysis was performed on an Agilent 1260 with a 7 minute gradient of 4% to 100% acetonitrile (containing 0.025% trifluoroacetic acid) in water (containing 0.05% trifluoroacetic acid) over 8 minute run time at a flow rate of 1 mL/min. A Phenomenex Luna C18 column (3 micron, 3×75 mm) was used at a temperature of 50° C. Purity determination was performed using an Agilent Diode Array Detector. Mass determination was performed using an Agilent 6130 mass spectrometer with electrospray ionization in the positive mode. All of the analogues for assay have purity greater than 95% based on both analytical methods. 1H and 13C NMR spectra were recorded on a Varian 400 (100) MHz spectrometer. High resolution mass spectrometry was recorded on Agilent 6210 Time-of-Flight LC/MS system. Method A: Amide coupling via acid chloride intermediate

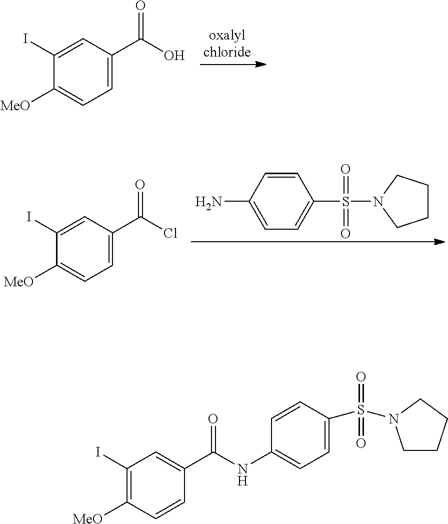 [SMILES: COc1ccc(C(=O)O)cc1I ; COc1ccc(C(=O)Cl)cc1I ; Nc1ccc(S(=O)(=O)N2CCCC2)cc1 ; COc1ccc(C(=O)Nc2ccc(S(=O)(=O)N3CCCC3)cc2)cc1I]

4-Bromo-3-iodobenzoic acid (0.25 g, 0.77 mmol), and oxalyl chloride (0.09 ml, 0.99 mmol) was stirred in DCM (5.00 mL) at room temperature (rt) before adding DMF (2.96 μl, 0.04 mmol) slowly. The mixture was stirred at rt for 72 h, at which time the reaction was concentrated to a white solid. The acid chloride product was reacted with 4-(pyrrolidin-1-ylsulfonyl)aniline to afford the amide product. An alternative reaction entails refluxing 1 equivalent of carboxylic acid with 1.2 equivalents of PCl₅ in CHCl₃ (1.0 mL). This reaction mixture is refluxed for 3 h then cooled and concentrated. This mixture is used neat for the acid chloride-amine coupling reaction.

Method B: Negishi coupling of organozinc and aryl halide

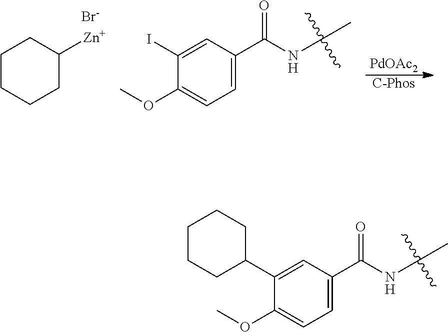

Method C: Sulfonamide formation by reaction with sulfonyl chloride intermediate, acetamide hydrolysis, and reaction between resulting amine and aromatic acid chloride

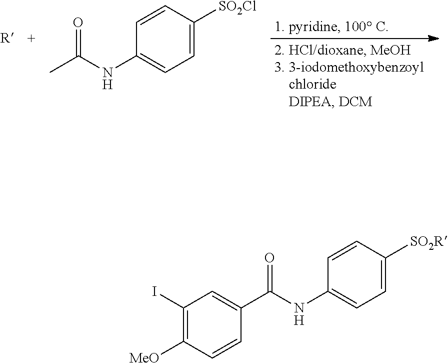

Method D: Sulfonamide formation by reaction with sulfonyl chloride intermediate, reduction of nitro to amine, and reaction between resulting amine and aromatic acid chloride

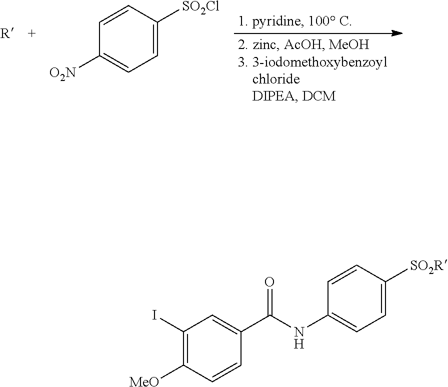

### N-(4-(N,N-diethylsulfamoyl)phenyl)-3-iodo-4-methoxybenzamide (4)

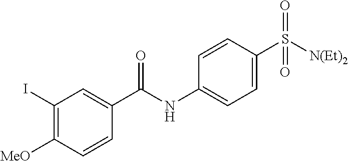

4-amino-N,N-diethylbenzenesulfonamide (0.35 mmol), in DIPEA (1.00 mmol) was stirred at rt in DCM (1.0 mL) before a 1 M solution of 3-iodo-4-methoxybenzoyl chloride (0.42 mL, 0.42 mmol) in DCM was added. This solution was stirred overnight and when complete the reaction was diluted with DCM, poured into 1 N HCl and extracted 3×'s with DCM. The organic layers were combined and wash 1× with saturated bicarb and 1× with brine. The organic layer was dried with Na₂SO₄, filtered and concentrated. The oil was the purified by reverse phase to give the named compound. 1H NMR (400 MHz, DMSO-d6) δ 10.47 (s, 1H), 8.39 (d, J=2.2 Hz, 1H), 8.14-7.89 (m, 3H), 7.88-7.65 (m, 2H), 7.13 (d, J=8.8 Hz, 1H), 3.90 (s, 3H), 3.13 (q, J=7.1 Hz, 4H), and 1.02 (t, J=7.1 Hz, 6H); LC-MS Retention Time=5.630 min; HRMS: m/z (M+Na)+=(Calculated for C18H21IN2NaO4S, 511.0159) found, 511.0157.

### 3-Iodo-4-methoxy-N-(4-(pyrrolidin-1-ylsulfonyl)phenyl)benzamide (6)

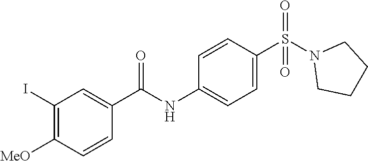 [SMILES: COc1ccc(C(=O)Nc2ccc(S(=O)(=O)N3CCCC3)cc2)cc1I]

Synthesize as in Method A using 4-(pyrrolidin-1-ylsulfonyl)aniline as the starting material instead of 4-amino-N,N-diethylbenzenesulfonamide. 1H NMR (400 MHz, DMSO-d6) δ 10.49 (s, 1H), 8.38 (d, J=2.2 Hz, 1H), 8.00 (dd, J=8.7, and 10.8 Hz, 3H), 7.81-7.73 (m, 2H), 7.13 (d, J=8.8 Hz, 1H), 3.90 (s, 3H), 3.17-3.07 (m, 4H), and 1.66-1.58 (m, 4H); LC-MS Retention Time=5.471 min; HRMS: m/z (M+H)+=(Calculated for C18H20IN204S, 487.0183) found, 487.0168.

### 3-Iodo-4-methoxy-N-(4-(piperidin-1-ylsulfonyl)phenyl)benzamide (3)

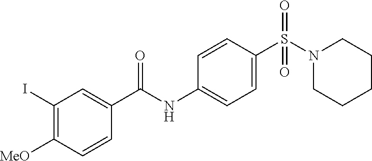 [SMILES: COc1ccc(C(=O)Nc2ccc(S(=O)(=O)N3CCCCC3)cc2)cc1I]

Synthesize as in Method A using 4-(piperidin-1-ylsulfonyl)aniline, HCl as the starting material instead of 4-amino-N,N-diethylbenzenesulfonamide. 1H NMR (400 MHz, DMSO-d6) δ 10.50 (s, 1H), 8.39 (d, J=2.2 Hz, 1H), 8.05-7.95 (m, 3H), 7.72-7.64 (m, 2H), 7.13 (d, J=8.8 Hz, 1H), 3.90 (s, 3H), 2.84 (t, J=5.5 Hz, 4H), 1.57-1.46 (m, 4H), and 1.34 (s, 2H); LC-MS Retention Time=5.826 min; HRMS: m/z (M+Na)+=(Calculated for C₁₉H₂₁IN₂NaO₄S, 523.0159) found, 523.0169.

### N-(4-(N,N-diethylsulfamoyl)phenyl)-4-methoxybenzamide (1)

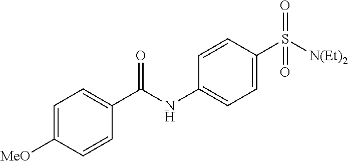

Synthesize as in Method A using 4-methoxybenzoyl chloride instead of 3-iodo-4-methoxybenzoyl chloride. ¹H NMR (400 MHz, Chloroform-d) δ 8.22 (s, 1H), 7.89-7.80 (m, 2H), 7.80-7.68 (m, 4H), 6.98-6.90 (m, 2H), 3.85 (s, 3H), 3.43 (s, 1H), 3.21 (q, J=7.12 Hz, 4H), and 1.11 (t, J=7.13 Hz, 6H); LC-MS Retention Time=5.084 min; HRMS: m/z (M+H)+=(Calculated for C₁₈H₂₃N₂O₄S, 363.1373) found, 363.1363.

### N-(4-(N,N-diethylsulfamoyl)phenyl)-3-iodo-4-methylbenzamide (16)

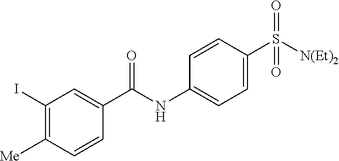

Synthesize as in Method A using 3-iodo-4-methylbenzoyl chloride instead of 3-iodo-4-methoxybenzoyl chloride. ¹H NMR (400 MHz, DMSO-d₆) δ 10.56 (s, 1H), 8.39 (d, J=1.9 Hz, 1H), 8.00-7.92 (m, 2H), 7.89 (dd, J=1.9, and 7.9 Hz, 1H), 7.80-7.71 (m, 2H), 7.48 (dd, J=0.8, and 7.9 Hz, 1H), 3.13 (q, J=7.1 Hz, 4H), 2.43 (s, 3H), and 1.02 (t, J=7.1 Hz, 6H); LC-MS Retention Time=6.381 min; HRMS: m/z (M+H)=(Calculated for C₁₈H₂₂IN₂O₃S, 473.0390) found, 473.0377.

### N-(4-(N,N-diethylsulfamoyl)phenyl)-4-hydroxy-3-iodobenzamide (19)

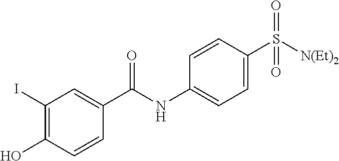

Synthesize as in Method A using 4-hydroxy-3-iodobenzoyl chloride instead of 3-iodo-4-methoxybenzoyl chloride. ¹H NMR (400 MHz, DMSO-d₆) δ 10.98 (s, 1H), 10.36 (s, 1H), 8.32 (d, J=2.2 Hz, 1H), 7.99-7.90 (m, 2H), 7.83 (dd, J=2.2, and 8.5 Hz, 1H), 7.78-7.69 (m, 2H), 6.94 (d, J=8.6 Hz, 1H), 3.13 (q, J=7.1 Hz, 4H), and 1.02 (t, J=7.1 Hz, 6H); LC-MS Retention Time=5.332 min; HRMS: m/z (M+Na)+=(Calculated for C₁₇H₁₉IN₂NaO₄S, 497.0002) found, 497.0025.

### N-(4-(N,N-diethylsulfamoyl)phenyl)-3-fluoro-4-methoxybenzamide (22)

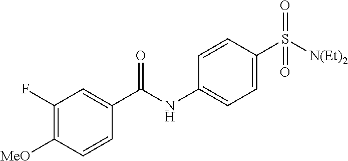

Synthesize as in Method A using 3-fluoro-4-methoxybenzoyl chloride instead of 3-iodo-4-methoxybenzoyl chloride. ¹H NMR (400 MHz, DMSO-d₆) δ 10.45 (s, 1H), 8.00-7.92 (m, 2H), 7.88-7.80 (m, 2H), 7.79-7.71 (m, 2H), 7.36-7.26 (m, 1H), 3.91 (s, 3H), 3.13 (q, J=7.1 Hz, 4H), and 1.02 (t, J=7.1 Hz, 6H); LC-MS Retention Time=5.542 min; HRMS: m/z (M+H)+=(Calculated for C₁₈H₂₂FN₂O₄S, 381.1279) found, 381.1274.

### N-(4-(N,N-diethylsulfamoyl)phenyl)-4-methoxy-3-(trifluoromethyl) benzamide (25)

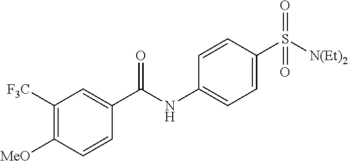

Synthesize as in Method A using 4-methoxy-3-trifluoromethylbenzoyl chloride instead of 3-iodo-4-methoxybenzoyl chloride. ¹H NMR (400 MHz, DMSO-d₆) δ 10.60 (s, 1H), 8.31-8.20 (m, 2H), 8.00-7.93 (m, 2H), 7.81-7.73 (m, 2H), 7.42 (d, J=8.8 Hz, 1H), 3.97 (s, 3H), 3.14 (q, J=7.1 Hz, 4H), and 1.02 (t, J=7.1 Hz, 6H); LC-MS Retention Time=6.012 min; HRMS: m/z (M+H)+=(Calculated for C₁₉H₂₂F₃N₂O₄S, 431.1247) found, 431.1234.

### N-(4-(N,N-diethylsulfamoyl)phenyl)-3-iodobenzamide (28)

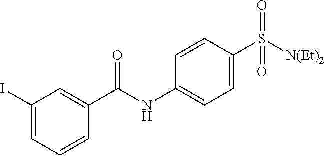

Synthesize as in Method A using 3-iodobenzoyl chloride instead of 3-iodo-4-methoxybenzoyl chloride. ¹H NMR (400 MHz, DMSO-d₆) δ 10.63 (s, 1H), 8.28 (t, J=1.5 Hz, 1H), 7.95 (tdd, J=0.8, 1.7, and 7.2 Hz, 4H), 7.81-7.72 (m, 2H), 7.38-7.29 (m, 1H), 3.13 (q, J=7.1 Hz, 4H), and 1.02 (t, J=7.1 Hz, 6H); Retention Time=6.109 min; HRMS: m/z (M+H)+=(Calculated for C₁₇H₂₀IN₂O₃S, 459.0234) found, 459.0216.

### 3-Bromo-N-(4-(N,N-diethylsulfamoyl)phenyl)-4-methoxybenzamide (31)

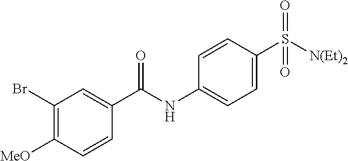

Synthesize as in Method A using 3-bromo-4-methoxybenzoyl chloride instead of 3-iodo-4-methoxybenzoyl chloride. ¹H NMR (400 MHz, DMSO-d₆) δ 10.48 (s, 1H), 8.22 (q, J=2.4 Hz, 1H), 7.98 (dd, J=8.3, and 17.8 Hz, 3H), 7.87-7.65 (m, 2H), 7.25 (dd, J=1.9, and 8.8 Hz, 1H), 3.92 (t, J=2.2 Hz, 3H), 3.13 (p, J=5.5, and 6.5 Hz, 4H), and 1.19-0.81 (m, 6H); Retention Time=5.882 min; HRMS: m/z (M+H)+=(Calculated for C₁₈H₂₂BrN₂O₄S, 442.0509) found, 442.0509.

### 3-Chloro-N-(4-(N,N-diethylsulfamoyl)phenyl)-4-methoxybenzamide (2)

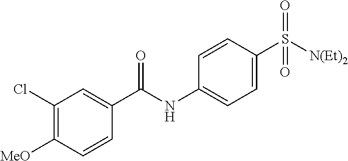

Synthesize as in Method A using 3-chloro-4-methoxybenzoyl chloride instead of 3-iodo-4-methoxybenzoyl chloride. ¹H NMR (400 MHz, DMSO-d₆) δ 10.48 (s, 1H), 8.07 (d, J=2.8 Hz, 1H), 8.01-7.91 (m, 3H), 7.79-7.69 (m, 2H), 7.44-7.11 (m, 1H), 3.93 (t, J=2.1 Hz, 3H), 3.27-2.97 (m, 4H), and 1.17-0.72 (m, 6H); Retention Time=5.798 min; HRMS: m/z (M+H)+=(Calculated for C₁₈H₂₂ClN₂O₄S, 397.0983) found, 397.0974.

### N-(3-(N,N-diethylsulfamoyl)phenyl)-3-iodo-4-methoxybenzamide (10)

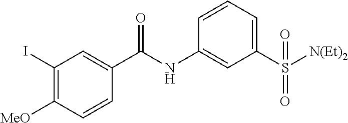

Synthesize as in Method A. ¹H NMR (400 MHz, DMSO-d₆) δ 10.42 (s, 1H), 8.41 (d, J=2.2 Hz, 1H), 8.25 (t, J=1.9 Hz, 1H), 8.08-7.99 (m, 2H), 7.60-7.51 (m, 1H), 7.47 (ddd, J=1.1, 1.8, and 7.8 Hz, 1H), 7.13 (d, J=8.8 Hz, 1H), 3.90 (s, 3H), 3.16 (q, J=7.1 Hz, 4H), and 1.04 (t, J=7.1 Hz, 6H); Retention Time=6.038 min; HRMS: m/z (M+H)+=(Calculated for C₁₈H₂₂IN₂O₄S, 489.0339) found, 489.0363.

### 4-Bromo-N-(4-(N,N-diethylsulfamoyl)phenyl)-3-iodobenzamide (44)

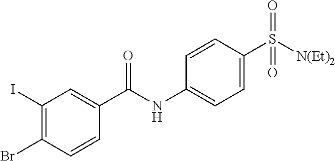

Synthesize as in Method A using 4-bromo-3-iodobenzoyl chloride instead of 3-iodo-4-methoxybenzoyl chloride. ¹H NMR (400 MHz, DMSO-d₆) δ 10.65 (s, 1H), 8.45 (dd, J=0.5, and 2.0 Hz, 1H), 7.99-7.91 (m, 2H), 7.91-7.82 (m, 2H), 7.79-7.71 (m, 2H), 3.12 (q, J=7.1 Hz, 4H), and 1.01 (t, J=7.1 Hz, 6H); Retention Time=6.573 min; HRMS: m/z (M+H)+=(Calculated for C₁₇H₁₉BrIN₂O₃S, 538.9319) found, 538.9310.

### 3-Iodo-4-methoxy-N-(4-sulfamoylphenyl)benzamide, NH₄⁺ (20)

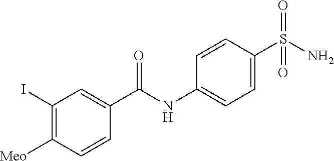

Synthesize as in Method A using 4-aminobenzenesulfonamide as the starting material. ¹H NMR (400 MHz, DMSO-d₆) δ 10.42 (s, 1H), 8.39 (d, J=2.2 Hz, 1H), 8.01 (dd, J=2.2, and 8.6 Hz, 1H), 7.97-7.82 (m, 2H), 7.84-7.70 (m, 2H), 7.23 (s, 2H), 7.13 (d, J=8.8 Hz, 1H), and 3.90 (s, 3H); Retention Time=4.470 min; HRMS: m/z (M+Na)+=(Calculated for C₁₄H₁₃IN₂NaO₄S, 454.9533) found, 454.9527.

### 3-Iodo-4-methoxy-N-(4-(methylsulfonamido)phenyl)benzamide (8)

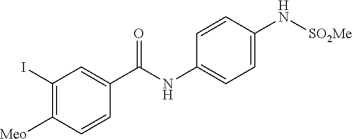

Synthesize as in Method A, N-(4-aminophenyl)methanesulfonamide (0.06 g, 0.34 mmol), and DIPEA (0.24 mL, 1.36 mmol) were combined in DCM (1.700 mL) before a 1 M solution of 3-iodo-4-methoxybenzoyl chloride (0.10 g, 0.34 mmol) in DCM was added. The reaction was allowed to stir overnight and was quenched with methanol before the reaction was purified by reverse phase to give final product. ¹H NMR (400 MHz, DMSO-d₆) δ 10.12 (s, 1H), 9.55 (s, 1H), 8.36 (d, J=2.2 Hz, 1H), 7.98 (dd, J=2.2, and 8.6 Hz, 1H), 7.72-7.63 (m, 2H), 7.20-7.07 (m, 3H), 3.88 (s, 3H), and 2.92 (s, 3H); Retention Time=4.792 min; HRMS: m/z (M+Na)+=(Calculated for C₁₅H₁₅IN₂NaO₄S, 468.9689) found, 468.9713.

### N-(4-(N-ethylsulfamoyl)phenyl)-3-iodo-4-methoxybenzamide (32)

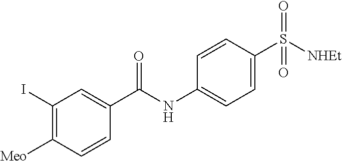

Synthesize as in Method A, using 4-amino-N-ethylbenzenesulfonamide as the starting material. ¹H NMR (400 MHz, DMSO-d₆) δ 10.45 (s, 1H), 8.39 (d, J=2.2 Hz, 1H), 8.01 (dd, J=2.2, and 8.7 Hz, 1H), 7.98-7.89 (m, 2H), 7.77-7.69 (m, 2H), 7.41 (t, J=5.8 Hz, 1H), 7.13 (d, J=8.7 Hz, 1H), 3.90 (s, 3H), 2.75 (qd, J=5.7, and 7.2 Hz, 2H), and 0.94 (t, J=7.2 Hz, 3H); Retention Time=5.061 min; HRMS: m/z (M+H)+=(Calculated for C₁₆H₁₈IN₂O₄S, 461.0026) found, 461.0049.

### 3-Bromo-4-methoxy-N-(4-(pyrrolidin-1-ylsulfonyl)phenyl)benzamide (18)

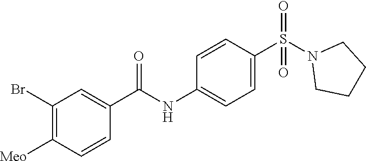

Synthesize as in Method A using 4-(pyrrolidin-1-ylsulfonyl)aniline as the starting material and 3-bromo-4-methoxybenzoyl chloride. ¹H NMR (400 MHz, DMSO-d₆) δ 10.51 (s, 1H), 8.22 (d, J=2.2 Hz, 1H), 8.04-7.95 (m, 3H), 7.81-7.72 (m, 2H), 7.25 (d, J=8.8 Hz, 1H), 3.92 (s, 3H), 3.15-3.06 (m, 4H), and 1.67-1.56 (m, 4H); Retention Time=5.774 min; HRMS: m/z (M+H)+=(Calculated for C₁₈H₂₀BrN₂O₄S, 441.0302) found, 441.0312.

### 3-Bromo-4-methyl-N-(4-(pyrrolidin-1-ylsulfonyl)phenyl)benzamide (30)

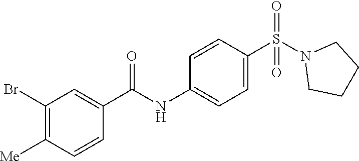 [SMILES: Cc1ccc(C(=O)Nc2ccc(S(=O)(=O)N3CCCC3)cc2)cc1Br]

Synthesize as in Method A using 4-(pyrrolidin-1-ylsulfonyl)aniline as the starting material and 3-bromo-4-methylbenzoyl chloride. ¹H NMR (400 MHz, DMSO-d₆) δ 10.61 (s, 1H), 8.17 (d, J=1.8 Hz, 1H), 8.04-7.95 (m, 2H), 7.87 (dd, J=1.8, and 7.9 Hz, 1H), 7.82-7.73 (m, 2H), 7.52 (dd, J=0.8, 7.9 Hz, 1H), 3.17-3.07 (m, 4H), 2.41 (s, 3H), and 1.67-1.56 (m, 4H); Retention Time=5.868 min; HRMS: m/z (M+H)+=(Calculated for C₁₈H₂₀BrN₂O₃S, 424.0403) found, 424.0407.

### 4-Chloro-3-iodo-N-(4-(pyrrolidin-1-ylsulfonyl)phenyl)benzamide (33)

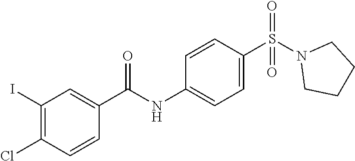 [SMILES: O=C(Nc1ccc(S(=O)(=O)N2CCCC2)cc1)c1ccc(Cl)c(I)c1]

Synthesize as in Method A using 4-(pyrrolidin-1-ylsulfonyl)aniline as the starting material and the 4-chloro-5-iodobenzoyl chloride was prepared using refⁱⁱ. ¹H NMR (400 MHz, DMSO-d₆) δ 10.68 (s, 1H), 8.48 (d, J=2.1 Hz, 1H), 8.02-7.91 (m, 3H), 7.83-7.70 (m, 3H), 3.17-3.07 (m, 4H), and 1.67-1.56 (m, 4H); Retention Time=6.033 min; HRMS: m/z (M+H)+=(Calculated for C₁₇H₁₇ClIN₂O₃S, 491.9718) found, 491.9729.

### 3-Bromo-4-isopropyl-N-(4-(pyrrolidin-1-ylsulfonyl)phenyl)benzamide (21)

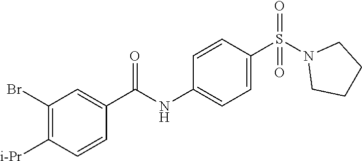 [SMILES: CC(C)c1ccc(C(=O)Nc2ccc(S(=O)(=O)N3CCCC3)cc2)cc1Br]

Synthesize as in Method A using 4-(pyrrolidin-1-ylsulfonyl)aniline as the starting material and 3-bromo-4-isopropylbenzoyl chloride. ¹H NMR (400 MHz, DMSO-d₆) δ 10.61 (s, 1H), 8.15 (d, J=1.9 Hz, 1H), 8.05-7.88 (m, 3H), 7.82-7.73 (m, 2H), 7.55 (d, J=8.2 Hz, 1H), 3.29 (s, 4H), 3.15-3.06 (m, 1H), 1.67-1.56 (m, 4H), and 1.22 (d, J=6.9 Hz, 6H); Retention Time=6.415 min; HRMS: m/z (M+H)+=(Calculated for C₂₀H₂₄BrN₂O₃S, 453.0667) found, 453.0654.

### 4-Bromo-3-iodo-N-(4-(pyrrolidin-1-ylsulfonyl)phenyl)benzamide (50)

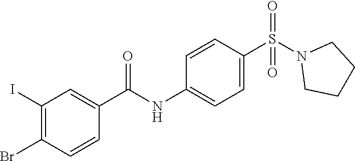 [SMILES: O=C(Nc1ccc(S(=O)(=O)N2CCCC2)cc1)c1ccc(Br)c(I)c1]

4-(Pyrrolidin-1-ylsulfonyl)aniline (0.17 g, 0.77 mmol), DIPEA (0.27 ml, 1.53 mmol) in DCM 2.5 mL was stirred for 3 min before the addition of 4-bromo-3-iodobenzoyl chloride (0.26 g, 0.77 mmol) in DCM 1 mL was added directly to round bottom. The reaction mixture was stirred overnight, concentrated, and taken up in MeOH at which time the solution was turbid. Water was added and heated until solution turned clear and then let sit for 1 h. A tan solid came out of solution, was filtered, washed with water, and dried to give 285 mg as a tan solid. ¹H NMR (400 MHz, DMSO-d₆) δ 10.68 (s, 1H), 8.46 (dd, J=0.4, and 2.0 Hz, 1H), 8.02-7.94 (m, 2H), 7.92-7.81 (m, 2H), 7.82-7.74 (m, 2H), 3.17-3.06 (m, 4H), and 1.67-1.56 (m, 4H); LC-MS retention time (Method 2) 6.052 min; HRMS: m/z (M+Na)+=(Calculated for C₁₇H₁₆BrIN₂NaO3S, 556.9002) found, 556.8965.

### 4-Bromo-3-methoxy-N-(4-(pyrrolidin-1-ylsulfonyl)phenyl)benzamide (37)

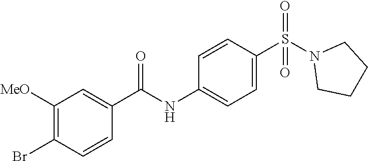 [SMILES: COc1cc(C(=O)Nc2ccc(S(=O)(=O)N3CCCC3)cc2)ccc1Br]

Synthesize as in Method A using 4-(pyrrolidin-1-ylsulfonyl)aniline as the starting material and 4-bromo-5-methoxybenzoyl chloride. ¹H NMR (400 MHz, DMSO-d₆): δ 10.62 (s, 1H), 8.03-7.95 (m, 2H), 7.83-7.71 (m, 3H), 7.58 (d, J=1.9 Hz, 1H), 7.48 (dd, J=2.0, 8.2 Hz, 1H), 3.93 (s, 3H), 3.15-3.07 (m, 4H), and 1.67-1.56 (m, 4H); Retention Time=5.577 min; HRMS: m/z (M+Na)+=(Calculated for C₁₈H₁₉BrN₂NaO₄S 463.0122) found, 463.0137.

### 3-Iodo-4-methyl-N-(4-(pyrrolidin-1-ylsulfonyl)phenyl)benzamide (40)

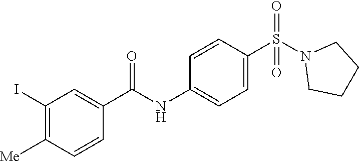 [SMILES: Cc1ccc(C(=O)Nc2ccc(S(=O)(=O)N3CCCC3)cc2)cc1I]

Synthesize as in Method A using 4-(pyrrolidin-1-ylsulfonyl)aniline as the starting material and 3-iodo-4-methylbenzoyl chloride. ¹H NMR (400 MHz, DMSO-d₆): δ 10.58 (s, 1H), 8.38 (d, J=1.8 Hz, 1H), 8.03-7.95 (m, 2H), 7.89 (dd, J=2.0, and 8.0 Hz, 1H), 7.81-7.73 (m, 2H), 7.48 (dd, J=0.8, and 7.9 Hz, 1H), 3.11 (td, J=3.5, and 7.0 Hz, 4H), 2.42 (s, 3H), and 1.67-1.56 (m, 4H); Retention Time=5.994 min; HRMS: m/z (M+H)+=(Calculated for C₁₈H₂₀IN₂O₃S, 471.0234) found, 471.0233.

### 3-Bromo-4-ethyl-N-(4-(pyrrolidin-1-ylsulfonyl)phenyl)benzamide (47)

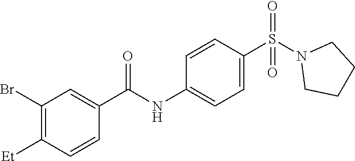 [SMILES: CCc1ccc(C(=O)Nc2ccc(S(=O)(=O)N3CCCC3)cc2)cc1Br]

Synthesize as in Method A using 4-(pyrrolidin-1-ylsulfonyl)aniline as the starting material and 3-bromo-4-ethylbenzoyl chloride. ¹H NMR (400 MHz, DMSO-d₆) δ 10.61 (s, 1H), 8.16 (d, J=1.8 Hz, 1H), 8.04-7.96 (m, 2H), 7.90 (dd, J=1.8, and 8.0 Hz, 1H), 7.82-7.70 (m, 2H), 7.51 (d, J=8.0 Hz, 1H), 3.25-2.91 (m, 4H), 2.76 (q, J=7.5 Hz, 2H), 1.81-1.50 (m, 4H), and 1.18 (t, J=7.5 Hz, 3H); Retention Time=6.161 min; FIRMS: m/z (M+H)+=(Calculated for C₁₉H₂₂BrN₂O₃S, 438.0559) found, 438.0538.

### 4-Acetamido-3-iodo-N-(4-(pyrrolidin-1-ylsulfonyl)phenyl)benzamide (52)

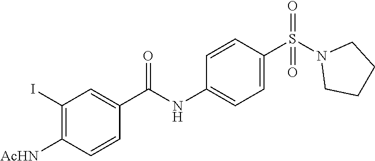 [SMILES: CC(=O)Nc1ccc(C(=O)Nc2ccc(S(=O)(=O)N3CCCC3)cc2)cc1I]

Synthesize as in Method A using 4-(pyrrolidin-1-ylsulfonyl)aniline as the starting material and 4-acetamido-3-iodobenzoyl chloride. Retention Time=4.721 min; HRMS: m/z (M+H)+=(Calculated for C₁₉H₂₁IN₃O₄S, 514.0292) found, 514.0307.

### 3,4-Dibromo-N-(4-(pyrrolidin-1-ylsulfonyl)phenyl)benzamide (59)

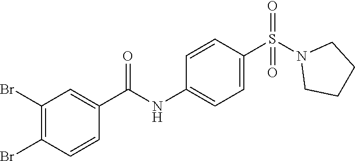 [SMILES: O=C(Nc1ccc(S(=O)(=O)N2CCCC2)cc1)c1ccc(Br)c(Br)c1]

Synthesize as in Method A using 4-(pyrrolidin-1-ylsulfonyl)aniline as the starting material and 3,4-dibromobenzoyl chloride. ¹H NMR (400 MHz, DMSO-d₆) δ 10.71 (s, 1H), 8.30 (d, J=2.1 Hz, 1H), 8.03-7.90 (m, 3H), 7.85 (dd, J=2.1, and 8.4 Hz, 1H), 7.83-7.74 (m, 2H), 3.17-3.06 (m, 4H), and 1.67-1.56 (m, 4H); Retention Time=5.951 min; HRMS: m/z (M+Na)+=(Calculated for C₁₇H₁₆Br₂N₂NaO₃S, 510.9121) found, 510.9134.

### 3-Fluoro-4-iodo-N-(4-(pyrrolidin-1-ylsulfonyl)phenyl)benzamide (64)

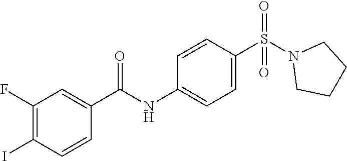 [SMILES: O=C(Nc1ccc(S(=O)(=O)N2CCCC2)cc1)c1ccc(I)c(F)c1]

Synthesize as in Method A using 4-(pyrrolidin-1-ylsulfonyl)aniline as the starting material and 3-fluoro-4-iodobenzoyl chloride. ¹H NMR (400 MHz, DMSO-d₆) δ 10.67 (s, 1H), 8.08-7.95 (m, 3H), 7.85-7.74 (m, 3H), 7.58 (dd, J=2.0, and 8.2 Hz, 1H), 3.15-3.06 (m, 4H), and 1.68-1.55 (m, 4H); Retention Time=5.682 min; HRMS: m/z (M+H)+=(Calculated for C₁₇H₁₇FIN₂O₃S, 476.0013) found, 475.9985.

### 4-Bromo-3-chloro-N-(4-(pyrrolidin-1-ylsulfonyl)phenyl)benzamide (67)

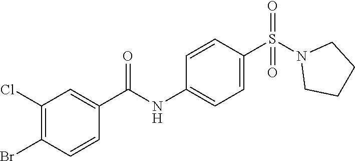 [SMILES: O=C(Nc1ccc(S(=O)(=O)N2CCCC2)cc1)c1ccc(Br)c(Cl)c1]

Synthesize as in Method A using 4-(pyrrolidin-1-ylsulfonyl)aniline as the starting material and 4-bromo-3-chlorobenzoyl chloride. ¹H NMR (400 MHz, DMSO-d₆) δ 10.72 (s, 1H), 8.18 (d, J=2.2 Hz, 1H), 8.03-7.91 (m, 3H), 7.86-7.75 (m, 3H), 3.17-3.07 (m, 4H), and 1.67-1.56 (m, 4H);); Retention Time=5.877 min; HRMS: m/z (M+H)+=(Calculated for C₁₇H₁₇BrClN₂O₃S, 444.9804) found, 444.9805.

### 3-Bromo-4-iodo-N-(4-(pyrrolidin-1-ylsulfonyl)phenyl)benzamide (35)

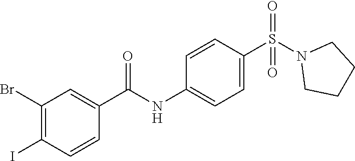 [SMILES: O=C(Nc1ccc(S(=O)(=O)N2CCCC2)cc1)c1ccc(I)c(Br)c1]

Synthesize as in Method A using 4-(pyrrolidin-1-ylsulfonyl)aniline as the starting material and 3-bromo-4-iodobenzoyl chloride. ¹H NMR (400 MHz, DMSO-d₆) δ 10.69 (s, 1H), 8.25 (d, J=2.0 Hz, 1H), 8.12 (d, J=8.2 Hz, 1H), 8.02-7.94 (m, 2H), 7.82-7.74 (m, 2H), 7.65 (dd, J=2.1, and 8.2 Hz, 1H), 3.17-3.06 (m, 4H), and 1.67-1.55 (m, 4H); Retention Time=5.026 min; HRMS: m/z (M+H)+=(Calculated for C₁₇H₁₇BrIN₂O₃S, 536.9163) found, 536.9158.

### 4-Chloro-3-fluoro-N-(4-(pyrrolidin-1-ylsulfonyl)phenyl)benzamide (38)

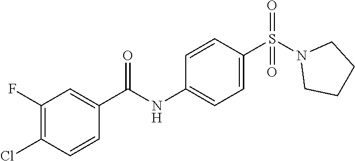 [SMILES: O=C(Nc1ccc(S(=O)(=O)N2CCCC2)cc1)c1ccc(Cl)c(F)c1]

Synthesize as in Method A using 4-(pyrrolidin-1-ylsulfonyl)aniline as the starting material and 3-fluoro-4-chlorobenzoyl chloride. NMR (400 MHz, DMSO-d₆) δ 10.69 (s, 1H), 8.03-7.94 (m, 3H), 7.86-7.74 (m, 4H), 3.17-3.07 (m, 4H), 1.68-1.55 (m, 4H); Retention Time=5.355 min; HRMS: m/z (M+H)+=(Calculated for C₁₇H₁₇ClFN₂O₃S, 383. 0627) found, 383.0620.

### 3-Bromo-4-chloro-N-(4-(pyrrolidin-1-ylsulfonyl)phenyl)benzamide (41)

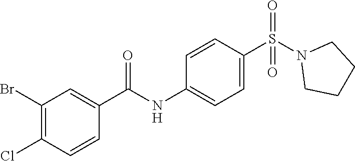 [SMILES: O=C(Nc1ccc(S(=O)(=O)N2CCCC2)cc1)c1ccc(Cl)c(Br)c1]

Synthesize as in Method A using 4-(pyrrolidin-1-ylsulfonyl)aniline as the starting material and 3-bromo-4-chlorobenzoyl chloride. ¹H NMR (400 MHz, DMSO-d₆) δ 10.71 (s, 1H), 8.33 (d, J=2.0 Hz, 1H), 8.03-7.92 (m, 3H), 7.85-7.75 (m, 3H), 3.11-3.05 (m, 4H), and 1.68-1.56 (m, 4H); Retention Time=5.879 min; HRMS: m/z (M+H)+=(Calculated for C17H₁₇BrClN₂O₃S, 444.9804) found, 444.9825.

### 3-Iodo-4-methoxy-N-(3-(pyrrolidin-1-ylsulfonyl)phenyl)benzamide (23)

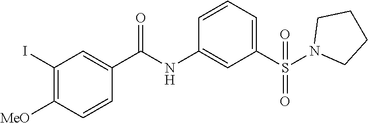 [SMILES: COc1ccc(C(=O)Nc2cccc(S(=O)(=O)N3CCCC3)c2)cc1I]

Synthesize using Method A using 3-(pyrrolidin-1-ylsulfonyl)aniline as the starting material. ¹H NMR (400 MHz, DMSO-d₆) δ 10.43 (s, 1H), 8.41 (d, J=2.2 Hz, 1H), 8.24 (t, J=1.9 Hz, 1H), 8.12-7.99 (m, 2H), 7.58 (t, J=8.0 Hz, 1H), 7.48 (ddd, J=1.0, 1.8, and 7.9 Hz, 1H), 7.13 (d, J=8.7 Hz, 1H), 3.90 (s, 3H), 3.18-3.10 (m, 4H), and 1.68-1.58 (m, 4H); Retention Time=5.629 min; HRMS: m/z (M+H)+=(Calculated for C₁₈H₂₀IN₂O₄S, 487.0183) found, 487.0180.

### 3-Iodo-4-methoxy-N-(4-(N-(thiazol-2-yl)sulfamoyl)phenyl)benzamide (17)

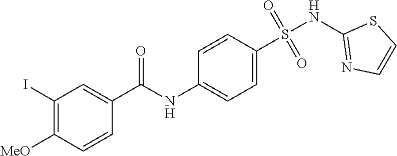 [SMILES: COc1ccc(C(=O)Nc2ccc(S(=O)(=O)Nc3nccs3)cc2)cc1I]

Synthesize using Method A using 4-amino-N-(thiazol-2-yl)benzene sulfonamide. ¹H NMR (400 MHz, DMSO-d₆) δ 12.65 (s, 1H), 10.41 (s, 1H), 8.37 (d, J=2.2 Hz, 1H), 8.00 (dd, J=2.2, and 8.6 Hz, 1H), 7.93-7.83 (m, 2H), 7.79-7.69 (m, 2H), 7.22 (d, J=4.6 Hz, 1H), 7.12 (d, J=8.8 Hz, 1H), 6.79 (d, J=4.6 Hz, 1H), and 3.89 (s, 3H); Retention Time=4.663 min; HRMS: m/z (M+Na)+=(Calculated for C₁₇H₁₄IN₃NaO₄S₂, 537.9363) found, 537.9377.

### 3,4-Dimethoxy-N-(4-(pyrrolidin-1-ylsulfonyl)phenyl)benzamide (53)

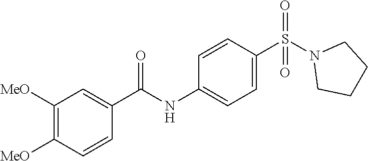 [SMILES: COc1ccc(C(=O)Nc2ccc(S(=O)(=O)N3CCCC3)cc2)cc1OC]

Synthesize as in Method A using 4-(pyrrolidin-1-ylsulfonyl)aniline as the starting material and 3,4-dimethoxybenzoyl chloride. ¹H NMR (400 MHz, DMSO-d₆) δ 10.40 (s, 1H), 8.03-7.95 (m, 2H), 7.81-7.72 (m, 2H), 7.61 (dd, J=2.1, and 8.4 Hz, 1H), 7.50 (d, J=2.1 Hz, 1H), 7.08 (d, J=8.5 Hz, 1H), 3.82 (d, J=1.2 Hz, 6H), 3.16-3.07 (m, 4H), and 1.67-1.56 (m, 4H); Retention Time=4.718 min; HRMS: m/z (M+Na)+=(Calculated for C₁₉H₂₂N₂NaO₅S, 413.1142) found, 413.1148.

### 4-Bromo-3-methyl-N-(4-(pyrrolidin-1-ylsulfonyl)phenyl)benzamide (56)

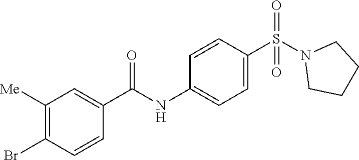 [SMILES: Cc1cc(C(=O)Nc2ccc(S(=O)(=O)N3CCCC3)cc2)ccc1Br]

Synthesize as in Method A using 4-(pyrrolidin-1-ylsulfonyl)aniline as the starting material and 4-bromo-5-methylbenzoyl chloride. ¹H NMR (400 MHz, DMSO-d₆): δ 10.62 (s, 1H), 8.03-7.95 (m, 2H), 7.94-7.89 (m, 1H), 7.82-7.72 (m, 3H), 7.69 (ddd, J=0.6, 2.3, and 8.3 Hz, 1H), 3.20-3.00 (m, 4H), 2.42 (d, J=0.6 Hz, 3H), 1.67-1.55 (m, 4H); Retention Time=5.766 min; HRMS: m/z (M+H)+=(Calculated for C₁₈H₂₀BrN₂O₃S, 423.0373) found, 423.0380.

### 4-Bromo-3-ethyl-N-(4-(pyrrolidin-1-ylsulfonyl)phenyl)benzamide (62)

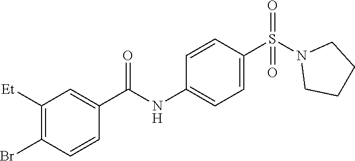 [SMILES: CCc1cc(C(=O)Nc2ccc(S(=O)(=O)N3CCCC3)cc2)ccc1Br]

Synthesize as in Method A using 4-(pyrrolidin-1-ylsulfonyl)aniline as the starting material and 4-bromo-5-ethylbenzoyl chloride. ¹H NMR (400 MHz, DMSO-d₆) δ 10.61 (s, 1H), 8.03-7.95 (m, 2H), 7.92-7.84 (m, 1H), 7.82-7.65 (m, 4H), 3.11 (td, J=2.3, and 4.7 Hz, 4H), 2.77 (q, J=7.5 Hz, 2H), 1.67-1.56 (m, 4H), and 1.25-1.09 (m, 3H). Retention Time=6.053 min; HRMS: m/z (M+H)+=(Calculated for C₁₉H₂₂BrN₂O₃S, 439.0510) found, 439.0522.

### 3-Isopropyl-4-methoxy-N-(4-(pyrrolidin-1-ylsulfonyl)phenyl) benzamide (36)

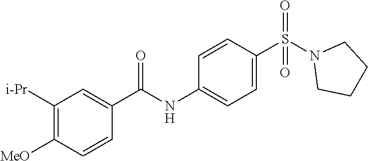 [SMILES: COc1ccc(C(=O)Nc2ccc(S(=O)(=O)N3CCCC3)cc2)cc1C(C)C]

Synthesize as in Method A using 4-(pyrrolidin-1-ylsulfonyl)aniline as the starting material and 3-isopropyl-4-methoxybenzoyl chloride. ¹H NMR (400 MHz, DMSO-d₆): δ 10.38 (s, 1H), 8.08-7.91 (m, 2H), 7.85 (dd, J=2.3, and 8.6 Hz, 1H), 7.81-7.78 (m, 1H), 7.78-7.74 (m, 2H), 7.06 (dd, J=8.6, and 11.1 Hz, 1H), 3.86 (s, 3H), 3.26-3.20 (m, 1H), 3.21-2.99 (m, 4H), 1.76-1.43 (m, 4H), 1.19 and (d, J=6.9 Hz, 6H); Retention Time=5.865 min; HRMS: m/z (M+H)+=(Calculated for C₂₁H₂₇N₂O₄S, 403.1698) found, 403.1698.

### 6-Methoxy-N-(4-(pyrrolidin-1-ylsulfonyl)phenyl)-[1,1′-biphenyl]-3-carboxamide (65)

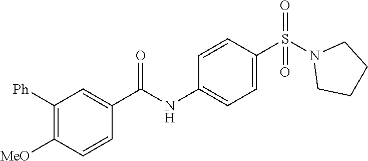 [SMILES: COc1ccc(C(=O)Nc2ccc(S(=O)(=O)N3CCCC3)cc2)cc1-c1ccccc1]

Synthesize as in Method A using 4-(pyrrolidin-1-ylsulfonyl)aniline as the starting material and 3-phenyl-4-methoxybenzoyl chloride. ¹H NMR (400 MHz, DMSO-d₆) δ 10.47 (s, 1H), 8.05-7.97 (m, 3H), 7.97-7.93 (m, 1H), 7.79-7.71 (m, 2H), 7.55-7.48 (m, 2H), 7.47-7.38 (m, 3H), 7.38-7.31 (m, 1H), 7.25 (d, J=8.8 Hz, 1H), 3.82 (s, 3H), 3.19-2.98 (m, 4H), and 1.72-1.38 (m, 4H); Retention Time=5.810 min; HRMS: m/z (M+H)+=(Calculated for C₂₁H₂₇N₂O₄S, 437.1530) found, 437.1544.

### 4-Methoxy-3-methyl-N-(4-(pyrrolidin-1-ylsulfonyl)phenyl)benzamide (68)

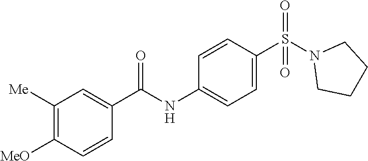 [SMILES: COc1ccc(C(=O)Nc2ccc(S(=O)(=O)N3CCCC3)cc2)cc1C]

Synthesize as in Method A using 4-(pyrrolidin-1-ylsulfonyl)aniline as the starting material and 3-methyl-4-methoxybenzoyl chloride. ¹H NMR (400 MHz, DMSO-d₆) δ 10.38 (s, 1H), 8.03-7.96 (m, 2H), 7.88-7.71 (m, 4H), 7.06 (d, J=8.6 Hz, 1H), 3.85 (s, 3H), 3.15-3.06 (m, 4H), 2.20 (s, 3H), and 1.69-1.56 (m, 4H). Retention Time=5.680 min; HRMS: m/z (M+H)+=(Calculated for C₁₉H₂₃N₂O₄S, 375.1373) found, 375.1359.

### 3-Cyclohexyl-4-methoxy-N-(4-(pyrrolidin-1-ylsulfonyl)phenyl) benzamide (46)

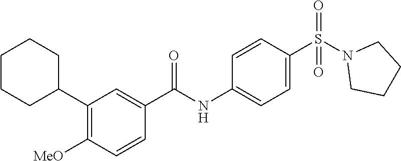 [SMILES: COc1ccc(C(=O)Nc2ccc(S(=O)(=O)N3CCCC3)cc2)cc1C1CCCCC1]

Method B. Starting with 3-iodo-4-methoxy-N-(4-(pyrrolidin-1-ylsulfonyl)phenyl)benzamide (0.05 g, 0.10 mmol), PdOAc₂ (2.00 mg, 10.28 μmol) and C-Phos (5.00 mg, 10.30 mmol) in degassed THF slowly add cyclohexylzinc(II) bromide (1.00 mL, 0.51 mmol). This mixture was stirred at rt until no starting material was observed by HPLC (1.0 h). The reaction was quenched with the addition of NH4Cl and extracted with EtOAc. A scavenger was added to the organic layer and stir for 6 h. The scavenger was filter concentrate and turn in for purification. ¹H NMR (400 MHz, DMSO-d₆): δ 10.37 (s, 1H), 8.03-7.95 (m, 2H), 7.84 (dd, J=2.3, and 8.6 Hz, 1H), 7.79-7.73 (m, 3H), 7.07 (d, J=8.7 Hz, 1H), 3.84 (s, 3H), 3.16-3.05 (m, 4H), 2.91 (t, J=6.8 Hz, 1H), 1.74 (q, J=14.8 Hz, 6H), 1.65-1.58 (m, 4H), and 1.49-1.16 (m, 4H); Retention Time=7.062 min; HRMS: m/z (M+H)+=(Calculated for C₂₄H₃₁N₂O₄S, 443.1999) found, 443.2004.

### 3-Cyclobutyl-4-methoxy-N-(4-(pyrrolidin-1-ylsulfonyl)phenyl) benzamide (49)

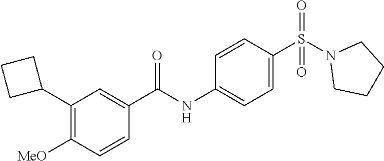 [SMILES: COc1ccc(C(=O)Nc2ccc(S(=O)(=O)N3CCCC3)cc2)cc1C1CCC1]

Synthesize as seen 3-iodo-4-methoxy-N-(4-(pyrrolidin-1-ylsulfonyl)phenyl)benzamide and follow Method B using cyclobutylzinc(II) bromide instead of cyclohexylzinc(II) bromide. ¹H NMR (400 MHz, DMSO-d₆): δ 10.43 (s, 1H), 8.04-7.96 (m, 2H), 7.85 (ddd, J=0.5, 2.4, and 8.5 Hz, 1H), 7.81-7.72 (m, 3H), 7.04 (d, J=8.7 Hz, 1H), 3.82 (s, 3H), 3.67 (p, J=8.7 Hz, 1H), 3.11 (td, J=3.6, 5.6, and 6.8 Hz, 4H), 2.33-2.18 (m, 2H), 2.17-1.87 (m, 3H), 1.83-1.73 (m, 1H), and 1.68-1.51 (m, 4H); Retention Time=6.493 min; HRMS: m/z (M+H)+=(Calculated for C₂₂H₂₇N₂O₄S, 415.1686) found, 415.1687.

### 3-Cyclopentyl-4-methoxy-N-(4-(pyrrolidin-1-ylsulfonyl)phenyl) benzamide (55)

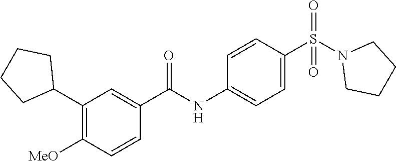 [SMILES: COc1ccc(C(=O)Nc2ccc(S(=O)(=O)N3CCCC3)cc2)cc1C1CCCC1]

Synthesize as seen 3-iodo-4-methoxy-N-(4-(pyrrolidin-1-ylsulfonyl)phenyl)benzamide and follow Method B using cyclopentylzinc(II) bromide instead of cyclohexylzinc(II) bromide. ¹H NMR (400 MHz, DMSO-d₆): δ 10.39 (s, 1H), 8.02-7.95 (m, 2H), 7.88-7.71 (m, 4H), 7.07 (d, J=8.7 Hz, 1H), 3.85 (s, 3H), 3.33-3.20 (m, 2H), 3.15-3.06 (m, 5H), 1.96 (s, 2H), 1.76 (q, J=3.3 Hz, 1H), and 1.74-1.49 (m, 7H); Retention Time=6.919 min; FIRMS: m/z (M+H)+=(Calculated for C₂₃H₂₉N₂O₄S, 429.1843) found, 429.1833.

### 4-Methoxy-3-(1-methylpiperidin-4-yl)-N-(4-(pyrrolidin-1-ylsulfonyl)phenyl)benzamide, TFA (58)

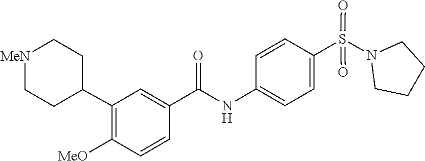 [SMILES: COc1ccc(C(=O)Nc2ccc(S(=O)(=O)N3CCCC3)cc2)cc1C1CCN(C)CC1]

Synthesize as seen 3-iodo-4-methoxy-N-(4-(pyrrolidin-1-ylsulfonyl)phenyl)benzamide and follow Method B using (1-methylpiperidin-4-yl)zinc(II) bromide instead of cyclohexylzinc(II)bromide. ¹H NMR (400 MHz, DMSO-d₆): δ 10.46 (s, 1H), 8.03-7.90 (m, 3H), 7.81-7.68 (m, 3H), 7.21-7.10 (m, 1H), 3.88 (s, 3H), 3.50 (d, J=12.1 Hz, 2H), 3.22-2.99 (m, 5H), 2.79 (d, J=4.6 Hz, 3H), 2.03-1.73 (m, 4H), and 1.69-1.52 (m, 4H); Retention Time=3.920 min; HRMS: m/z (M+H)+=(Calculated for C₂₄H₃₂N₃O₄S, 458.2108) found, 458.2126.

### 3-Iodo-4-methoxy-N-(4-((2-methylpiperidin-1-yl)sulfonyl)phenyl) benzamide (39)

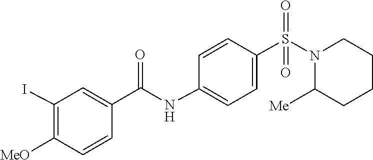 [SMILES: COc1ccc(C(=O)Nc2ccc(S(=O)(=O)N3CCCCC3C)cc2)cc1I]

To a stirred solution of 2-methylpiperidine (0.48 mL, 4.72 mmol) in pyridine (2.10 mL, 25.70 mmol) the 4-acetamidobenzene-1-sulfonyl chloride (1.00 g, 4.28 mmol) was added slowly. The reaction was heated for 3 h at 100° C., then let stir overnight at rt. Concentrated crude reaction, dissolved residue in EtOAc, and washed with 1N HCl (1×). Extract the acidic layer with EtOAc (2×'s), combined the organic layers and washed with saturated bicarb, and brine. Dried the organic layer with MgSO₄, filtered, concentrated, and used as is in the next reaction. The glass like oil was taken up in methanol (21.0 mL), treated with 4 M HCl/dioxanes (3 mL), and heated to reflux for 2 h. Let reaction mixture cool to rt and concentrate to a glass like oil which was used as is in the next reaction. 4-((2-methylpiperidin-1-yl)sulfonyl)aniline (1 equiv) was treated with DIPEA (3 equiv) in DCM (0.2M) and 1 M solution of 3-iodo-4-methoxybenzoyl chloride (1.5 equiv) in DCM was added to the reaction at rt. This mixture was allowed to stir overnight and was quenched after 18 hr with MeOH. The reaction was concentrated and purified to give the targeted compound. The enantiomers were separated using CHIRALPAK AS column, at 35 mL/min, isocratic MeOH, to give ee's of >99% for the positive, and 98.7% of the negative compound. ¹H NMR (400 MHz, DMSO-d₆): δ 10.46 (s, 1H), 8.38 (d, J=2.2 Hz, 1H), 8.04-7.90 (m, 3H), 7.79-7.70 (m, 2H), 7.13 (d, J=8.8 Hz, 1H), 4.07 (d, J=6.3 Hz, 1H), 3.90 (s, 3H), 3.57 (d, J=10.5 Hz, 1H), 2.93 (td, J=2.7, and 13.0 Hz, 1H), 1.56-1.34 (m, 5H), 1.25-1.10 (m, 1H), and 0.97 (d, J=6.9 Hz, 3H); Retention Time=6.329 min; HRMS: m/z (M+H)+=(Calculated for C₂₀H₂₄IN₂O₄S, 515.0496) found, 515.0491.

### 3-Iodo-4-methoxy-N-(3-(piperidin-1-ylsulfonyl)phenyl)benzamide (42)

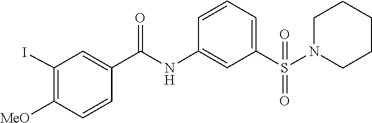 [SMILES: COc1ccc(C(=O)Nc2cccc(S(=O)(=O)N3CCCCC3)c2)cc1I]

Starting with commercially available 3-(piperidin-1-ylsulfonyl)aniline and freshly made 3-iodo-4-methoxybenzoyl chloride 1 M solution. Follow procedure for above compound (39). H NMR (400 MHz, DMSO-d₆): δ 10.44 (s, 1H), 8.41 (d, J=2.2 Hz, 1H), 8.17 (t, J=1.9 Hz, 1H), 8.12-7.98 (m, 2H), 7.58 (t, J=8.0 Hz, 1H), 7.40 (ddd, J=1.0, 1.8, and 7.8 Hz, 1H), 7.13 (d, J=8.8 Hz, 1H), 3.90 (s, 3H), 3.34 (s, 2H), 2.92-2.78 (m, 4H), and 1.53 (p, J=5.6 Hz, 5H);); Retention Time=6.076 min; HRMS: m/z (M+H)+=(Calculated for C₁₉H₂₁IN₂O₄S, 501.0339) found, 501.0356.

### 3,4-Dibromo-N-(4-((2-methylpiperidin-1-yl)sulfonyl)phenyl)benzamide

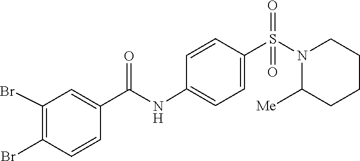 [SMILES: CC1CCCCN1S(=O)(=O)c1ccc(NC(=O)c2ccc(Br)c(Br)c2)cc1]

Synthesize using Method C: 4-((2-methylpiperidin-1-yl)sulfonyl)aniline HCl (1 equiv) was stirred with DIPEA (3 equiv), in DCM (0.2M) before the addition of 3,4-dibromobenzoyl chloride as the acid chloride as a 1 M solution in DCM. The reaction mixture was stirred overnight and quenched with MeOH when reaction was complete. The dibromobenzoyl chloride was synthesized the same as previously described for the 3-iodo-4-methyoxybenzolyl chloride. ¹H NMR (400 MHz, DMSO-d₆): δ 10.69 (s, 1H), 8.30 (d, J=2.1 Hz, 1H), 7.89 (m, 3H), 7.84 (dd, J=2.1, and 8.3 Hz, 1H), 7.81-7.70 (m, 2H), 4.07 (dd, J=3.7, and 7.3 Hz, 1H), 3.57 (dd, J=3.8, and 13.4 Hz, 1H), 2.92 (td, J=2.6, and 13.0 Hz, 1H), 1.53-1.33 (m, 5H), 1.23-1.09 (m, 1H), and 0.96 (d, J=6.9 Hz, 3H); Retention Time=6.524 min; HRMS: m/z (M+H)⁺=(Calculated for C₁₉H₂₁Br₂N₂O₃S, 518.9596) found, 518.9598.

### 6-Chloro-5-methoxy-N-(4-((2-methylpiperidin-1-yl)sulfonyl)phenyl) picolinamide, NH₄⁺ (100)

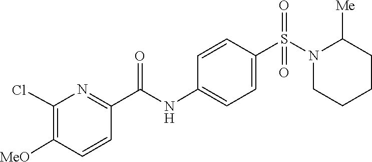 [SMILES: COc1ccc(C(=O)Nc2ccc(S(=O)(=O)N3CCCCC3C)cc2)nc1Cl]

Synthesize using Method C, and synthesize the acid chloride using 6-chloro-5-methoxypicolinic acid (0.13 g, 0.47 mmol), and oxalyl chloride (0.10 mL, 1.14 mmol) was stirred in DCM (0.50 mL) at rt before DMF (2.0 μL, 0.02 mmol) was added. The mixture was stirred at rt for 72 h, at which time the reaction was concentrate to a white solid. The white solid was used as is in the next reaction by making a 1 M solution in dry DCM. 4-((2-methylpiperidin-1-yl)sulfonyl)aniline (1.0 equiv) was treated with DIPEA (3.0 equiv) in DCM (0.2M) and 1 M solution of 3-chloro-4-methoxybenzoyl chloride (1.5 equiv) in DCM was added to the reaction at rt. This mixture was allowed to stir overnight and was quenched after 18 h with MeOH. The reaction was concentrated and purified to give the targeted compound. ¹H NMR (400 MHz DMSO-d₆): δ 10.61 (s, 1H), 8.16-8.03 (m, 3H), 7.80-7.71 (m, 3H), 4.08 (dq, J=3.9, and 7.5 Hz, 1H), 3.98 (s, 3H), 3.62-3.53 (m, 1H), 2.94 (td, J=2.7, and 13.1 Hz, 1H), 1.59-1.30 (m, 5H), 1.26-1.07 (m, 1H), and 0.98 (d, J=6.9 Hz, 3H); Retention Time=6.017 min; HRMS: m/z (M+H)+=(Calculated for C₂₀H₂₄IN₂O₄S 515.0496) found, 515.0491.

### 6-Iodo-5-methoxy-N-(4-((2-methylpiperidin-1-yl)sulfonyl)phenyl)picolinamide, TFA (102)

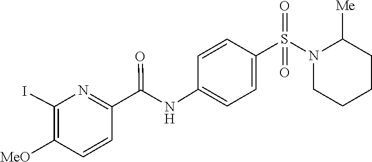 [SMILES: COc1ccc(C(=O)Nc2ccc(S(=O)(=O)N3CCCCC3C)cc2)nc1I]

Synthesize using Method C, followed by an amide coupling. 4-((2-methylpiperidin-1-yl)sulfonyl)aniline, HCl (0.11 g, 0.37 mmol), 6-iodo-5-methoxypicolinic acid (0.10 g, 0.37 mmol), propane phosphonic acid anhydride in DMF (0.35 mL, 0.55 mmol), and TEA (0.15 mL, 1.10 mmol) was heated to 60° C. for 2 hr. in DMF (1.80 mL). The reaction mixture was cooled to rt, poured into EtOAc, and washed with saturated NaHCO₃, and brine. The organic layer was dried over MgSO4, filtered, concentrated, and purified to give the desired compound. ¹H NMR (400 MHz DMSO-d₆): δ 10.54 (s, 1H), 8.11-8.01 (m, 3H), 7.79-7.71 (m, 2H), 7.50 (d, J=8.6 Hz, 1H), 4.07 (td, J=3.7, and 7.1 Hz, 1H), 3.95 (s, 3H), 3.29 (s, 2H), 2.93 (td, J=2.7, and 13.1 Hz, 1H), 1.49 (dd, J=3.7, and 12.4 Hz, 1H), 1.48-1.33 (m, 1H), 1.37 (s, 2H), 1.23-1.11 (m, 1H), and 0.97 (d, J=6.9 Hz, 3H); Time=6.128 min; HRMS: m/z (M+H)+=(Calculated for C₁₉H₂₃IN₃O₄S, 516.0448) found, 516.0438.

### 3-Iodo-4-methoxy-N-(4-(thiomorpholinosulfonyl)phenyl)benzamide (51)

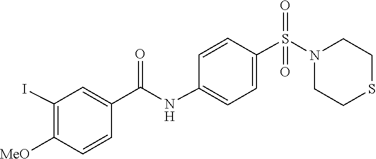 [SMILES: COc1ccc(C(=O)Nc2ccc(S(=O)(=O)N3CCSCC3)cc2)cc1I]

Synthesize using Method C and thiomorpholine as the starting material. ¹H NMR (400 MHz, DMSO-d₆): δ 10.52 (s, 1H), 8.39 (d, J=2.2 Hz, 1H), 8.05-7.97 (m, 3H), 7.74-7.67 (m, 2H), 7.13 (d, J=8.8 Hz, 1H), 3.90 (s, 3H), 3.17 (dd, J=3.7, and 6.4 Hz, 4H), and 2.68-2.60 (m, 4H); Retention Time=5.853 min; HRMS: m/z (M+H)+=(Calculated for C₁₈H₂₀IN₂O₄S₂, 518.9904) found, 518.9924.

### 3-Iodo-4-methoxy-N-(4-((3-methylthiomorpholino)sulfonyl)phenyl) benzamide (92)

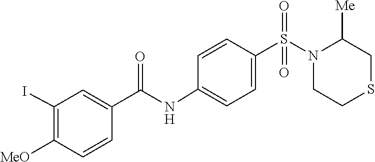 [SMILES: COc1ccc(C(=O)Nc2ccc(S(=O)(=O)N3CCSCC3C)cc2)cc1I]

Synthesize using Method C and 3-methylthiomorpholine as the starting material. ¹H NMR (400 MHz, DMSO-d₆): δ 10.49 (s, 1H), 8.38 (d, J=2.2 Hz, 1H), 8.05-7.93 (m, 3H), 7.80-7.73 (m, 2H), 7.13 (d, J=8.7 Hz, 1H), 4.27 (tq, J=3.4, and 6.7 Hz, 1H), 3.90 (s, 4H), 3.34-3.21 (m, 1H), 3.19-3.07 (m, 1H), 2.81-2.72 (m, 1H), 2.43 (s, 1H), 2.34 (dt, J=2.2, and 13.6 Hz, 1H), and 1.10 (dd, J=0.6, and 6.7 Hz, 3H); Retention Time=6.031 min; HRMS: m/z (M+H)+=(Calculated for C₁₉H₂₂IN₂O₄S₂, 531.0060) found, 531.0070.

### 4-((4-(3-Iodo-4-methoxybenzamido)phenyl)sulfonyl) thiomorpholine-3-carboxylic acid (98)

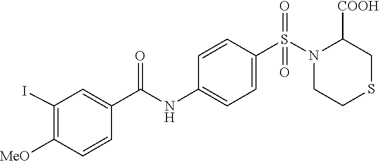 [SMILES: COc1ccc(C(=O)Nc2ccc(S(=O)(=O)N3CCSCC3C(=O)O)cc2)cc1I]

Synthesize using Method C and ethyl thiomorpholine-3-carboxylate, HCl as the starting material. The final step was a basic hydrolysis from the ester to the acid using 1N LiOH/EtOH (1:1) heated to 60° C. for 5 hr. ¹H NMR (400 MHz, DMSO-d₆): δ 13.07 (s, 1H), 10.47 (s, 1H), 8.39 (d, J=2.2 Hz, 1H), 8.05-7.90 (m, 3H), 7.81-7.72 (m, 2H), 7.13 (d, J=8.8 Hz, 1H), 4.86 (d, J=3.7 Hz, 1H), 3.90 (s, 4H), 3.36 (ddd, J=5.7, 9.4, and 14.4 Hz, 1H), 3.14 (d, J=5.1 Hz, 1H), 2.95-2.86 (m, 1H), 2.76 (dd, J=4.1, and 13.7 Hz, 1H), and 2.48-2.41 (m, 1H); Retention Time=5.191 min; HRMS: m/z (M+H)+=(Calculated for C₁₉H₂₀IN₂O₆S₂, 562.9802) found, 562.9795.

### 4-((4-(3-Iodo-4-methoxybenzamido)phenyl)sulfonyl)thiomorpholine-3-carboxamide (77)

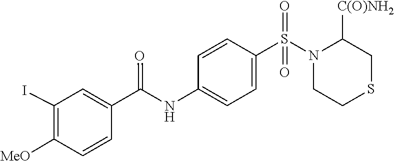

Synthesize using Method C and ethyl thiomorpholine-3-carboxylate, follow the procedure to make the carboxylic acid. 4-((4-(3-Iodo-4-methoxybenzamido)phenyl) sulfonyl) thiomorpholine-3-carboxylic acid (65.0 mg, 0.12 mmol), was treated with HOBt (18.0 mg, 0.12 mmol), ammonium hydroxide (52 uL, 0.52 mmol), and EDC (100 mg, 0.52 mmol) in DMF (600 uL) and stirred for 5 h at rt. When the starting material was consumed the reaction was concentrated and purified to give the desired material. ¹H NMR (400 MHz, DMSO-d₆): δ 10.48 (s, 1H), 8.39 (d, J=2.2 Hz, 1H), 8.05-7.92 (m, 3H), 7.83-7.74 (m, 2H), 7.25 (d, J=11.5 Hz, 2H), 7.14 (d, J=8.8 Hz, 1H), 4.64 (s, 1H), 3.99-3.91 (m, 1H), 3.90 (s, 3H), 3.51 (ddd, J=6.3, 8.9, and 14.5 Hz, 1H), 2.93 (dd, J=2.8, and 14.1 Hz, 1H), 2.60 (dd, J=4.2, and 13.9 Hz, 1H), and 2.41-2.30 (m, 2H); Retention Time=4.938 min; HRMS: m/z (M+H)+=(Calculated for C₁₉H₂₁IN₃O₅S₂, 561.9962) found, 561.9943.

### 4-((4-(3-Iodo-4-methoxybenzamido)phenyl)sulfonyl)-N-methylthiomorpholine-3-carboxamide (72)

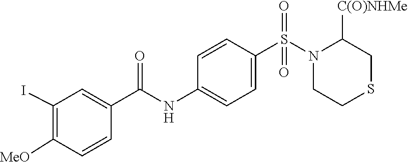

Synthesize using Method C and procedure for the synthesis of 4-((4-(3-Iodo-4-methoxybenzamido)phenyl)sulfonyl)thiomorpholine-3-carboxylic acid. 4-((4-(3-iodo-4-methoxybenzamido)phenyl)sulfonyl)thiomorpholine-3-carboxylic acid (35.0 mg, 0.062 mmol), TEA (30 uL, 0.19 mmol), HOBt (10 mg, 0.063 mmol), and methylamine hydrochloride (9.0 mg, 0.13 mmol) were stirred in DMF (0.500 mL), at rt before the addition of HATU (35.0 mg, 0.09 mmol). This reaction mixture was stirred for 18 h and diluted with EtOAc, and saturated NaHCO₃ to quench and separated. The organic layer was washed with brine, dried over MgSO4, filtered, and concentrated to give the desired product which was purified. ¹H NMR (400 MHz, DMSO-d₆): δ 10.49 (s, 1H), 8.39 (d, J=2.2 Hz, 1H), 8.06-7.93 (m, 4H), 7.85-7.75 (m, 4H), 7.14 (d, J=8.8 Hz, 1H), 4.65 (s, 1H), 4.00 (dt, J=3.1, and 14.4 Hz, 1H), 3.90 (s, 3H), 3.45 (ddd, J=4.1, 11.0, and 14.8 Hz, 1H), 2.90 (dt, J=2.4, and 13.5 Hz, 1H), 2.59 (d, J=4.5 Hz, 4H), 2.51 (d, J=4.2 Hz, 1H), 2.33 (s, 1H), and 2.39-2.24 (m, 1H); Retention Time=5.134 min; HRMS: m/z (M+H)+=(Calculated for C₂₀H₂₃IN₃O₅S₂, 576.0118) found, 576.0142.

### 4-((4-(3-Iodo-4-methoxybenzamido)phenyl)sulfonyl)-N,N-dimethylthiomorpholine-3-carboxamide (74)

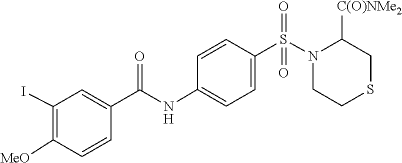

Synthesize using Method C and the procedure for 4-((4-(3-iodo-4-methoxybenzamido)phenyl)sulfonyl)-N-methylthiomorpholine-3-carboxamide. ¹H NMR (400 MHz, DMSO-d₆): δ 10.46 (s, 1H), 8.39 (d, J=2.2 Hz, 1H), 8.02 (dd, J=2.2, and 8.6 Hz, 1H), 7.97-7.89 (m, 2H), 7.74-7.67 (m, 2H), 7.13 (d, J=8.8 Hz, 1H), 5.07 (dd, J=3.4, and 4.6 Hz, 1H), 3.95 (ddd, J=3.6, 11.4, and 13.2 Hz, 1H), 3.90 (s, 3H), 3.79 (dt, J=3.4, and 13.2 Hz, 1H), 3.01 (s, 3H), 2.94-2.75 (m, 3H), 2.69 (s, 3H), and 2.56-2.40 (m, 1H); Retention Time=5.309 min; HRMS: m/z (M+Na)+=(Calculated for C₂₁H₂₄IN₃NaO₅S₂, 612.0094) found, 612.0109.

### Ethyl4-((4-(4-bromo-3-iodobenzamido)phenyl)sulfonyl)thiomorpholine-3-carboxylate (76)

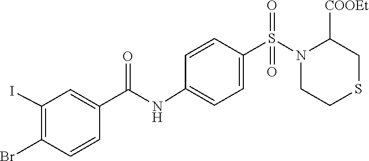 [SMILES: CCOC(=O)C1CSCCN1S(=O)(=O)c1ccc(NC(=O)c2ccc(Br)c(I)c2)cc1]

Synthesize using Method C with ethyl thiomorpholine-3-carboxylate as the starting material, and 4-bromo-3-iodobenzoyl chloride as the acid chloride. ¹H NMR (400 MHz, DMSO-d6): δ 10.67 (s, 1H), 8.46 (dd, J=0.6, and 1.9 Hz, 1H), 7.97-7.92 (m, 2H), 7.89-7.85 (m, 2H), 7.80-7.74 (m, 2H), 5.00 (t, J=3.5 Hz, 1H), 4.16-3.83 (m, 3H), 3.27-3.20 (m, 1H), 2.92 (dd, J=3.3, and 13.6 Hz, 1H), 2.80 (dd, J=4.0, and 13.9 Hz, 1H), 2.52-2.48 (m, 1H), and 1.09 (t, J=7.1 Hz, 3H); Retention Time=6.530 min; HRMS: m/z (M+H)+=(Calculated for C₂₀H₂₁BrIN₂O₅S₂, 638.9114) found, 638.9123.

### 4-((4-(4-bromo-3-iodobenzamido)phenyl)sulfonyl)thiomorpholine-3-carboxylic acid (105)

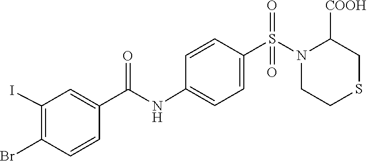 [SMILES: O=C(Nc1ccc(S(=O)(=O)N2CCSCC2C(=O)O)cc1)c1ccc(Br)c(I)c1]

Starting with ethyl 4-((4-(4-bromo-3-iodobenzamido)phenyl)sulfonyl)thiomorpholine-3-carboxylate (0.14 g, 0.22 mmol), in a 1M solution of LiOH (1.1 mL, 1.1 mmol) in EtOH (1.1 mL) was heated to 60° C. for 1.5 hr. The reaction was allowed to cool to room temperature and the pH adjusted to 1 with 1 N HCl to give the desired material at the carboxylic acid. The reaction mixture was concentrated and sent for reverse phase purification. ¹H NMR (400 MHz, DMSO-d₆) δ: 13.14 (bs, 1H), 10.66 (s, 1H), 8.45 (d, J=1.9 Hz, 1H), 7.97-7.81 (m, 4H), 7.82-7.73 (m, 2H), 4.84 (s, 1H), 3.89 (d, J=13.9 Hz, 1H), 2.90 (dd, J=2.7, and 13.6 Hz, 1H), 2.74 (dd, J=4.1, and 13.7 Hz, 1H), and 2.47-2.38 (m, 2H); Retention Time=5.616 min; HRMS: m/z (M+H)+=(Calculated for C₁₈H₁₇BrIN₂O₅S₂, 612.8781) found, 612.8781.

### 4-((4-(4-Bromo-3-iodobenzamido)phenyl)sulfonyl)thiomorpholine-3-carboxamide (108)

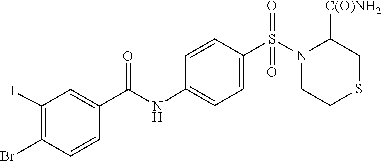

Synthesize using Method C and thiomorpholine-3-carboxamide as the starting material, and 4-bromo-3-iodobenzoyl chloride as the acid chloride. ¹H NMR (400 MHz, DMSO-d6): δ 10.69 (s, 1H), 8.46 (dd, J=0.4, and 2.0 Hz, 1H), 7.99-7.76 (m, 6H), 7.28 (d, J=15.5 Hz, 2H), 4.64 (t, J=3.4 Hz, 1H), 3.99-3.91 (m, 1H), 3.50 (ddd, J=5.0, 10.1, and 14.6, Hz, 1H), 2.92 (dd, J=2.8, and 14.0, Hz, 1H), 2.63-2.49 (m, 1H), and 2.45-2.29 (m, 2H); Retention Time=5.298 min; HRMS: m/z (M+H)+=(Calculated for C₁₈H₁₈BrIN₃O₄S₂, 611.8941) found, 611.8936.

### 4-Bromo-3-iodo-N-(4-(thiomorpholinosulfonyl)phenyl)benzamide, TFA (71)

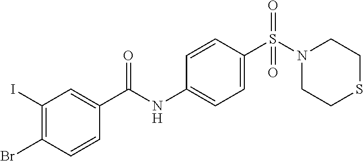 [SMILES: O=C(Nc1ccc(S(=O)(=O)N2CCSCC2)cc1)c1ccc(Br)c(I)c1]

Synthesize using Method C and thiomorpholine as the starting material, and 4-bromo-3-iodobenzoyl chloride as the acid chloride. ¹H NMR (400 MHz, DMSO-d₆): δ 10.71 (s, 1H), 8.46 (dd, J=0.5, and 2.0 Hz, 1H), 8.04-7.96 (m, 2H), 7.93-7.82 (m, 2H), 7.77-7.68 (m, 2H), 3.21-3.14 (m, 4H), and 2.68-2.60 (m, 4H); Retention Time=6.347 min; HRMS: m/z (M+H)+=(Calculated for C₁₇H₁₇BrIN₂O₃S₂, 568.8883) found, 568.8906.

### N-(4-((3-(tert-butyl)thiomorpholino)sulfonyl)phenyl)-3-iodo-4-methoxybenzamide (104)

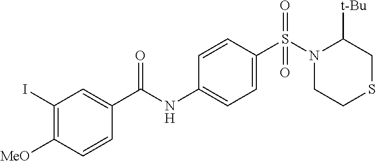 [SMILES: COc1ccc(C(=O)Nc2ccc(S(=O)(=O)N3CCSCC3C(C)(C)C)cc2)cc1I]

Synthesize using Method C and 3-(tert-butyl)thiomorpholine as the starting material, and 3-iodo-4-methoxybenzoyl chloride as the acid chloride. ¹H NMR (400 MHz DMSO-d₆): δ 10.50 (s, 1H), 8.39 (d, J=2.2 Hz, 1H), 8.05-7.90 (m, 3H), 7.91-7.78 (m, 2H), 7.13 (d, J=8.8 Hz, 1H), 4.03-3.93 (m, 1H), 3.89 (s, 3H), 3.38 (ddd, J=3.7, 12.3, and 15.7 Hz, 1H), 2.99 (s, 1H), 2.76-2.66 (m, 1H), 2.57-2.47 (m, 1H), 2.29 (d, J=14.3 Hz, 1H), 1.98 (td, J=4.6, and 12.6 Hz, 1H), and 1.01 (s, 9H); Retention Time=6.413 min; HRMS: m/z (M+H)+=(Calculated for C₂₂H₂₈IN₂O₄S₂, 575.0530) found, 575.0544.

### N-(4-((2-ethylthiomorpholino)sulfonyl)phenyl)-3-iodo-4-methoxybenzamide (103)

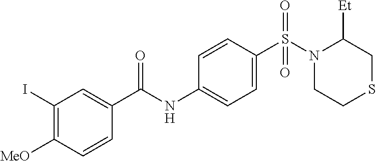 [SMILES: CCC1CSCCN1S(=O)(=O)c1ccc(NC(=O)c2ccc(OC)c(I)c2)cc1]

Synthesize using Method C and 2-ethylthiomorpholine as the starting material. ¹H NMR (400 MHz DMSO-d₆): δ 10.51 (s, 1H), 8.39 (d, J=2.2 Hz, 1H), 8.00 (dq, J=2.5, and 9.5, Hz, 3H), 7.76-7.67 (m, 2H), 7.13 (d, J=8.7 Hz, 1H), 3.90 (s, 3H), 3.61 (td, J=4.7, 11.5, and 12.1 Hz, 2H), 2.77-2.62 (m, 4H), 2.56-2.48 (m, 1H), 1.62-1.48 (m, 1H), 1.39 (dq, J=7.5, and 14.2 Hz, 1H), and 0.91 (t, J=7.4 Hz, 3H); Retention Time=6.149 min; HRMS: m/z (M+H)+=(Calculated for C₂₀H₂₄IN₂O₄S₂, 547.0217) found, 547.0224.

### N-(4-((2,3-dimethylthiomorpholino)sulfonyl)phenyl)-3-iodo-4-methoxybenzamide (106)

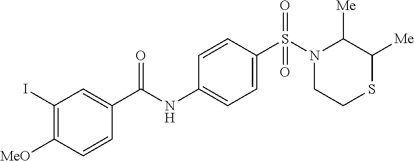 [SMILES: COc1ccc(C(=O)Nc2ccc(S(=O)(=O)N3CCSC(C)C3C)cc2)cc1I]

Synthesize using Method C and 2,3-dimethylthiomorpholine as the starting material. ¹H NMR (400 MHz, DMSO-d6,): δ 10.48 (s, 1H), 8.38 (d, J=2.2 Hz, 1H), 8.06-7.91 (m, 3H), 7.80-7.69 (m, 2H), 7.13 (d, J=8.8 Hz, 1H), 4.10 (qd, J=3.2, and 6.6 Hz, 1H), 3.89 (s, 3H), 3.85 (dt, J=3.2, and 14.0 Hz, 1H), 3.07-2.93 (m, 3H), 2.57 (td, J=3.3, 12.5, and 13.1 Hz, 1H), and 0.96 (dd, J=6.9, and 9.7 Hz, 6H); Retention Time=6.050 min; HRMS: m/z (M+H)+=(Calculated for C₂₀H₂₄IN₂O₄S₂, 547.0217) found, 547.0216.

### 3-Iodo-4-methoxy-N-(4-((2-methylthiomorpholino)sulfonyl)phenyl) benzamide (107)

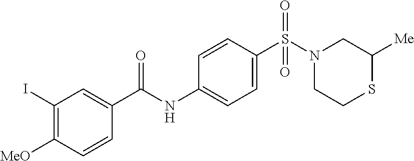 [SMILES: COc1ccc(C(=O)Nc2ccc(S(=O)(=O)N3CCSC(C)C3)cc2)cc1I]

Synthesize using Method C and 3-methylthiomorpholine as the starting material. ¹H NMR (400 MHz DMSO-d₆): δ 10.52 (s, 1H), 8.38 (d, J=2.1 Hz, 1H), 8.00 (dq, J=2.5, and 9.6 Hz, 3H), 7.75-7.67 (m, 2H), 7.13 (d, J=8.7 Hz, 1H), 3.90 (s, 3H), 3.73 (dd, J=3.0, and 12.3 Hz, 2H), 2.89 (ddt, J=3.5, 7.0, and 10.9 Hz, 1H), 2.78-2.64 (m, 2H), 2.60-2.49 (m, 1H), 2.30 (dd, J=9.6, and 12.2 Hz, 1H), and 1.10 (d, J=6.8 Hz, 3H); Retention Time=5.880 min; HRMS: m/z (M+H)+=(Calculated for C₁₉H₂₂IN₂O₄S₂, 533.0060) found, 533.0072.

### N-(4-((1,1-dioxidothiomorpholino)sulfonyl)phenyl)-3-iodo-4-methoxybenzamide (89)

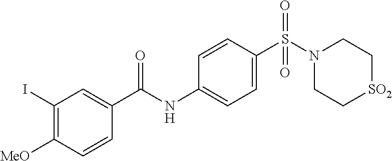 [SMILES: COc1ccc(C(=O)Nc2ccc(S(=O)(=O)N3CCS(=O)(=O)CC3)cc2)cc1I]

Synthesize using Method C and thiomorpholine 1,1-dioxide as the starting material. ¹H NMR (400 MHz, DMSO-d₆): δ 10.56 (s, 1H), 8.39 (d, J=2.2 Hz, 1H), 8.08-7.98 (m, 3H), 7.83-7.75 (m, 2H), 7.14 (d, J=8.8 Hz, 1H), 3.90 (s, 3H), 3.41 (dd, J=3.7, and 7.1 Hz, 4H), and 3.27-3.19 (m, 4H); Retention Time=5.177 min; HRMS: m/z (M+H)+=(Calculated for C₁₈H₂₀IN₂O₆S, 550.9802) found, 550.9811.

### N-(4-(N-cyclopropylsulfamoyl)phenyl)-3-iodo-4-methoxybenzamide (11)

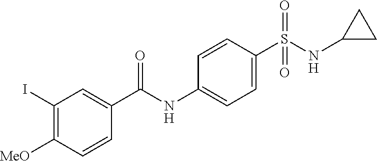 [SMILES: COc1ccc(C(=O)Nc2ccc(S(=O)(=O)NC3CC3)cc2)cc1I]

Synthesize using Method C and cyclopropanamine as the starting material. ¹H NMR (400 MHz, DMSO-d₆) δ 10.47 (s, 1H), 8.39 (d, J=2.2 Hz, 1H), 8.12-7.92 (m, 3H), 7.87-7.66 (m, 3H), 7.13 (d, J=8.7 Hz, 1H), 3.90 (s, 3H), 2.22-1.93 (m, 1H), and 0.56-0.03 (m, 4H); Retention Time=5.337 min; HRMS: m/z (M+H)+=(Calculated for C₁₇H₁₈IN₂O₄S, 473.0026) found, 473.0047.

### N-(4-(N,N-dipropylsulfamoyl)phenyl)-3-iodo-4-methoxybenzamide (48)

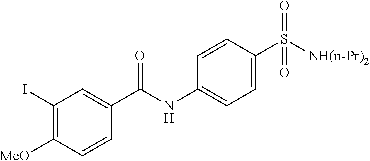

Synthesize using Method C and dipropylamine as the starting material. ¹H NMR (400 MHz, DMSO-d₆): δ 10.46 (s, 1H), 8.38 (d, J=2.2 Hz, 1H), 8.05-7.91 (m, 3H), 7.79-7.70 (m, 2H), 7.13 (d, J=8.8 Hz, 1H), 3.90 (s, 3H), 3.03-2.94 (m, 4H), 1.51-1.37 (m, 4H), and 0.79 (t, J=7.4 Hz, 6H); Retention Time=6.522 min; HRMS: m/z (M+H)+=(Calculated for C₂₀H₂₆IN₂O₄S, 517.0652) found, 517.0642.

### 3-Iodo-4-methoxy-N-(4-(N-(pentan-3-yl)sulfamoyl)phenyl)benzamide (15)

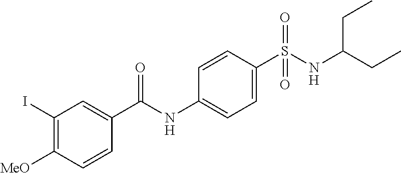 [SMILES: CCC(CC)NS(=O)(=O)c1ccc(NC(=O)c2ccc(OC)c(I)c2)cc1]

Synthesize using Method C and pentan-3-amine as the starting material. ¹H NMR (400 MHz, DMSO-d₆) δ 10.42 (s, 1H), 8.38 (d, J=2.2 Hz, 1H), 8.01 (dd, J=2.2, and 8.6 Hz, 1H), 7.95-7.87 (m, 2H), 7.78-7.69 (m, 2H), 7.35 (d, J=8.0 Hz, 1H), 7.13 (d, J=8.8 Hz, 1H), 3.89 (s, 3H), 2.90 (h, J=6.6 Hz, 1H), 1.39-1.13 (m, 4H), and 0.64 (t, J=7.4 Hz, 6H); Retention Time=5.971 min; HRMS: m/z (M+Na)+=(Calculated for C₁₉H₂₃IN₂NaO₄S, 525.0315) found, 525.0318.

### 3-Iodo-4-methoxy-N-(4-((2-(methoxymethyl)pyrrolidin-1-yl)sulfonyl)phenyl)benzamide (79)

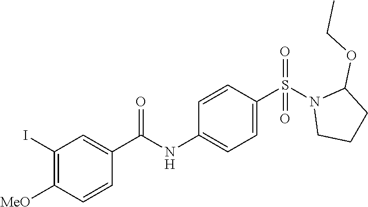 [SMILES: CCOC1CCCN1S(=O)(=O)c1ccc(NC(=O)c2ccc(OC)c(I)c2)cc1]

Synthesize using Method C and 2-(methoxymethyl)pyrrolidine as starting material. ¹H NMR (400 MHz, DMSO-d6,): δ 10.49 (s, 1H), 8.38 (d, J=2.2 Hz, 1H), 8.05-7.94 (m, 3H), 7.83-7.76 (m, 2H), 7.13 (d, J=8.8 Hz, 1H), 3.90 (s, 3H), 3.64 (tt, J=3.4, and 7.5 Hz, 1H), 3.45 (dd, J=3.8, and 9.3 Hz, 1H), 3.36-3.20 (m, 2H), 3.25 (s, 3H), 3.05 (dt, J=7.0, and 10.0 Hz, 1H), 1.82-1.62 (m, 2H), 1.49-1.37 (m, 1H), and 1.43 (s, 1H); Retention Time=5.783 min; HRMS: m/z (M+Na)+=(Calculated for C₂₀H₂₃IN₂NaO₅S, 553.0265) found, 553.0269.

### 3-Iodo-4-methoxy-N-(4-((2-phenylpiperidin-1-yl)sulfonyl)phenyl)benzamide (90)

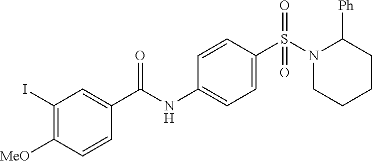 [SMILES: COc1ccc(C(=O)Nc2ccc(S(=O)(=O)N3CCCCC3c3ccccc3)cc2)cc1I]

Synthesize using Method C and 2-phenylpiperidine as the starting material. ¹H NMR (400 MHz, DMSO-d₆): δ 10.44 (s, 1H), 8.40 (d, J=2.2 Hz, 1H), 8.08-7.94 (m, 3H), 7.89-7.78 (m, 2H), 7.42-7.29 (m, 4H), 7.27-7.22 (m, 1H), 7.13 (d, J=8.7 Hz, 1H), 5.15 (d, J=5.1 Hz, 1H), 3.91 (s, 3H), 3.79-3.65 (m, 1H), 3.03-2.83 (m, 1H), 2.16 (d, J=14.0 Hz, 1H), 1.60-1.29 (m, 3H), and 1.29-0.99 (m, 2H); Retention Time=6.715 min; HRMS: m/z (M+H)+=(Calculated for C₂₅H₂₆IN₂O₄S, 577.0652) found, 577.0666.

### 3-Iodo-N-(4-((2-isopropylpiperidin-1-yl)sulfonyl)phenyl)-4-methoxybenzamide (85)

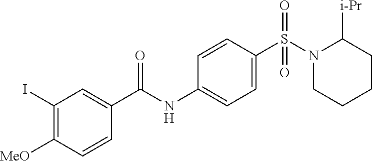 [SMILES: COc1ccc(C(=O)Nc2ccc(S(=O)(=O)N3CCCCC3C(C)C)cc2)cc1I]

Synthesize using Method C and 2-isopropylpiperidine as the starting material. ¹H NMR (400 MHz, DMSO-d₆): δ 10.41 (s, 1H), 8.39 (d, J=2.2 Hz, 1H), 8.05-7.90 (m, 3H), 7.81-7.74 (m, 2H), 7.13 (d, J=8.9 Hz, 1H), 3.90 (s, 3H), 3.67 (dd, J=4.4, and 14.5 Hz, 1H), 3.44 (dd, J=4.9, and 10.6 Hz, 1H), 3.04-2.78 (m, 1H), 2.05 (dq, J=6.6, and 10.7 Hz, 1H), 1.59 (d, J=13.9 Hz, 1H), 1.49-1.27 (m, 3H), 1.16-0.89 (m, 2H), and 0.84 (dd, J=6.0, and 18.0 Hz, 6H); Retention Time=6.686 min; HRMS: m/z (M+H)+=(Calculated for C₂₃H₂₈IN₂O₄S, 543.0809) found, 543.0805.

### 3-Iodo-N-(4-((2-isopropylpyrrolidin-1-yl)sulfonyl)phenyl)-4-methoxybenzamide (88)

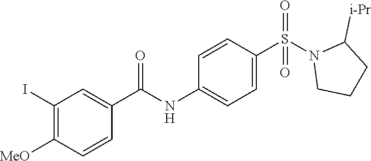 [SMILES: COc1ccc(C(=O)Nc2ccc(S(=O)(=O)N3CCCC3C(C)C)cc2)cc1I]

Synthesize using Method C and isopropylpyrrolidine as the starting material. ¹H NMR (400 MHz, DMSO-d₆): δ 10.48 (s, 1H), 8.38 (d, J=2.2 Hz, 1H), 8.05-7.93 (m, 3H), 7.83-7.74 (m, 2H), 7.13 (d, J=8.9 Hz, 1H), 3.90 (s, 3H), 3.42 (ddd, J=4.6, 5.7, and 8.1 Hz, 1H), 3.30-3.11 (m, 2H), 2.04-1.89 (m, 1H), 1.67-1.49 (m, 2H), 1.42-1.28 (m, 1H), 1.27-1.14 (m, 1H), and 0.84 (dd, J=6.9, and 18.8 Hz, 6H); Retention Time=6.494 min; HRMS: m/z (M+H)+=(Calculated for C₂₁H₂₆IN₂O₄S, 529.0652) found, 529.0659.

### N-(4-((2-ethylpiperidin-1-yl)sulfonyl)phenyl)-3-iodo-4-methoxybenzamide (82)

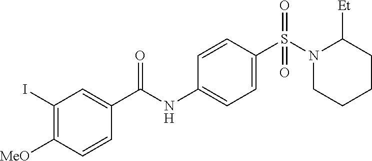 [SMILES: CCC1CCCCN1S(=O)(=O)c1ccc(NC(=O)c2ccc(OC)c(I)c2)cc1]

Synthesize using Method C and 2-ethylpiperidine as the starting material. ¹H NMR (400 MHz, DMSO-d₆): δ 10.46 (s, 1H), 8.39 (d, J=2.2 Hz, 1H), 8.05-7.91 (m, 3H), 7.81-7.74 (m, 2H), 7.13 (d, J=8.8 Hz, 1H), 3.90 (s, 3H), 3.80 (p, J=7.1 Hz, 1H), 3.63 (dd, J=4.2, and 14.3 Hz, 1H), 3.00-2.88 (m, 1H), 1.65-1.46 (m, 1H), 1.50-1.36 (m, 3H), 1.35 (s, 1H), 1.35-1.15 (m, 2H), 1.08-0.93 (m, 1H), and 0.78 (t, J=7.4 Hz, 3H); Retention Time=6.409 min; HRMS: m/z (M+H)+=(Calculated for C₂₁H₂₆IN₂O₄S, 529.0652) found, 529.0673. The enantiomers were separated using Column: CHIRALPAK AS, Mobile Phase: MeOH 100%, at 35 mL/min to give the enantiomers at a >95% purity.

### Methyl-1-((4-(3-iodo-4-methoxybenzamido)phenyl)sulfonyl)piperidine-2-carboxylate (87)

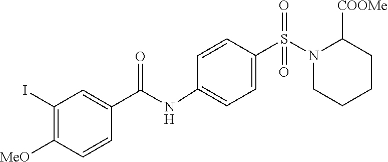 [SMILES: COC(=O)C1CCCCN1S(=O)(=O)c1ccc(NC(=O)c2ccc(OC)c(I)c2)cc1]

Synthesize using Method C and methylpiperidine-2-carboxylate as the starting material. ¹H NMR (400 MHz, DMSO-d₆): δ 10.45 (d, J=17.0 Hz, 1H), 8.39 (d, J=2.2 Hz, 1H), 8.09-7.90 (m, 3H), 7.79-7.63 (m, 2H), 7.13 (d, J=8.8 Hz, 1H), 4.68-4.52 (m, 1H), 3.90 (d, J=1.8 Hz, 3H), 3.62 (d, J=12.8 Hz, 1H), 3.51 (d, J=3.1 Hz, 3H), 3.18-3.01 (m, 1H), 1.94 (d, J=13.4 Hz, 1H), 1.62-1.46 (m, 2H), and 1.31-1.06 (m, 3H); Retention Time=5.959 min; HRMS: m/z (M+Na)+=(Calculated for C₂₁H₂₃IN₂NaO₆S, 581.0234) found, 581.0214.

### N-(4-((2-ethylpyrrolidin-1-yl)sulfonyl)phenyl)-3-iodo-4-methoxybenzamide (91)

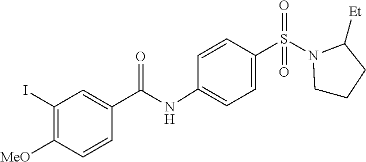 [SMILES: CCC1CCCN1S(=O)(=O)c1ccc(NC(=O)c2ccc(OC)c(I)c2)cc1]

Synthesize using Method C and 2-ethylpyrrolidine as the stating material. ¹H NMR (400 MHz, DMSO-d₆): δ 10.48 (s, 1H), 8.38 (d, J=2.2 Hz, 1H), 8.05-7.93 (m, 3H), 7.82-7.74 (m, 2H), 7.13 (d, J=8.8 Hz, 1H), 3.90 (s, 3H), 3.45 (tt, J=4.6, and 9.0 Hz, 1H), 3.32-3.19 (m, 1H), 3.18-3.06 (m, 1H), 1.75-1.60 (m, 1H), 1.55-1.43 (m, 2H), 1.47-1.26 (m, 3H), and 0.84 (t, J=7.4 Hz, 3H); Retention Time=6.201 min; HRMS: m/z (M+H)+=(Calculated for C₂₀H₂₄IN₂O₄S, 515.0496) found, 515.0489.

### 3-Iodo-4-methoxy-N-(4-((4-methylpiperazin-1-yl)sulfonyl)phenyl)benzamide, TFA (9)

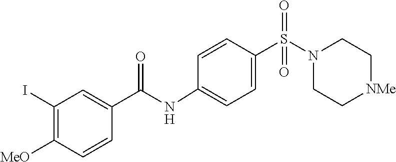 [SMILES: COc1ccc(C(=O)Nc2ccc(S(=O)(=O)N3CCN(C)CC3)cc2)cc1I]

Synthesize using Method C and 4-methylpiperazine. ¹H NMR (400 MHz, DMSO-d₆): δ 10.52 (s, 1H), 8.39 (d, J=2.2 Hz, 1H), 8.01 (dt, J=2.0, and 8.9 Hz, 3H), 7.69 (d, J=8.6 Hz, 2H), 7.13 (d, J=8.8 Hz, 1H), 3.90 (d, J=1.6 Hz, 3H), 2.85 (s, 4H), 2.34 (s, 4H), and 2.12 (s, 3H); Retention Time=4.003 min; HRMS: m/z (M+H)+=(Calculated for C₁₉H₂₃IN₃O₄S, 516.0448) found, 516.0469.

### N-(4-((3,4-dimethylpiperazin-1-yl)sulfonyl)phenyl)-3-iodo-4-methoxy benzamide (93)

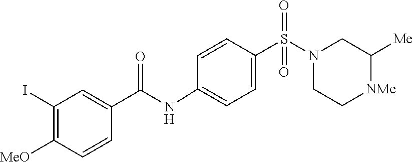 [SMILES: COc1ccc(C(=O)Nc2ccc(S(=O)(=O)N3CCN(C)C(C)C3)cc2)cc1I]

Synthesize using Method C and 3,4-dimethylpiperazine as the starting material. ¹H NMR (400 MHz, DMSO-d₆): δ 10.51 (s, 1H), 8.39 (d, J=2.2 Hz, 1H), 8.06-7.97 (m, 3H), 7.73-7.66 (m, 2H), 7.14 (d, J=8.7 Hz, 1H), 3.90 (s, 3H), 3.43-3.28 (m, 2H), 2.71 (dt, J=2.9, and 11.6 Hz, 1H), 2.39-2.29 (m, 1H), 2.10 (s, 3H), 2.19-2.02 (m, 1H), 1.95 (dd, J=9.8, and 11.0 Hz, 1H), and 0.92 (d, J=6.1 Hz, 3H); Retention Time=4.449 min; HRMS: m/z (M+Na)+=(Calculated for C₂₀H₂₄IN₃NaO₄S, 552.0424) found, 552.0447.

### N-(4-((4-ethyl-2-methylpiperazin-1-yl)sulfonyl)phenyl)-3-iodo-4-methoxybenzamide, NH₄⁺ (95)

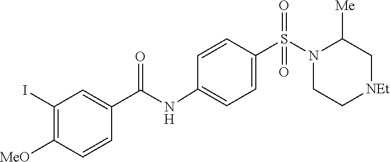

Synthesize using Method C and 4-ethyl2-methylpiperazine as the starting material. ¹H NMR (400 MHz, DMSO-d6): δ 10.47 (s, 1H), 8.39 (d, J=2.2 Hz, 1H), 8.05-7.92 (m, 3H), 7.78-7.71 (m, 2H), 7.13 (d, J=8.8 Hz, 1H), 3.90 (s, 3H), 3.53-3.44 (m, 1H), 3.18-3.02 (m, 1 H), 2.72-2.64 (m, 1H), 2.53 (dt, J=2.0, and 11.3 Hz, 1H), 2.26-2.11 (m, 2H), 1.88 (dd, J=3.7, and 11.2 Hz, 1H), 1.75 (td, J=3.4 and 11.5, Hz, 2H), 1.04 (d, J=6.7 Hz, 3H), and 0.90 (t, J=7.2 Hz, 3H); Retention Time=4.204 min; HRMS: m/z (M+H)+=(Calculated for C₂₁H₂₇IN₃O₄S, 544.0761) found, 544.0766.

### N-(4-((2,4-dimethylpiperazin-1-yl)sulfonyl)phenyl)-3-iodo-4-methoxybenzamide, NH₄⁺ (96)

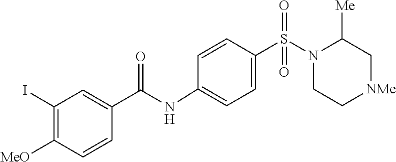 [SMILES: COc1ccc(C(=O)Nc2ccc(S(=O)(=O)N3CCN(C)CC3C)cc2)cc1I]

Synthesize using Method C and 2,4-dimethylpiperazine as the starting material. ¹H NMR (400 MHz, DMSO-d₆): δ 10.47 (s, 1H), 8.39 (d, J=2.2 Hz, 1H), 8.05-7.92 (m, 3H), 7.79-7.71 (m, 2H), 7.13 (d, J=8.8 Hz, 1H), 3.90 (s, 3H), 3.52-3.44 (m, 1H), 3.18-3.02 (m, 1H), 2.60 (d, J=11.5 Hz, 1H), 2.48-2.41 (m, 1H), 2.05 (s, 3H), 1.87 (dd, J=3.8, and 11.3 Hz, 1H), 1.72 (td, J=3.5, and 11.5 Hz, 2H), and 1.04 (d, J=6.7 Hz, 3H); Retention Time=4.378 min; HRMS: m/z (M+H)+=(Calculated for C₂₀H₂₅IN₃O₄S, 530.0605) found, 530.0613.

### 3-Iodo-4-methoxy-N-(4-(N-(tetrahydro-2H-pyran-4-yl)sulfamoyl)phenyl)benzamide (84)

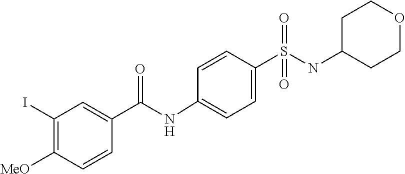 [SMILES: COc1ccc(C(=O)Nc2ccc(S(=O)(=O)NC3CCOCC3)cc2)cc1I]

Synthesize using Method C and tetrahydro-2H-pyran-4-amine, 2HCl as the starting material. ¹H NMR (400 MHz, DMSO-d₆): δ 10.44 (s, 1H), 8.38 (d, J=2.2 Hz, 1H), 8.01 (dd, J=2.2, and 8.6 Hz, 1H), 7.96-7.89 (m, 2H), 7.81-7.74 (m, 2H), 7.65 (d, J=7.3 Hz, 1H), 7.13 (d, J=8.8 Hz, 1H), 3.90 (s, 3H), 3.69 (dt, J=3.8, and 11.7 Hz, 2H), 3.32-3.07 (m, 4H), 1.53-1.44 (m, 1H), and 1.40-1.25 (m, 2H); Retention Time=4.977 min; HRMS: m/z (M+H)+=(Calculated for C₁₉H₂₂IN₂O₅S, 517.0289) found, 517.0295.

### 3-Iodo-4-methoxy-N-(4-((3-methyl-3,8-diazabicyclo[3.2.1]octan-8-yl)sulfonyl)phenyl) benzamide, NH₄⁺ (81)

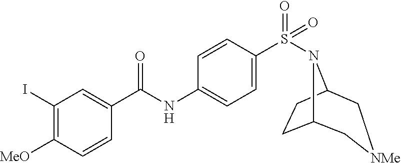 [SMILES: COc1ccc(C(=O)Nc2ccc(S(=O)(=O)N3C4CCC3CN(C)C4)cc2)cc1I]

Synthesize using Method C and 3-methyl-3,8-diazabicyclo[3.2.1]octane as the starting material. ¹H NMR (400 MHz, DMSO-d₆): δ 10.54 (s, 1H), 9.23 (s, 1H), 8.39 (d, J=2.2 Hz, 1H), 8.00 (td, J=1.8, and 8.9 Hz, 3H), 7.86 (d, J=8.5 Hz, 2H), 7.14 (d, J=8.8 Hz, 1H), 4.37 (s, 2H), 3.90 (s, 3H), 3.18 (s, 2H), 2.75 (s, 3H), 2.20 (s, 1H), 1.76 (d, J=9.9 Hz, 2H), and 1.41 (s, 2H); Retention Time=4.398 min; HRMS: m/z (M+H)+=(Calculated for C₂₁H₂₅IN₃O₄S, 542.0605) found, 542.0596.

### N-(4-((2,6-dimethylpiperidin-1-yl)sulfonyl)phenyl)-3-iodo-4-methoxybenzamide, NH₄⁺ (111)

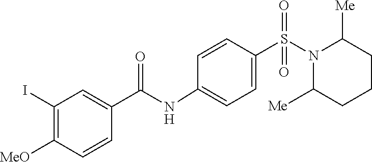 [SMILES: COc1ccc(C(=O)Nc2ccc(S(=O)(=O)N3C(C)CCCC3C)cc2)cc1I]

Synthesize using Method C and 2,6-dimethylpiperidine as the starting material. ¹H NMR (400 MHz, DMSO-d₆) δ: 10.46 (s, 1H), 8.38 (d, J=2.2 Hz, 1H), 8.01 (dd, J=2.3, and 8.6 Hz, 1H), 7.98-7.90 (m, 2H), 7.80-7.72 (m, 2H), 7.13 (d, J=8.8 Hz, 1H), 4.06 (h, J=6.4 Hz, 2H), 3.90 (s, 3H), 3.33-3.23 (m, 1H), 1.74-1.58 (m, 1H), 1.37 (d, J=13.3 Hz, 2H), and 1.24 (d, J=7.1 Hz, 8H); Retention Time=6.417 min; HRMS: m/z (M+Na)+=(Calculated for C₂₁H₂₅IN₂NaO₄S, 551.0472) found, 551.0481.

### 3-Iodo-4-methoxy-N-(4-((2-methyl-4-oxopiperidin-1-yl)sulfonyl)phenyl)benzamide

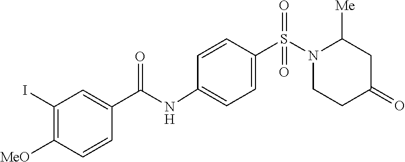 [SMILES: COc1ccc(C(=O)Nc2ccc(S(=O)(=O)N3CCC(=O)CC3C)cc2)cc1I]

Synthesize using Method C and 2-methylpiperidin-4-one as the starting material. ¹H NMR (400 MHz, DMSO-d₆): δ 10.50 (s, 1H), 8.38 (d, J=2.2 Hz, 1H), 8.04-7.90 (m, 3H), 7.87-7.79 (m, 2H), 7.11 (dd, J=8.7, and 13.3 Hz, 1H), 4.43-4.35 (m, 1H), 3.89 (d, J=3.7 Hz, 4H), 3.41-3.30 (m, 1H), 2.53 (dd, J=6.5, and 14.5 Hz, 1H), 2.37 (ddd, J=7.1, 11.3, and 15.3 Hz, 1H), 2.18 (d, J=15.7 Hz, 1H), 2.12-2.02 (m, 1H), and 0.95 (d, J=6.8 Hz, 3H); Retention Time=5.199 min; HRMS: m/z (M+H)+=(Calculated for C₂₀H₂₂IN₂O₅S, 529.0289) found, 529.0298.

### N-(4-((4-hydroxy-2-methylpiperidin-1-yl)sulfonyl)phenyl)-3-iodo-4-methoxybenzamide (110)

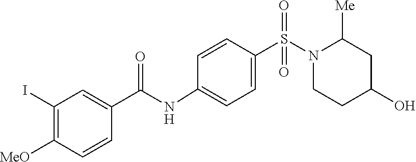 [SMILES: COc1ccc(C(=O)Nc2ccc(S(=O)(=O)N3CCC(O)CC3C)cc2)cc1I]

Follow the synthesis for 3-iodo-4-methoxy-N-(4-((2-methyl-4-oxopiperidin-1-yl)sulfonyl) phenyl)benzamide. 3-Iodo-4-methoxy-N-(4-((2-methyl-4-oxopiperidin-1-yl)sulfonyl)phenyl) benzamide (0.13 g, 0.25 mmol), was stirred in EtOH (2.5 mL) and treated with sodium borohydride (0.03 g, 0.75 mmol) at room temp. This reaction was stirred for 3 hrs. at which time the reaction pH was adjusted with 1 N HCl. The reaction was concentrated and purified on reverse phase to give the desired compound as a mixture of diastereomers. ¹H NMR (400 MHz, DMSO-d₆): δ 10.46 (d, J=3.0 Hz, 1H), 8.38 (d, J=2.2 Hz, 1H), 8.04-7.90 (m, 3H), 7.75 (dd, J=1.8, and 8.9 Hz, 2H), 7.12 (d, J=8.7 Hz, 1H), 4.61 (dd, J=2.1, and 3.7 Hz, 1H), 3.89 (s, 3H), 3.86-3.61 (m, 2H), 3.34 (t, J=5.8 Hz, 1H), 1.61-1.48 (m, 2H), 1.48-1.39 (m, 2H), 1.30-1.18 (m, 1H), and 1.17-0.91 (m, 3H); Retention Time=4.877 and 4.964 min; HRMS: m/z (M+H)+=(Calculated for C₂₀H₂₄IN₂O₅S, 531.0445) found, 531.0459.

### 4-Bromo-N-(4-(N,N-dipropylsulfamoyl)phenyl)-3-iodobenzamide (94)

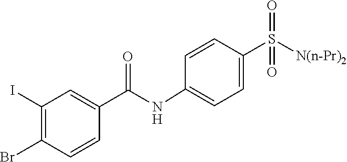

Synthesize using Method C and N,N-dipropylamine HCl as the starting material, and 4-bromo-3-iodobenzoyl chloride as the acid chloride. ¹H NMR (400 MHz, DMSO-d₆): δ 10.65 (s, 1H), 8.46 (dd, J=0.5, and 2.0 Hz, 1H), 7.98-7.91 (m, 2H), 7.95-7.81 (m, 2H), 7.80-7.72 (m, 2H), 3.03-2.94 (m, 4H), 1.51-1.37 (m, 4H), and 0.79 (t, J=7.4 Hz, 6H); Retention Time=6.906 min; HRMS: m/z (M+H)+=(Calculated for C₁₉H₂₃BrIN₂O₃S, 564.9652) found, 564.9663.

### 4-Bromo-3-iodo-N-(4-((2-methylpiperidin-1-yl)sulfonyl)phenyl)benzamide (73)

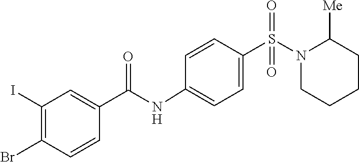 [SMILES: CC1CCCCN1S(=O)(=O)c1ccc(NC(=O)c2ccc(Br)c(I)c2)cc1]

Synthesize using Method C and 2-methylpiperidine as the starting material and 4-bromo-3-iodobenzoyl chloride as the acid chloride. ¹H NMR (400 MHz, DMSO-d₆): δ 10.65 (s, 1H), 8.45 (dd, J=0.5, and 2.0 Hz, 1H), 7.98-7.90 (m, 2H), 7.95-7.81 (m, 2H), 7.81-7.72 (m, 2H), 4.06 (s, 1H), 3.57 (d, J=11.0 Hz, 1H), 2.93 (td, J=2.7, and 13.0 Hz, 1H), 1.54-1.45 (m, 1H), 1.49-1.35 (m, 1H), 1.38 (s, 3H), 1.24-1.10 (m, 1H), and 0.97 (d, J=6.9 Hz, 3H); Retention Time=6.646 min; HRMS: m/z (M+H)+=(Calculated for C₁₉H₂₁BrIN₂O₃S, 564.9476) found, 564.9479.

### 4-Bromo-3-iodo-N-(4-((2-(trifluoromethyl)piperidin-1-yl)sulfonyl)phenyl)benzamide (109)

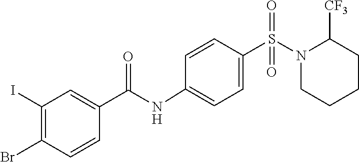 [SMILES: O=C(Nc1ccc(S(=O)(=O)N2CCCCC2C(F)(F)F)cc1)c1ccc(Br)c(I)c1]

Synthesize using Method C and 2-trifluoromethylpiperidine as the starting material and 4-bromo-3-iodobenzoyl chloride as the acid chloride. ¹H NMR (DMSO-d6, 400 MHz): δ 10.71 (s, 1H), 8.45 (d, J=2.0 Hz, 1H), 8.02-7.93 (m, 2H), 7.94-7.81 (m, 4H), 4.74 (s, 1H), 3.69 (dd, J=4.4, and 14.5 Hz, 1H), 3.03 (t, J=13.9 Hz, 1H), 1.80 (d, J=11.8 Hz, 1H), 1.40 (d, J=14.6 Hz, 4H), and 0.77 (d, J=12.2 Hz, 1H); Retention Time=6.765 min; HRMS: m/z (M+H)+=(Calculated for C₁₉H₁₈BrF₃IN₂O₃S, 618.9194) found, 618.9168.

### 4-Bromo-3-iodo-N-(4-((2-propylpiperidin-1-yl)sulfonyl)phenyl)benzamide (97)

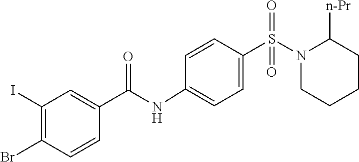

Synthesize using Method C and 2-propylpiperidine as the starting material and 4-bromo-3-iodobenzoyl chloride as the acid chloride. ¹H NMR (400 MHz, DMSO-d₆): δ 10.65 (s, 1H), 8.46 (dd, J=0.5, and 1.9 Hz, 1H), 7.98-7.90 (m, 2H), 7.92-7.81 (m, 2H), 7.83-7.74 (m, 2H), 3.93-3.85 (m, 1H), 3.66-3.57 (m, 2H), 3.36-3.23 (m, 2H), 2.95 (t, J=12.4 Hz, 1H), 1.61-1.11 (m, 6H), 0.99 (ddt, J=4.4, 8.7, and 13.2 Hz, 1H), and 0.84 (t, J=7.3 Hz, 3H); Retention Time=7.158 min; HRMS: m/z (M+H)+=(Calculated for C₂₁H₂₅BrIN₂O₃S, 592.7803) found, 592.9790.

### 4-Bromo-N-(4-((4,4-difluoropiperidin-1-yl)sulfonyl)phenyl)-3-iodobenzamide (112)

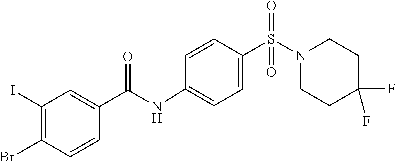 [SMILES: O=C(Nc1ccc(S(=O)(=O)N2CCC(F)(F)CC2)cc1)c1ccc(Br)c(I)c1]

Synthesize using Method C and 4,4-difluoropiperidine as the starting material and 4-bromo-3-iodobenzoyl chloride as the acid chloride. ¹H NMR (400 MHz, DMSO-d₆): δ 10.72 (s, 1H), 8.45 (dd, J=0.4, and 2.0, Hz, 1H), 8.05-7.97 (m, 2H), 7.92-7.81 (m, 2H), 7.80-7.72 (m, 2H), 3.03 (d, J=5.9 Hz, 4H), and 2.03 (ddt, J=5.8, 13.5, and 19.7 Hz, 4H); Retention Time=6.299 min; HRMS: m/z (M+H)+=(Calculated for C₂₁H₂₅BrIN₂O₃S, 592.7803) found, 592.9790.

### 3-Iodo-4-methoxy-N-(4-(morpholinosulfonyl)phenyl)benzamide (5)

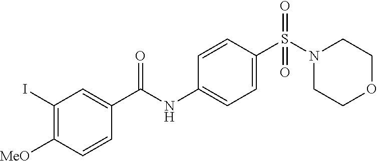 [SMILES: COc1ccc(C(=O)Nc2ccc(S(=O)(=O)N3CCOCC3)cc2)cc1I]

Synthesize using Method C and morpholine as the starting material and 4-methoxy-3-iodobenzoyl chloride as the acid chloride. ¹H NMR (400 MHz DMSO-d₆): δ 10.53 (s, 1H), 8.39 (d, J=2.3 Hz, 1H), 8.07-7.98 (m, 3H), 7.74-7.66 (m, 2H), 7.14 (d, J=8.8 Hz, 1H), 3.90 (s, 3H), 3.61 (dd, J=5.5, 3.8 Hz, 4H), and 2.83 (dd, J=3.8, and 5.7 Hz, 4H):); Retention Time=5.342 min; HRMS: m/z (M+Na)+=(Calculated for C₁₈H₁₉IN₂NaO₅S, 524.9952) found, 524.9974.

### 4-Bromo-3-iodo-N-(4-((3-methylmorpholino)sulfonyl)phenyl)benzamide (113)

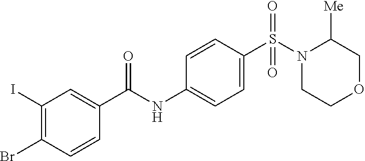 [SMILES: CC1COCCN1S(=O)(=O)c1ccc(NC(=O)c2ccc(Br)c(I)c2)cc1]

Synthesize using Method C and 3-methyllmorpholine as the starting material and 4-bromo-3-iodobenzoyl chloride as the acid chloride. Retention Time=5.955 min; HRMS: m/z (M+H)+=(Calculated for C₁₈H₁₉BrIN₂O₄S, 566.9269) found, 566.9254.

### 4-bromo-3-iodo-N-(4-((3-isopropylmorpholino)sulfonyl)phenyl)benzamide (114)

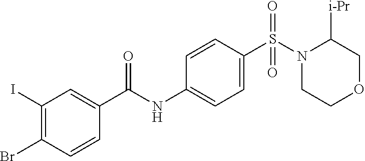 [SMILES: CC(C)C1COCCN1S(=O)(=O)c1ccc(NC(=O)c2ccc(Br)c(I)c2)cc1]

Synthesize using Method C and 3-isopropylmorpholine as the starting material and 4-bromo-3-iodobenzoyl chloride as the acid chloride. ¹H NMR (400 MHz DMSO-d₆): δ 10.67 (s, 1H), 8.46 (dd, J=0.5, and 1.9 Hz, 1H), 8.00-7.91 (m, 2H), 7.92-7.78 (m, 3H), 7.77 (s, 1H), 3.70 (d, J=12.0 Hz, 1H), 3.54-3.43 (m, 1H), 3.30-3.11 (m, 2H), 2.97-2.79 (m, 2H), 2.22-2.07 (m, 1H), 2.04 (s, 1H), and 0.87 (dd, J=6.7, and 9.0 Hz, 6H); Retention Time=6.426 min; FIRMS: m/z (M+H)+=(Calculated for C₂₀H₂₃BrIN₂O₄S, 594.9582) found, 594.9583.

### 3-Iodo-4-methoxy-N-(4-(N-phenylsulfamoyl)phenyl)benzamide (29)

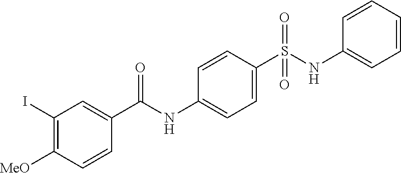 [SMILES: COc1ccc(C(=O)Nc2ccc(S(=O)(=O)Nc3ccccc3)cc2)cc1I]

Synthesize using Method C and aniline as the starting material. ¹H NMR (400 MHz, DMSO-d₆) δ 7.80-7.68 (m, 1H), 7.49-7.38 (m, 3H), 7.38-7.26 (m, 3H), 7.24-7.06 (m, 2H), 6.80 (d, J=8.8 Hz, 1H), 6.65-6.50 (m, 2H), 6.25 (s, 2H), and 3.74 (s, 3H); Retention Time=5.378 min; HRMS: m/z (M+H)+=(Calculated for C₂₀H₁₈IN₂O₄S, 509.0026) found, 509.0044.

### 3-Iodo-4-methoxy-N-(4-(N-(3-(trifluoromethyl)phenyl)sulfamoyl)phenyl)benzamide, NH₄⁺ (24)

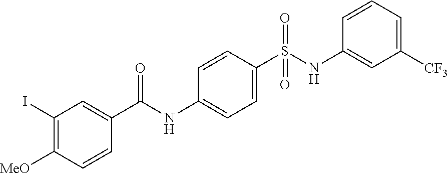 [SMILES: COc1ccc(C(=O)Nc2ccc(S(=O)(=O)Nc3cccc(C(F)(F)F)c3)cc2)cc1I]

Synthesize using Method C and 3-trifluoromethylaniline as the starting material. ¹H NMR (DMSO-d6, 400 MHz) δ 7.80 (d, J=2.2 Hz, 1H), 7.74 (s, 1H), 7.77-7.66 (m, 1H), 7.56-7.38 (m, 4H), 7.39-7.31 (m, 1H), 6.82 (d, J=8.8 Hz, 1H), 6.62-6.54 (m, 2H), 6.31 (s, 2H), and 3.75 (s, 3H); Retention Time=5.774 min; HRMS: m/z (M+Na)+=(Calculated for C₂₁H₁₆F₃IN₂NaO₄S, 598.9720) found, 598.9734.

### N-(4-(N-(2,3-dimethylphenyl)sulfamoyl)phenyl)-3-iodo-4-methoxybenzamide (27)

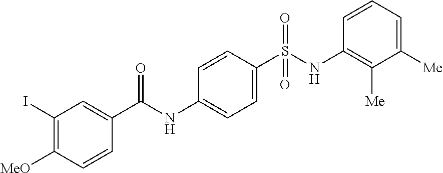 [SMILES: COc1ccc(C(=O)Nc2ccc(S(=O)(=O)Nc3cccc(C)c3C)cc2)cc1I]

Synthesize using Method C and 2,3-dimethylaniline as the starting material. ¹H NMR (400 MHz, DMSO-d₆) δ 7.60 (d, J=2.2 Hz, 1H), 7.55-7.47 (m, 2H), 7.28 (dd, J=2.2, and 8.7 Hz, 1H), 7.17-7.10 (m, 1H), 7.04 (t, J=7.7 Hz, 1H), 6.91 (d, J=7.4 Hz, 1H), 6.78 (d, J=8.7 Hz, 1H), 6.64-6.55 (m, 2H), 6.28 (s, 2H), 3.73 (s, 3H), 2.17 (s, 3H), and 2.04 (s, 3H); Retention Time=5.733 min; HRMS: m/z (M+H)+=(Calculated for C₂₂H₂₂IN₂O₄S, 537.0339) found, 537.0356.

### 3-Iodo-4-methoxy-N-(4-((2-methylpiperidin-1-yl)sulfonyl)phenyl)benzimidamide, TFA (117)

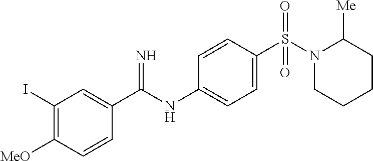 [SMILES: COc1ccc(C(=N)Nc2ccc(S(=O)(=O)N3CCCCC3C)cc2)cc1I]

Synthesize using Method C and 2-methylpiperidine as the starting material. To a stirred solution of 4-((2-methylpiperidin-1-yl)sulfonyl)aniline, HCl (0.16 g, 0.55 mmol), in DMF (1.0 mL), was added NaH (95%) (0.04 g, 1.65 mmol) and let stir at rt for 30 min before adding 3-iodo-4-methoxybenzonitrile (0.17 g, 0.66 mmol). The reaction mixture was stirred for 8 h, and quenched with water followed by EtOAc, the organic layer was washed with brine, dried over MgSO4, filtered, concentrated, and purified to give the desired material. ¹H NMR (400 MHz, DMSO-d₆): δ 8.33 (s, 1H), 7.92 (s, 1H), 7.63 (d, J=8.5 Hz, 2H), 7.02 (d, J=8.7 Hz, 1H), 6.94 (d, J=8.1 Hz, 2H), 4.06 (p, J=5.5 Hz, 1H), 3.85 (s, 3H), 3.68-3.44 (m, 1H), 2.93 (t, J=8.0 Hz, 1H), 1.56-1.32 (m, 6H), 1.31-1.12 (m, 1H), and 0.99 (d, J=6.9 Hz, 3H); Retention Time=4.471 min; HRMS: m/z (M+H)+=(Calculated for C₂₀H₂₅IN₃O₃S, 514.0656) found, 514.0650.

### N-(3-Iodo-4-methoxybenzyl)-4-((2-methylpiperidin-1-yl)sulfonyl)aniline, TFA (116)

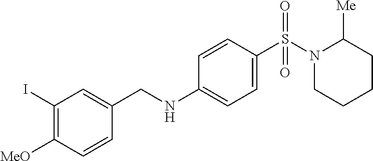 [SMILES: COc1ccc(CNc2ccc(S(=O)(=O)N3CCCCC3C)cc2)cc1I]

Synthesize using Method C using 2-methylpiperidine as the starting material. 4-((2-methylpiperidin-1-yl)sulfonyl)aniline (0.17 g, 0.67 mmol), and 3-iodo-4-methoxybenzaldehyde (0.26 g, 1.00 mmol), in EtOH (4.00 mL) underwent a rapid reflux for 18 h to form the imine. The reaction was cooled to rt before the addition of NaBH₄ (0.08 g, 2.00 mmol) and let stir for 4 h before quenching with saturated bicarb and MeOH. The mixture was allowed to stir for 30 min before concentrating. The solid was taken up in EtOAc, filtered, and washed with water and brine. The organic layer was dried with MgSO4, filtered, concentrated, and purified to give the desired product. ¹H NMR (400 MHz, DMSO-d₆): δ 7.71 (d, J=2.1 Hz, 1H), 7.45-7.35 (m, 2H), 7.31 (dd, J=2.2, and 8.4 Hz, 1H), 7.02 (t, J=6.0 Hz, 1H), 6.94 (d, J=8.5 Hz, 1H), 6.67-6.56 (m, 2H), 4.22 (d, J=5.9 Hz, 2H), 3.97 (dd, J=4.5, and 7.1 Hz, 1H), 3.77 (s, 3H), 3.44 (dd, J=3.8, and 12.6, Hz, 1H), 2.84 (m, 2H), 1.50-1.32 (m, 3H), 1.27-1.10 (m, 2H), and 0.94 (d, J=6.9 Hz, 3H); Retention Time=6.431 min; HRMS: m/z (M+H)+=(Calculated for C₂₀H₂₆IN₂O₃S, 501.0703) found, 501.0728.

### 4-Bromo-3-cyano-N-(4-((2-methylpiperidin-1-yl)sulfonyl)phenyl)benzamide (115)

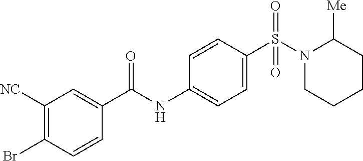 [SMILES: CC1CCCCN1S(=O)(=O)c1ccc(NC(=O)c2ccc(Br)c(C#N)c2)cc1]

Synthesize using Method C using 2-methylpiperidine as the starting material and 4-bromo-3-cyanobenzoyl chloride as the acid chloride. This acid chloride was synthesized in the following manner. 4-bromo-3-cyanobenzoic acid (0.10 g, 0.44 mmol), and oxalyl chloride (0.05 mL, 0.58 mmol) was stirred in DCM (0.44 mL) at rt before DMF (2.0 μl, 0.03 mmol) was added. The mixture was stirred at rt for 72 h, at which time the reaction was concentrate to a white solid. The white solid was used as is in the next reaction by making a 1 M solution in dry DCM. 1H NMR (400 MHz DMSO-d₆): δ 10.75 (s, 1H), 8.68-8.32 (m, 1H), 8.13 (dd, J=2.2, and 8.5 Hz, 1H), 8.08-8.03 (m, 1H), 7.97-7.90 (m, 2H), 7.81-7.75 (m, 2H), 4.08 (dd, J=3.7, and 7.1 Hz, 1H), 3.73-3.47 (m, 1H), 2.93 (td, J=2.7, and 13.0 Hz, 1H), 1.61-1.30 (m, 5H), 1.30-1.01 (m, 1H), and 0.97 (d, J=6.9 Hz, 3H); Retention Time=6.234 min; HRMS: m/z (M+H)+=(Calculated for C₂₀H₂₁BrN₃O₃S, 464.0463) found, 464.0451.

### 4-Bromo-3-ethynyl-N-(4-((2-methylpiperidin-1-yl)sulfonyl)phenyl)benzamide (119)

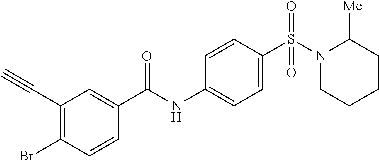 [SMILES: C#Cc1cc(C(=O)Nc2ccc(S(=O)(=O)N3CCCCC3C)cc2)ccc1Br]

Synthesize using Method C using 2-methylpiperidine as the starting material and 4-bromo-3-iodobenzoyl chloride as the acid chloride. Chill 4-bromo-3-iodo-N-(4-((2-methylpiperidin-1-yl)sulfonyl)phenyl)benzamide (0.20 g, 0.36 mmol), bis(triphenylphosphine)palladium(II) chloride (7.50 mg, 10.65 μmol), copper(I) iodide (4.00 mg, 0.02 mmol), TEA (0.500 mL, 3.55 mmol), and triphenylphosphine (5.60 mg, 0.02 mmol) in degasses THF (1.00 mL). Add ethynyltrimethylsilane (0.05 mL, 0.37 mmol), at 0° C. and take out of ice bath and let stir for 4 h at rt. When the reaction was complete thiol resin was added and stirred for 2 h at rt. The reaction was filtered through celite, and concentrated. The crude material was placed on normal phase silica column with Hex/EtOAc 0 to 70%. 4-Bromo-N-(4-((2-methylpiperidin-1-yl)sulfonyl)phenyl)-3-((trimethylsilyl)ethynyl)benzamide (0.12 g, 0.23 mmol), and K₂CO₃ (0.03 g, 0.23 mmol) was stirred in MeOH (3.0 mL) for 3 hr at rt. The reactions was concentrated and turned in for purification. ¹H NMR (400 MHz, DMSO-d₆) δ: 10.67 (s, 1H), 8.15 (dd, J=0.6, and 2.1 Hz, 1H), 7.99-7.91 (m, 2H), 7.96-7.81 (m, 2H), 7.81-7.72 (m, 2H), 4.71 (s, 1H), 4.10-4.03 (m, 1H), 3.61-3.51 (m, 1H), 2.92 (td, J=2.6, and 13.1 Hz, 1H), 1.62-1.24 (m, 5H), 1.23-1.09 (m, 1H), and 0.96 (d, J=6.9 Hz, 3H); Retention Time=5.914 min; HRMS: m/z (M+H)+=(Calculated for C₂₁H₂₂BrN₂O₃S, 463.051) found, 463.0502.

### N-(4-((4-hydroxypiperidin-1-yl)sulfonyl)phenyl)-3-iodo-4-methoxybenzamide (54)

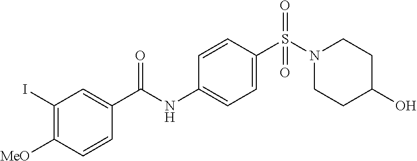 [SMILES: COc1ccc(C(=O)Nc2ccc(S(=O)(=O)N3CCC(O)CC3)cc2)cc1I]

Piperidin-4-ol (0.05 g, 0.45 mmol), and pyridine (0.15 mL, 1.81 mmol) were stirred in THF (2.30 mL) before 4-nitrobenzene-1-sulfonyl chloride (0.10 g, 0.45 mmol) was added to the mixture. This mixture was stirred for 3 hr, concentrated under a stream of nitrogen to give 1-((4-nitrophenyl)sulfonyl)piperidin-4-ol and used as is for the next reaction. 1-((4-nitrophenyl)sulfonyl)piperidin-4-ol (0.13 g, 0.45 mmol), AcOH (0.08 mL, 1.35 mmol), zinc (0.09 g, 1.35 mmol) in MeOH (2.250 mL) were stirred for 18 h, filter wash with acetonitrile and place on 10/90 gradient water/acetonitrile (0.1% TFA) reverse phase for purification. Retention Time=2.148 min. 1-((4-aminophenyl)sulfonyl)piperidin-4-ol, TFA (1 equiv) was treated with DIPEA (3 equiv) in DCM (0.2M) and 1 M solution of 3-iodo-4-methoxybenzoyl chloride (1.5 equiv) in DCM was added to the reaction at rt. This mixture was allowed to stir overnight and was quenched after 18 h with MeOH. The reaction was concentrated and purified to give the targeted compound. ¹H NMR (400 MHz, DMSO-d₆): δ 10.50 (s, 1H), 8.39 (d, J=2.2 Hz, 1H), 8.05-7.95 (m, 3H), 7.73-7.65 (m, 2H), 7.13 (d, J=8.8 Hz, 1H), 4.63 (d, J=3.9 Hz, 1H), 3.90 (s, 3H), 3.49 (dq, J=3.8, and 7.7 Hz, 1H), 3.22-2.99 (m, 2H), 2.68 (ddd, J=3.2, 8.3, and 11.4 Hz, 2H), 1.71 (ddd, J=3.7, 6.0, and 12.6 Hz, 2H), and 1.40 (dtd, J=3.6, 8.1, and 12.2 Hz, 2H); Retention Time=4.884 min; HRMS: m/z (M+Na)+=(Calculated for C₁₉H₂₁IN₂NaO₅S, 539.0108) found, 539.0121.

### N-(4-(1,4-dioxa-8-azaspiro[4.5]decan-8-ylsulfonyl)phenyl)-3-iodo-4-methoxybenzamide (60)

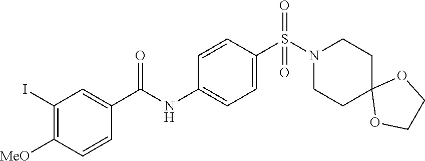 [SMILES: COc1ccc(C(=O)Nc2ccc(S(=O)(=O)N3CCC4(CC3)OCCO4)cc2)cc1I]

Synthesize using Method D and 1,4-dioxa-8-azaspiro[4.5]decane as the starting material. Retention Time=4.884 min; HRMS: m/z (M+H)+=(Calculated for C₂₁H₂₄IN₂O₆S, 559.0394) found, 559.0390.

### N-(4-((4-ethylpiperazin-1-yl)sulfonyl)phenyl)-3-iodo-4-methoxybenzamide, NH₄⁺ (57)

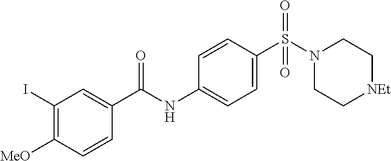

Synthesize using Method D and 1-ethylpiperazine as the starting material. The reduction of the nitro to the amine was done on an H-Cube pro flow reactor on a 70 mm Catcart of 10% Pd/C at 50° C. and 50 Barr at 0.9 mL/min on 0.1 M solution MeOH/EtOAc (1/1) for 2 h. The solvent was concentrated and the material was used as is in the next reaction. ¹H NMR (400 MHz, DMSO-d₆): δ 10.52 (s, 1H), 8.39 (t, J=1.7 Hz, 1H), 8.05-7.97 (m, 3H), 7.73-7.65 (m, 2H), 7.17-7.10 (m, 1H), 3.90 (d, J=1.5 Hz, 3H), 2.84 (s, 4H), 2.38 (s, 4H), 2.32-2.23 (m, 2H), and 0.90 (t, J=7.2 Hz, 3H); Retention Time=4.161 min; HRMS: m/z (M+H)+=(Calculated for C₂₀H₂₅IN₃O₄S, 530.0605) found, 530.0610.

### N-(4-(N-(2-(cyclohex-1-en-1-yl)ethyl)sulfamoyl)phenyl)-3-iodo-4-methoxybenzamide (45)

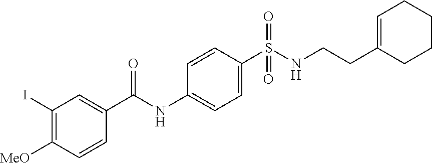 [SMILES: COc1ccc(C(=O)Nc2ccc(S(=O)(=O)NCCC3=CCCCC3)cc2)cc1I]

Synthesize using Method D and 2-(cyclohex-1-en-1-yl)ethanamine as the starting material and the zinc reduction of the nitro group. ¹H NMR (400 MHz, DMSO-d₆): δ 10.45 (s, 1H), 8.39 (d, J=2.2 Hz, 1H), 8.05-7.89 (m, 3H), 7.78-7.69 (m, 2H), 7.38 (t, J=5.9 Hz, 1H), 7.13 (d, J=8.8 Hz, 1H), 5.30 (dd, J=1.7, and 3.3, Hz, 1H), 3.90 (s, 3H), 2.81-2.71 (m, 2H), 1.96 (s, 1H), 2.00-1.91 (m, 1H), 1.90-1.84 (m, 2H), 1.77-1.69 (m, 2H), and 1.54-1.37 (m, 4H); Retention Time=6.455 min; HRMS: m/z (M+H)+=(Calculated for C₂₂H₂₆IN₂O₄S, 541.0652) found, 541.0656.

### N-(4-((2-(hydroxymethyl)pyrrolidin-1-yl)sulfonyl)phenyl)-3-iodo-4-methoxybenzamide (99)

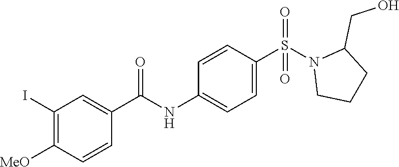 [SMILES: COc1ccc(C(=O)Nc2ccc(S(=O)(=O)N3CCCC3CO)cc2)cc1I]

Synthesize using Method D and pyrrolidin-2-ylmethanol as the starting material, and the zinc reduction. ¹H NMR (400 MHz, DMSO-d₆): δ 10.49 (s, 1H), 8.39 (d, J=2.2 Hz, 1H), 8.05-7.94 (m, 3H), 7.84-7.73 (m, 2H), 7.13 (d, J=8.8 Hz, 1H), 4.78 (d, J=5.9 Hz, 1H), 3.90 (s, 3H), 3.57-3.44 (m, 2H), 3.28-3.20 (m, 2H), 3.03 (dt, J=7.2, and 10.0 Hz, 1H), 1.86-1.63 (m, 2H), and 1.48-1.32 (m, 1H); Retention Time=5.030 min; HRMS: m/z (M+H)+=(Calculated for C₁₉H₂₁IN₂O₅S, 517.0289) found, 517.0296.

### 3-Iodo-4-methoxy-N-(4-(N-(2-(pyrrolidin-1-yl)ethyl)sulfamoyl)phenyl)benzamide, TFA (69)

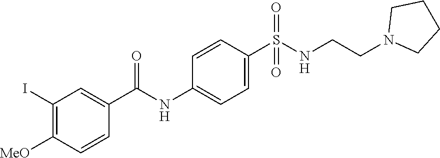 [SMILES: COc1ccc(C(=O)Nc2ccc(S(=O)(=O)NCCN3CCCC3)cc2)cc1I]

Synthesize using Method D and 2-(pyrrolidin-1-yl)ethanamine as the starting material. Retention Time=4.066 min; HRMS: m/z (M+H)+=(Calculated for C₂₀H₂₅IN₃O₄S, 530.0605) found, 530.0618.

### 3-Iodo-4-methoxy-N-(4-(N-(2-(piperidin-1-yl)ethyl)sulfamoyl)phenyl)benzamide, TFA (63)

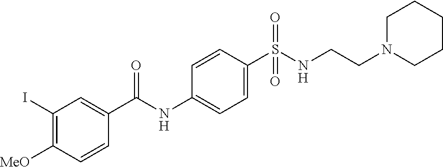 [SMILES: COc1ccc(C(=O)Nc2ccc(S(=O)(=O)NCCN3CCCCC3)cc2)cc1I]

Synthesize using Method D and 2-(piperidin-1-yl)ethanamine as the starting material. ¹H NMR (400 MHz, DMSO-d₆): δ 10.49 (s, 1H), 9.03 (s, 1H), 8.39 (d, J=2.2 Hz, 1H), 8.05-7.93 (m, 3H), 7.78 (d, J=8.9 Hz, 2H), 7.13 (d, J=8.8 Hz, 1H), 3.90 (s, 3H), 3.46-3.35 (m, 2H), 3.14-3.01 (m, 4H), 2.96-2.82 (m, 2H), 1.83-1.72 (m, 2H), 1.60 (d, J=15.6 Hz, 3H), and 1.33 (d, J=12.5 Hz, 1H); Retention Time=4.155 min; HRMS: m/z (M+H)+=(Calculated for C₂₁H₂₇IN₃O₄S, 544.0786 found, 544.0786.

### N-(4-(N-(2-(diethylamino)ethyl)sulfamoyl)phenyl)-3-iodo-4-methoxybenzamide, TFA (66)

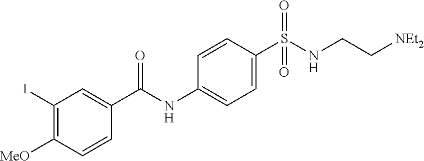

Synthesize using Method D and N,N-diethylethane-1,2-diamine as the starting material. ¹H NMR (400 MHz, DMSO-d₆): δ 10.50 (s, 1H), 9.05 (s, 1H), 8.39 (d, J=2.3 Hz, 1H), 8.05-7.94 (m, 3H), 7.79 (d, J=8.7 Hz, 2H), 7.13 (d, J=8.8 Hz, 1H), 3.90 (s, 3H), 3.23-2.91 (m, 8H), and 1.23-1.05 (m, 6H); Retention Time=4.140 min; HRMS: m/z (M+H)+=(Calculated for C₂₀H₂₇IN₃O₄S, 532.0761) found, 532.0758.

### N-(4-(azepan-1-ylsulfonyl)phenyl)-3-iodo-4-methoxybenzamide (70)

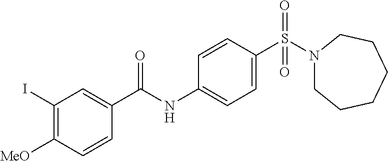 [SMILES: COc1ccc(C(=O)Nc2ccc(S(=O)(=O)N3CCCCCC3)cc2)cc1I]

Synthesize using Method D and azepane as the starting material. ¹H NMR (400 MHz, DMSO-d6): δ 10.46 (s, 1H), 8.38 (d, J=2.2 Hz, 1H), 8.12-7.88 (m, 3H), 7.81-7.65 (m, 2H), 7.13 (d, J=8.8 Hz, 1H), 3.90 (s, 3H), 3.21-3.12 (m, 4H), 1.63-1.55 (m, 4H), and 1.47 (ddd, J=2.5, 3.5, and 7.1 Hz, 4H); Retention Time=6.292 min; HRMS: m/z (M+Na)+=(Calculated for C₂₀H₂₃IN₂NaO₄S, 537.0315) found, 537.0332.

### 3-Iodo-4-methoxy-N-(4-(N-(2-morpholinoethyl)sulfamoyl)phenyl)benzamide, TFA (78)

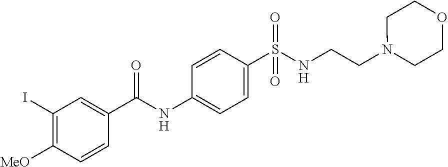 [SMILES: COc1ccc(C(=O)Nc2ccc(S(=O)(=O)NCCN3CCOCC3)cc2)cc1I]

Synthesize using Method D and 2-morpholinoethan amine as the starting material. ¹H NMR (400 MHz, DMSO-d₆): δ 10.49 (s, 1H), 9.68 (s, 1H), 8.39 (d, J=2.2 Hz, 1H), 8.05-7.93 (m, 3H), 7.83-7.74 (m, 2H), 7.13 (d, J=8.8 Hz, 1H), 3.90 (s, 5H), 3.73-3.56 (m, 2H), and 3.23-3.13 (m, 8H); Retention Time=4.022 min; HRMS: m/z (M+Na)+=(Calculated for C₂₀H₂₄IN₃NaO₅S, 568.0374) found, 568.0387.

### 3-Iodo-4-methoxy-N-(4-((4-(pyrimidin-2-yl)piperazin-1-yl)sulfonyl)phenyl)benzamide, NH₄⁺ (75)

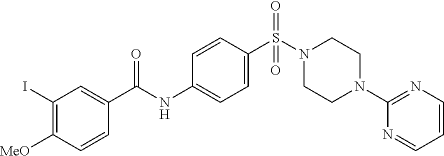 [SMILES: COc1ccc(C(=O)Nc2ccc(S(=O)(=O)N3CCN(c4ncccn4)CC3)cc2)cc1I]

Synthesize using Method D and 2-(piperazin-1-yl)pyrimidine, 2HCl as starting material. ¹H NMR (400 MHz, DMSO-d₆): δ 10.50 (s, 1H), 8.37 (d, J=2.2 Hz, 1H), 8.30 (d, J=4.8 Hz, 2H), 8.03-7.95 (m, 3H), 7.74-7.67 (m, 2H), 7.12 (d, J=8.8 Hz, 1H), 6.60 (t, J=4.7 Hz, 1H), 3.89 (s, 3H), 3.80 (t, J=5.1 Hz, 4H), and 2.92 (t, J=5.1 Hz, 4H); Retention Time=5.645 min; HRMS: m/z (M+Na)+=(Calculated for C₂₂H₂₂IN₅NaO₄S, 602.0329) found, 602.0347.

### 3-Iodo-4-methoxy-N-(4-(piperazin-1-ylsulfonyl)phenyl)benzamide, TFA (61)

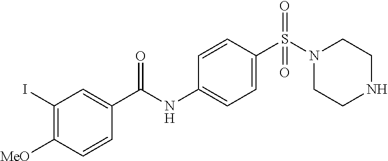 [SMILES: COc1ccc(C(=O)Nc2ccc(S(=O)(=O)N3CCNCC3)cc2)cc1I]

Synthesize using Method D and N-boc-piperazine as starting material. The Boc group was removed using 4 M HCl/dioxanes (3 equiv) stirred at rt for 1 h and concentrated to give desired product. ¹H NMR (400 MHz, DMSO-d₆) δ 10.58 (s, 1H), 8.49 (s, 1H), 8.40 (d, J=2.2 Hz, 1H), 8.09-7.98 (m, 3H), 7.80-7.71 (m, 2H), 7.14 (d, J=8.8 Hz, 1H), 3.90 (s, 3H), 3.18 (t, J=5.1 Hz, 4H), and 3.06 (d, J=5.4 Hz, 4H); Retention Time=4.386 min; HRMS: m/z (M+H)+=(Calculated for C₁₈H₂₁IN₃O₄S, 502.0311) found, 502.0310.

### 4-Bromo-3-iodo-N-(4-((2-methylpiperazin-1-yl)sulfonyl)phenyl)benzamide, TFA (86)

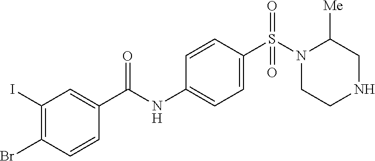 [SMILES: CC1CNCCN1S(=O)(=O)c1ccc(NC(=O)c2ccc(Br)c(I)c2)cc1]

Synthesize using Method D and tert-butyl-3-methylpiperazine-1-carboxylate as the starting material and 4-bromo-3-iodobenzoyl chloride as the acid chloride. After the amide formation the boc group was removed with 4 M HCl in dioxanes, at rt for 1 h. ¹H NMR (400 MHz, DMSO-d₆): δ 10.72 (s, 1H), 8.67 (s, 2H), 8.45 (d, J=2.0 Hz, 1H), 8.03-7.96 (m, 2H), 7.93-7.79 (m, 2H), 4.13 (s, 1H), 3.29 (s, 3H), 3.27-3.16 (m, 1H), 3.14 (s, 2H), 2.93 (dd, J=4.3, and 13.0 Hz, 1H), 2.85-2.74 (m, 1H), and 1.09 (d, J=7.0 Hz, 2H); Retention Time=4.489 min; HRMS: m/z (M+Na)+=(Calculated for C₁₈H₁₉BrIN₃NaO₃S, 587.9248) found, 587.9237.

### 4-Bromo-3-iodo-N-(4-((3-methylpiperazin-1-yl)sulfonyl)phenyl)benzamide, TFA (83)

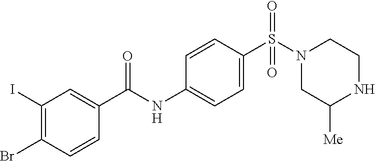 [SMILES: CC1CN(S(=O)(=O)c2ccc(NC(=O)c3ccc(Br)c(I)c3)cc2)CCN1]

Synthesize using Method D and tert-butyl-2-methylpiperazine-1-carboxylate as the starting material and 4-bromo-3-iodobenzoyl chloride as the acid chloride. After the amide formation the boc group was removed with 4 M HCl in dioxanes, at rt for 1 h. ¹H NMR (400 MHz, DMSO-d₆): δ 10.75 (s, 1H), 8.46 (dd, J=0.4, and 2.0 Hz, 1H), 8.06-8.01 (m, 2H), 7.92-7.84 (m, 2H), 7.80-7.74 (m, 2H), 3.71-3.54 (m, 2H), 3.42-3.23 (m, 2H), 3.17-3.04 (m, 2H), 2.56-2.48 (m, 1H), 2.33-2.27 (m, 1H), and 1.16 (d, J=6.5 Hz, 3H); Retention Time=4.503 min; HRMS: m/z (M+H)+=(Calculated for C₁₈H₂₀BrIN₃O₃S, 565.9429) found, 565.9406.

### 4-Bromo-3-iodo-N-(4-(piperazin-1-ylsulfonyl)phenyl)benzamide, HC (121)

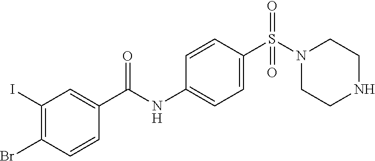 [SMILES: O=C(Nc1ccc(S(=O)(=O)N2CCNCC2)cc1)c1ccc(Br)c(I)c1]

Synthesize using Method D and tert-butyl piperazine-1-carboxylate as the starting material and 4-bromo-3-iodobenzoyl chloride as the acid chloride. After the amide formation the boc group was removed with 4 M HCl in dioxanes, at rt for 1 hr. ¹H NMR (400 MHz, DMSO-d₆) δ: 10.82 (s, 1H), 8.48 (t, J=1.2 Hz, 1H), 8.11-8.01 (m, 2H), 7.88 (d, J=1.2 Hz, 2H), 7.80-7.71 (m, 2H), and 3.19-3.06 (m, 8H); Retention Time=4.437 min; HRMS: m/z (M+H)+=(Calculated for C₁₇H₁₈BrIN₃O₃S, 551.9272) found, 551.929.

### 1-((4-(3-iodo-4-methoxybenzamido)phenyl)sulfonyl)piperazine-2-carboxamide, TFA (122)

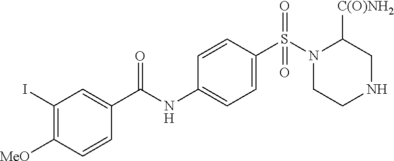

Synthesize using Method D and 1-(tert-butyl) 3-methyl piperazine-1,3-dicarboxylate as the starting material, followed by reduction using a H-Cube Pro with a Pd/C Catcart at 50° C., and 50 Barr at 0.1 M in methanol and ethyl acetate (1/1). Once the reaction was complete the solvents were concentrated and the reaction was carried through without further purification. The amide coupling was done as previously described using 3-iodo-4-methoxybenzoyl chloride. The ester hydrolysis was done using 1 M LiOH/MeOH (1:1) heating to 70° C. for 1 hr. The carboxamide was done under standard conditions with EDC, HOBt, and ammonium hydroxide in DMF at rt overnight. When reaction was complete by LCMS it was poured into EtOAc and water. The organic layer was washed with water and brine, dried over Na₂SO₄, filtered, and concentrated. The crude boc protected piperazine was deprotected using 4 M HCl/dioxanes 1 h, at rt. This crude material was purified by reverse phase to give the desired material. ¹H NMR (400 MHz DMSO-d6): δ (10.57 (s, 1H), 8.39 (d, J=2.3 Hz, 1H), 8.15-7.88 (m, 3H), 7.93-7.75 (m, 2H), 7.72-7.45 (m, 2H), 7.14 (d, J=8.8 Hz, 1H), 4.59 (d, J=4.7 Hz, 1H), 3.90 (s, 4H), 3.53 (t, J=12.0 Hz, 2H), 3.19-2.94 (m, 2H), 2.81 (dd, J=5.0, and 13.4 Hz, 1H), and 2.72-2.55 (m, 1H); Retention Time=3.922 min; HRMS: m/z (M+H)+=(Calculated for C₁₉H₂₂IN₄O₅S, 545.035) found, 545.0344.

### 3,4-Dibromo-N-(4-((3-methylpiperazin-1-yl)sulfonyl)phenyl)benzamide, TFA (120)

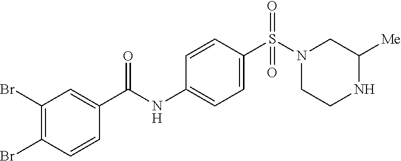 [SMILES: CC1CN(S(=O)(=O)c2ccc(NC(=O)c3ccc(Br)c(Br)c3)cc2)CCN1]

Synthesize using Method D and tert-butyl-2-methylpiperazine-1-carboxylate as the starting material and 3,4-dibromobenzoyl chloride as the acid chloride. The dibromobenzoyl chloride was synthesized the same as previously described for the 3-iodo-4methyoxybenzolyl chloride. After the amide formation the boc group was removed with 4 M HCl in dioxanes, at rt for 1 h. ¹H NMR (400 MHz DMSO-d₆): δ 10.78 (s, 1H), 8.31 (d, J=2.1 Hz, 1H), 8.07-7.99 (m, 2H), 7.95 (d, J=8.4 Hz, 1H), 7.86 (dd, J=2.1, and 8.4 Hz, 1H), 7.82-7.73 (m, 2H), 3.61 (t, J=12.9 Hz, 2H), 3.27 (s, 1H), 3.07 (t, J=12.0 Hz, 1H), 2.69-2.59 (m, 2H), 2.25 (t, J=11.4 Hz, 1H), and 1.13 (d, J=6.4 Hz, 3H); Retention Time=4.439 min; HRMS: m/z (M+H)+=(Calculated for C₁₈H₂₀Br₂N₃O₃S, 517.9548) found, 517.9569.

### N-(4-((3,3-dimethylpiperazin-1-yl)sulfonyl)phenyl)-3-iodo-4-methoxybenzamide, TFA (123)

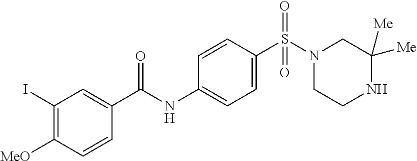 [SMILES: COc1ccc(C(=O)Nc2ccc(S(=O)(=O)N3CCNC(C)(C)C3)cc2)cc1I]

Synthesize using Method D and tert-butyl-2,2-dimethylpiperazine-1-carboxylate as the starting material. After the amide formation the boc group was removed with 4 M HCl in dioxanes, at rt for 1 hr. ¹H NMR (400 MHz, DMSO-d6) δ: 10.56 (s, 1H), 8.68 (s, 2H), 8.39 (d, J=2.2 Hz, 1H), 8.14-7.94 (m, 3H), 7.82-7.65 (m, 2H), 7.13 (d, J=8.7 Hz, 1H), 3.90 (s, 3H), 3.29-3.17 (m, 2H), 3.05 (s, 2H), 2.87 (s, 2H), and 1.29 (s, 6H); Retention Time=4.334 min; HRMS: m/z (M+H)+=(Calculated for C₁₈H₂₀Br₂N₃O₃S, 519.9548) found, 519.9569.

### N-(4-((3,5-dimethylpiperazin-1-yl)sulfonyl)phenyl)-3-iodo-4-methoxybenzamide, TFA (125)

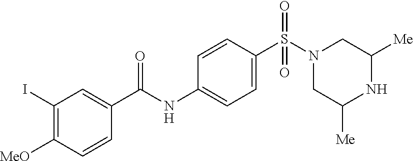 [SMILES: COc1ccc(C(=O)Nc2ccc(S(=O)(=O)N3CC(C)NC(C)C3)cc2)cc1I]

Synthesize using Method D and tert-butyl-2,2-dimethylpiperazine-1-carboxylate as the starting material. After the amide formation the boc group was removed with 4 M HCl in dioxanes, at rt for 1 h. ¹H NMR (400 MHz, DMSO-d6) δ: 10.56 (s, 1H), 9.03 (s, 1H), 8.39 (d, J=2.2 Hz, 1H), 8.07-7.98 (m, 3H), 7.80-7.73 (m, 2H), 7.13 (d, J=8.7 Hz, 1H), 4.05 (s, 1H), 3.90 (s, 3H), 3.76 (d, J=11.4 Hz, 2H), 3.13 (s, 1H), 2.14 (t, J=11.9 Hz, 2H), and 1.15 (d, J=6.4 Hz, 6H); Retention Time=4.371 min; HRMS: m/z (M+H)+=(Calculated for C₁₈H₂₀Br₂N₃O₃S, 519.9548) found, 519.9569.

### N-(4-((1H-pyrrolo[2,3-c]pyridin-1-yl)sulfonyl)phenyl)-3-iodo-4-methoxybenzamide, TFA (14)

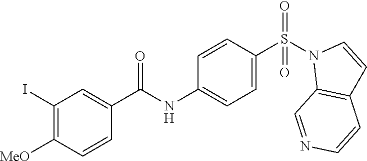 [SMILES: COc1ccc(C(=O)Nc2ccc(S(=O)(=O)n3ccc4ccncc43)cc2)cc1I]

Synthesize using Method D and 1H-pyrrolo[2,3-c]pyridine as the starting material. ¹H NMR (400 MHz, DMSO-d₆) δ 10.56 (s, 1H), 9.32 (d, J=1.0 Hz, 1H), 8.43 (d, J=5.6 Hz, 1H), 8.33 (d, J=2.3 Hz, 1H), 8.24 (d, J=3.5 Hz, 1H), 8.15-8.06 (m, 2H), 8.01-7.91 (m, 3H), 7.85 (d, J=5.7 Hz, 1H), 7.11 (d, J=8.8 Hz, 1H), 7.03 (dd, J=0.8, and 3.6 Hz, 1H), and 3.88 (s, 3H); Retention Time=4.671 min; HRMS: m/z (M+H)+=(Calculated for C₂₁H₁₇IN₃O₄S, 533.9979) found, 534.0000.

### 3-Iodo-4-methoxy-N-(4-(pyrrolidine-1-carbonyl)phenyl)benzamide (26)

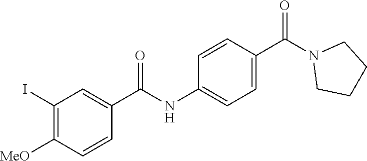 [SMILES: COc1ccc(C(=O)Nc2ccc(C(=O)N3CCCC3)cc2)cc1I]

(4-aminophenyl)(pyrrolidin-1-yl)methanone (0.06 g, 0.33 mmol), stirred in DCM (1.65 mL) with DIPEA (0.23 mL, 1.32 mmol) for 5 min before the addition of 3-iodo-4-methoxybenzoyl chloride (0.33 mL, 0.33 mmol) as a 1 M solution in DCM. This reaction stirred at rt for 18 h and was quenched with MeOH, concentrated, and was purified by reverse phase chromatography. ¹H NMR (400 MHz, DMSO-d₆) δ 10.28 (s, 1H), 8.38 (d, J=2.2 Hz, 1H), 8.00 (dd, J=2.3, and 8.6 Hz, 1H), 7.83-7.74 (m, 2H), 7.54-7.46 (m, 2H), 7.12 (d, J=8.7 Hz, 1H), 3.89 (s, 3H), 3.58-3.16 (m, 4H), and 1.82 (dt, J=6.7, and 13.4 Hz, 4H); Retention Time=4.995 min; HRMS: m/z (M+H)+=(Calculated for C₁₉H₂₀IN₂O₃, 451.0513) found, 451.0521.

### N-(4-(diethylcarbamoyl)phenyl)-3-iodo-4-methoxybenzamide (7)

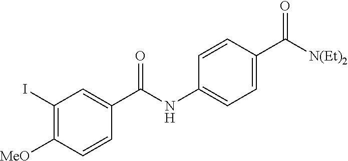

Synthesize using Method A and 4-amino-N,N-diethylbenzamide. ¹H NMR (400 MHz, DMSO-d₆) δ 10.26 (s, 1H), 8.38 (d, J=2.2 Hz, 1H), 8.01 (dd, J=2.2, and 8.6 Hz, 1H), 7.83-7.75 (m, 2H), 7.36-7.28 (m, 2H), 7.13 (d, J=8.8 Hz, 1H), 3.90 (s, 3H), 3.29-3.11 (m, 4H) and 1.07 (d, J=8.2 Hz, 6H); Retention Time=5.154 min; HRMS: m/z (M+H)+=(Calculated for C₁₉H₂₂IN₂O₃, 453.0670) found, 453.0674.

### N-(4-((diethylamino)methyl)phenyl)-3-iodo-4-methoxybenzamide (43)

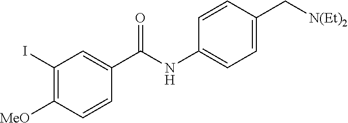

Tert-butyl (4-aminobenzyl)carbamate (73 mg, 0.33 mmol), DIPEA (0.23 mL, 1.32 mmol) was stirred in DCM (1.65 mL) for about 5 min before the addition of a 1 M solution of 3-iodo-4-methoxybenzoyl chloride (0.330 mL, 0.33 mmol) in DCM. The reaction mixture was allowed to stir for 18 h, poured into 10% citric acid solution, and extracted 3 times with DCM. The organic layers were combined and washed 1 time with saturated NaHCO₃ and one time with brine, dried over Na₂SO₄, filtered, and concentrated under reduced pressure. This crude material was used as is in the next reaction without further purification. The crude material was treated with 4 M HCl in dioxanes and stir at rt for 1 hr and concentrated, titrated with diethyl ether and dried under reduced pressure to give the product as an HCl. This product was used as is in the next reaction. N-(4-(aminomethyl)phenyl)-3-iodo-4-methoxybenzamide, HCl (0.14 g, 0.33 mmol), Cs₂CO₃ (0.11 g, 0.33 mmol), and iodoethane (0.07 mL, 0.83 mmol) were stirred in DMF (2.0 mL) overnight. The crude mixture was purified on reverse phase chromatography to give the desired compound with an overall yield of 14% over 3 steps. ¹H NMR (400 MHz, DMSO-d₆) δ 10.29 (s, 1H), 8.37 (d, J=2.2 Hz, 1H), 8.00 (dd, J=2.2, and 8.6 Hz, 1H), 7.92-7.72 (m, 2H), 7.58-7.35 (m, 2H), 7.12 (d, J=8.7 Hz, 1H), 4.24 (d, J=5.3 Hz, 2H), 3.89 (s, 3H), 3.04 (tt, J=5.8, and 11.8 Hz, 4H), and 1.20 (t, J=7.2 Hz, 6H); Retention Time=4.038 min; HRMS: m/z (M+Na)+=(Calculated for C₁₉H₂₃IN₂NaO₂, 461.0696) found, 461.0771.

### N-(4-bromo-3-iodophenyl)-4-((2-methylpiperidin-1-yl)sulfonyl)benzamide (127)

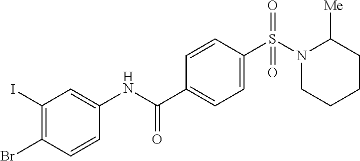 [SMILES: CC1CCCCN1S(=O)(=O)c1ccc(C(=O)Nc2ccc(Br)c(I)c2)cc1]

4-(Chlorosulfonyl)benzoic acid (0.50 g, 2.27 mmol), 2-methylpiperidine (0.53 mL, 4.53 mmol), TEA (0.32 mL, 2.27 mmol), was stirred in DCM (11.30 mL) overnight. The reaction was diluted with DCM and washed with 1 N HCl. The acidic layer was extracted 2×'s with DCM, all organic layers were combine and washed with NaHCO₃, and brine. The organic layer was dried over Na₂SO₄, filtered, and concentrated to give an oil which was used as is in the next reaction. 4-((2-methylpiperidin-1-yl)sulfonyl)benzoic acid (0.12 g, 0.41 mmol), 4-bromo-3-iodoaniline (0.12 g, 0.41 mmol), TEA (0.17 mL, 1.23 mmol), and propane phosphonic acid anhydride (0.39 mL, 0.61 mmol) in DMF (2.1 mL) was heated to 70° C. for 24 hr. The reaction was cooled to rt, poured into EtOAc and washed with 1 N HCl, bicarb, brine, dried over Na₂SO₄, filtered, concentrated, and turned in for purification and testing. ¹H NMR (400 MHz, DMSO-d₆): δ 10.58 (s, 1H), 8.41 (d, J=2.4 Hz, 1H), 8.14-8.05 (m, 2H), 7.98-7.90 (m, 2H), 7.78-7.65 (m, 2H), 4.15 (tq, J=3.5, and 7.1 Hz, 1H), 3.64 (dd, J=4.0, and 13.5 Hz, 1H), 2.99 (td, J=2.6, and 13.1 Hz, 1H), 1.57-1.42 (m, 2H), 1.40 (dd, J=3.8, and 7.8 Hz, 3H), 1.24-1.09 (m, 1H), and 1.00 (d, J=6.9 Hz, 3H); Retention Time=5.514 min; HRMS: m/z (M+Na)+=(Calculated for C₁₉H₂₀BrIN₂NaO₃S, 586.9296) found, 586.9271.

### N-(3-iodo-4-methoxyphenyl)-4-((2-methylpiperidin-1-yl)sulfonyl)benzamide (128)

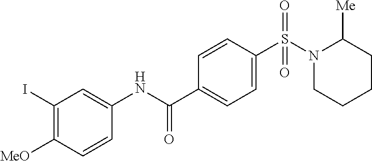 [SMILES: COc1ccc(NC(=O)c2ccc(S(=O)(=O)N3CCCCC3C)cc2)cc1I]

4-((2-methylpiperidin-1-yl)sulfonyl)benzoic acid (0.05 g, 0.18 mmol), DIPEA (0.10 mL, 0.529 mmol), HOBt (8.00 mg, 0.05 mmol), and 3-iodo-4-methoxyaniline (0.05 g, 0.21 mmol), and HATU (0.10 g, 0.27 mmol) was stirred in DMF (1.00 mL) for 3 h at rt. The reaction was poured into EtOAc and washed with 1 N HCl, 1 N NaOH, and 2×'s with brine. The solution was dried over Na₂SO₄, filtered, concentrated, and sent to purification. ¹H NMR (400 MHz, DMSO-d6): δ 10.37 (s, 1H), 8.20 (d, J=2.5 Hz, 1H), 8.11-8.04 (m, 2H), 7.95-7.88 (m, 2H), 7.72 (dd, J=2.5, and 8.9 Hz, 1H), 7.00 (d, J=9.0 Hz, 1H), 4.12 (dq, J=3.7, and 7.1 Hz, 1H), 3.78 (s, 3H), 3.63 (dd, J=4.3, and 14.7, Hz, 1H), 2.97 (td, J=2.6, and 13.1, Hz, 1H), 1.60-1.30 (m, 5H), 1.29-1.03 (m, 1H), and 0.98 (d, J=6.9 Hz, 3H); Retention Time=6.049 min; HRMS: m/z (M+Na)+=(Calculated for C₂₀H₂₃IN₂NaO₄S, 537.0315) found, 537.0318.

### N-(3-iodo-4-methoxyphenyl)-4-((3-methylthiomorpholino)sulfonyl)benzamide (126)

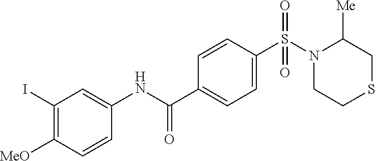 [SMILES: COc1ccc(NC(=O)c2ccc(S(=O)(=O)N3CCSCC3C)cc2)cc1I]

Follow the procedure for N-(4-bromo-3-iodophenyl)-4-((2-methylpiperidin-1-yl)sulfonyl)benzamide (127) using 3-methylthiomorpholine instead of 2-methylpiperidine. ¹H NMR (400 MHz, DMSO-d₆): δ 10.40 (s, 1H), 8.22 (d, J=2.5 Hz, 1H), 8.15-8.07 (m, 2H), 7.98-7.90 (m, 2H), 7.74 (dd, J=2.5, and 8.9 Hz, 1H), 7.01 (d, J=9.0 Hz, 1H), 4.34 (qt, J=2.9, and 6.6 Hz, 1H), 3.93 (dt, J=3.1, and 14.0 Hz, 1H), 3.80 (s, 3H), 3.18 (ddd, J=4.1, 10.6, and 14.3, Hz, 1H), 2.82-2.73 (m, 1H), 2.49-2.33 (m, 3H), and 1.16-1.09 (m, 3H); Retention Time=5.718 min; HRMS: m/z (M+H)+=(Calculated for C₁₉H₂₂IN₂O₄S₂, 533.0060) found, 533.0065.

### N-(4-bromo-3-iodophenyl)-4-((3-ethylmorpholino)sulfonyl)benzamide (129)

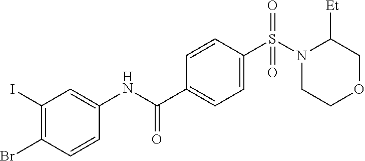 [SMILES: CCC1COCCN1S(=O)(=O)c1ccc(C(=O)Nc2ccc(Br)c(I)c2)cc1]

Follow synthesis for N-(4-bromo-3-iodophenyl)-4-((2-methylpiperidin-1-yl)sulfonyl)benzamide (127) using 3-ethyl morpholine as the starting material. For the amide formation use the following procedure. 4-((3-Ethylmorpholino)sulfonyl)benzoic acid (0.10 g, 0.334 mmol), DIPEA (0.18 mL, 1.00 mmol), HOBt (0.02 g, 0.10 mmol), and 4-bromo-3-iodoaniline (0.12 g, 0.40 mmol) were all stirred at rt before HATU (0.19 g, 0.50 mmol) was added. The reaction was stirred at rt for 4 hr and quenched with water and EtOAc. Wash EtOAc layer with water and brine, dry over Na₂SO₄, filter, concentrate and turn in for purification. ¹H NMR (400 MHz DMSO-d₆): δ 10.59 (s, 1H), 8.40 (d, J=2.4 Hz, 1H), 8.14-8.06 (m, 2H), 8.02-7.94 (m, 2H), 7.77-7.64 (m, 2H), 3.68-3.49 (m, 4H), 3.20 (ddd, J=3.4, 12.2, and 14.1 Hz, 1H), 3.17-3.07 (m, 1H), 2.99 (td, J=3.0, and 11.8 Hz, 1H), 1.64-1.48 (m, 2H), and 0.80 (t, J=7.4 Hz, 3H); Retention Time=6.215 min; HRMS: m/z (M+Na)+=(Calculated for C₁₉H₂₀BrIN₂NaO₄S, 602.9245) found, 602.9246.

### 4-((3-Ethylmorpholino)sulfonyl)-N-(3-iodo-4-methoxyphenyl)benzamide (132)

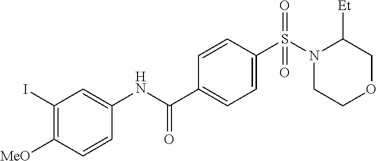 [SMILES: CCC1COCCN1S(=O)(=O)c1ccc(C(=O)Nc2ccc(OC)c(I)c2)cc1]

Follow synthesis for N-(4-bromo-3-iodophenyl)-4-((3-ethylmorpholino)sulfonyl)benzamide (127) using 3-iodo-4-methoxyaniline as the starting material for the amide coupling. ¹H NMR (400 MHz, DMSO-d₆): δ 10.39 (s, 1H), 8.21 (d, J=2.5 Hz, 1H), 8.14-8.06 (m, 2H), 8.01-7.93 (m, 2H), 7.73 (dd, J=2.6, and 8.9 Hz, 1H), 7.00 (d, J=9.0 Hz, 1H), 3.79 (s, 3H), 3.69-3.47 (m, 4H), 3.26-3.06 (m, 2H), 2.99 (td, J=3.0, and 11.8 Hz, 1H), 1.65-1.46 (m, 2H), and 0.80 (t, J=7.4 Hz, 3H); Retention Time=5.610 min; HRMS: m/z (M+H)+=(Calculated for C₂₀H₂₄IN₂O₅S, 531.0445) found, 531.0442.

### (+)-(S)-3-iodo-4-methoxy-N-(4-((2-methylpiperazin-1-yl)sulfonyl)phenyl)benzamide, HCl (130)

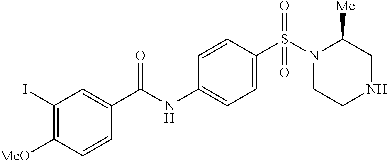 [SMILES: COc1ccc(C(=O)Nc2ccc(S(=O)(=O)N3CCNC[C@@H]3C)cc2)cc1I]

Positive enantiomer ¹H NMR (400 MHz, DMSO-d₆): δ 10.47 (s, 1H), 8.39 (d, J=2.2 Hz, 1H), 8.05-7.91 (m, 3H), 7.79-7.71 (m, 2H), 7.13 (d, J=8.8 Hz, 1H), 4.12-4.04 (m, 1H), 3.90 (s, 3H), 3.61-3.52 (m, 1H), 2.93 (td, J=2.6, and 13.0, Hz, 1H), 1.54-1.36 (m, 2H), 1.38 (s, 3H), 1.25-1.10 (m, 1H), and 0.98 (d, J=6.9 Hz, 3H); Retention Time=6.152 min; HRMS: m/z (M+H)+=(Calculated for C₂₀H₂₄IN₂O₄S 515.0496) found, 515.0498. The enantiomers were separated using CHIRALPAK AS column, at 35 mL/min, isocratic MeOH, to give ee's of >99% for the positive, and 98.7% of the negative compound.

### (−)-(R)-3-iodo-4-methoxy-N-(4-((2-methylpiperazin-1-yl)sulfonyl)phenyl)benzamide, HCl (131)

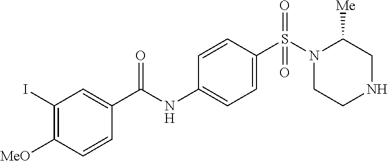 [SMILES: COc1ccc(C(=O)Nc2ccc(S(=O)(=O)N3CCNC[C@H]3C)cc2)cc1I]

1st negative enantiomer ¹H NMR (DMSO-d6400 MHz): δ 10.47 (s, 1H), 8.39 (d, J=2.2 Hz, 1H), 8.05-7.91 (m, 3H), 7.79-7.71 (m, 2H), 7.13 (d, J=8.8 Hz, 1H), 4.12-4.04 (m, 1H), 3.90 (s, 3H), 3.61-3.52 (m, 1H), 2.93 (td, J=2.6, and 13.0 Hz, 1H), 1.54-1.36 (m, 2H), 1.38 (s, 3H), 1.25-1.10 (m, 1H), and 0.98 (d, J=6.9 Hz, 3H); Retention Time=6.157 min; HRMS: m/z (M+H)+=(Calculated for C₂₀H₂₄IN₂O₄S, 515.0496) found, 515.0516. The enantiomers were separated using CHIRALPAK AS column, at 35 mL/min, isocratic MeOH, to give ee's of >99% for the positive, and 98.7% of the negative compound.

### (S)-4-((4-(3-iodo-4-methoxybenzamido)phenyl)sulfonyl)thiomorpholine-3-carboxamide (133)

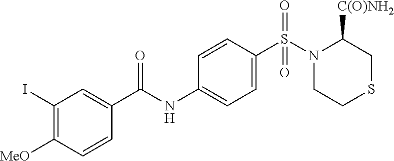

1^{st} negative enantiomer ¹H NMR (400 MHz, DMSO-d₆): δ 10.49 (s, 1H), 8.39 (d, J=2.2 Hz, 1H), 8.05-7.92 (m, 3H), 7.83-7.74 (m, 2H), 7.26 (d, J=12.1 Hz, 2H), 7.14 (d, J=8.8 Hz, 1H), 4.64 (t, J=3.4 Hz, 1H), 3.99-3.91 (m, 1H), 3.90 (s, 3H), 3.51 (ddd, J=5.9, 9.2, and 14.5 Hz, 1H), 2.93 (dd, J=2.9, and 14.1 Hz, 1H), 2.59 (dd, J=4.1, and 13.9 Hz, 1H), and 2.41-2.33 (m, 2H); Retention Time=4.930 min; HRMS: m/z (M+H)+=(Calculated for C₁₉H₂₁IN₃O₅S₂, 561.9962) found, 561.9954. The enantiomers were separated using CHIRALPAK. IA Mobile Phase: MeCN/IPA 80:20, flow rate: 35 mL/min; 1st_neg: ee>99%, 2nd_pos: ee 93.9%.

### (R)-4-((4-(3-iodo-4-methoxybenzamido)phenyl)sulfonyl)thiomorpholine-3-carboxamide (134)

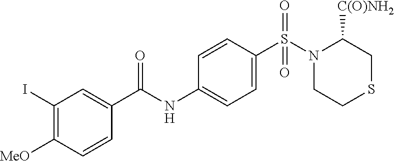

2^{nd} positive enantiomer ¹H NMR (400 MHz, DMSO-d₆): δ 10.49 (s, 1H), 8.39 (d, J=2.2 Hz, 1H), 8.05-7.92 (m, 3H), 7.83-7.74 (m, 2H), 7.26 (d, J=12.1 Hz, 2H), 7.14 (d, J=8.8 Hz, 1H), 4.64 (t, J=3.4 Hz, 1H), 3.99-3.91 (m, 1H), 3.90 (s, 3H), 3.51 (ddd, J=5.9, 9.2, and 14.5 Hz, 1H), 2.93 (dd, J=2.8, and 14.1 Hz, 1H), 2.59 (dd, J=4.1, and 13.9 Hz, 1H), and 2.41-2.33 (m, 2H); Retention Time=4.928 min; HRMS: m/z (M+H)+=(Calculated for C₁₉H₂₁IN₃O₅S₂, 561.9962) found, 561.9981.

### Example 2

### Remodilins Inhibit TGFβ-Induced Myofibroblast Transformation (MFT)

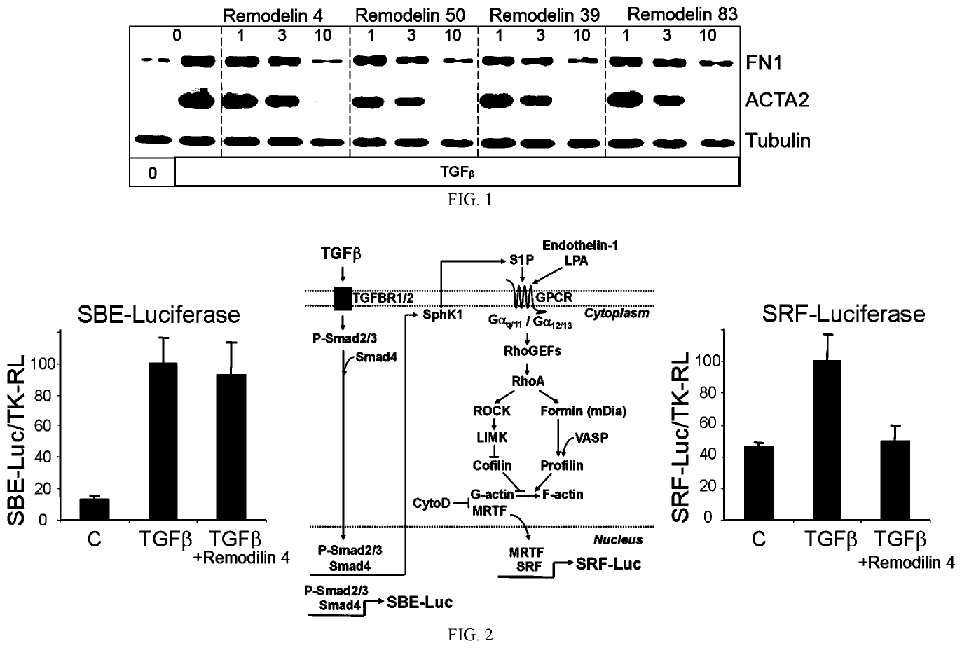

Remodilins 4, 39, 50, and 83 were found to blunt TGFβ-stimulated transformation of human lung-derived fibroblasts to the fibrosis-promoting myofibroblast phenotype in a dose-dependent fashion (FIG. 1).

In cultured human lung fibroblasts, remodilins act downstream of Smad signaling, which remains intact, though TGFβ-induced SRF activation is blocked (FIG. 2); SRF is necessary for expression of smooth muscle α-actin and other contractile proteins. Remodilins also suppress TGFβ-induced HIF1α expression in human lung fibroblasts and HIF1α-stimulated pathways in human airway myocytes.

### Example 3

### Remodilins Inhibit Cancer Cell Migration and Invasion In Vitro

TGFβ plays a role in breast cancer metastasis, and remodilins suppress aspects of TGFβ signaling related to cytoskeletal function. The effect of remodilins on the invasive and migratory properties of in vitro breast cancer cells was examined.

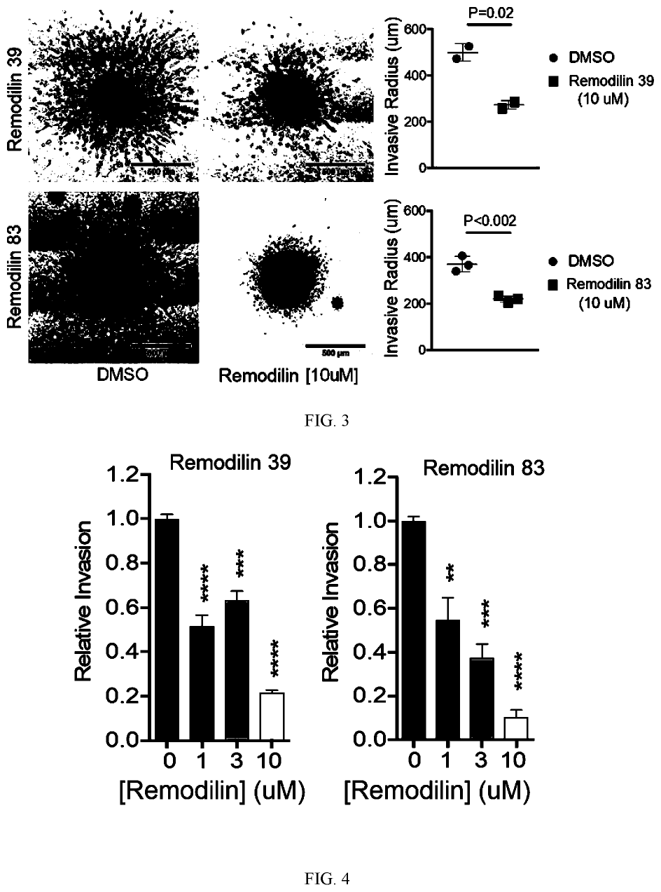

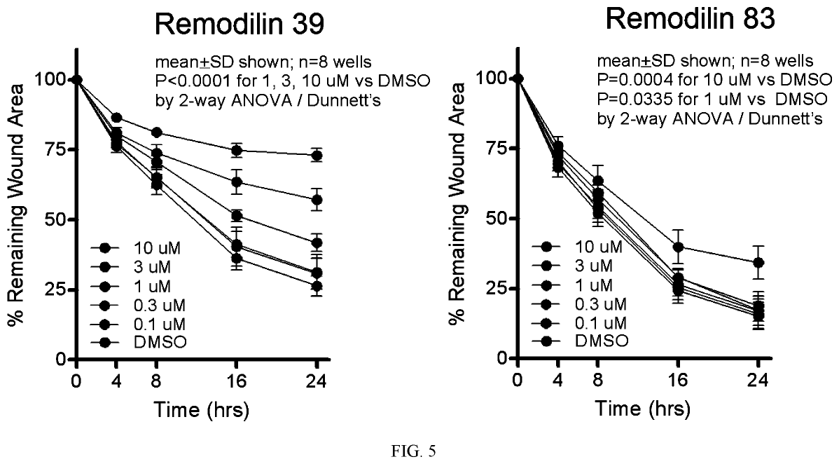

Remodilins 39 and 83 each slowed the invasion of triple negative MDA-MB-231 human breast cancer cells from spheroids into a surrounding collagen gel (FIG. 3), and also slowed the serum-directed invasion MDA-MB-231-derived BM1 cells through Matrigel-coated transwells (FIG. 4). Remodilins 39 and 83 also slowed the migration of MDA-MB-231 cells in a scratch wound healing assay (FIG. 5). Neither remodilin is growth-inhibitory or cytotoxic for MDA-MB-231 cells at concentrations to at least 10 uM by Alamar blue assay. The data indicates that remodilins represent a potential new anti-metastasis treatment for breast cancer and other cancers, as remodilins inhibited migration of ovarian cancer, lung cancer, and osteosarcoma cells; data not shown.

### Example 4

Remodilin in vivo pharmacokinetics (PK)

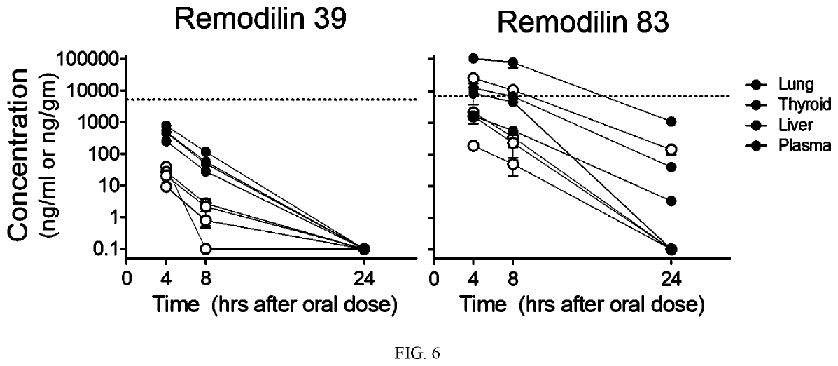

The in vivo bioavailability, plasma half-life, Cmax, and area under the curve (AUC) were determined after administration of single 10 mg/kg doses of remodilins 39 and 83 to mice (Table 1). Selected tissue concentrations of remodilins 39 and 83 after single oral doses of 10 or 50 mg/kg (FIG. 6). The PK properties of these remodilins differ substantially. Remodilin 83 is more orally bioavailable and has a longer plasma half-life, but it is highly preferentially distributed to the lung.

TABLE 1
In vivo PK and oral bioavailablity of
remodilins in wild-type mice (10 mg/kg single dose)

| | Cmax | Half-life | AUC | Oral |
|---|---|---|---|---|
| Remodilin | (uM) | (hrs) | (hr*ng/ml) | Bioavailability |
| 39 | 0.24 | 1.3 | 224 | 4% |
| 83 | 0.38 | 8.4 | 1303 | 22% |

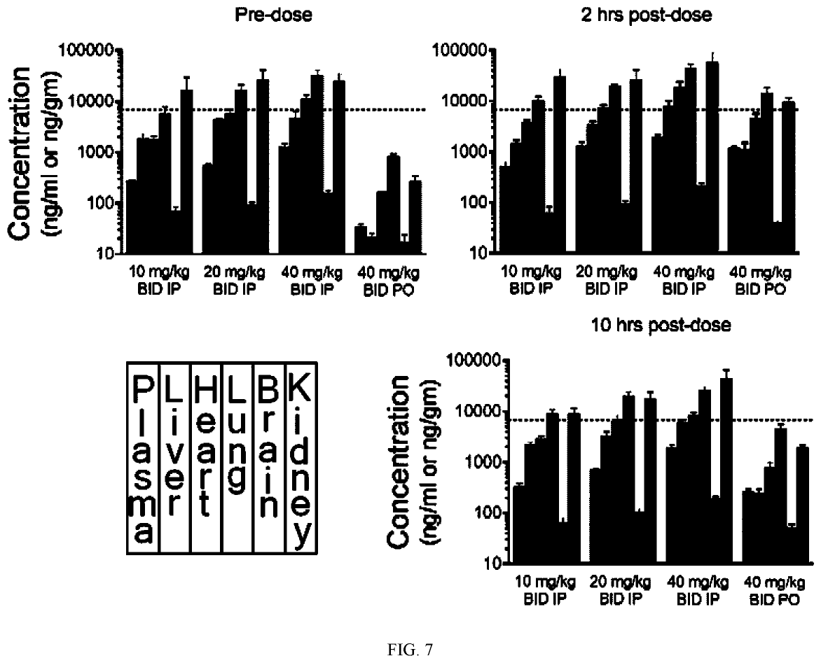

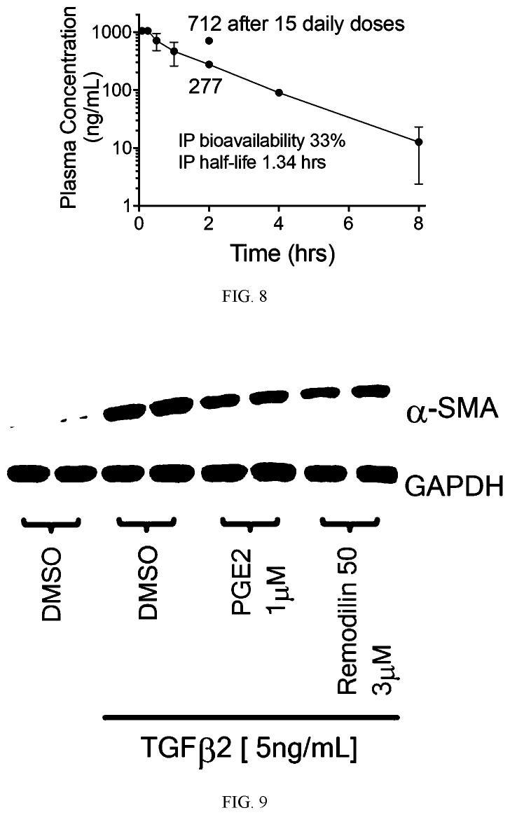

By contrast, remodilin 39 exhibits lower oral bioavailability and has a short plasma half-life, but it is distributed much more uniformly across tissues. Even with its longer half-life, single oral doses of remodilin 83 did not maintain tissue or plasma levels above 10 μM throughout the day (FIG. 6). PK was re-evaluated after 14 days of intraperitoneal (IP) or oral BID dosing. As shown in FIG. 7, 40 mg/kg BID IP was sufficient to maintain remodilin 83 levels near or above 10 μM in liver, heart, lung, and kidney. Brain levels of remodilin 83 were low, indicating that it may not cross the blood-brain barrier. Plasma levels after single or 15 consecutive once-daily IP 10 mg/kg doses of remodilin 39 were examined (FIG. 8). The plasma concentration of 10 μM remodilin 39 corresponds to 5314 ng/mL.

### Example 5

### Dosing Regimens Required to Maintain Anticipated Therapeutic Plasma and Tissue Concentrations

Remodilin 83 is lipophilic and is readily dissolved in either 20% Solutol (macrogol [15]-hydroxystearate, polyethylene glycol [15]-hydroxystearate, polyoxyethylated 12-hydroxystearic acid, Kolliphor; Solutol is a clinically acceptable excipient) or DMSO. In the experiments corresponding to FIG. 7, remodilin 83 was dissolved in 20% Solutol in PBS. The data in FIG. 7 demonstrate that 14-day 40 mg/kg BID IP dose regimen of remodilin 83 (in 20% Solutol) maintains liver and lung concentrations in female Balb/c mice just below 10 μM.

This study is replicated in female Balb/c including a measurement of mammary fat pad remodilin 83 levels, and doses of 30, 40, and 50 mg/kg BID IP to confirm results of FIG. 7, determine whether mammary fat pad remodilin 83 levels reach or exceed 10 μM, and verify if plasma concentrations increase appreciably with higher doses. Because remodilins 83 and 39 are highly lipid soluble, high mammary fat pad levels are expected. Given that BID dosing maintains high tissue levels throughout the 12 hour interdose interval (FIG. 7), continuous delivery through an Alzet pump is not expected to be required. Because of its marked redistribution out of plasma and into tissues, 10 μM remodilin 83 concentration in plasma may require higher doses. High plasma levels are easier to achieve with remodilin 39 because it is much more evenly distributed (FIG. 6). Having established the preferred dosing regimen in Balb/c mice, this regimen will be examined in athymic nude mice, adjusting it if necessary to maintain high tissue levels.

The experimental procedure examines remodilin effects on three mice at each timepoint (immediately before and 2 hrs after the last dose), all on day 14 of the dosing regimen. The use of 3 mice at each timepoint/condition allows for identification of outlier datapoints while minimizing animal use and cost.

### Example 6

### Remodilin Treatment Inhibits Breast Cancer Metastasis in Syngeneic Mouse or Human Xenograft Models

Mouse 4T1.2luc3 cells, which are stably transduced with the firefly luciferase gene, are used for syngeneic injection into syngeneic Balb/c mice; human MDA-MB-436 cells harboring firefly luciferase are injected into athymic nude mice. These highly metastatic cell lines lack expression of estrogen and progesterone receptors and of HER2 (EGFR2) and mimic “triple negative” breast cancer.

5×105 mouse or 2×106 human tumor cells are injected into the fourth left mammary fat pad of lightly anesthetized mice. Tumor volumes are measured by caliper twice per week and calculated as volume=(π/6)×width2×length; tumor growth is monitored by Xenogen imaging weekly (as the tumor cells harbor luciferase). All mice receive luciferin (luciferase substrate; 100 μL of 15 mg/mL solution IP) 15 minutes prior to Xenogen bioluminescence imaging. Experiments continue for 6 weeks to allow for development of lung, liver, and bone metastasis.

In mice exhibiting excessive tumor growth and morbidity, the primary tumor is be excised (on the same day from all animals in both remodilin-treated and vehicle-treated parallel groups) and experiments continue until mouse sacrifice at 6 weeks. At the time of sacrifice or earlier excision, primary tumor size, vascularization, and evidence of local invasion is evaluated by gross and microscopic pathology examination. In mice where remodilins inhibit primary tumor growth, then 106 cancer cells are given to additional mice by tail vein or left ventricular injection to induce lung or bone metastases, respectively.

At the time of sacrifice 6 weeks after tumor cell implantation, intravasation is assessed by assessing the relative abundances of circulating tumor cells. For human cancer cells, the human and mouse isoforms of GAPDH provide relative markers of intravasated tumor and endogenous circulating leukocytes, respectively; these are quantified by real-time PCR, using the ΔΔCt method. For mouse cancer cells, a distinguishing feature is the luciferase gene with which they are transduced (the human cells also have this); as such, the relative blood abundances of firefly luciferase and mouse GAPDH RNAs are measured by real-time PCR to reveal intravasation intensity. Comparison between human GAPDH/mouse GAPDH and luciferase/mouse GAPDH results in xenografted is be used to validate the latter method. Weekly Xenogen imaging, as described above, is used to monitor the growth of distant metastases in lung, liver, and bone. At the time of sacrifice, these organs and brain are examined for metastasis by gross inspection (after inflation with and fixation in Bouin's solution to highlight lung metastases and by bisection of each organ to allow for gross visual identification) and by microscopic quantification, incorporating methods of quantitative stereology to estimate total metastatic volume. Histologic features of metastases are also examined to reveal any potential differences between remodilin-vs vehicle-treated mice. Treatment with remodilin or its corresponding vehicle/delivery control are initiated 2 days prior to tumor cell implantation, in order to ensure that remodilin is present throughout the potential metastasis development period.

### Example 7

### Remodilins Inhibit Fibrogenic Activities in Trabecular Meshwork Cells

In primary open angle glaucoma (POAG) patients, the aqueous humor contains an elevated level of transforming growth factor β2 (TGF-β2) as compared with normal. TGF-β2 activates fibrogenic activities of two key cell types in conventional outflow pathway: trabecular meshwork (TM) cells and Schlemm's canal endothelial (SC) cells. In both TM and SC cells, TGF-β2 induces elevated expression of extracellular proteins (collagen, fibronectin and laminin) and contractile protein such as alpha-smooth muscle actin (α-SMA). These changes in extracellular matrix and contraction in conventional outflow pathway decrease outflow facility and thus elevate intraocular pressure. A novel class of small molecules (remodilins) inhibit TGF-β1 induced myofibroblast differentiation in vitro in human lung fibroblasts and human airway smooth muscle cells. The effect of remodilins on inhibition of TGF-β2 induced fibrogenic activities in the human outflow pathway is examined below.

In POAG, the increased resistance to aqueous humor outflow may be caused by increased extracellular matrix deposition by TM cells, leading to increased TM stiffness, and such fibrogenic change in trabecular meshwork is likely controlled by TGF-β2 pathways. Remodilins' effect on inhibiting fibrogenic activities in TGF-β2 treated TM cells was examined. Two different remodilins in TGF-β2 treated TM cells isolated from a normal post-mortem eye were examined. Expression of extracellular matrix proteins (collagen, fibronectin, laminin) and contractile protein (α-SMA) together with cellular contractile force were examined.

### Example 8

### Remodilins Inhibit TGF-β2 Induced Pro-Fibrogenic Activation in TM Cells

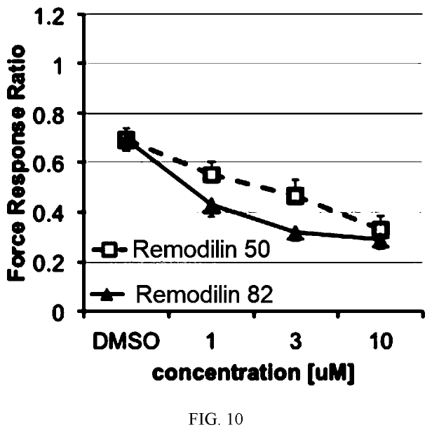

TM cells were cultured in 6-well plates and serum-deprived for one day before drug treatment. Cells were treated with prostaglandin E2 (PGE2, 1 μM), remodilin [3 μM] or vehicle control (dimethyl sulfoxide, DMSO) together with human recombinant TGF-β2 [5 ng/mL] for 48 hours. PGE2 was chosen as a positive control due to its known anti-fibrogenic effects. Compared to no treatment group, TGF-β2 treatment alone elevated α-SMA expressions and pre-treatment of PGE2 inhibited α-SMA expression induced by TGF-β2. Remodilin treatment inhibited α-SMA expression induced by TGF-β2 (FIG. 9). The effect of remodilin treatment alone on cellular contractile force in TM cells was examined. The effects of remodilin treatment using “force response ratio” which is a normalized average contractile force compared to baseline, was examined. Average contractile force was measured using Fourier-Transform Traction Microscopy. After 1 hr treatment, compared to DMSO, remodilins reduced average contractile force of TM cells in a dose dependent manner. (FIG. 10) These preliminary results suggest that remodilins not only inhibit profibrogenic activation of TM cells by TGF-β2 but also induce relaxation of TM cells. Therefore, remodilins may change structural compositions and mechanical properties of TM and therefore, may help increase outflow facility in glaucomatous eyes with elevated TGF-β2 level.

### Example 9

### Remodilins Inhibit TGF-β2 Induced Pro-Fibrogenic Transformation and Stiffening in SC Cells

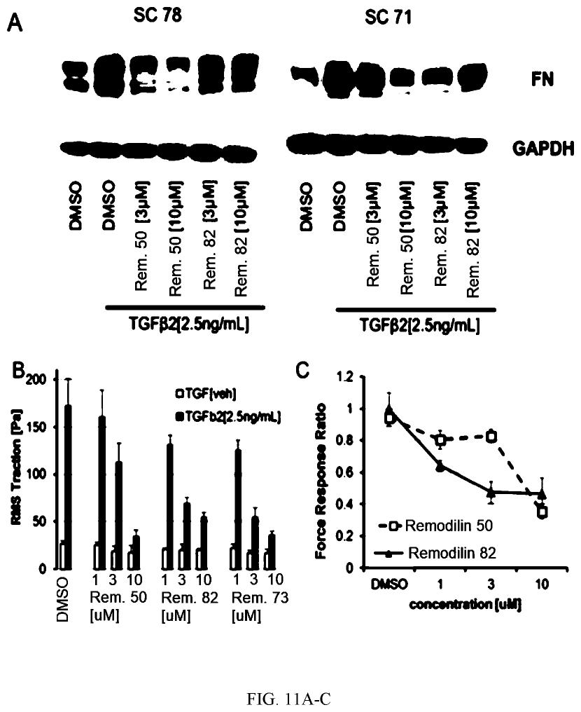

Remodilins' effect on SC cells, another key cell type in conventional outflow pathway, was examined. As described above, TGF-β2 also promotes pro-fibrogenic activation in SC cells and such activation accompanies elevation of cell contraction. Protein expression in TGF-β2 treated SC cells was examined first. SC cells from two different donors were cultured in 6-well plates and serum-deprived for 1 day before drug treatment. Cells were treated with Remodilin [3 or 0 μM] or DMSO together with human recombinant TGF-β2 [2.5 ng/mL] for 48 hours. Compared to no treatment, TGF-β2 treatment induced elevated expression of fibronectin, a key extracellular matrix protein secreted by SC cells. Pre-treatment of remodilins partially inhibited fibronectin expression in TGF-β2 treated SC cells (FIG. 11A).

Remodilins' effect on elevation of SC cell contraction by TGF-β2 treatment was then examined. Cellular contractile force was measured using Fourier-Transform Traction Microscopy. Compared to no treatment, TGF-β2 treatment elevated average cellular contractile force by almost seven-fold. When SC cells were pre-treated with three different remodilins, remodilins partially inhibited the elevation of contractile force in TGF-β2 treated SC cells in a dose dependent manner (FIG. 11B). Then, the effect of remodilin treatment alone on cellular contractile force in SC cells was investigated. After 1 hr treatment, compared to DMSO, remodilins reduced cellular contractile force in a dose dependent manner (FIG. 11C). These preliminary results suggest that remodilin not only inhibits pro-fibrogenic transformation of SC cells by TGF-β2 but also reduces the average contractile force of SC cells both with and without TGF-β2 treatment. Taken together, the anti-fibrogenic effects and relaxation effects of remodilins could be beneficial to reduce outflow resistance potentially through promoting pore formation in SC inner wall.

### Example 10

Remodilins Inhibit Pathways Regulated by Hypoxia-Inducible Factor-1 Alpha (HIF1α) in TGF-Beta (TFGβ)-Stimulated Human Airway Myocytes

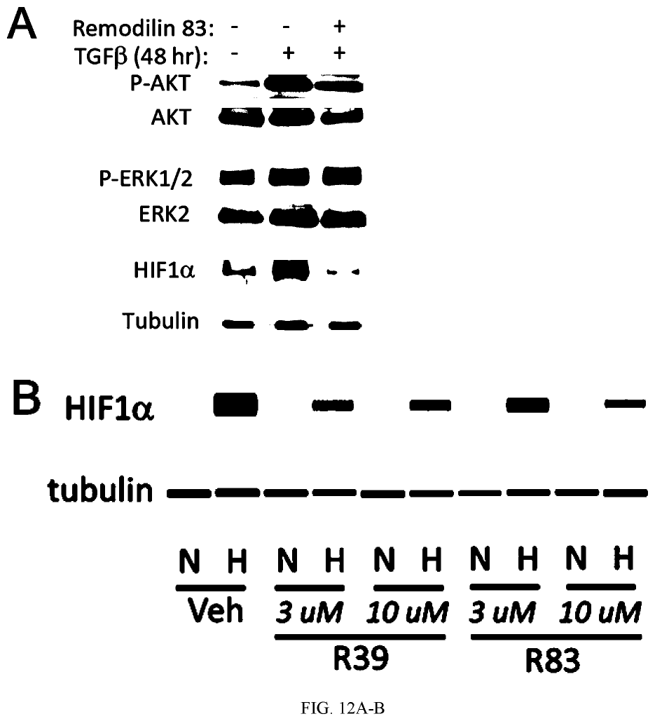

RNAseq analysis suggested that remodilins inhibit pathways regulated by hypoxia-inducible factor-1 alpha (HIF1α) in TGF-beta (TFGP)-stimulated human airway myocytes. The influence of remodilin 83 on HIF-1α accumulation in TGFβ-treated fibroblasts was examined. As shown in FIG. 12A, 48 hrs TGFβ treatment increased HIF1α abundance in cells treated with vehicle, and this response was absent cells treated with remodilin 83. Remodilin 83 also inhibited TGFβ-induced AKT phosphorylation, but not TGF0-induced ERK1/2 phosphorylation. Similarly, both remodilins 39 and 83 inhibited HIF1α accumulation in HEK293 cells exposed to 6 hrs of hypoxia (1% 02; FIG. 12B). Six hours of steady hypoxia (1% oxygen) induced HIF1α accumulation in vehicle (Veh)-treated HEK293 cells, but treatment with remodilin 39 (R39) or remodilin 83 (R83) at 3 μM or 10 μM (as indicated) blunted HIF1α accumulation substantially. N—normoxia; H—hypoxia.

### Example 11

### Effect of Remodilins on Oxygen Consumption Rate (OCR) and Extracellular Acidification Rate (ECAR)

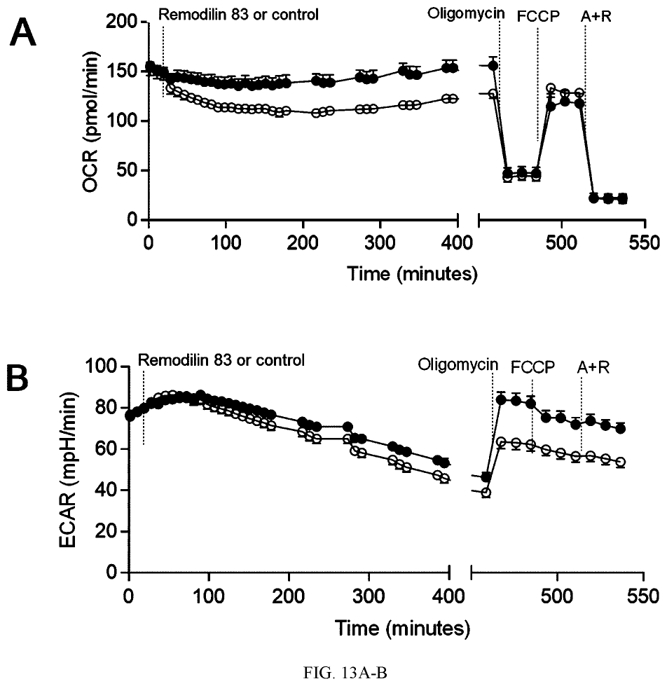

In light of the ability of remodilins to inhibit TGFβ-induced and hypoxia-induced HIF1α accumulation, and in light of additional RNAseq data suggesting that remodilins can inhibit glycolytic pathways, the effect of remodilin 83 on oxygen consumption rate (OCR, FIG. 13A) and extracellular acidification rate (ECAR, FIG. 13B), a marker of glycolysis, in cultured A549 lung adenocarcinoma cells was measured. Results indicate that remodilin 83 decreases both mitochondrial respiration and glycolysis. A549 cells were treated with remodilin 83 (open circles) or its vehicle (filled circles) at time=30 min, and oxygen consumption rate (OCR) and extracellular acidification rate (ECAR) were measured continuously over time. Cells were sequentially treated with oligomycin (ATP synthase inhibitor), FCCP (uncoupler) and antimycin A/rotenone (A+R) (complex III and I inhibitor). Results indicate that remodilins decrease both mitochondrial respiration and glycolysis.

Together, the results in FIGS. 12A-12B and 13A-13B indicate that remodilins inhibit HIF1α accumulation and decrease both mitochondrial respiration and glycolysis. These results imply that remodilins could find therapeutic roles in a wide range of diseases in which HIF signaling plays an important role, diseases in which tissue or systemic hypoxia plays an important role, and/or diseases in which cellular metabolism is dysregulated. Examples include renal cell cancer, pulmonary hypertension, retinopathy of the newborn, coronary vascular disease, tissue ischemia, mountain sickness characterized by pulmonary and brain edema, obstructive and/or central sleep apnea, and many others.

### Example 12

### Effect of Remodilins on Intermittent Hypoxia (IH) Induced by Obstructive Sleep Apnea (OSA)

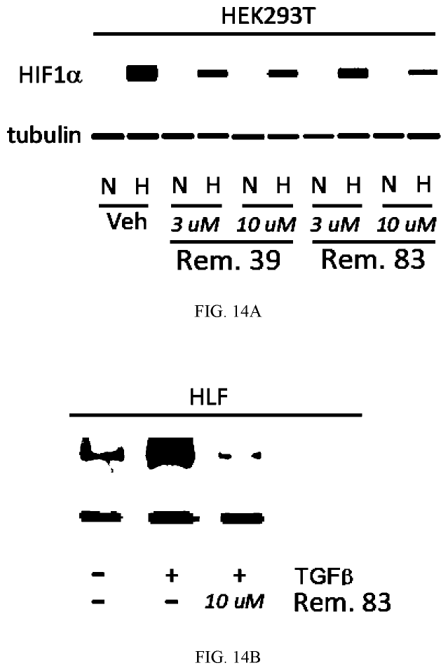

OSA is a chronic, morbid disease affecting about 10% of the adult population, in which frequent episodes of upper airway collapse obstruct inspiratory airflow and cause IH. These episodes of IH disrupt sleep but perhaps more importantly accelerate multiple cardiometabolic abnormalities, including systemic hypertension, cardiovascular disease, stroke, and abnormal glucose metabolism. Therefore, remodilins were examined for their ability on inhibit hypoxia-induced accumulation of HIF1α, a transcription factor that mediates many of the adverse responses to IH. The experiments represented in FIGS. 14A-14B demonstrate that two remodilins (39 and 83 at 3 or 10 μM as indicated) each inhibit the accumulation of HIF1α in cultured HEK293 T cells.

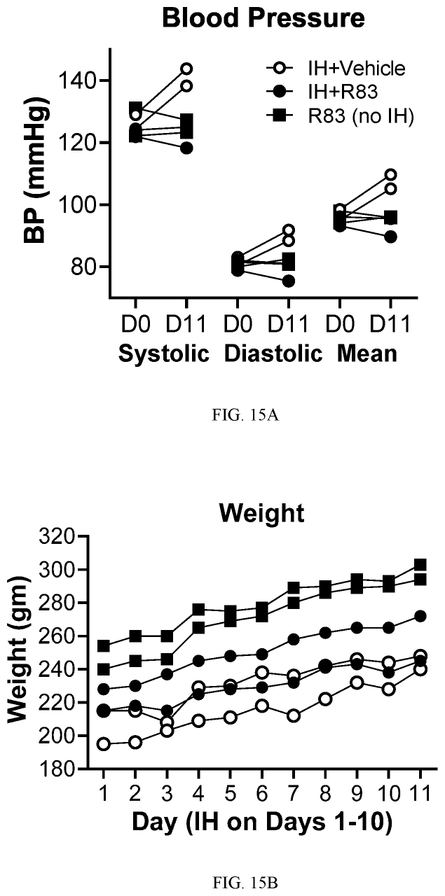

Remodilins were tested for their ability to inhibit the systemic hypertension induced in rats subjected to IH in vivo. As depicted in FIG. 15A, remodilin 83 blocks the systemic hypertension otherwise induced by 10 days of IH (filled circles) in Sprague Dawley rats in vehicle-treated rats (empty circles). Remodilin 83 had little effect on blood pressure in rats unexposed to IH (filled squares). Remodilins had no obvious effect on health as judged by clinical observation or weight gain (FIG. 15B).

Adverse cardiometabolic consequences of OSA are treated individually and without specific accounting for the fact that they are promoted (or entirely induced) by the intermittent hypoxia caused by OSA. Hypertension in OSA is treated with antihypertensives, cardiovascular disease prevention follows standard of care (statins, etc) to prevent heart disease and stroke, and glucose intolerance is treated with anti-hyperglycemics, including insulin. Critically, none of these current treatments addresses their more root cause in OSA—that is, the abnormal HIF1α accumulation and signaling induced by intermittent hypoxia. Remodelins may be used to blunt OSA/IH-induced HIF1α accumulation and signaling by interrupting the common pathogenetic pathway upstream of each of these cardiometabolic disturbances. Remodelins may therefore be used to prevent the induction of multiple disorders that currently require multiple drugs.

### Example 13

### Evaluation of Potential Remodilin Mechanisms of Action In Vivo

In vitro, remodilins inhibit serum response factor (SRF) activation by TGFβ, inhibit TGFβ-induced myofibroblast transformation (MFT), and inhibit proximal TGFβ-Smad signaling in breast cancer cells (not in fibroblasts). TGFβ facilitates breast cancer metastasis at multiple key steps including MFT, and SRF is overexpressed in multiple breast cancer types and contributes to sternness.

To test whether remodilins inhibit SRF activation in vivo, the effects of remodilins on luciferase expression (monitored by Xenogen imaging) in 4T1.2 and MDA-MB-436 tumors harboring a SRF-luc transgene (whose luciferase expression depends solely on SRF activity) will be compared to that of tumors in other mice created from corresponding breast cancer cells that constitutively express luciferase. Greater inhibition of SRF-dependent luciferase expression by remodilins would demonstrate their in vivo suppression of SRF activity. Mice will receive a remodilin (39 or 83) or its corresponding vehicle as control. Tumors, metastases, and surrounding tissues will be immunostained for MFT to determine whether modulation of MFT signaling contributes to remodilin effects in vivo. Primary tumors, metastases, and surrounding tissues will be immunostained for Smad4 to determine whether and in which cells Smad4 has translocated to the nucleus (reflecting active proximal TGFβ signaling) and for smooth muscle 3-actin as a marker of myofibroblast transformation, and scored semi-quantitatively for relative expression on a 0-4 scale. Immunostaining for 3-actin will be examined to allow detection and control for any non-specific effect of remodilin treatment.

All of the methods and compositions disclosed and claimed herein can be made and executed without undue experimentation in light of the present disclosure. While the compositions and methods of this invention have been described in terms of preferred embodiments, it will be apparent to those of skill in the art that variations may be applied to the methods and apparatuses and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of inhibiting serum response factor activity in a cell comprising administering to the cell a composition comprising an effective amount of a compound of Formula I: 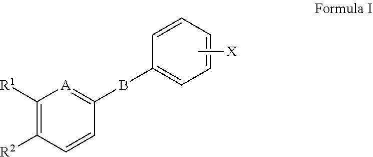
where:
A is —CH— or —N—;
B is —C(O)—NH—, —NH—C(O)—, —CH2-NH—, or —C(NH)—NH—;
X is —(Y)—NR³N⁴ OR NHSO₂Me;
Y is —SO₂—, —C(O)—, or —(CH₂)—;
R¹ and R² are each independently hydrogen, hydroxyl, substituted or unsubstituted alkyl, substituted or unsubstituted alkynyl, alkoxy, halide, nitrile, amine, acylamine, substituted or unsubstituted aryl, 4-6 member carbocycle, substituted or unsubstituted heterocycle, and
R³ and R⁴ are each independently hydrogen, substituted or unsubstituted alkyl, substituted or unsubstituted aromatic, substituted or unsubstituted carbocycle, substituted or unsubstituted heterocycle, substituted or unsubstituted bicyclic, or may join to form a carbocycle or heterocycle;
or a pharmaceutically acceptable salt, enantiomer, diastereomer, or prodrug thereof.

2. A method of inhibiting smooth muscle contractile protein accumulation in a cell comprising administering to the cell a composition comprising an effective amount of a compound of Formula I: 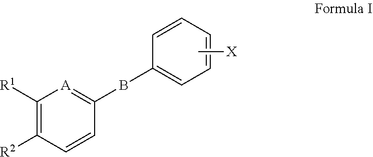
where:
A is —CH— or —N—;
B is —C(O)—NH—, —NH—C(O)—, —CH₂—NH—, or —C(NH)—NH—;
X is —(Y)—NR³R⁴ or NHSO₂Me;
Y is —SO₂—, —C(O)—, or —(CH₂)—;
R¹ and R² are each independently hydrogen, hydroxyl, substituted or unsubstituted alkyl, substituted or unsubstituted alkynyl, alkoxy, halide, nitrile, amine, acylamine, substituted or unsubstituted aryl, 4-6 member carbocycle, substituted or unsubstituted heterocycle, and
R³ and R⁴ are each independently hydrogen, substituted or unsubstituted alkyl, substituted or unsubstituted aromatic, substituted or unsubstituted carbocycle, substituted or unsubstituted heterocycle, substituted or unsubstituted bicyclic, or may join to form a carbocycle or heterocycle;
or a pharmaceutically acceptable salt, enantiomer, diastereomer, or prodrug thereof.

3. The method of claim 2, wherein the compound of Formula I inhibits localized accumulation of smooth muscle myosin heavy chains.

4. The method of claim 2 or 3, wherein the compound of Formula I inhibits localized accumulation of smooth muscle alpha actin.

5. The method of any of claims 1 to 4, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

6. The method of any of claims 1 to 5, wherein administering the composition is done prior to, concurrently with, or subsequent to chemotherapy, surgical treatment, immunotherapy, or radiation treatment.

7. A method of inhibiting smooth muscle contractile protein expression in a cell comprising administering to the cell a composition comprising an effective amount of a compound of Formula I: 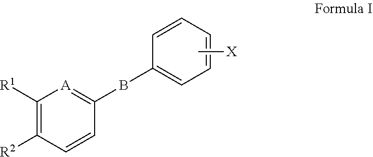
where:
A is —CH— or —N—;
B is —C(O)—NH—, —NH—C(O)—, —CH₂—NH—, or —C(NH)—NH—;
X is —(Y)—NR³R⁴ or NHSO₂Me;
Y is —SO₂—, —C(O)—, or —(CH₂)—;
R¹ and R² are each independently hydrogen, hydroxyl, substituted or unsubstituted alkyl, substituted or unsubstituted alkynyl, alkoxy, halide, nitrile, amine, acylamine, substituted or unsubstituted aryl, 4-6 member carbocycle, substituted or unsubstituted heterocycle, and
R³ and R⁴ are each independently hydrogen, substituted or unsubstituted alkyl, substituted or unsubstituted aromatic, substituted or unsubstituted carbocycle, substituted or unsubstituted heterocycle, substituted or unsubstituted bicyclic, or may join to form a carbocycle or heterocycle;
or a pharmaceutically acceptable salt, enantiomer, diastereomer, or prodrug thereof.

8. The method of claim 7, wherein the compound of Formula I inhibits expression of smooth muscle myosin heavy chains.

9. The method of claim 7 or 8, wherein the compound of Formula I inhibits expression of smooth muscle alpha actin.

10. A method of inhibiting tumor cell growth comprising administering to a subject in need of treatment a composition comprising an effective amount of a compound of Formula I: 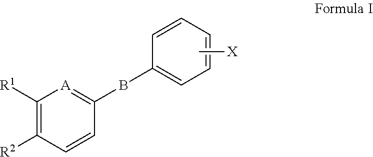
where:
A is —CH— or —N—;
B is —C(O)—NH—, —NH—C(O)—, —CH₂—NH—, or —C(NH)—NH—;
X is —(Y)—NR³R⁴ or NHSO₂Me;
Y is —SO₂—, —C(O)—, or —(CH₂)—;
R¹ and R² are each independently hydrogen, hydroxyl, substituted or unsubstituted alkyl, substituted or unsubstituted alkynyl, alkoxy, halide, nitrile, amine, acylamine, substituted or unsubstituted aryl, 4-6 member carbocycle, substituted or unsubstituted heterocycle, and
R³ and R⁴ are each independently hydrogen, substituted or unsubstituted alkyl, substituted or unsubstituted aromatic, substituted or unsubstituted carbocycle, substituted or unsubstituted heterocycle, substituted or unsubstituted bicyclic, or may join to form a carbocycle or heterocycle;
or a pharmaceutically acceptable salt, enantiomer, diastereomer, or prodrug thereof.

11. The method of claim 10, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

12. The method of either of claim 10 or 11, wherein the administering the composition is done prior to, concurrently with, or subsequent to chemotherapy, surgical treatment, immunotherapy, or radiation treatment.

13. The method of either of claims 10 to 12, wherein the compound of Formula I inhibits human serum response factor (SRF) activity.

14. The method of claim 13, wherein inhibition of SRF activity affects at least one of cell cycle regulation, apoptosis, cell growth, and cell differentiation.

15. A method of inhibiting metastasis in a subject having a tumor comprising administering to the subject a composition comprising a compound of Formula I: 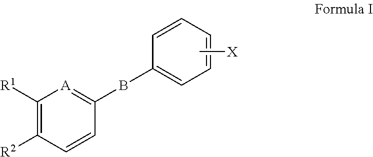
where:
A is —CH— or —N—;
B is —C(O)—NH—, —NH—C(O)—, —CH₂—NH—, or —C(NH)—NH—;
X is —(Y)—NR³R⁴ or NHSO₂Me;
Y is —SO₂—, —C(O)—, or —(CH₂)—;
R¹ and R² are each independently hydrogen, hydroxyl, substituted or unsubstituted alkyl, substituted or unsubstituted alkynyl, alkoxy, halide, nitrile, amine, acylamine, substituted or unsubstituted aryl, 4-6 member carbocycle, substituted or unsubstituted heterocycle, and
R³ and R⁴ are each independently hydrogen, substituted or unsubstituted alkyl, substituted or unsubstituted aromatic, substituted or unsubstituted carbocycle, substituted or unsubstituted heterocycle, substituted or unsubstituted bicyclic, or may join to form a carbocycle or heterocycle;
or a pharmaceutically acceptable salt, enantiomer, diastereomer, or prodrug thereof.

16. The method of claim 15, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

17. The method of either of claim 15 or 16, wherein the administering the composition is done prior to, concurrently with, or subsequent to chemotherapy, surgical treatment, immunotherapy, or radiation treatment.

18. A method of treating glaucoma comprising administering to a subject in need of treatment a composition comprising an effective amount of a compound of Formula I: 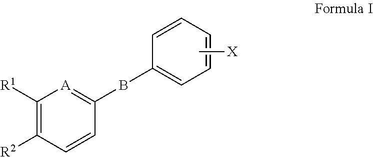
where:
A is —CH— or —N—;
B is —C(O)—NH—, —NH—C(O)—, —CH₂—NH—, or —C(NH)—NH—;
X is —(Y)—NR³R⁴ or NHSO₂Me;
Y is —SO₂—, —C(O)—, or —(CH₂)—;
R¹ and R² are each independently hydrogen, hydroxyl, substituted or unsubstituted alkyl, substituted or unsubstituted alkynyl, alkoxy, halide, nitrile, amine, acylamine, substituted or unsubstituted aryl, 4-6 member carbocycle, substituted or unsubstituted heterocycle, and
R³ and R⁴ are each independently hydrogen, substituted or unsubstituted alkyl, substituted or unsubstituted aromatic, substituted or unsubstituted carbocycle, substituted or unsubstituted heterocycle, substituted or unsubstituted bicyclic, or may join to form a carbocycle or heterocycle;
or a pharmaceutically acceptable salt, enantiomer, diastereomer, or prodrug thereof.

19. The method of claim 18, wherein the compound of Formula I stimulates generation of trabecular meshwork cells.

20. The method of claim 18 or 19, wherein the compound of Formula I stimulates generation of Schlemm's Canal cells.

21. The method of any of claims 18 to 20, wherein the compound of Formula I inhibits fibronectin expression.

22. The method of any of claims 18 to 21, wherein the composition is administered ocularly, parenterally, transmucosally, transdermally, intramuscularly, intravenously, intradermally, intravascularly, or subcutaneously, intraperitoneally, by topical drops or ointment, periocular injection, systemically by intravenous injection or orally, intracamerally into the anterior chamber or vitreous, via a depot attached to the intraocular lens implant inserted during surgery, or via a depot placed in the eye sutured in the anterior chamber or vitreous.

23. A method of reducing cellular metabolism, comprising administering to a subject in need of treatment a composition comprising an effective amount of a compound of Formula I: 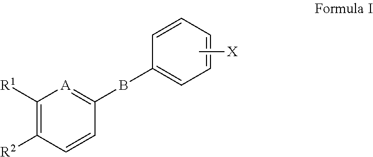
where:
A is —CH— or —N—;
B is —C(O)—NH—, —NH—C(O)—, —CH₂—NH—, or —C(NH)—NH—;
X is —(Y)—NR³R⁴ or NHSO₂Me;
Y is —SO₂—, —C(O)—, or —(CH₂)—;
R¹ and R² are each independently hydrogen, hydroxyl, substituted or unsubstituted alkyl, substituted or unsubstituted alkynyl, alkoxy, halide, nitrile, amine, acylamine, substituted or unsubstituted aryl, 4-6 member carbocycle, substituted or unsubstituted heterocycle, and
R³ and R⁴ are each independently hydrogen, substituted or unsubstituted alkyl, substituted or unsubstituted aromatic, substituted or unsubstituted carbocycle, substituted or unsubstituted heterocycle, substituted or unsubstituted bicyclic, or may join to form a carbocycle or heterocycle;
or a pharmaceutically acceptable salt, enantiomer, diastereomer, or prodrug thereof.

24. The method of claim 23, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

25. The method of either of claim 23 or 24, wherein administering the composition is done prior to, concurrently with, or subsequent to chemotherapy, surgical treatment, immunotherapy, or radiation treatment.

26. A method of attenuating hypoxia-induced response, comprising administering to a subject in need of treatment a composition comprising an effective amount of a compound of Formula I: 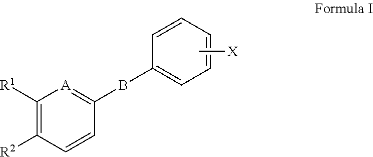
where:
A is —CH— or —N—;
B is —C(O)—NH—, —NH—C(O)—, —CH₂—NH—, or —C(NH)—NH—;
X is —(Y)—NR³R⁴ or NHSO₂Me;
Y is —SO₂—, —C(O)—, or —(CH₂)—;
R¹ and R² are each independently hydrogen, hydroxyl, substituted or unsubstituted alkyl, substituted or unsubstituted alkynyl, alkoxy, halide, nitrile, amine, acylamine, substituted or unsubstituted aryl, 4-6 member carbocycle, substituted or unsubstituted heterocycle, and
R³ and R⁴ are each independently hydrogen, substituted or unsubstituted alkyl, substituted or unsubstituted aromatic, substituted or unsubstituted carbocycle, substituted or unsubstituted heterocycle, substituted or unsubstituted bicyclic, or may join to form a carbocycle or heterocycle;
or a pharmaceutically acceptable salt, enantiomer, diastereomer, or prodrug thereof.

27. The method of claim 26, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

28. The method of either of claim 26 or 27, wherein administering the composition is done prior to, concurrently with, or subsequent to chemotherapy, surgical treatment, immunotherapy, or radiation treatment.

29. A method of inhibiting serum response factor activity in a cell comprising administering to the cell a composition comprising an effective amount of a compound of Formula II: 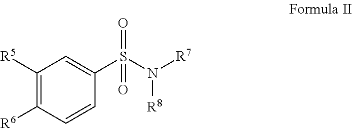
where:
where R⁵ and R⁶ are each independently hydrogen, halide, substituted or unsubstituted alkyl, alkoxy, amine, alkylamine, sulfonamide, or join together to form a 5 or 6 member carbocycle or heterocycle; and
R⁷ and R⁸ are each independently hydrogen, alkyl, or substituted or unsubstituted aryl, wherein the substituted aryl may be substituted with amide, sulfonamide, substituted or unsubstituted alkyl, or two adjacent carbon atoms on the substituted aryl ring form a carbocycle or heterocycle ring;
or a pharmaceutically acceptable salt, enantiomer, diastereomer, or prodrug thereof.

30. The method of claim 29, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

31. The method of either of claim 29 or 30, wherein administering the composition is done prior to, concurrently with, or subsequent to chemotherapy, surgical treatment, immunotherapy, or radiation treatment.

32. A method of inhibiting smooth muscle contractile protein accumulation in a cell comprising administering to the cell a composition comprising an effective amount of a compound Formula II: 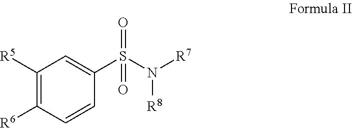
where:
where R⁵ and R⁶ are each independently hydrogen, halide, substituted or unsubstituted alkyl, alkoxy, amine, alkylamine, sulfonamide, or join together to form a 5 or 6 member carbocycle or heterocycle; and
R⁷ and R⁸ are each independently hydrogen, alkyl, or substituted or unsubstituted aryl, wherein the substituted aryl may be substituted with amide, sulfonamide, substituted or unsubstituted alkyl, or two adjacent carbon atoms on the substituted aryl ring form a carbocycle or heterocycle ring;
or a pharmaceutically acceptable salt, enantiomer, diastereomer, or prodrug thereof.

33. The method of claim 32, wherein the compound of Formula II inhibits localized accumulation of smooth muscle myosin heavy chains.

34. The method of claim 32 or 33, wherein the compound of Formula II inhibits localized accumulation of smooth muscle alpha actin.

35. The method of any of claims 32 to 34, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

36. The method of any of claims 32 to 35, wherein administering the composition is done prior to, concurrently with, or subsequent to chemotherapy, surgical treatment, immunotherapy, or radiation treatment.

37. A method of inhibiting smooth muscle contractile protein expression in a cell comprising administering to the cell a composition comprising an effective amount of a compound of Formula II: 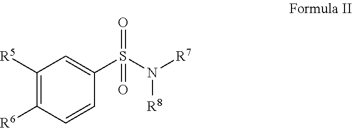
where:
where R⁵ and R⁶ are each independently hydrogen, halide, substituted or unsubstituted alkyl, alkoxy, amine, alkylamine, sulfonamide, or join together to form a 5 or 6 member carbocycle or heterocycle; and
R⁷ and R⁸ are each independently hydrogen, alkyl, or substituted or unsubstituted aryl, wherein the substituted aryl may be substituted with amide, sulfonamide, substituted or unsubstituted alkyl, or two adjacent carbon atoms on the substituted aryl ring form a carbocycle or heterocycle ring;
or a pharmaceutically acceptable salt, enantiomer, diastereomer, or prodrug thereof.

38. The method of claim 37, wherein the compound of Formula II inhibits expression of smooth muscle myosin heavy chains.

39. The method of claim 37 or 38, wherein the compound of Formula II inhibits expression of smooth muscle alpha actin.

40. The method of any of claims 37 to 39, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

41. The method of any of claims 37 to 40, wherein administering the composition is done prior to, concurrently with, or subsequent to chemotherapy, surgical treatment, immunotherapy, or radiation treatment.

42. A method of inhibiting tumor cell growth comprising administering to a subject in need of treatment a composition comprising an effective amount of a compound of Formula II: 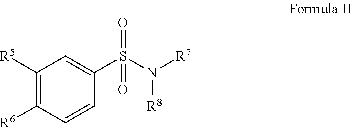
where:
where R⁵ and R⁶ are each independently hydrogen, halide, substituted or unsubstituted alkyl, alkoxy, amine, alkylamine, sulfonamide, or join together to form a 5 or 6 member carbocycle or heterocycle; and
R⁷ and R⁸ are each independently hydrogen, alkyl, or substituted or unsubstituted aryl, wherein the substituted aryl may be substituted with amide, sulfonamide, substituted or unsubstituted alkyl, or two adjacent carbon atoms on the substituted aryl ring form a carbocycle or heterocycle ring;
or a pharmaceutically acceptable salt, enantiomer, diastereomer, or prodrug thereof.

43. The method of claim 42, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

44. The method of either of claim 42 or 43, wherein the administering the composition is done prior to, concurrently with, or subsequent to chemotherapy, surgical treatment, immunotherapy, or radiation treatment.

45. The method of either of claims 42 to 44, wherein the compound of Formula II inhibits human serum response factor (SRF) activity.

46. The method of claim 45, wherein inhibition of SRF activity affects at least one of cell cycle regulation, apoptosis, cell growth, and cell differentiation.

47. A method of inhibiting metastasis in a subject having a tumor comprising administering to the subject a composition comprising a compound of Formula II: 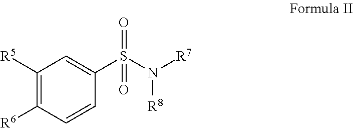
where:
where R⁵ and R⁶ are each independently hydrogen, halide, substituted or unsubstituted alkyl, alkoxy, amine, alkylamine, sulfonamide, or join together to form a 5 or 6 member carbocycle or heterocycle; and
R⁷ and R⁸ are each independently hydrogen, alkyl, or substituted or unsubstituted aryl, wherein the substituted aryl may be substituted with amide, sulfonamide, substituted or unsubstituted alkyl, or two adjacent carbon atoms on the substituted aryl ring form a carbocycle or heterocycle ring;
or a pharmaceutically acceptable salt, enantiomer, diastereomer, or prodrug thereof.

48. The method of claim 47, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

49. The method of claim 47 or 48, wherein the administering the composition is done prior to, concurrently with, or subsequent to chemotherapy, surgical treatment, immunotherapy, or radiation treatment.

50. A method of treating glaucoma comprising administering to a subject in need of treatment a composition comprising an effective amount of a compound of Formula II: 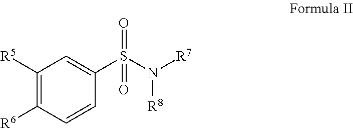
where:
where R⁵ and R⁶ are each independently hydrogen, halide, substituted or unsubstituted alkyl, alkoxy, amine, alkylamine, sulfonamide, or join together to form a 5 or 6 member carbocycle or heterocycle; and
R⁷ and R⁸ are each independently hydrogen, alkyl, or substituted or unsubstituted aryl, wherein the substituted aryl may be substituted with amide, sulfonamide, substituted or unsubstituted alkyl, or two adjacent carbon atoms on the substituted aryl ring form a carbocycle or heterocycle ring;
or a pharmaceutically acceptable salt, enantiomer, diastereomer, or prodrug thereof.

51. The method of claim 50, wherein the compound of Formula II stimulates generation of trabecular meshwork cells.

52. The method of claim 50 or 51, wherein the compound of Formula II stimulates generation of Schlemm's Canal cells.

53. The method of any of claims 50 to 529, wherein the compound of Formula II inhibits fibronectin expression.

54. The method of any of claims 50 to 40, wherein the composition is administered ocularly, parenterally, transmucosally, transdermally, intramuscularly, intravenously, intradermally, intravascularly, or subcutaneously, intraperitoneally, by topical drops or ointment, periocular injection, systemically by intravenous injection or orally, intracamerally into the anterior chamber or vitreous, via a depot attached to the intraocular lens implant inserted during surgery, or via a depot placed in the eye sutured in the anterior chamber or vitreous.

55. A method of reducing cellular metabolism, comprising administering to a subject in need of treatment a composition comprising an effective amount of a compound of Formula II: 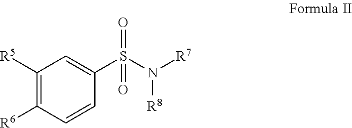
where:
where R⁵ and R⁶ are each independently hydrogen, halide, substituted or unsubstituted alkyl, alkoxy, amine, alkylamine, sulfonamide, or join together to form a 5 or 6 member carbocycle or heterocycle; and
R⁷ and R⁸ are each independently hydrogen, alkyl, or substituted or unsubstituted aryl, wherein the substituted aryl may be substituted with amide, sulfonamide, substituted or unsubstituted alkyl, or two adjacent carbon atoms on the substituted aryl ring form a carbocycle or heterocycle ring;
or a pharmaceutically acceptable salt, enantiomer, diastereomer, or prodrug thereof.

56. The method of claim 55, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

57. The method of claim 55 or 56, wherein the administering the composition is done prior to, concurrently with, or subsequent to chemotherapy, surgical treatment, immunotherapy, or radiation treatment.

58. A method of attenuating hypoxia-induced response, comprising administering to a subject in need of treatment a composition comprising an effective amount of a compound of Formula II: 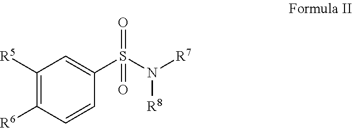
where:
where R⁵ and R⁶ are each independently hydrogen, halide, substituted or unsubstituted alkyl, alkoxy, amine, alkylamine, sulfonamide, or join together to form a 5 or 6 member carbocycle or heterocycle; and
R⁷ and R⁸ are each independently hydrogen, alkyl, or substituted or unsubstituted aryl, wherein the substituted aryl may be substituted with amide, sulfonamide, substituted or unsubstituted alkyl, or two adjacent carbon atoms on the substituted aryl ring form a carbocycle or heterocycle ring;
or a pharmaceutically acceptable salt, enantiomer, diastereomer, or prodrug thereof.

59. The method of claim 58, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

60. The method of claim 58 or 59, wherein the administering the composition is done prior to, concurrently with, or subsequent to chemotherapy, surgical treatment, immunotherapy, or radiation treatment.

61. The method of any of claims 1 to 28, wherein the compound of Formula I is further defined as at least one of 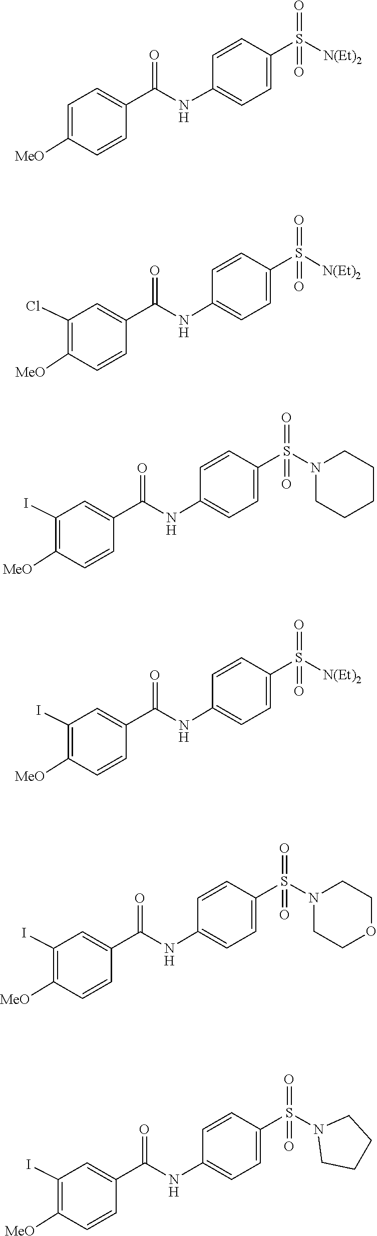 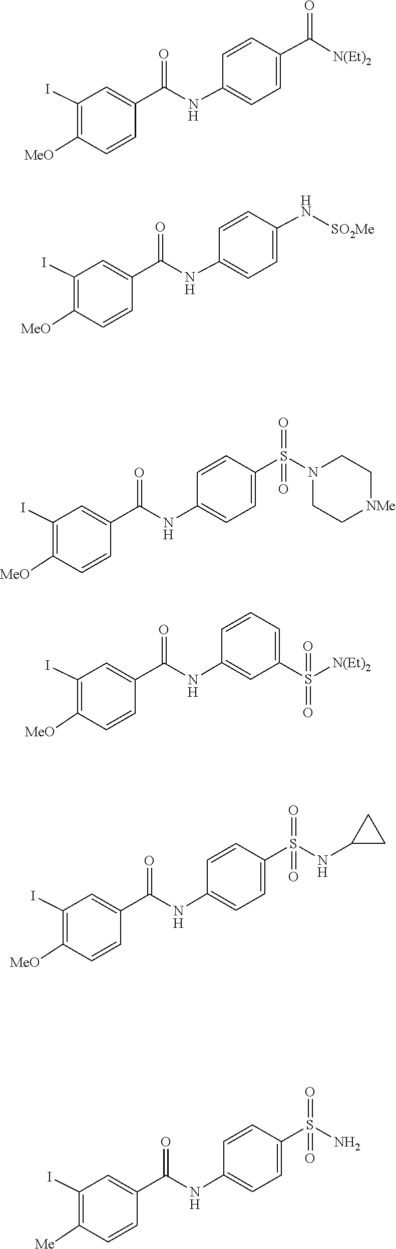 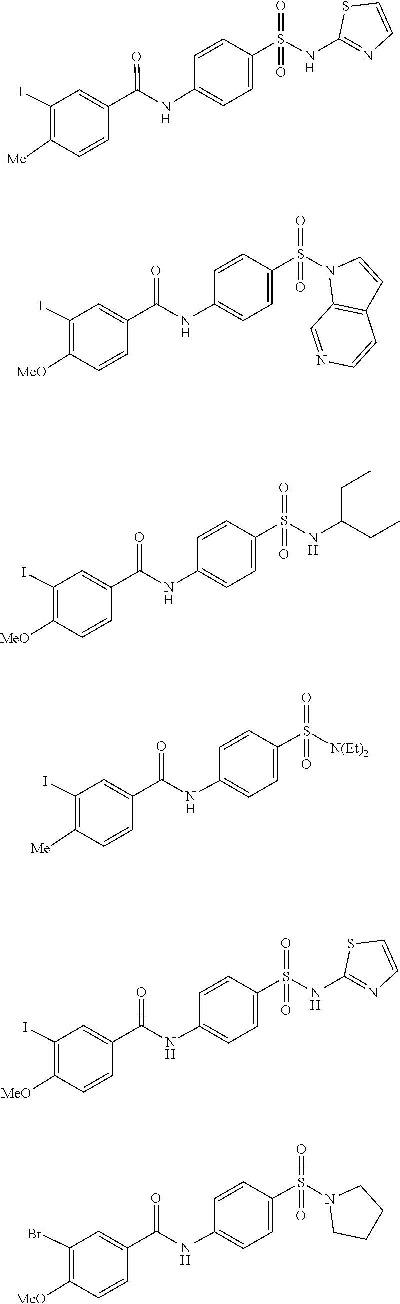 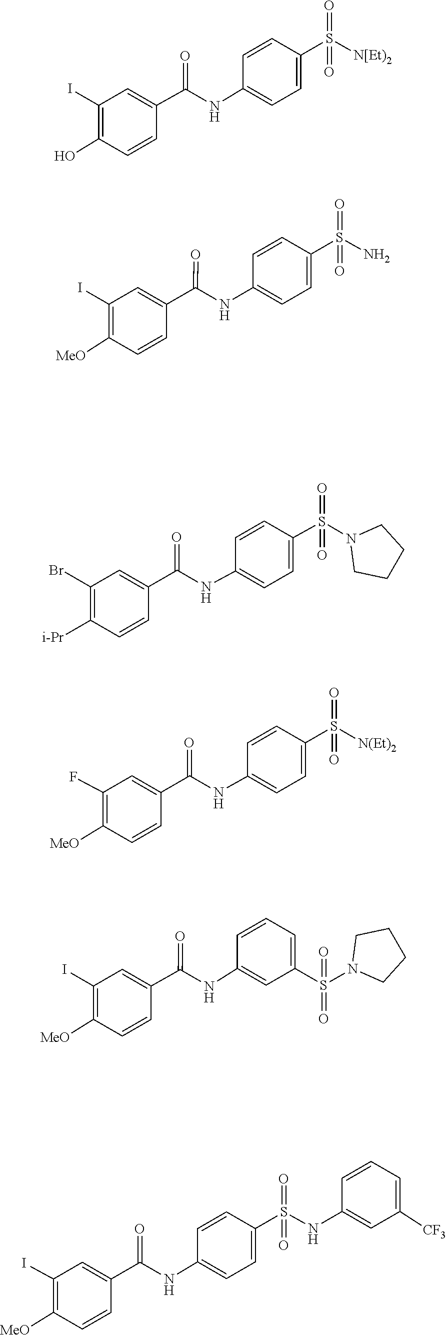 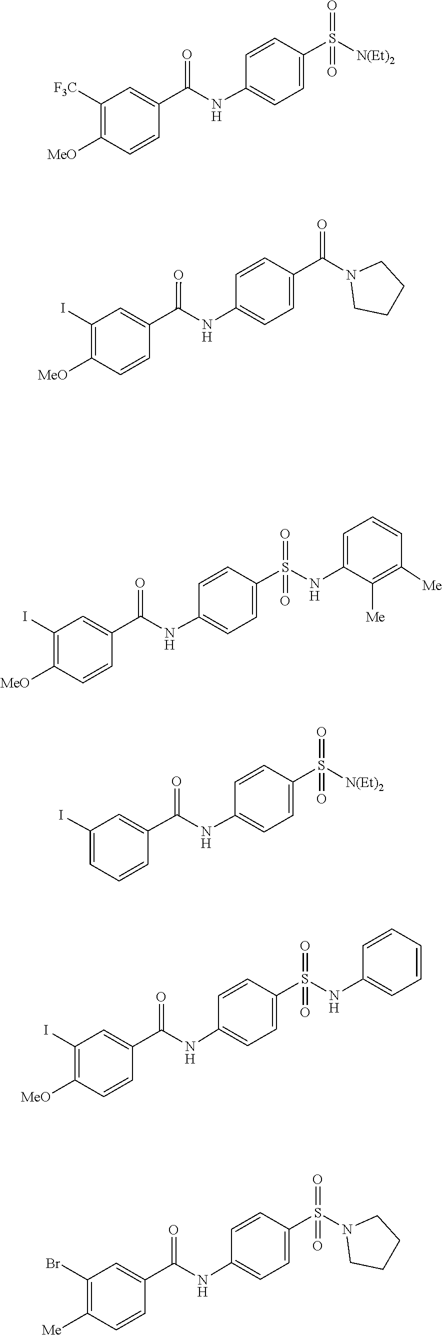 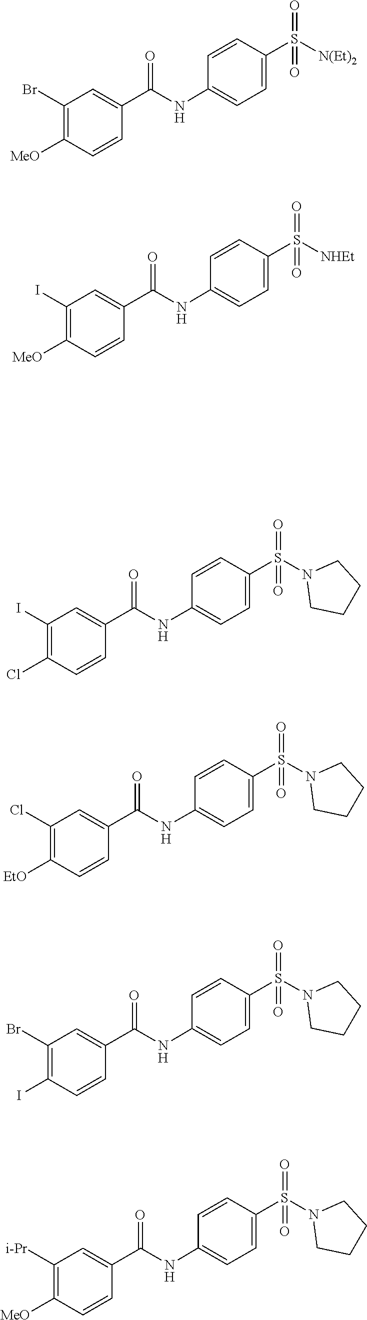 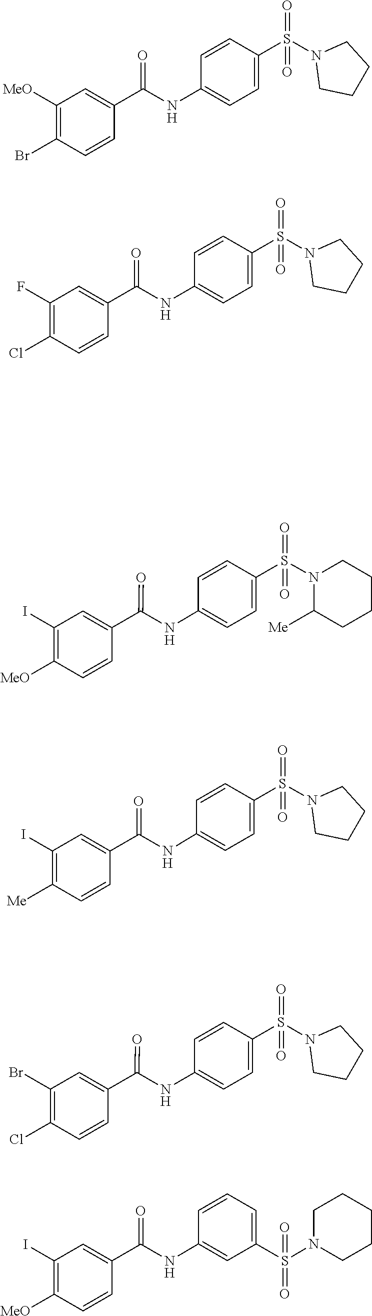 [SMILES: COc1cc(C(=O)Nc2ccc(S(=O)(=O)N3CCCC3)cc2)ccc1Br ; O=C(Nc1ccc(S(=O)(=O)N2CCCC2)cc1)c1ccc(Cl)c(F)c1 ; COc1ccc(C(=O)Nc2ccc(S(=O)(=O)N3CCCCC3C)cc2)cc1I ; Cc1ccc(C(=O)Nc2ccc(S(=O)(=O)N3CCCC3)cc2)cc1I ; O=C(Nc1ccc(S(=O)(=O)N2CCCC2)cc1)c1ccc(Cl)c(Br)c1 ; COc1ccc(C(=O)Nc2cccc(S(=O)(=O)N3CCCCC3)c2)cc1I] 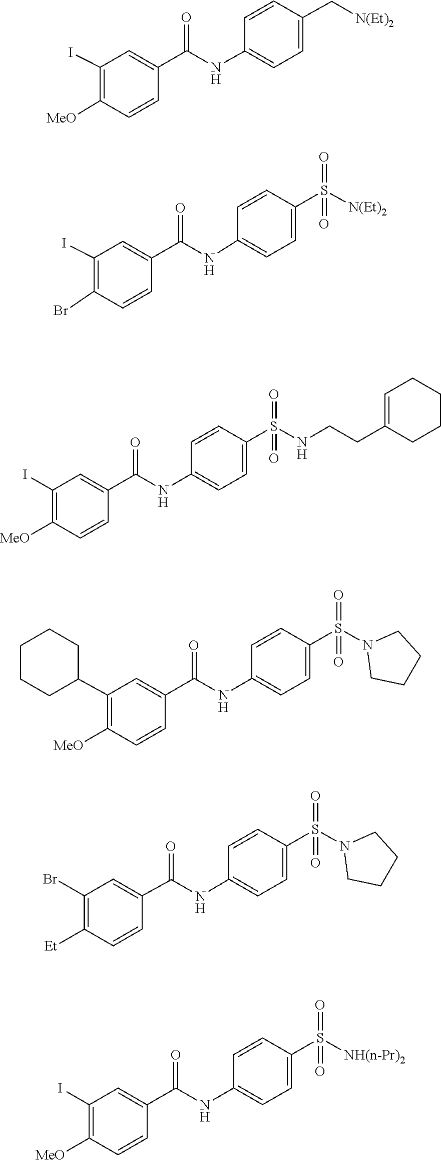 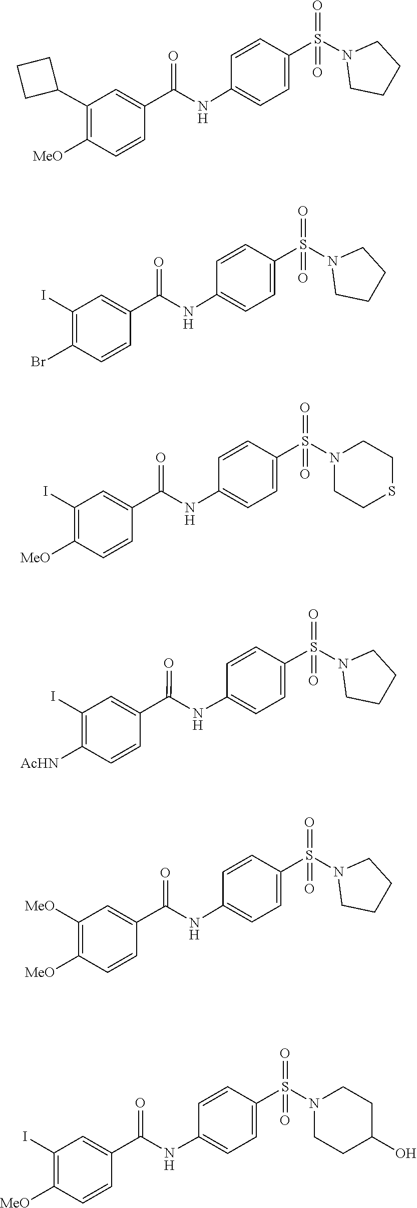 [SMILES: COc1ccc(C(=O)Nc2ccc(S(=O)(=O)N3CCCC3)cc2)cc1C1CCC1 ; O=C(Nc1ccc(S(=O)(=O)N2CCCC2)cc1)c1ccc(Br)c(I)c1 ; COc1ccc(C(=O)Nc2ccc(S(=O)(=O)N3CCSCC3)cc2)cc1I ; CC(=O)Nc1ccc(C(=O)Nc2ccc(S(=O)(=O)N3CCCC3)cc2)cc1I ; COc1ccc(C(=O)Nc2ccc(S(=O)(=O)N3CCCC3)cc2)cc1OC ; COc1ccc(C(=O)Nc2ccc(S(=O)(=O)N3CCC(O)CC3)cc2)cc1I] 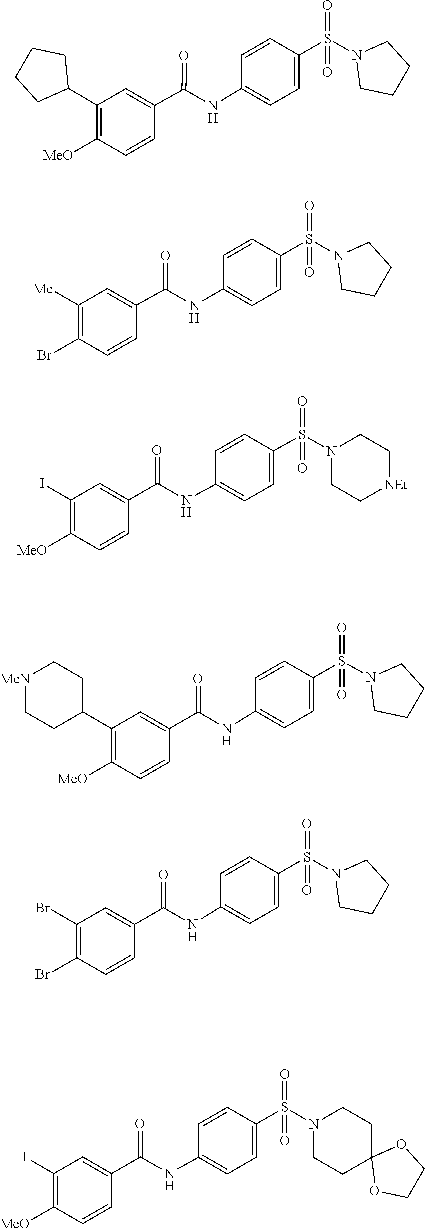 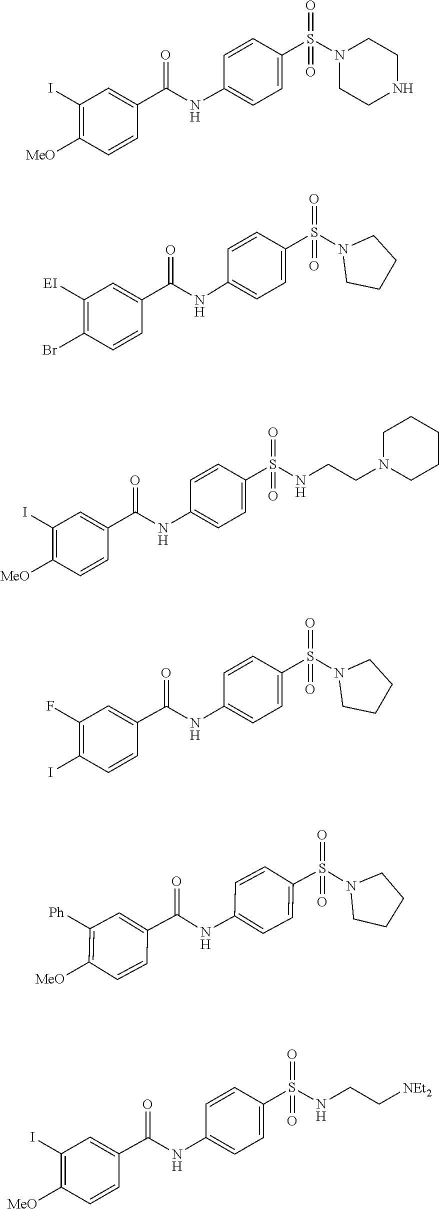 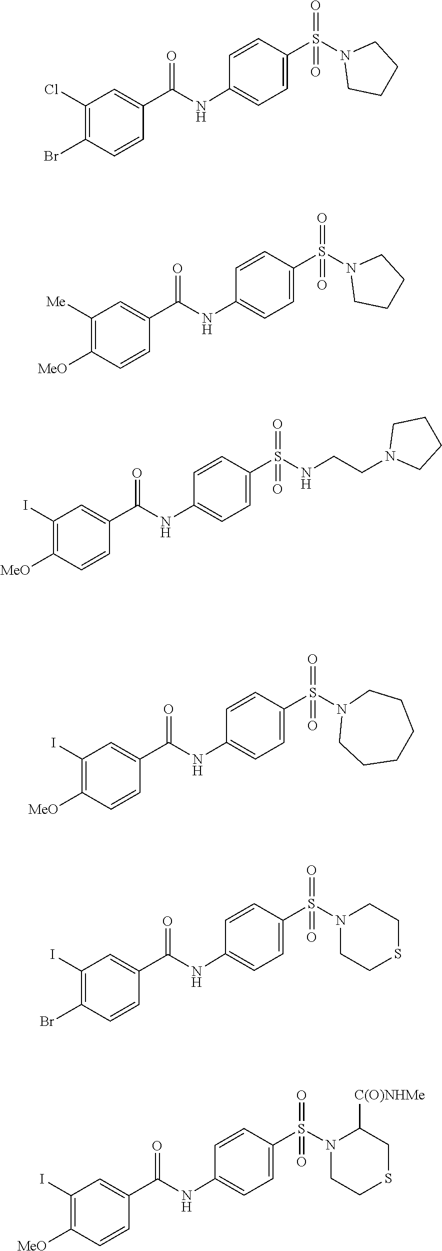 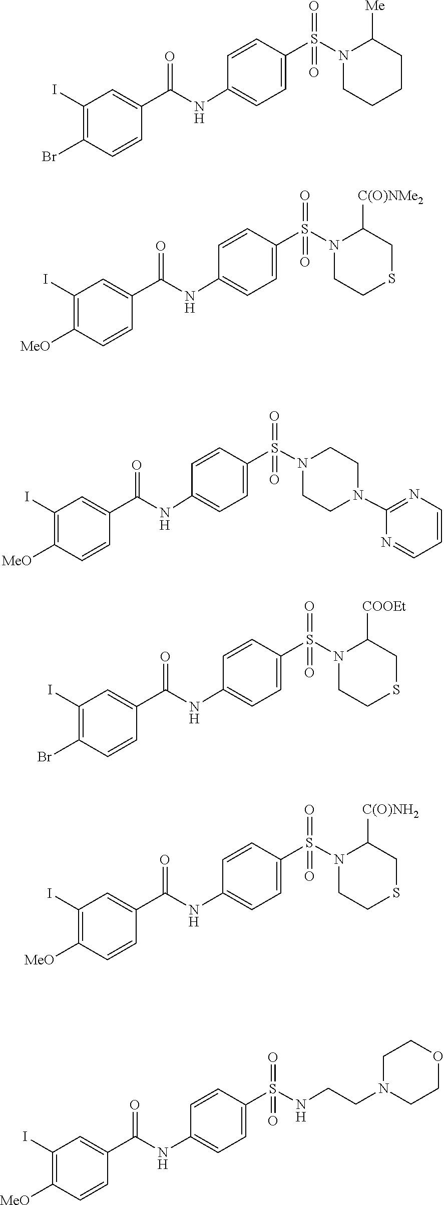 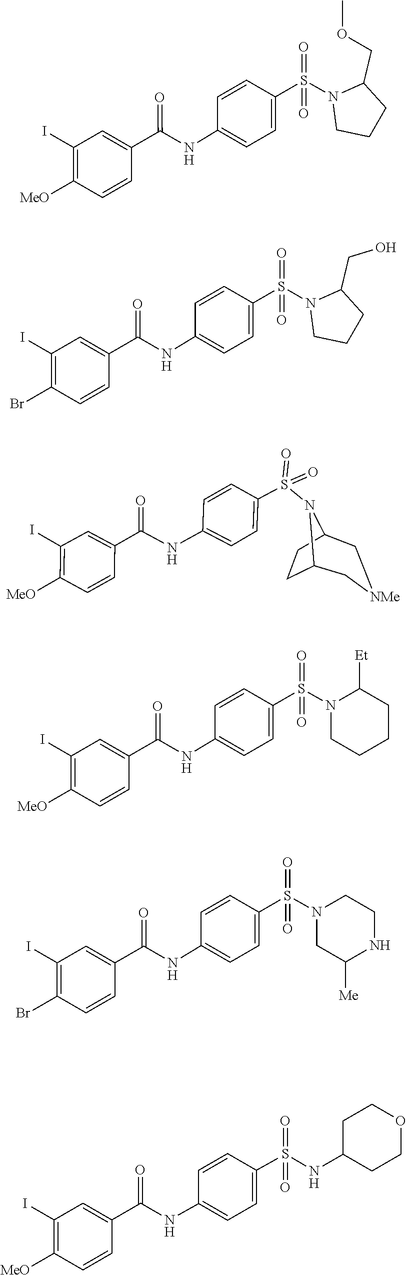 [SMILES: COCC1CCCN1S(=O)(=O)c1ccc(NC(=O)c2ccc(OC)c(I)c2)cc1 ; O=C(Nc1ccc(S(=O)(=O)N2CCCC2CO)cc1)c1ccc(Br)c(I)c1 ; COc1ccc(C(=O)Nc2ccc(S(=O)(=O)N3C4CCC3CN(C)C4)cc2)cc1I ; CCC1CCCCN1S(=O)(=O)c1ccc(NC(=O)c2ccc(OC)c(I)c2)cc1 ; CC1CN(S(=O)(=O)c2ccc(NC(=O)c3ccc(Br)c(I)c3)cc2)CCN1 ; COc1ccc(C(=O)Nc2ccc(S(=O)(=O)NC3CCOCC3)cc2)cc1I] 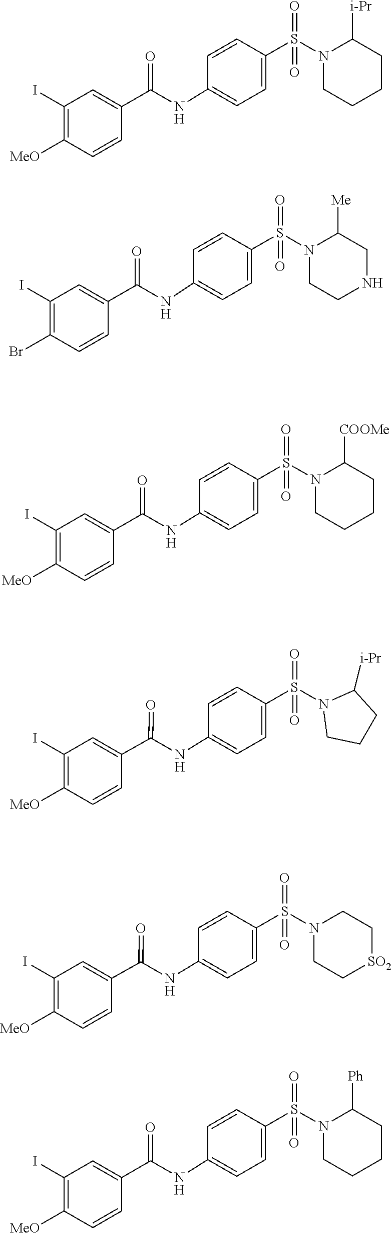 [SMILES: COc1ccc(C(=O)Nc2ccc(S(=O)(=O)N3CCCCC3C(C)C)cc2)cc1I ; CC1CNCCN1S(=O)(=O)c1ccc(NC(=O)c2ccc(Br)c(I)c2)cc1 ; COC(=O)C1CCCCN1S(=O)(=O)c1ccc(NC(=O)c2ccc(OC)c(I)c2)cc1 ; COc1ccc(C(=O)Nc2ccc(S(=O)(=O)N3CCCC3C(C)C)cc2)cc1I ; COc1ccc(C(=O)Nc2ccc(S(=O)(=O)N3CCS(=O)(=O)CC3)cc2)cc1I ; COc1ccc(C(=O)Nc2ccc(S(=O)(=O)N3CCCCC3c3ccccc3)cc2)cc1I] 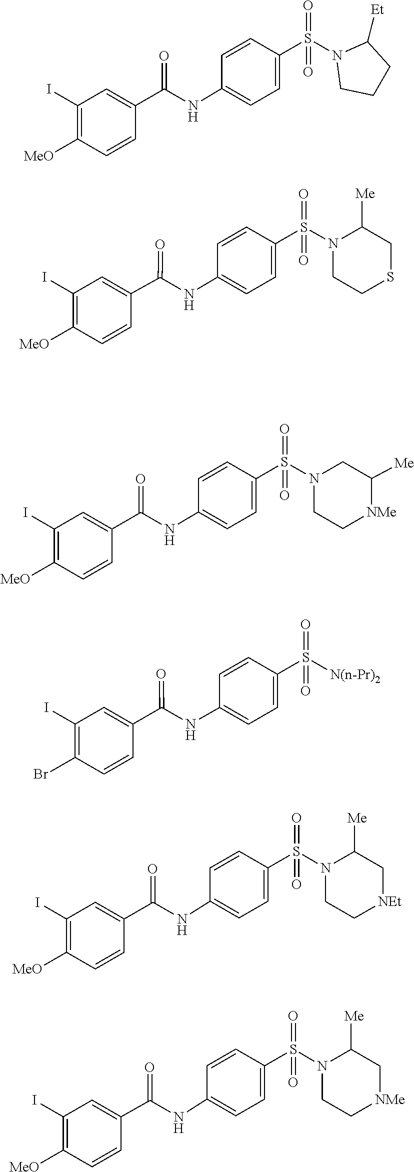 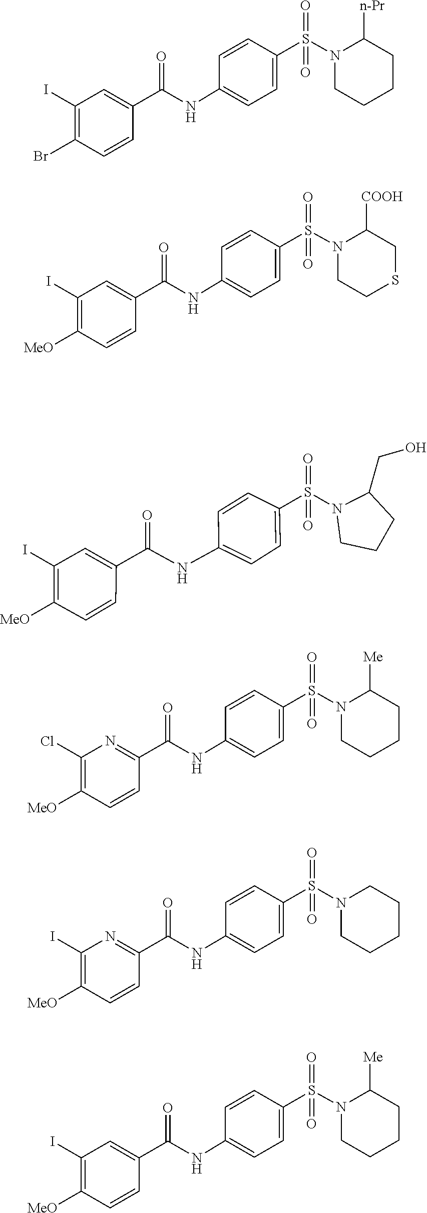 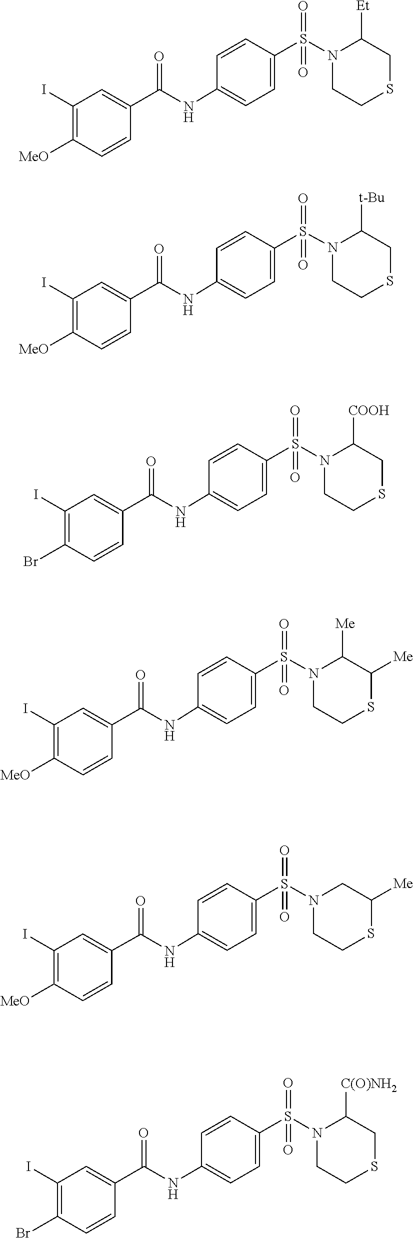 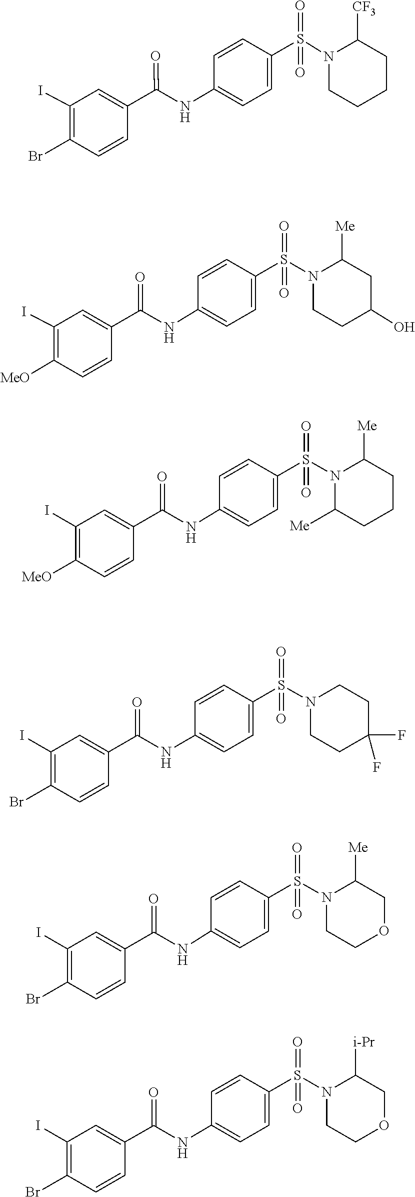 [SMILES: O=C(Nc1ccc(S(=O)(=O)N2CCCCC2C(F)(F)F)cc1)c1ccc(Br)c(I)c1 ; COc1ccc(C(=O)Nc2ccc(S(=O)(=O)N3CCC(O)CC3C)cc2)cc1I ; COc1ccc(C(=O)Nc2ccc(S(=O)(=O)N3C(C)CCCC3C)cc2)cc1I ; O=C(Nc1ccc(S(=O)(=O)N2CCC(F)(F)CC2)cc1)c1ccc(Br)c(I)c1 ; CC1COCCN1S(=O)(=O)c1ccc(NC(=O)c2ccc(Br)c(I)c2)cc1 ; CC(C)C1COCCN1S(=O)(=O)c1ccc(NC(=O)c2ccc(Br)c(I)c2)cc1] 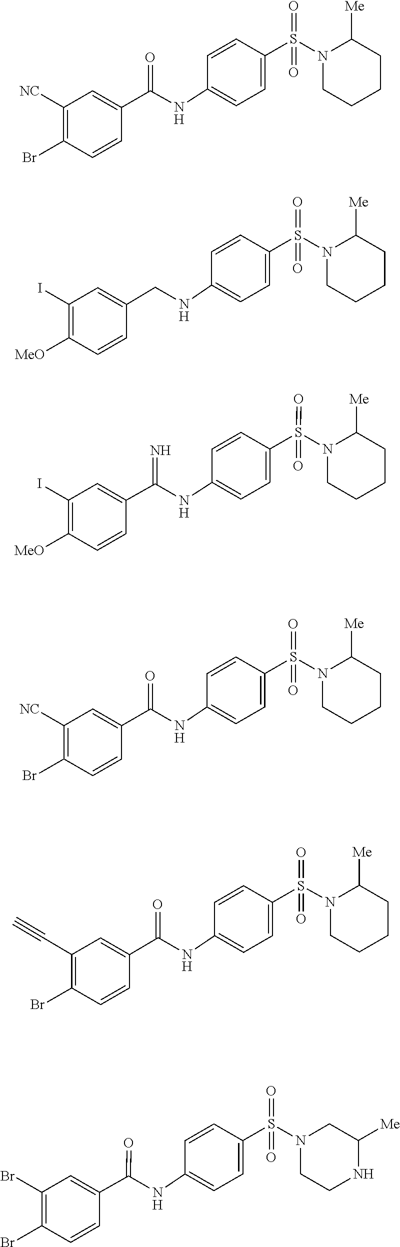 [SMILES: CC1CCCCN1S(=O)(=O)c1ccc(NC(=O)c2ccc(Br)c(C#N)c2)cc1 ; COc1ccc(CNc2ccc(S(=O)(=O)N3CCCCC3C)cc2)cc1I ; COc1ccc(C(=N)Nc2ccc(S(=O)(=O)N3CCCCC3C)cc2)cc1I ; CC1CCCCN1S(=O)(=O)c1ccc(NC(=O)c2ccc(Br)c(C#N)c2)cc1 ; C#Cc1cc(C(=O)Nc2ccc(S(=O)(=O)N3CCCCC3C)cc2)ccc1Br ; CC1CN(S(=O)(=O)c2ccc(NC(=O)c3ccc(Br)c(Br)c3)cc2)CCN1] 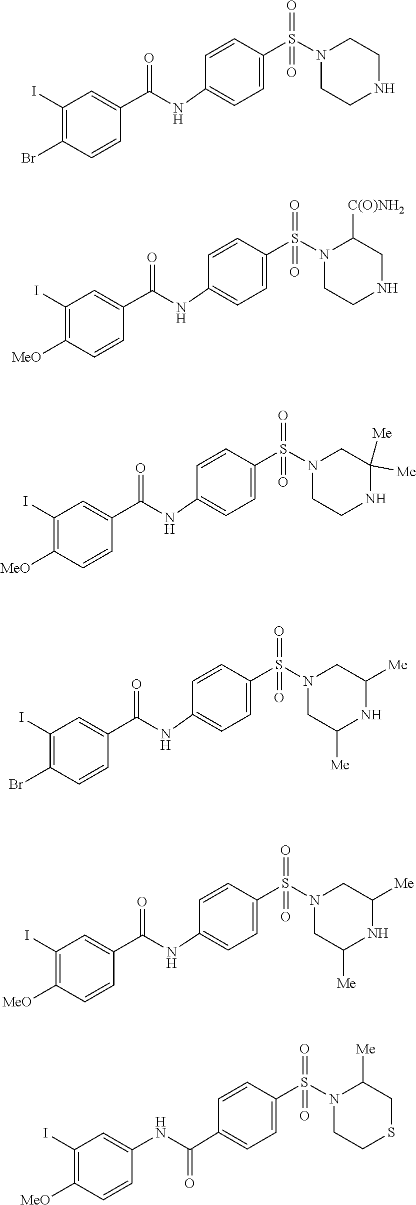 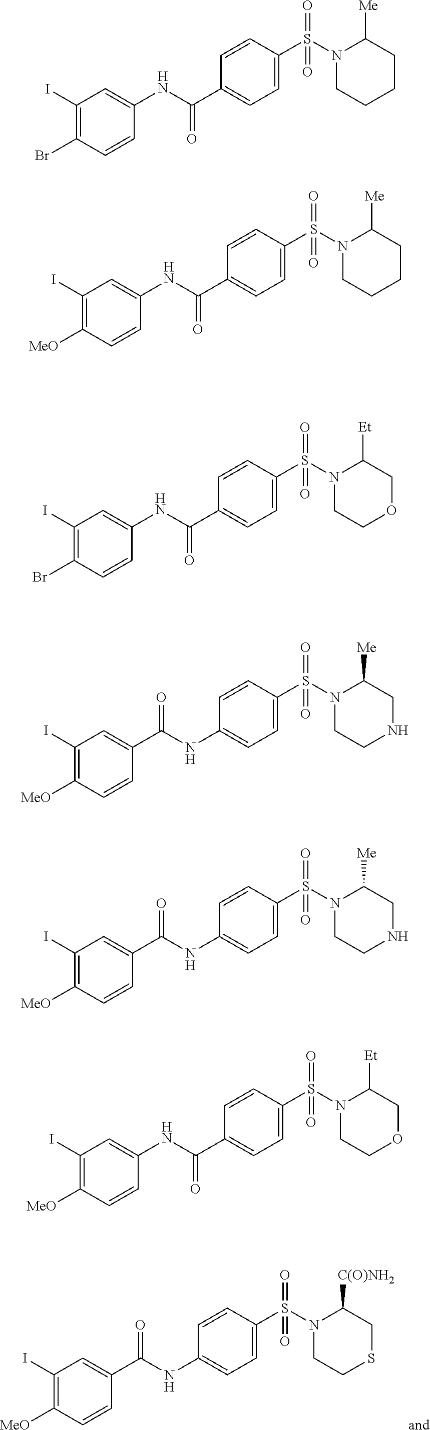 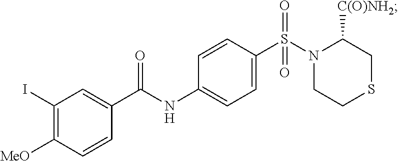
or a prodrug, salt, enantiomer, or diastereomer thereof.

61. The method of any of claims 29 to 60, wherein the wherein the compound of Formula II is further defined as at least one of: 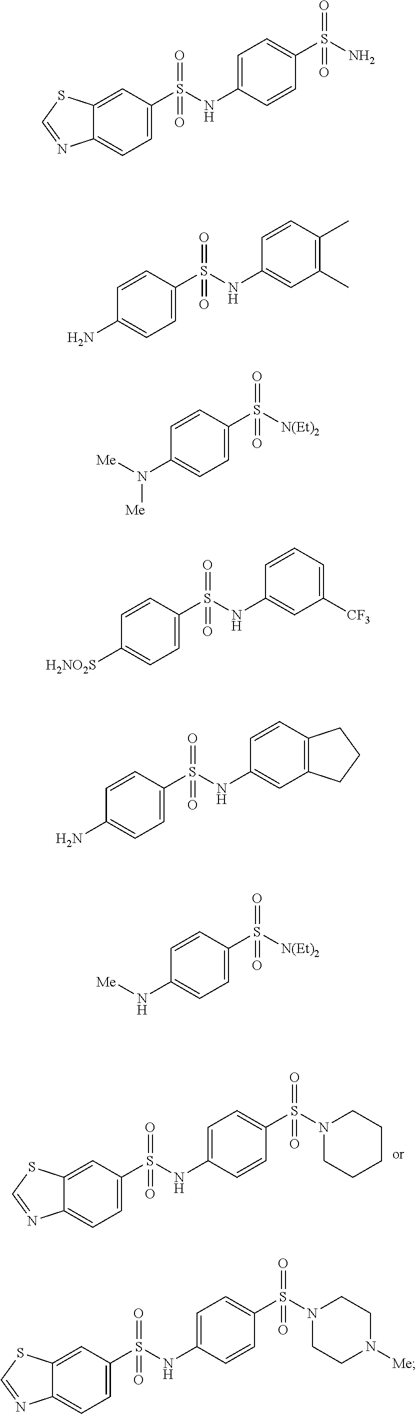
or a prodrug, salt, enantiomer, or diastereomer thereof.

62. A composition comprising a compound of Formula 1: 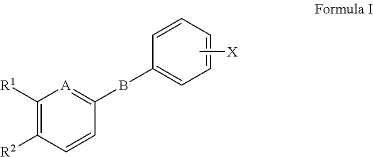
where:
A is —CH— or —N—;
B is —C(O)—NH—, —NH—C(O)—, —CH2-NH—, or —C(NH)—NH—;
X is —(Y)—NR³R⁴ or NHSO₂Me;
Y is —SO₂—, —C(O)—, or —(CH₂)—;
R¹ and R² are each independently hydrogen, hydroxyl, substituted or unsubstituted alkyl, substituted or unsubstituted alkynyl, alkoxy, halide, nitrile, amine, acylamine, substituted or unsubstituted aryl, 4-6 member carbocycle, substituted or unsubstituted heterocycle, and
R³ and R⁴ are each independently hydrogen, substituted or unsubstituted alkyl, substituted or unsubstituted aromatic, substituted or unsubstituted carbocycle, substituted or unsubstituted heterocycle, substituted or unsubstituted bicyclic, or may join to form a carbocycle or heterocycle.

63. The composition of claim 62, wherein the compound of Formula I is further defined as: 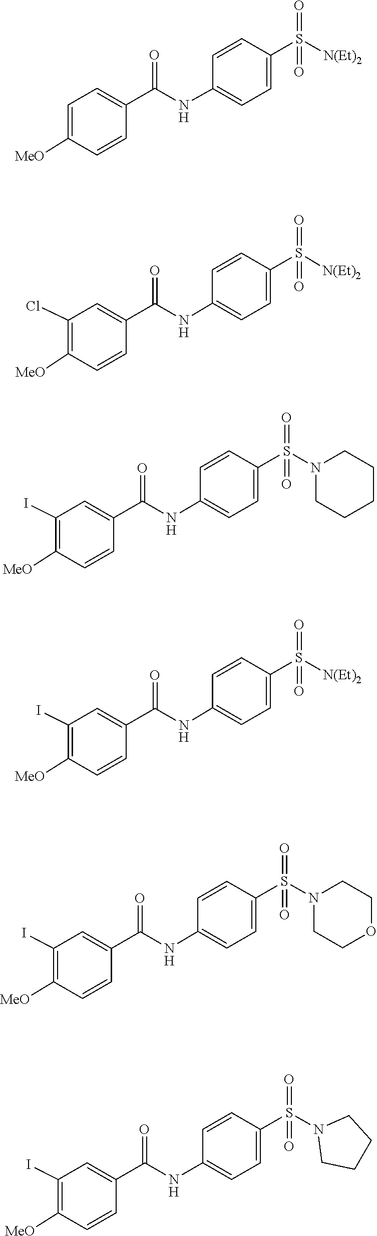 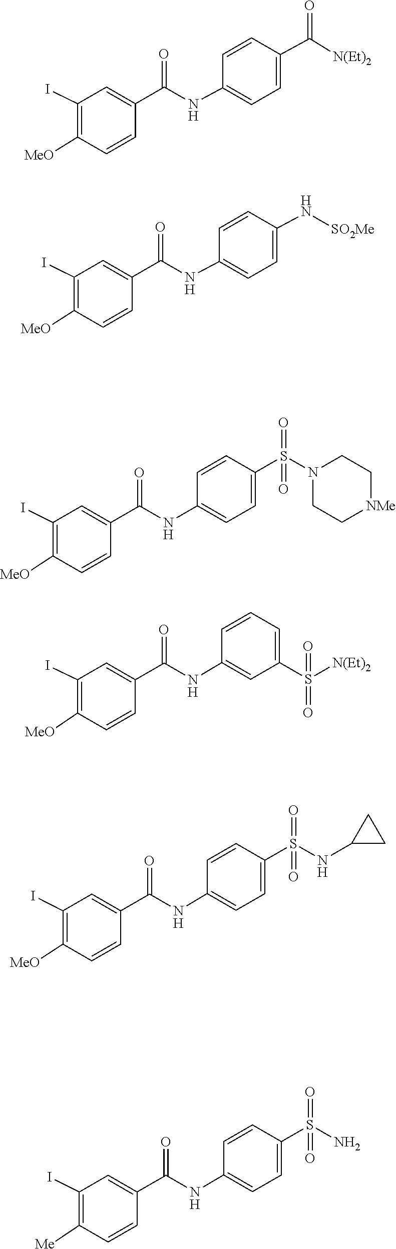 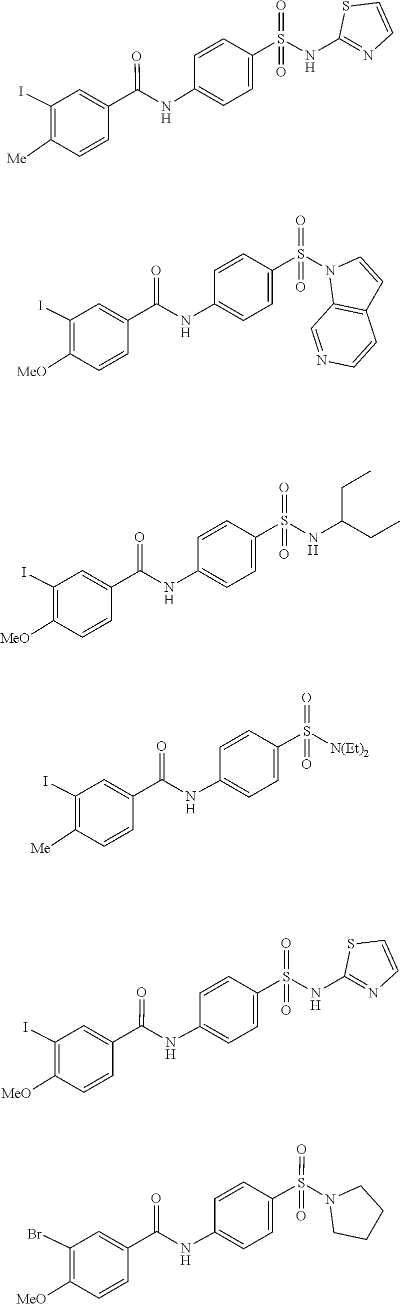 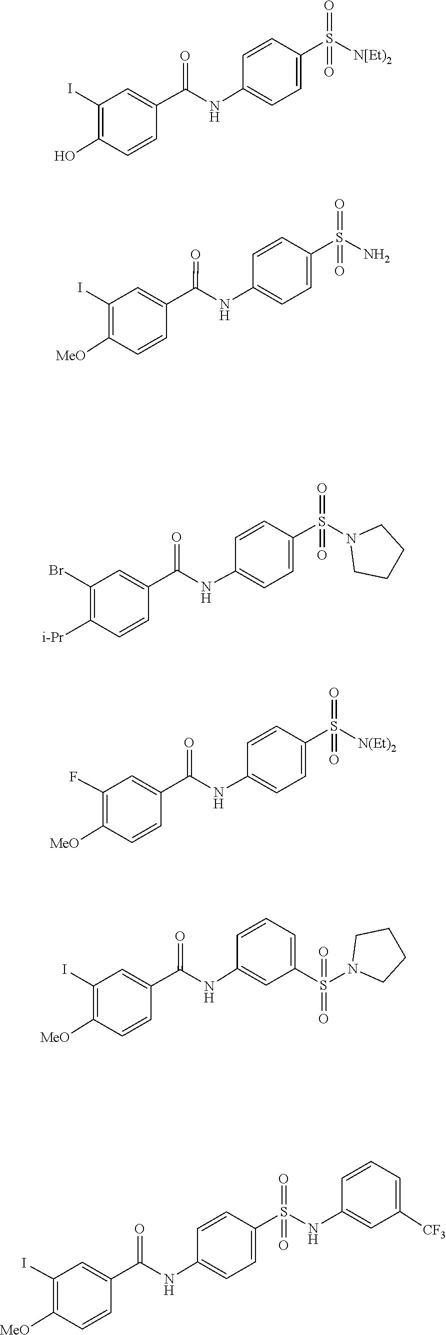 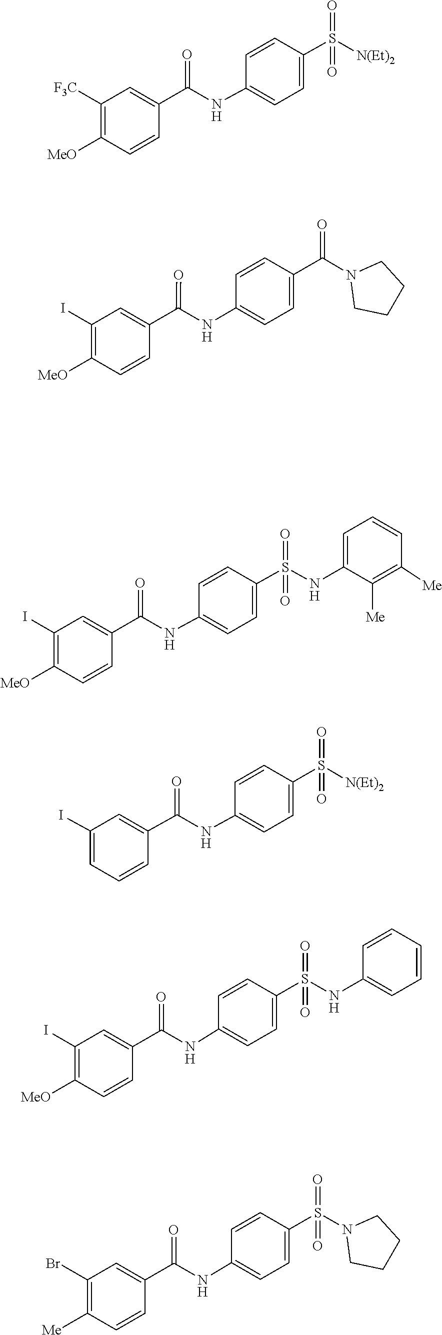 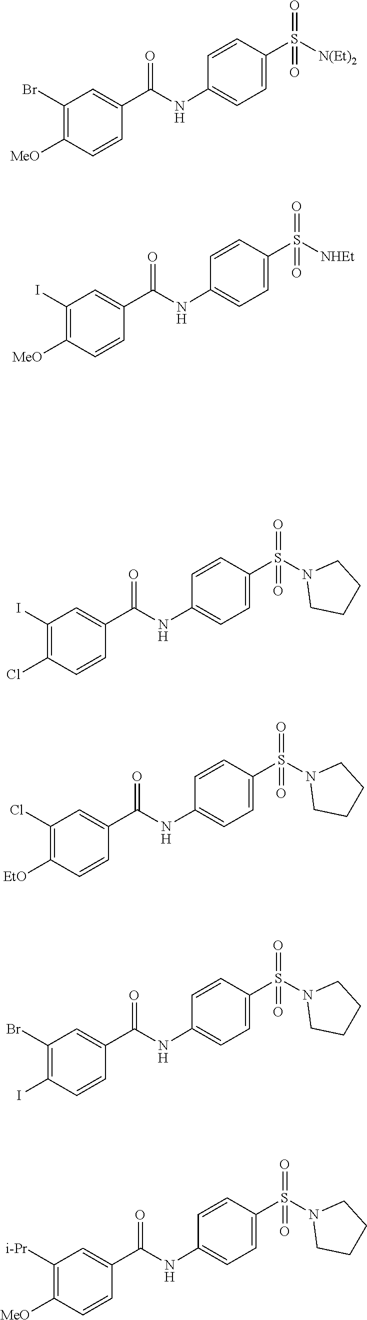 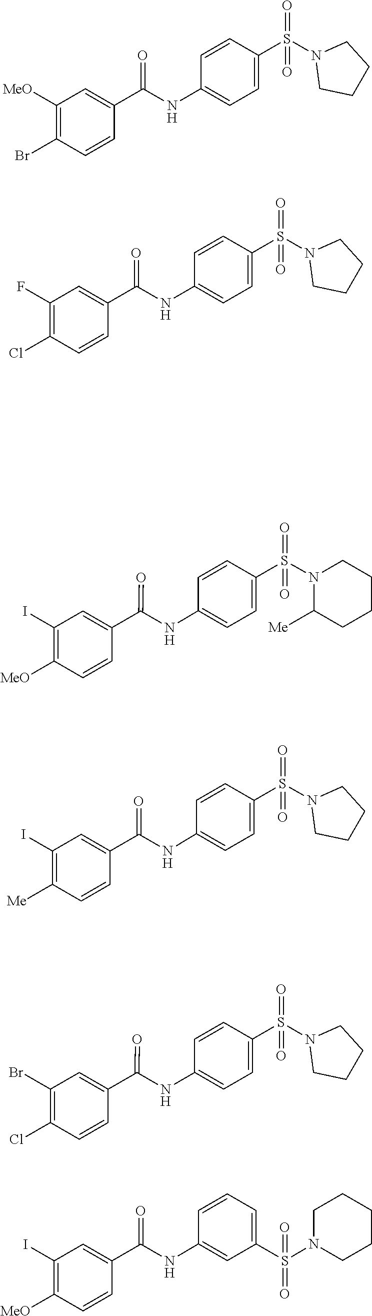 [SMILES: COc1cc(C(=O)Nc2ccc(S(=O)(=O)N3CCCC3)cc2)ccc1Br ; O=C(Nc1ccc(S(=O)(=O)N2CCCC2)cc1)c1ccc(Cl)c(F)c1 ; COc1ccc(C(=O)Nc2ccc(S(=O)(=O)N3CCCCC3C)cc2)cc1I ; Cc1ccc(C(=O)Nc2ccc(S(=O)(=O)N3CCCC3)cc2)cc1I ; O=C(Nc1ccc(S(=O)(=O)N2CCCC2)cc1)c1ccc(Cl)c(Br)c1 ; COc1ccc(C(=O)Nc2cccc(S(=O)(=O)N3CCCCC3)c2)cc1I] 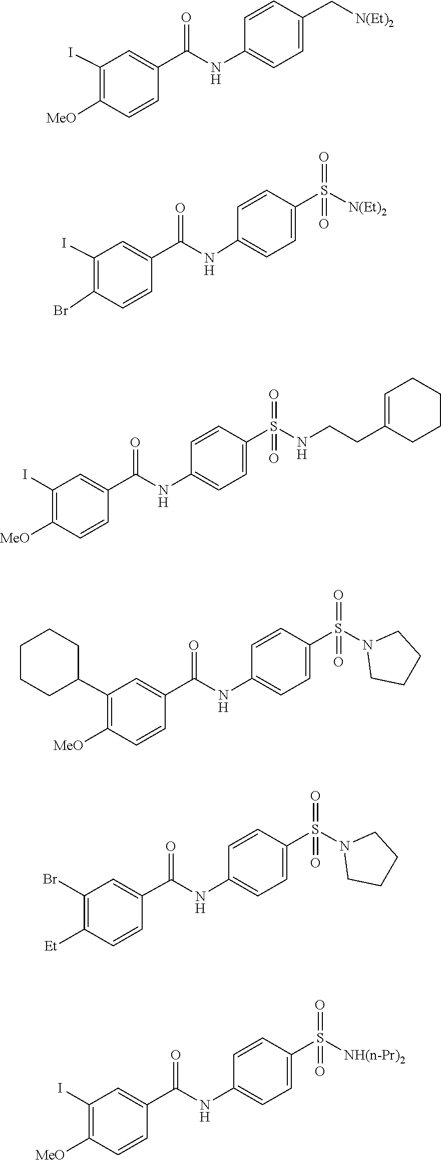 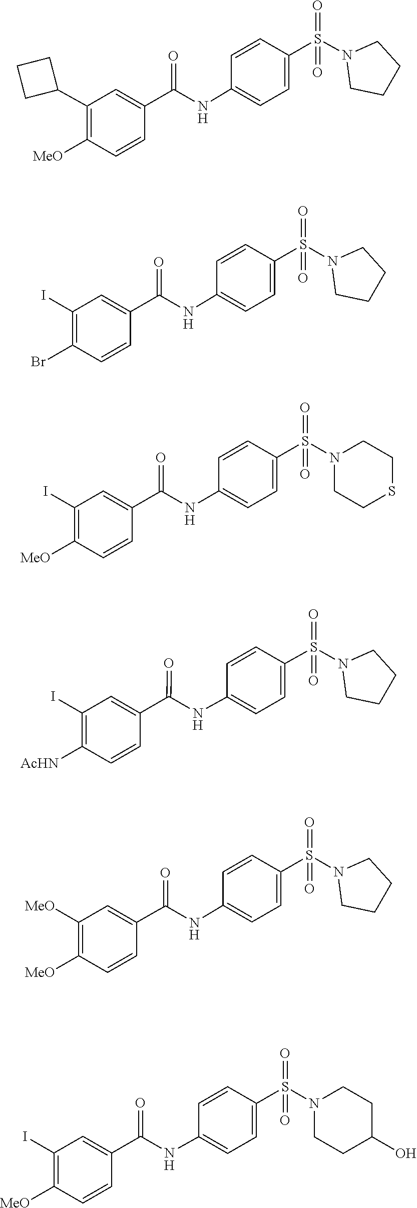 [SMILES: COc1ccc(C(=O)Nc2ccc(S(=O)(=O)N3CCCC3)cc2)cc1C1CCC1 ; O=C(Nc1ccc(S(=O)(=O)N2CCCC2)cc1)c1ccc(Br)c(I)c1 ; COc1ccc(C(=O)Nc2ccc(S(=O)(=O)N3CCSCC3)cc2)cc1I ; CC(=O)Nc1ccc(C(=O)Nc2ccc(S(=O)(=O)N3CCCC3)cc2)cc1I ; COc1ccc(C(=O)Nc2ccc(S(=O)(=O)N3CCCC3)cc2)cc1OC ; COc1ccc(C(=O)Nc2ccc(S(=O)(=O)N3CCC(O)CC3)cc2)cc1I] 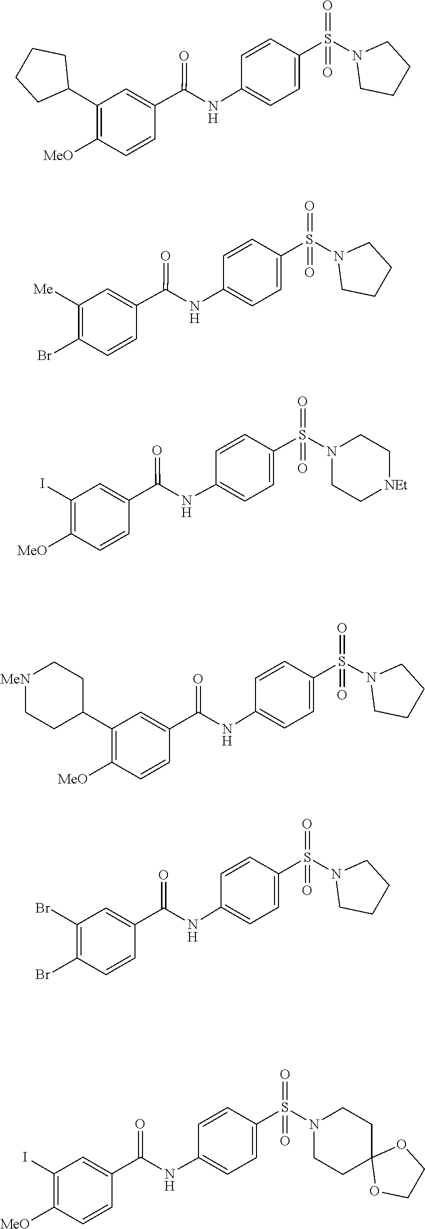 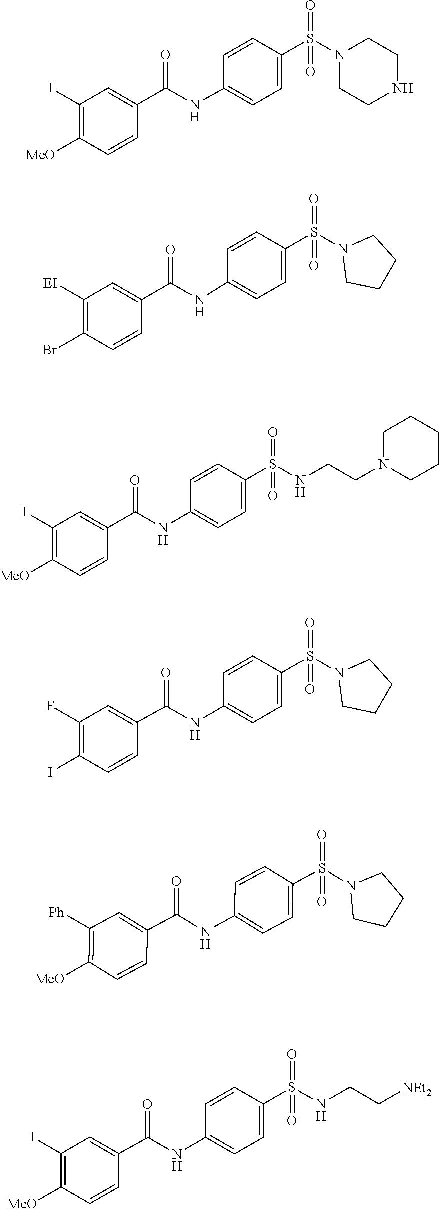 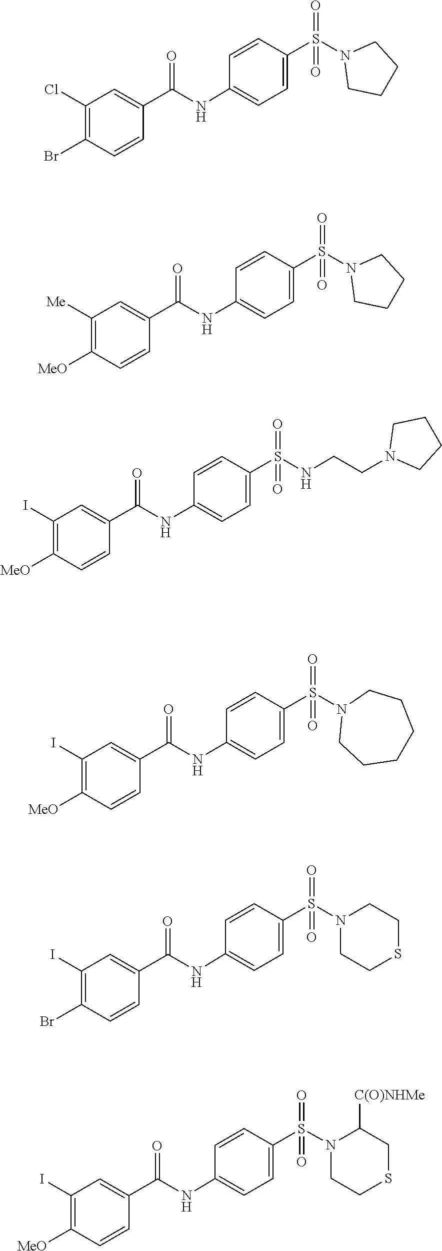 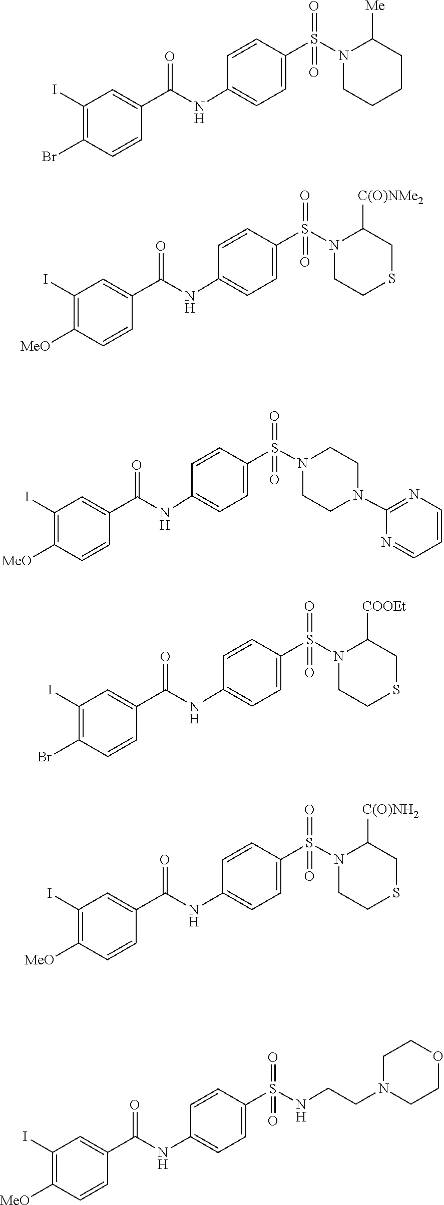 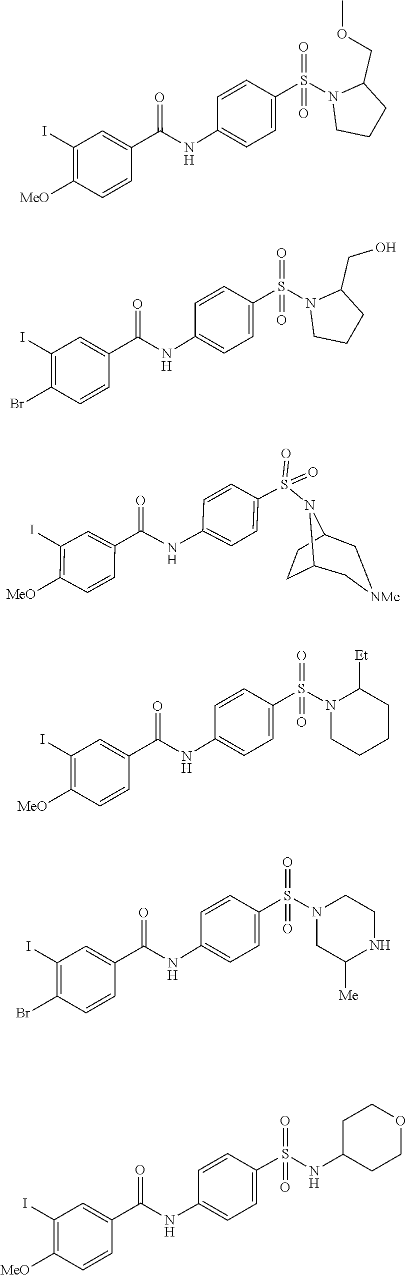 [SMILES: COCC1CCCN1S(=O)(=O)c1ccc(NC(=O)c2ccc(OC)c(I)c2)cc1 ; O=C(Nc1ccc(S(=O)(=O)N2CCCC2CO)cc1)c1ccc(Br)c(I)c1 ; COc1ccc(C(=O)Nc2ccc(S(=O)(=O)N3C4CCC3CN(C)C4)cc2)cc1I ; CCC1CCCCN1S(=O)(=O)c1ccc(NC(=O)c2ccc(OC)c(I)c2)cc1 ; CC1CN(S(=O)(=O)c2ccc(NC(=O)c3ccc(Br)c(I)c3)cc2)CCN1 ; COc1ccc(C(=O)Nc2ccc(S(=O)(=O)NC3CCOCC3)cc2)cc1I] 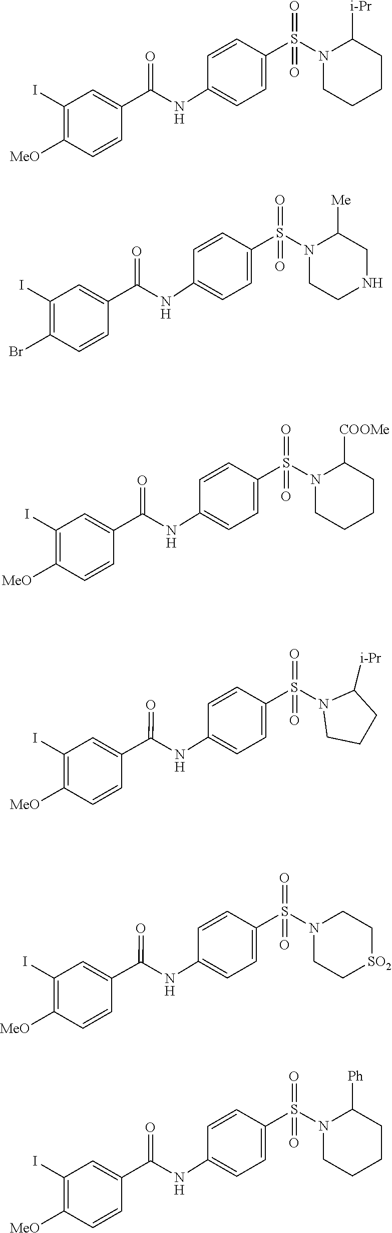 [SMILES: COc1ccc(C(=O)Nc2ccc(S(=O)(=O)N3CCCCC3C(C)C)cc2)cc1I ; CC1CNCCN1S(=O)(=O)c1ccc(NC(=O)c2ccc(Br)c(I)c2)cc1 ; COC(=O)C1CCCCN1S(=O)(=O)c1ccc(NC(=O)c2ccc(OC)c(I)c2)cc1 ; COc1ccc(C(=O)Nc2ccc(S(=O)(=O)N3CCCC3C(C)C)cc2)cc1I ; COc1ccc(C(=O)Nc2ccc(S(=O)(=O)N3CCS(=O)(=O)CC3)cc2)cc1I ; COc1ccc(C(=O)Nc2ccc(S(=O)(=O)N3CCCCC3c3ccccc3)cc2)cc1I] 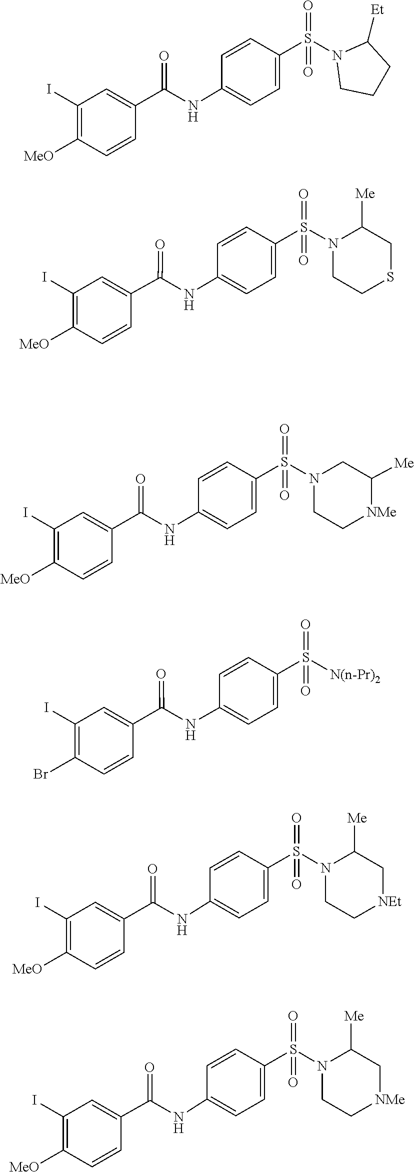 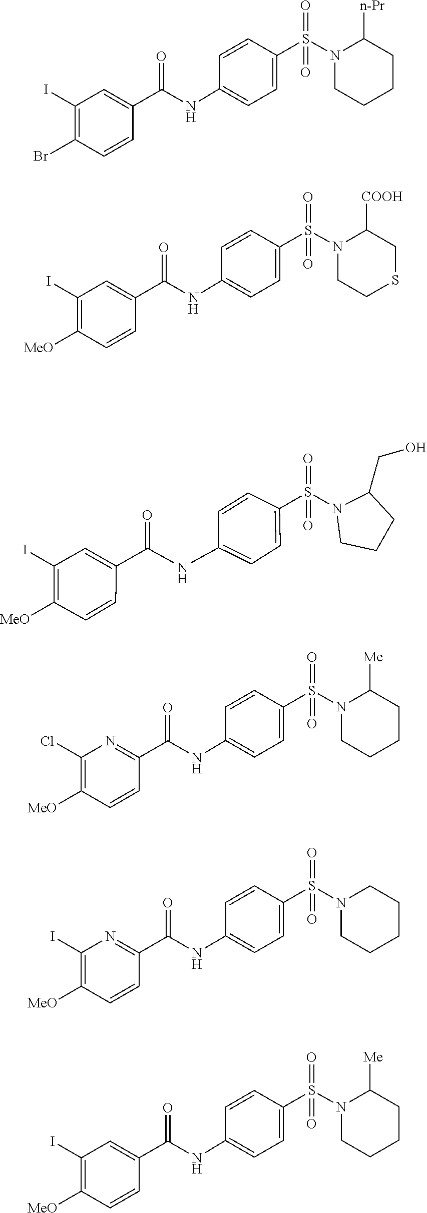 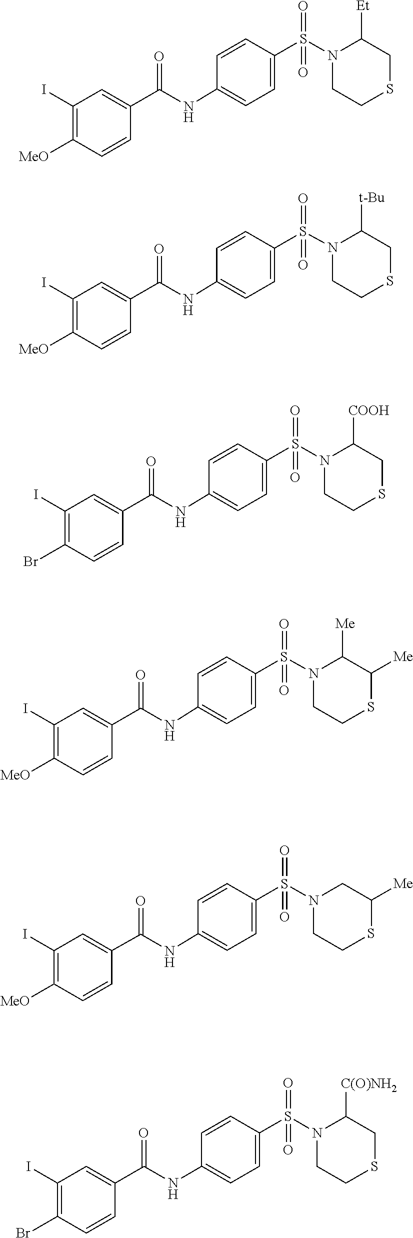 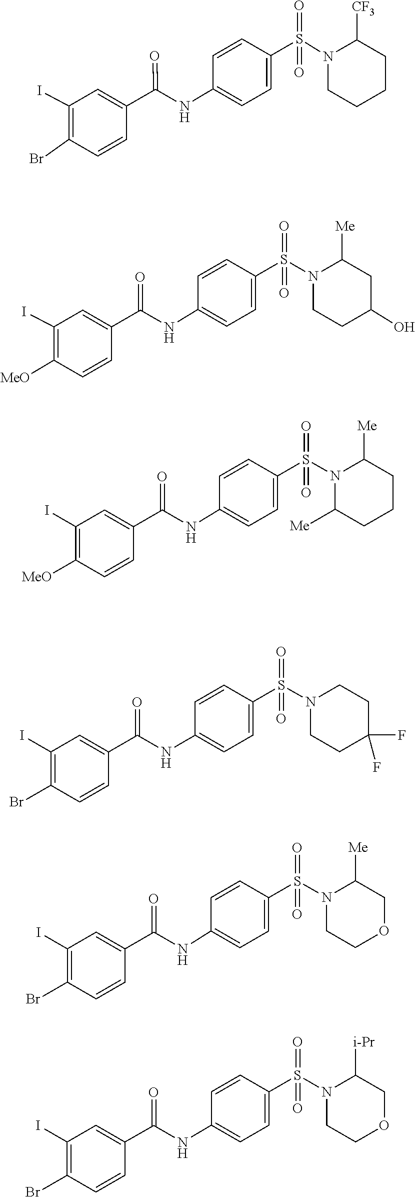 [SMILES: O=C(Nc1ccc(S(=O)(=O)N2CCCCC2C(F)(F)F)cc1)c1ccc(Br)c(I)c1 ; COc1ccc(C(=O)Nc2ccc(S(=O)(=O)N3CCC(O)CC3C)cc2)cc1I ; COc1ccc(C(=O)Nc2ccc(S(=O)(=O)N3C(C)CCCC3C)cc2)cc1I ; O=C(Nc1ccc(S(=O)(=O)N2CCC(F)(F)CC2)cc1)c1ccc(Br)c(I)c1 ; CC1COCCN1S(=O)(=O)c1ccc(NC(=O)c2ccc(Br)c(I)c2)cc1 ; CC(C)C1COCCN1S(=O)(=O)c1ccc(NC(=O)c2ccc(Br)c(I)c2)cc1] 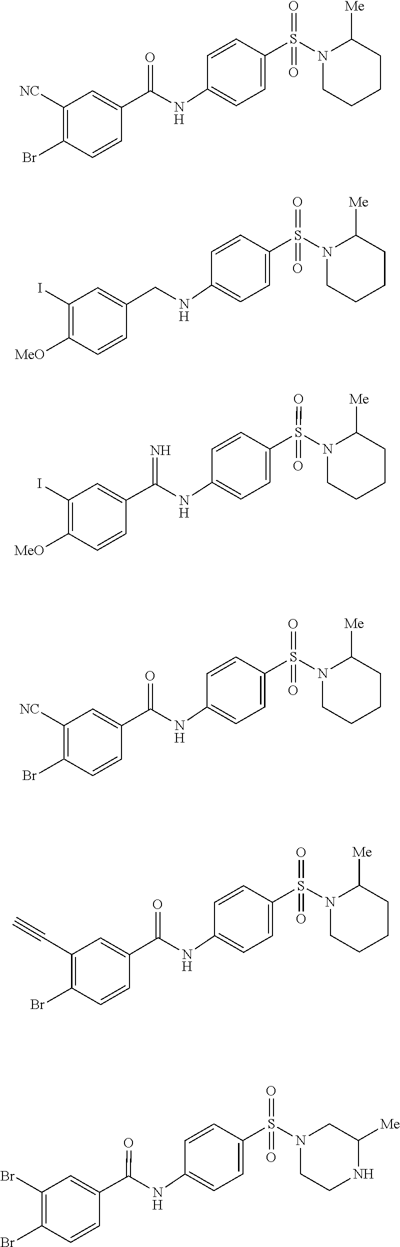 [SMILES: CC1CCCCN1S(=O)(=O)c1ccc(NC(=O)c2ccc(Br)c(C#N)c2)cc1 ; COc1ccc(CNc2ccc(S(=O)(=O)N3CCCCC3C)cc2)cc1I ; COc1ccc(C(=N)Nc2ccc(S(=O)(=O)N3CCCCC3C)cc2)cc1I ; CC1CCCCN1S(=O)(=O)c1ccc(NC(=O)c2ccc(Br)c(C#N)c2)cc1 ; C#Cc1cc(C(=O)Nc2ccc(S(=O)(=O)N3CCCCC3C)cc2)ccc1Br ; CC1CN(S(=O)(=O)c2ccc(NC(=O)c3ccc(Br)c(Br)c3)cc2)CCN1] 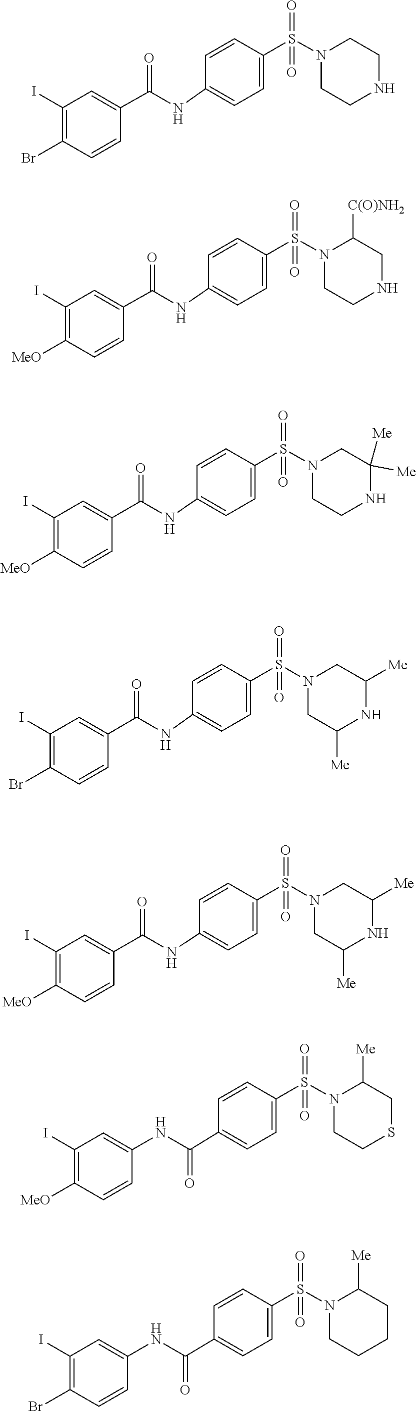 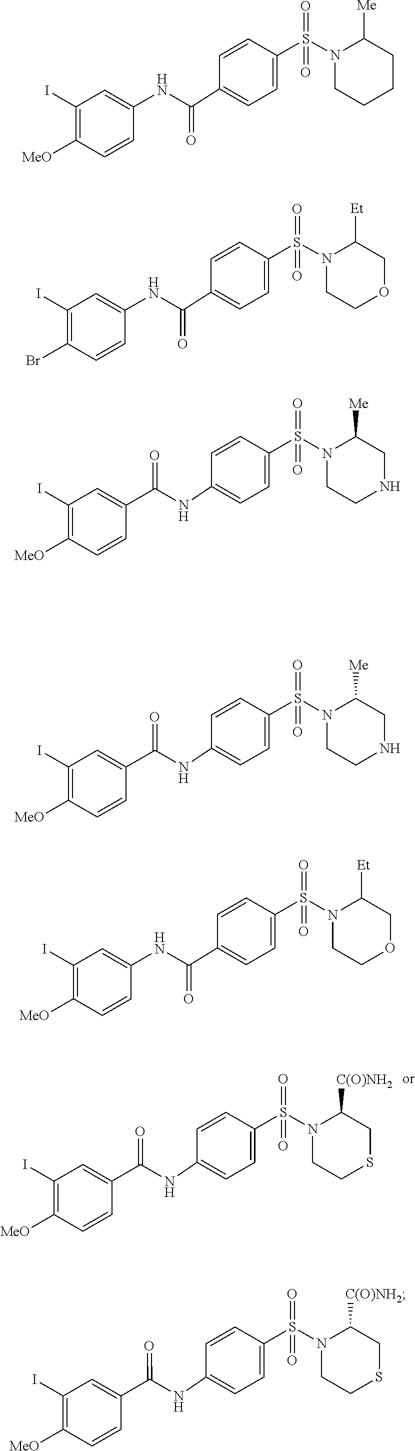
or a salt, enantiomer, or diastereomer thereof.

64. A composition comprising a compound of Formula II: 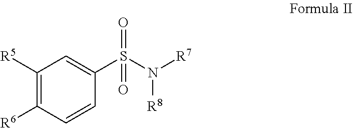
where:
where R⁵ and R⁶ are each independently hydrogen, halide, substituted or unsubstituted alkyl, alkoxy, amine, alkylamine, sulfonamide, or join together to form a 5 or 6 member carbocycle or heterocycle; and
R⁷ and R⁸ are each independently hydrogen, alkyl, or substituted or unsubstituted aryl, wherein the substituted aryl may be substituted with amide, sulfonamide, substituted or unsubstituted alkyl, or two adjacent carbon atoms on the substituted aryl ring form a carbocycle or heterocycle ring;
or a salt, enantiomer, or diastereomer thereof.

65. The composition of claim 64, wherein the composition is further defined as: 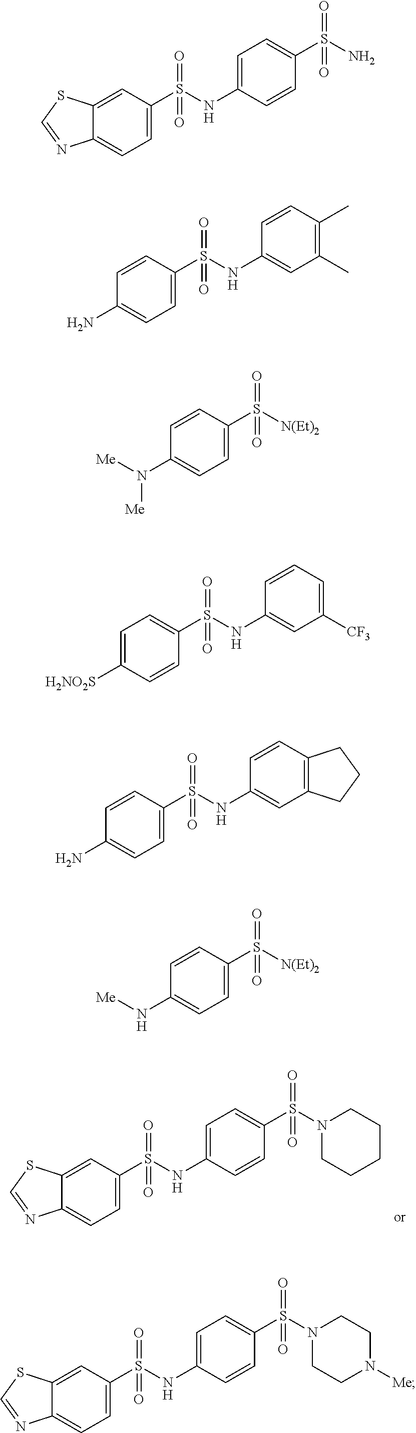
or a salt, enantiomer, or diastereomer thereof.
